(19) United States
(12) Patent Application Publication
(10) Pub. No.: US 2022/0380853 A1
(43) Pub. Date: Dec. 1, 2022

(54) PROSTATE CANCER DETECTION METHODS

(71) Applicant: Cancer Research Technology Limited, London (GB)

(72) Inventors: Anjui Wu, London (GB); Gerhardt Attard, London (GB); Paolo Cremaschi, London (GB); Daniel Wetterskog, London (GB)

(73) Assignee: Cancer Research Technology Limited, London (GB)

(21) Appl. No.: 17/770,974

(22) PCT Filed: Oct. 23, 2020
(86) PCT No.: PCT/GB2020/052706

§ 371 Date: Apr. 21, 2022

(30) Foreign Application Priority Data
Oct. 24, 2019 (GB) 1915469.9

(51) Int. Cl. C12Q 1/6886

(52) U.S. Cl. CPC C12Q 1/6886; C12Q 2600/154; C12Q 2600/106; C12Q 1/6869

(57) ABSTRACT

The present invention provides methods of detecting, screening, monitoring, staging, classification, selecting treatment for, ascertaining whether treatment is working in, and/or prognostication of prostate cancer comprising determining the average methylation ratio at 10 or more genomic regions as set out in the application, and associated methods of selecting a treatment or ascertaining whether a treatment is effective. The present invention also provides a method for determining a solid cancer circulating free DNA (cfDNA) methylome signature for use in the detecting, screening, monitoring, staging, classification, selecting treatment for, ascertaining whether treatment is working in, and/or prognostication of the solid cancer in a sample obtained from a subject comprising determining the average methylation ratio at 10 or more genomic regions as set out in the application.

### INTRODUCTION

The present invention relates to methods of detecting, screening, monitoring, staging, classification, selecting treatment for, ascertaining whether treatment is working in, and/or prognostication of prostate cancer, and associated methods of selecting a treatment or ascertaining whether a treatment is effective. The present invention also relates to a method for determining a solid cancer circulating free DNA (cfDNA) methylome signature for use in the detecting, screening, monitoring, staging, classification, selecting treatment for, ascertaining whether treatment is working in, and/or prognostication of the solid cancer in a sample obtained from a subject.

### BACKGROUND OF THE INVENTION

Prostate cancer is the most common cancer among men in many parts of the world. Prostate cancer is the second leading cause of cancer death in men in the United States.

Currently, the most frequently used methods for detecting prostate cancer are a digital rectal examination and a blood test to determine levels of prostate-specific antigen (PSA) produced by the prostate gland. However, these diagnostic tools can lack the sensitivity required to detect very early prostate lesions or to detect progression. Biopsies are invasive, and can lead to false-negatives and repeat biopsies, as they do not sample the entire prostate. As the cancer progresses, metastasis can occur and currently metastatic prostate cancer is generally diagnosed using further PSA testing together with MRI/PSMA imaging. PSMA imaging involves the use of a radiolabelled monoclonal antibody for prostate-specific membrane antigen. Detection of the radiolabelled antibody enables the clinician to identify if cancerous cells have spread in the body. These methods of detection and diagnosis have various disadvantages: they are expensive to use; PSA has come under much scrutiny recently for unreliable results and over diagnosis; and imaging modalities are only able to detect a secondary tumour once it has reached a certain size.

Plasma tumour DNA tests have shown clinical utility for cancer detection, risk stratification and response assessment. Molecular analysis of circulating cell-free DNA (cfDNA) and cell-free RNA (cfRNA) has been found to be a useful approach in some circumstances. It is particularly convenient as samples can be obtained without any invasive procedure being necessary. A common approach is to detect or measure the abundance of genomic alterations that are used to distinguish tumour from normal DNA. However, this approach can be limited by the low prevalence of recurrent genomic changes, the relatively small number that are tumour specific and the low abundance in circulation of these aberrations that can overlap with other non-tumour aberrations, for example those resulting from clonal haematopoiesis. Overall these factors limit the sensitivity of genomic tests for screening for prostate cancer.

Methylation changes are tissue- or cancer-specific. Detection of methylation changes thus provides a promising approach for the diagnosis and assessment of cancers, including prostate cancer. In WO2014/043763 and WO2017/212428, there are described methods for the assessment of diseases, in particular cancers by the analysis of methylation patterns in cell-free DNA.

Regarding prostate cancer in particular, for example Kirby et al. (BMC Cancer (2017), 17:273) reported that DNA methylation patterns are altered in prostate cancer tissue in comparison to benign-adjacent tissue. They noted patterns of DNA methylation that can distinguish prostate cancers with good specificity and sensitivity in multiple patient tissue cohorts. The authors also identified transcription factors binding in these differentially methylated regions that may play a role in prostate cancer development. The methods developed by Kirby and by others require a very large amount of DNA to be sequenced and analysed in order for a reliable assessment to be made.

Metastatic castration-resistant prostate cancer (mCRPC) patients with a range of genomic aberrations, including androgen receptor (AR) copy number gain or TP53 mutations, detected in plasma prior to androgen receptor (AR) targeting with abiraterone or enzalutamide have a shorter duration of treatment benefit and overall survival. mCRPC exhibits a variable clinical course and biomarkers to stratify patients are urgently required to optimize management. As tumour biopsies from metastatic sites can be difficult to obtain and repeated sampling of multiple metastases is usually not feasible a minimally-invasive liquid biopsy-based analysis method would be helpful for clinicians. There thus remains a need for improved methods of detection and screening in this field.

### SUMMARY OF THE INVENTION

The present invention provides a method for detecting, screening, monitoring, staging, classification, selecting treatment for, ascertaining whether treatment is working in, and/or prognostication of prostate cancer in a sample obtained from a subject, wherein the sample comprises circulating free DNA (cfDNA), the method comprising
characterizing the methylome sequence of a plurality of cfDNA molecules in the sample, wherein the methylome sequence of a cfDNA molecule is the DNA sequence and the methylation profile of the molecule;
determining the average methylation ratio at 10 or more genomic regions, each genomic region being selected from the group consisting of:
a 100 to 200 bp region comprising or having a genomic location defined in Tables 1 to 4, and
a 2 to 99 bp region within a genomic location defined in Tables 1 to 4 and comprising at least one CpG locus,
and wherein each genomic region is covered by at least one sequence read of at least one characterized methylome sequence;
calculating a methylation score using the average methylation ratio for each genomic region;
analyzing the methylation score to determine the level of prostate cancer fraction in the cfDNA sample.

To concurrently study the plasma genome and methylome and overcome the inherent challenges of methylation analysis resulting from the high variance in methylation data, the inventors selected plasma samples from a focused cohort of mCRPC patients with genomic information. The inventors surprisingly found that methylation data obtained by analysis of metastatic cancer patients' cell-free DNA in the plasma samples could very accurately estimate tumour fraction and can be used, for example, to improve liquid biopsy patient stratification.

The present invention provides a method for detecting, screening, monitoring, staging, classification, selecting treatment for, ascertaining whether treatment is working in, and/or prognostication of prostate cancer in a sample obtained from a subject, wherein the sample comprises circulating free DNA (cfDNA), the method comprising:

characterizing the methylome sequence of a plurality of cfDNA molecules in the sample, wherein the methylome sequence of a cfDNA molecule is the DNA sequence and the methylation profile of the molecule;
determining the average methylation ratio at 10 or more genomic regions, each genomic region being selected from the group consisting of:
a 100 to 200 bp region comprising or having a genomic location defined in Table 8, and
a 2 to 99 bp region within a genomic location defined in Table 8 and comprising at least one CpG locus,
and wherein each of the genomic regions is covered by at least one sequence read of at least one characterized methylome sequence;
calculating a methylation score using the average methylation ratio for each of the genomic regions;
analyzing the methylation score to determine whether the sample comprises cfDNA derived from a prostate cancer subtype.

The inventors surprisingly found that methylation data extracted from metastatic cancer patient plasma DNA could identify clinically-relevant subtypes, and in particular a sub-group of cancers characterized by a more aggressive clinical course and enriched for AR copy number gain, and thus can be used, for example, to improve liquid biopsy patient stratification.

The present invention also provides an in-vitro diagnostic kit for use in the detection, screening, monitoring, staging, classification and prognostication of prostate cancer, comprising one or more reagents for detecting the presence or absence of at least 25 DNA molecules having a DNA sequence corresponding to all or part of a genomic location defined in Tables 1 to 4 and/or Table 8.

The present invention further provides a computer product comprising a non-transitory computer readable medium storing a plurality of instructions that when executed control a computer system to perform the method of the present invention for detecting, screening, monitoring, staging, classification, selecting treatment for, ascertaining whether treatment is working in, and/or prognostication of prostate cancer in a sample obtained from a subject.

The present invention further provides a computer-implemented method for detection, screening, monitoring, staging, classification and/or prognostication of prostate cancer in a sample obtained from a subject, wherein the sample comprises cfDNA.

The present invention further provides a computer-implemented method for classifying a prostate cancer patient into one or more of a plurality of treatment categories, the method comprising determining the level of prostate cancer fraction of cfDNA in a sample obtained from a subject, wherein the sample comprises cfDNA.

The present invention further provides a method for treating prostate cancer comprising treating the subject using a therapeutic agent for the treatment of prostate cancer, surgery, and/or radiotherapy.

The present invention further provides a method of determining one or more suitable therapeutic agents for the treatment of prostate cancer for a subject having prostate cancer.

The present invention further provides a method of determining a suitable treatment regimen for a subject having prostate cancer.

The present invention further provides a method for determining a solid cancer circulating free DNA (cfDNA) methylome signature for use in detecting, screening, monitoring, staging, classification, selecting treatment for, ascertaining whether treatment is working in, prognostication and/or treatment of the solid cancer, the method comprising:

(i) characterizing the methylome sequence of a plurality of cfDNA molecules in a first sample comprising cfDNA from a subject known to have the solid cancer, wherein the methylome sequence of a cfDNA molecule is the DNA sequence and the methylation profile of the molecule;
(ii) determining the respective number of characterised cfDNA molecules corresponding to a CpG locus or a genomic region of 2 to 10,000 bp (preferably 2 to 200 bp) in the first sample by aligning the methylome sequences;
(iii) determining the methylation ratio of each CpG locus and/or average methylation ratio of each genomic region of 2 to 10,000 bp (preferably 2 to 200 bp) in the first sample;
repeating steps (i) to (iii) for one or more further samples comprising cfDNA each from subjects known to have the solid cancer;
performing a variance analysis of all or a selection of the methylation ratios of the CpG loci and/or all or a selection of average methylation ratios of the genomic regions of the samples;
selecting a group of CpG loci and/or genomic regions associated with a feature of the samples; and
selecting CpG loci and/or genomic regions in the group to provide the cfDNA methylome signature.

### DESCRIPTION OF THE DRAWINGS

FIGS. 1 and 2 shows the patient characteristics of the plasma samples used in Example 1.

FIG. 3 shows the targeted methylome sequencing matrix (total reads, mapped reads, % mapped reads, % bisulfite conversion).

FIG. 4 shows the LP-WGBS matrix (total reads, mapped reads, % mapped reads, % bisulfite conversion).

FIG. 5 shows a plot showing coverage distribution in target regions by bisulfite high-coverage next-generation sequencing (NGS) in plasma samples.

FIG. 6 shows the fraction of data to drop on different window sizes (10 bp, 100 bp, 1000 bp, 10000 bp).

FIG. 7 shows the distribution of methylation ratio by different segment size (10 bp, 100 bp, 1,000 bp, 10,000 bp).

FIG. 8 shows the correlation of median methylation ratio of selected segments with genomically-determined tumour fraction. Y-axis shows the correlation value and the X-axis denotes the number of top correlated segments.

FIG. 9 shows the correlation of methylation ratios of selected segments. Y-axis shows the standard deviation and the X-axis denotes the number of top correlated segments.

FIG. 10 shows a schematic overview of the work-flow for integrating NGS of the plasma methylome and genome of Example 1.

FIG. 11 shows the genomically-determined tumour fraction in baseline and progression samples from pre- and post-chemotherapy patients receiving abiraterone or enzalutamide.

FIG. 12 shows a box plot showing methylation ratio distribution for baseline (A) and progression (B) plasma samples and white blood cells (C) presented separately.

FIG. 13 shows the genomic annotation based on location of methylation segments in the custom targeted panel and in segments covered >10× in all 19 baseline samples.

FIG. 14 shows the methylation ratio density (upper panel) and Quantile-Quantile plot (bottom panel) analysis based on the genomic annotation of methylation segments in promoter or other regions. Data from white blood cells (WBC) or plasma collected at baseline (BL) or progression (PD) from mCRPC patients or from healthy volunteers (HV) are presented separately. In the bottom panel, the upper line in both plots that diverges from the course of the other two lines corresponds to the HV data, the other two tracking lines in both plots correspond to the BL and PD data.

FIG. 15 shows a schematic workflow of methylation data analysis of Example 1.

FIG. 16 shows a bar-chart showing the variance associated to each Principal Component (PC) (black columns show significant principal components); and a scree plot (the dotted line) indicating cumulative explained variance.

FIG. 17 shows the correlation between PCs and tumour fraction (bottom panel). Size and the colour of each circle show Pearson correlation and background shading denotes P value).

FIG. 18 shows the correlation of genomically determined tumour fraction (y-axis) and principal component 1 (PC1) values (x-axis) from high-coverage targeted methylation sequencing on 19 baseline, 16 progression plasma samples, and control samples (n=4 healthy volunteer plasma samples, LNCaP prostate cancer cell line).

FIG. 19 shows the functional enrichment analysis of genes (n=231) in ct-MethSig segments. The p-value was corrected for multiple statistical testing (Benjamini-Hochberg).

FIG. 20 shows the Bland-Altman plot showing agreement for tumour fraction estimation by genomically-determined tumour fraction and on LP-WGBS.

FIG. 21 shows the top 1000 segments (ct-MethSig) with the highest correlation coefficient between PC1 and methylation ratio.

FIG. 22 shows the ct-MethSig methylation ratio distribution by patient plasma sample split by negatively correlated (i.e. hypermethylated) and positively correlated (i.e. hypomethylated) segments.

FIGS. 23A to 23C shows the methylation ratios of GSTP1 (FIG. 23A), APC (FIG. 23B), and RASSF1A (FIG. 23C) across different tissue types—healthy volunteer plasma, white blood cells, CRPC plasma samples, LNCaP cell line.

FIGS. 24A and 24B show the ct-MethSig segment methylation ratio derived from mCRPC tissues lined by tumour fraction (A: negatively correlated (i.e. hypermethylated) segments; B: positively correlated (i.e. hypomethylated) segments).

FIG. 25 shows the correlation between HSPC tissue tumour fraction estimation by ct-MethSig and molecularly-defined tumour fraction.

FIG. 26 shows a Venn diagram showing the overlap of negatively (dark blue/darker shading) correlated genes in ct-MethSig segments with targets of EED, SUZ12, and ES (Embryonic Stem cells) with H3K27ME3 marks. The numbers highlighted in white bold denote the number of genes in the ct-MethSig negatively correlated group.

FIG. 27 shows the permutation test on genes overlapping with ct-MethSig; the dot represents the gene enrichment test (Fisher Exact test) P value in genes overlapping with ct-MethSig and the box represents P values of the permutation test with 1000-time iteration.

FIG. 28 shows the circulating tumour fraction methylation signature comprises segments specific to either normal or malignant prostate epithelium. Left panel: Methylation ratios of ct-MethSig negatively (i.e. hypermethylated, N=520) and positively (i.e. hypomethylated, N=480) correlated group from LNCaP (N=4), healthy volunteer (H.V., N=4), and normal prostate epithelium (PrEC). The right panel shows ct-MethSig negative (i.e. hypermethylated) and positive (i.e. hypomethylated) groups can be split into prostate cancer specific segments and prostate epithelium specific.

FIG. 29 shows the CASCADE patient and sample characteristics.

FIGS. 30A and B shows the methylation ratio distribution of circulating normal prostate specific or prostate cancer specific component in localized prostate cancer from TCGA.

FIG. 31 shows the top 1000 segments with highest correlation coefficient between the third principal component (PC3) and methylation ratio.

FIG. 32 shows the methylation ratio of top 1000 segments highly correlated with PC3 values derived from plasma, white blood cell, HSPC tissue, and CRPC metastases (CASCADE trial).

FIG. 33 shows the comparison of intra-individual changes in the top correlated segments defined by targeted methylation NGS on plasma DNA and changes in tumour fraction. Y-axis denotes the difference (A) of mean methylation ratio of the top correlated segments between baseline and progression samples and the X-axis denotes the difference (A) in tumour fraction.

FIG. 34 shows the median methylation ratio of the top correlated segments of different metastatic sites by patient from the CASCADE rapid warm autopsy program.

FIG. 35 shows the median methylation ratio of 993 MethSig3 segments positively (i.e. hypomethylated) correlated with PC3 values across different sample types—plasma, white blood cells, cell lines (LNCaP, LNCaP95, VCaP), CASCADE tumours (mCRPC biopsies) and CSPC tumours are plotted against the median methylation ratio of top correlated segments with ct-MethSig.

FIG. 36A shows the genes overlapping with AR-MethSig; and FIG. 36B shows the functional enrichment of top correlated segments with principal component 3 (PC3).

FIG. 37 shows the AR binding motif that is over-represented in regions adjacent to the top correlated segments (top panel). The consensus AR binding motif is shown as a reference (bottom panel).

FIG. 38 shows the performance of Gaussian Mixture Model (k-fold cross-validation, k=100).

FIG. 39 shows copy number alteration plots from LP-WGS on plasma DNA with and without bisulfite treatment.

FIG. 40 shows the prevalence of gain and loss events lined by chromosome position extracted from LP-WGBS on mCRPC plasma samples.

FIG. 41 shows the analysis of copy number profiles on low-pass whole genome bisulfite sequencing. Matrix shows gains (red) and losses (blue) ordered by chromosomal position (columns) for individual patient samples (one per row) ordered by tumour fraction. Bar chart on the left shows tumour fraction per sample. Bar chart on the right shows the number of gain (red) or loss (blue) events per sample.

FIG. 42 shows the contingency tables showing ct-MethSig and AR-MethSig segments in copy number aberrant regions.

FIG. 43 shows a Manhattan plot showing the level of significance of the association between PC1 value distribution and copy number alterations ordered by chromosome position. The segment containing AR is circled with a dotted line (not significant, P=0.18). Dark dots represent that the segment belongs to the odd numbered chromosome indicated, and light dots represent that the segment belongs to the even numbered chromosome indicated.

FIG. 44 shows a Manhattan plot showing the level of significance of the association between PC3 value distribution and copy number alterations ordered by chromosome position. The segment containing AR is highlighted circled with a dotted line (P=0.018, Kruskal-Wallis test). Dark dots represent that the segment belongs to the odd numbered chromosome indicated, and light dots represent that the segment belongs to the even numbered chromosome indicated.

FIG. 45 shows the methylation ratio of AR-MethSig segments of AR gain and non-gain groups

FIG. 46 shows a Bland-Altman plot showing agreement between targeted methylation NGS and LP-WGBS on AR-MethSig median methylation ratio.

FIG. 47 shows the overall survival analysis (start of ADT to death) for AR-MethSig low group versus AR-MethSig high group (Mantel-Cox log-rank test). The line in the graph that extends beyond 150 months on ADT corresponds to AR-MethSig high, the other line corresponds to AR-MethSig low.

FIG. 48 shows the correlation of genomically-determined tumour fraction and PC1 values derived from PCA on different window sizes (10 bp, 100 bp, 1000 bp, 10000 bp).

FIG. 49 shows a schematic of the workflow of building a classification model of Example 1.

FIG. 50A shows the accuracy of Random Forest Classification model (=10) on 1000-time cross validation; FIG. 50B shows the accuracy of RFC (number of trees in the forest=100) on 1000-time cross validation.

FIGS. 51A to 51D show the accuracy of RFC model (number of trees in the forest=100) on 1000-time cross validation trained on 1 (FIG. 51A), 10 (FIG. 51B), or 100 (FIG. 51C), randomly (rdm) selected ct-MethSig segments or the ct-MethSig segments (FIG. 51D).

### DEFINITIONS

Unless defined otherwise, all technical and scientific terms used herein have the same meaning as commonly recognized by one of ordinary skill in the art to which this invention belongs.

As used herein “DNA methylation” refers to the addition of a methyl group to a DNA nucleotide. DNA methylation most commonly occurs on the 5′ carbon of cytosine residues (i.e. 5-methylcytosines) of a CpG dinucleotide (referred to herein as a “CpG locus”). DNA methylation may also occur in cytosines in other contexts, for example CHG and CHH, where H is adenine, cytosine or thymine. Cytosine methylation may also be in the form of 5-hydroxymethylcytosine. Non-cytosine methylation, such as N6-methyladenine, may also occur.

As used herein, the term “CpG locus” refers to a region of DNA where a cytosine nucleotide is followed by a guanine nucleotide in the linear sequence of bases along its 5′ to 3′ direction. A CpG site can become methylated in human and other animal DNA.

As used herein, a “methylome” is the set of nucleic acid methylation modifications in a subject's genome in a particular cell, tissue or cancer. The methylome may correspond to all of the genome, a substantial part of the genome, or relatively small portion(s) of the genome.

As used herein the term “plasma methylome” is the methylome determined from the plasma or serum of a subject (e.g., a human). The plasma methylome is an example of a “cell-free DNA methylome” since plasma includes cfDNA. The plasma methylome is an example of a mixed methylome because the plasma may comprise cfDNA from a variety of sources, for example, cfDNA from different tissues, non-cancerous and cancerous tissues.

As used herein the term “methylation profile” is the information related to DNA methylation for a DNA molecule. Information related to DNA methylation can include, but not limited to, a methylation index of a CpG locus, a methylation density of CpG sites in a DNA molecule, a distribution of CpG sites over a contiguous region, a pattern or level of methylation for each individual CpG site within a region that contains more than one CpG site, and non-CpG methylation.

As used herein the term “methylome sequence” is the DNA sequence and the methylation profile of the whole or a portion of a DNA molecule, for example a cfDNA molecule. For example, the methylome sequence may be the methylome sequence of the whole or a portion of a cfDNA molecule. The methylome sequence may correspond to all of the genome, a substantial part of the genome, or portion(s) of the genome.

As used herein the term “circulating free DNA” (cfDNA) means the DNA fragments that have been released into the blood plasma and are found freely circulating the blood stream, as well as in the urine. cfDNA is generally double-stranded DNA consisting of small fragments (70 to 200 bp).

As used herein the term “sequence read” refers to a sequence of the base pairs inferred from the whole or a portion of single molecule of DNA, for example the whole or a portion of a single molecule of cfDNA. A single read may be of 20 to 500 base pairs, or even up to 1500 base pairs. The sequence of a specific single molecule of DNA may be read once or read multiple times and each sequence is taken to be representative of a single molecule of DNA.

As used herein the term “tumour fraction cfDNA” is cfDNA derived from DNA of a cancer cell. As used herein the term “prostate cancer fraction cfDNA” is cfDNA derived from DNA of a prostate cancer cell.

As used herein, the term “genomic region” refers to a region of a genome, e.g. the genome of a subject, for example a human. A genomic region may also be referred to as a “segment”. It may be referred to using the genomic location of the region, for example using the coordinates of the start position and end position of the location in a specific chromosome. For a human subject a genomic region is suitably described by a genomic location, and in particular a genomic location with reference to a reference genome (for example, a digital nucleic acid sequence database, assembled a representative example of a species' set of genes).

As used herein, the term “genomic location” refers to the location of a region of a genome, e.g. the genome of a subject, for example a human. It may be referred to using the coordinates of the start position and end position of the location in a specific chromosome. For a human subject a genomic location is suitably described by reference to a reference genome (for example, a digital nucleic acid sequence database, assembled from a representative example of a species' set of genes). For example, for a human subject, with reference to the human reference genome GRCh37 (also referred to as Human Genome 19 (hg19)) or human reference genome GRCh38 (also referred to as Human Genome 38 (hg38)). For the present inventions, preferably the reference genome is human reference genome GRCh37 (also known as hg19). As such, a genomic location for a human may be described using the coordinates of the start position and end position of the location in a specific chromosome with reference to the Genome Reference Consortium Human Build 37 (GRCh37) (also referred to as Human Genome 19 (hg19)). Suitably, a genomic location according to the present invention is a location that covers 2 to 200 bp of DNA. A genomic location according to the present invention preferably includes at least one CpG locus, and suitably includes at least two CpG loci, for example 2, 3, 4, 5, 6, 7 or 8 CpG loci, and preferably 2, 3, 4, 5 or 6 CpG loci.

As used herein the term “plurality” is at least 2, for example at least 10, at least 100, at least 1000, at least 10,000, at least 100,000, at least 10⁶, at least 10⁷, at least 10⁸ or at least 10⁹ or more.

As used herein the term “a level of prostate cancer fraction” is the level of cfDNA derived from prostate cancer cells in a cfDNA sample compared to the cfDNA that is not derived from prostate cancer cells. cfDNA that is not derived from prostate cancer cells in a cfDNA sample may be derived from blood cells, for example white blood cells (leukocyte), and other non-prostate tissues.

As used herein the term “blood cell fraction cfDNA” is cfDNA derived from DNA of a blood cell, for example a white blood cell (leukocyte).

As used herein, a “subject” refers to an animal, including mammals such as humans. Preferably, the subject is a human subject. As used herein, an “individual” can be a subject. As used herein, a “patient” refers to a human subject. In one embodiment, the subject is known or suspected to have a cancer (for example prostate cancer), and/or is known or suspected to have a risk of developing cancer (for example prostate cancer), or is known to have cancer and is known or suspected to have metastatic cancer (for example prostate cancer) or to have a risk of developing metastatic cancer (for example prostate cancer). In some embodiments, the subject is a subject who has been identified as being at risk of developing a cancer, in particular at risk of developing a prostate cancer.

As used herein, a “healthy subject” refers to a subject that has not been diagnosed with a type of cancer (for example prostate cancer), and preferably has not been diagnosed with any type of cancer. Thus, for example, a method of relating to prostate cancer, a “healthy subject” has no prostate cancer, and preferably no other type of cancer. Preferably, a healthy subject has not been diagnosed with a type of cancer (for example prostate cancer), and is not suspected of having a type of cancer, and suitably has not been diagnosed with any type of cancer (for example prostate cancer), and is not suspected of having any type of cancer.

The term “sample” as used herein means a biological sample derived from a patient to be screened in a method of the invention. The biological sample may be any suitable sample known in the art in which cfDNA can be detected and/or isolated. Included are individual cells and cell populations obtained from bodily tissues or fluids. Examples of suitable body fluids that may be used as samples according to the present invention are plasma, blood, and urine.

As used herein the term “methylation ratio” refers to the proportion of cytosine residues (C) that are methylated at all sequence reads covering a CpG locus (“G” is a guanine residue) within a population or pool of DNA, such as a sample of cfDNA obtained from the plasma of a subject. When the methylation profile is measured using bisulfite conversion the un-methylated CpG loci are converted to UpG (“U” is a uracil residue), while methylated CpG sites remain the same. The uracil residues are read as thymine residues during the DNA sequencing step following bisulfite conversion. The methylation ratio may be calculated using formula (X), which take the cytosine (C) and thymidine (T) counts from multiple sequence reads of a specific CpG locus:

$$Methylation Ratio = \frac{\begin{matrix}cytosine ( C ) counts from \\ \begin{matrix}all sequence reads \\ of a CpG locus\end{matrix}\end{matrix}}{\begin{matrix}cytosine ( C ) and thymidine \\ \begin{matrix}( T ) counts from all seqeunce \\ reads of the CpG loci\end{matrix}\end{matrix}} \qquad ( X )$$

For example, a CpG locus having a methylation ratio of 0.5 is methylated in 50% of the sequencing reads covering the specific CpG locus and unmethylated in 50% of the reads covering the specific CpG. A CpG locus having a methylation ratio of 0.75 is methylated in 75% of the sequencing reads covering the specific CpG locus and unmethylated in 25% of the reads covering the specific CpG. A CpG locus having a methylation ratio of 1.0 is methylated in 100% of the sequencing reads covering the specific CpG locus and unmethylated in 0% of the reads covering the specific CpG.

The methylation ratio of a specific CpG locus describes the degree of methylation of that specific CpG locus in the population or pool of DNA (for example the degree of methylation of that specific CpG locus in a sample of cfDNA obtained from the plasma of a subject).

Tools such as BSMAP (PMID: 19635165), Bismark (PMID: 21493656), gemBS (PMID: 30137223), Arioc (PMID: 29554207), BS-Seeker2 (PMID: 24206606), MethylCoder (PMID: 21724594) or BatMeth2 (PMID: 30669962) can be used to determine methylation ratios. These programs can also align the sequencing reads from bisulfite sequencing before determining methylation ratios.

As used herein the term “reference methylation ratio” is the methylation ratio of a CpG locus in a reference sample or reference methylome, for example the methylation ratio of a CpG locus in one of the following:
a cfDNA sample from a healthy subject, for example a healthy age-matched subject (for example, a sample from a subject before they have developed cancer);
a tissue sample from a healthy subject, for example a prostate tissue sample from a healthy subject;
a cancer biopsy sample from a cancer patient, for example a prostate cancer biopsy sample from a prostate cancer patient;
a cancer cell line sample, for example a prostate cancer cell line sample from a prostate cancer cell line;
a sample of white blood cells from a subject, for example the subject or a healthy subject;
a cfDNA sample from a different subject having a cancer (for example prostate cancer), preferably wherein the level of cancer fraction in the cfDNA sample from the different subject is known (more preferably multiple cfDNA samples (for example at least 2, 3, 4, 5, 10, 20, 40, 50, 100, 200 or 500 samples) each from a different subject having cancer, preferably wherein the level of cancer fraction in each cfDNA sample from the different subjects is known, and more preferably wherein each cfDNA sample has a different level of cancer fraction);
a cfDNA sample from a different subject having cancer (for example prostate cancer), wherein preferably the sample is known to comprise cfDNA derived from a cancer subtype (more preferably multiple cfDNA samples (for example at least 2, 3, 4, 5, 10, 20, 40, 50, 100, 200, 300 or 500 samples) each from a different subject having cancer, wherein preferably the each sample is known to comprise cfDNA derived from the cancer subtype, and for example wherein each cfDNA sample has a different level of cfDNA derived from the cancer subtype);
a characterized methylome sequence of a white blood cell;
a characterized methylome sequence of a cancer cell line (for example prostate cancer cell line);
a characterized methylome sequence of a cancerous cell; and/or
a characterized methylome sequence of a non-cancerous cell.

As regards using a cfDNA sample from a different subject having prostate cancer, wherein the level of prostate cancer fraction in the cfDNA sample from the different subject is known, the level of prostate cancer fraction in a cfDNA sample from a different subject can be determined by, for example, using methods that estimate tumour fraction using genomic markers. Due to the low sensitivity of such methods, generally the lowest percentage level of tumour fraction in a cfDNA sample that can be detected are around 5 to 10% tumour fraction.

As used herein the term “average methylation ratio” is the average of the methylation ratios of all the CpGs within a given genomic region. The average methylation ratio can be calculated by determining the sum of the methylation ratios of all CpGs within a given genomic region and dividing the sum by the number of CpGs within the given genomic region. The average methylation ratio may also be referred to as the mean methylation ratio. If a genomic region has only 1 CpG locus, the average methylation is the same as the methylation ratio for the single CpG locus in the genomic region. Programs such as methylKit R package v1.6.2 (Akalin, A. et al. Genome Biol 13, R87 (2012)) can be used to calculate average methylation ratio.

The average methylation ratio of a specific genomic region describes the degree of methylation of that specific genomic region in the population or pool of DNA (for example the degree of methylation of that specific genomic region in a sample of cfDNA obtained from the plasma of a subject).

The term “hypermethylated region” as used herein refers to a genomic region of cfDNA that is indicative of cancer when there is an increase in the average methylation ratio in the region (i.e. hypermethylation) compared to the average methylation ratio of the same genomic region in one or more of the following:
a cfDNA sample from a healthy subject, for example a healthy age-matched subject (for example, a sample from a subject before they have developed cancer);
a sample of white blood cells from a subject, for example the subject or a healthy subject;
a characterized methylome sequence of a white blood cell;
a cfDNA sample from a different subject having prostate cancer, wherein the level of prostate cancer fraction in the cfDNA sample from the different subject is known, and wherein the level of prostate cancer fraction in the cfDNA sample from a different subject is lower (for example at least 1%, 2%, 3%, 4%, 5%, 10%, 20%, 30%, 40% or 50% lower) compared to the sample from the subject (preferably multiple cfDNA samples (for example at least 2, 3, 4, 5, 10, 20, 40, 50, 100, 200, 300 or 500 samples) each from a different subject having prostate cancer, wherein the level of prostate cancer fraction in each cfDNA sample from the different subjects is known and wherein the level of prostate cancer fraction in each cfDNA sample from the different subjects is lower (for example at least 1%, 2%, 3%, 4%, 5%, 10%, 20%, 30%, 40% or 50% lower) compared to the sample from the subject).

The term “hypomethylated region” as used herein refers to a genomic region of cfDNA that is indicative of cancer when there is a decrease in the average methylation ratio in the region (i.e. hypomethylation) compared to the average methylation ratio of the same genomic region in one or more of the following:
a cfDNA sample from a healthy subject, for example a healthy age-matched subject (for example, a sample from a subject before they have developed cancer);
a sample of white blood cells from a subject, for example the subject or a healthy subject;
a characterized methylome sequence of a white blood cell;
a cfDNA sample from a different subject having prostate cancer, wherein the level of prostate cancer fraction in the cfDNA sample from the different subject is known, and wherein the level of prostate cancer fraction in the cfDNA sample from a different subject is higher (for example at least 1%, 2%, 3%, 4%, 5%, 10%, 20%, 30%, 40% or 50% higher) compared to the sample from the subject (preferably multiple cfDNA samples (for example at least 2, 3, 4, 5, 10, 20, 40, 50, 100, 200, 300 or 500 samples) each from a different subject having prostate cancer, wherein the level of prostate cancer fraction in each cfDNA sample from the different subjects is known and wherein the level of prostate cancer fraction in each cfDNA sample from the different subjects is higher (for example at least 1%, 2%, 3%, 4%, 5%, 10%, 20%, 30%, 40% or 50% higher) compared to the sample from the subject).

The term “methylation score” as used herein is a value that is indicative of the methylation state of a sub-population or fraction of DNA in a sample. For example a “methylation score” may be indicative of the methylation state of the genomic regions in a sample that have the average methylation ratio determined. The methylation score may be, for example:
the median or the mean of the average methylation ratios for the genomic regions that have had average methylation ratios determined;
the median or the mean of the average methylation ratios for a first group of genomic regions that have had average methylation ratios determined (resulting in a first methylation score) and/or the median or the mean of the average methylation ratios for a second group of genomic regions that have had average methylation ratios determined (resulting in a second methylation score) (for example wherein the first group of genomic regions are all of the hypermethylated genomic regions, and the second group of genomic regions are all of the hypomethylated genomic regions); or
the methylation ratio score for each genomic region that have the average methylation ratio determined, wherein a methylation ratio score is determined by comparing the average methylation ratio at each genomic region to a reference methylation ratio for each genomic region.

In certain embodiments, preferably the methylation score is, for example:
the median of the average methylation ratios for the genomic regions that have the average methylation ratio determined;
the median of the average methylation ratios for a first group of genomic regions that have had average methylation ratios determined (resulting in a first methylation score), and/or the median of the average methylation ratios for a second group of genomic regions that have had average methylation ratios determined (resulting in a second methylation score) (for example wherein the first group of genomic regions are all of the hypermethylated genomic regions, and the second group of genomic regions are all of the hypomethylated genomic regions)

The term “reference methylation score” as used herein is a methylation score for a reference sample or a reference methylome. The reference sample or reference methylome may selected from the group consisting of:
a cfDNA sample from a healthy subject, for example a healthy age-matched subject (for example, a sample from a subject before they have developed cancer);
a tissue sample from a healthy subject, for example a prostate tissue sample from a healthy subject;
a cancer biopsy sample from a cancer patient, for example a prostate cancer biopsy sample from a prostate cancer patient;
a cancer cell line sample, for example a prostate cancer cell line sample from a prostate cancer cell line;
a sample of white blood cells from a subject, for example the subject or a healthy subject;
a cfDNA sample from a different subject having cancer (for example prostate cancer), preferably wherein the level of cancer fraction in the cfDNA sample from the different subject is known (more preferably multiple cfDNA samples (for example at least 2, 3, 4, 5, 10, 20, 40, 50, 100, 200, 300 or 500 samples) each from a different subject having cancer, wherein preferably the level of cancer fraction in each cfDNA sample from the different subjects is known, and more preferably wherein each cfDNA sample has a different level of cancer fraction);
a cfDNA sample from a different subject having cancer (for example prostate cancer), wherein preferably the sample is known to comprise cfDNA derived from a cancer subtype (more preferably multiple cfDNA samples (for example at least 2, 3, 4, 5, 10, 20, 40, 50, 100, 200, 300 or 500 samples) each from a different subject having cancer, wherein preferably the each sample is known to comprise cfDNA derived from the cancer subtype, and for example wherein each cfDNA sample has a different level of cfDNA derived from the cancer subtype);
a characterized methylome sequence of a white blood cell;
a characterized methylome sequence of a cancer cell line;
a characterized methylome sequence of a cancerous cell; and
a characterized methylome sequence of a non-cancerous cell.

The reference methylation score is preferably calculated (for example calculated using the average methylation ratio) for the same genomic regions as the genomic regions for a methylation score to which the reference methylation score is to be compared with.

For example, if a methylation score is the median of the average methylation ratios for all genomic regions that have had the average methylation ratios determined, then preferably the reference methylation score is the median of the average methylation ratios for the same genomic regions in a reference sample or reference methylome. If a methylation score is the mean of the average methylation ratios for all genomic regions that have had the average methylation ratios determined, then preferably the reference methylation score is the mean of the average methylation ratios for the same genomic regions in a reference sample or reference methylome

If a methylation score is the median of the average methylation ratios for a first group of genomic regions (resulting in a first methylation score) and/or the median of the average methylation for a second group of genomic regions (resulting in a second methylation score), then preferably the reference methylation score is the median of the average methylation ratios for the same first group of genomic regions (resulting in a first reference methylation score) and/or the median of the average methylation ratios for the same second group of genomic regions (resulting in a second reference methylation score) in a reference sample or reference methylome.

If a methylation score is the mean of the average methylation ratios for a first group of genomic regions (resulting in a first methylation score) and/or the mean of the average methylation for a second group of genomic regions (resulting in a second methylation score), then preferably the reference methylation score is the mean of the average methylation ratios for the same first group of genomic regions (resulting in a first reference methylation score) and/or the mean of the average methylation ratios for the same second group of genomic regions (resulting in a second reference methylation score) in a reference sample or reference methylome.

If a methylation score is the methylation ratio score for each genomic region that have the average methylation ratio determined, wherein a methylation ratio score is determined by comparing the average methylation ratio at each genomic region to a reference methylation ratio for each genomic region, the reference methylation score is preferably the reference methylation ratio score for each of the same genomic regions in a reference sample.

As used herein an “abnormal level of PSA” is a level of PSA in the blood indicative of a risk of a patient having prostate cancer. For example an abnormal level of PSA in the blood may be a level of at least 4.0 ng/mL. An “abnormal level of PSA” may additionally be an increase in the level of PSA in the blood compared to the level at initial diagnosis or the level at the previous time PSA was tested in the subject (for example an increase of 0.1 ng/mL or more, 0.2 ng/mL or more, 0.5 ng/mL or more, 1.0 ng/mL or more compared to the level at initial diagnosis or the level at the previous time PSA was tested in the subject).

The term “oligonucleotide(s)” are nucleic acids that are usually between 5 and 100 contiguous bases, for example between 5-10, 5-20, 10-20, 10-50, 15-50, 15-100, 20-50, or 20-100 contiguous bases. An oligonucleotide may be capable of hybridising to a target of interest, e.g., a sequence that is at least 10 nucleotides in length. An oligonucleotide for hybridising to a target may comprise at least 10, at least 15 nucleotides, at least 20 nucleotides, at least 30 nucleotides, at least 40 nucleotides, at least 50 nucleotides or at least 60 nucleotides. An oligonucleotide can be used as a primer, a probe, included in a microarray, or used in polynucleotide-based identification methods. An oligonucleotide may be capable of hybridising to a DNA genomic region of the invention, for example a DNA genomic region as defined in Tables 1 to 4, or DNA genomic region comprising a DNA genomic region as defined in Tables 1 to 4, or a 2 to 99 bp DNA genomic region within a DNA genomic region defined in Tables 1 to 4 and comprising at least one CpG locus.

The term “comprising” as used in this specification and claims means “consisting at least in part of” or “consisting of”, that is to say when interpreting statements in this specification and claims which include the term, the features, prefaced by that term in each statement, all need to be present but other features can also be present. Related terms such as “comprise” and “comprised” are to be interpreted in a similar manner.

As used herein a “subtype of a cancer” (for example a “subtype of prostate cancer”) is a subset of a type of cancer based on characteristics of the cancer cells, and in particular molecular and genetic characteristics of the cells. Different cancer subtypes can have different disease progression and can respond or not respond to different treatments. The subtype of a cancer is, for example, used to assist in planning treatment and determine prognosis of the patient having that cancer subtype.

As used herein a “solid cancer cfDNA methylome signature” is a set of CpG loci and/or genomic regions that have a certain state of methylation in cfDNA derived from solid cancer cells. The pattern or fingerprint of methylation at the set of CpG loci and/or genomic regions is indicative of the solid cancer, and can provide information relating to the solid cancer, for example the level of solid cancer fraction in the cfDNA sample, a subtype of cancer (for example a genomic subtype), the aggression of the cancer, the prognosis of the cancer, and/or the tumour response to a treatment. A CpG locus or genomic region of a solid cancer cfDNA methylome signature may be tissue specific (for example, a certain state of methylation present in a particular tissue type, i.e. the tissue from which the cancer is derived) and/or cancer specific (for example, a certain state of methylation present in a particular cancer type). A CpG locus or genomic region of a solid cancer cfDNA methylome signature may have increased methylation compared to, for example, the methylation of the same locus or genomic region in a white blood cell and/or non-tumour cell and/or a different tissue to the cancer tissue, and especially compared to the methylation of the same locus or genomic region in a white blood cell. A CpG locus or genomic region of a solid cancer cfDNA methylome signature may have decreased methylation compared to, for example, the methylation of the same locus or genomic region in a white blood cell and/or non-tumour cell and/or a different tissue to the cancer tissue, and especially compared to the methylation of the same locus or genomic region in a white blood cell.

### DETAILED DESCRIPTION OF THE INVENTION

Tumour DNA circulates in the plasma of cancer patients admixed with DNA from non-cancerous cells. The genomic landscape of plasma DNA has been characterized in prostate cancer, for example, metastatic castration-resistant prostate cancer (mCRPC) but the plasma methylome has not been extensively explored. The identification of circulating methylation biomarkers can be challenging due to the heterogeneities of methylation. The traditional way to identify methylation markers started with the comparison between cancer tissue and normal tissue methylation patterns, and cancer-specific methylation loci are chosen and later validated in plasma samples. The present inventors have used an innovative approach and workflow to characterize the plasma methylome in mCRPC and identify a unique set of methylation markers due to the innovative experimental design which uses an unbiased approach to investigate the methylation profile of tumour derived cfDNA. The inventors' approach starts from profiling plasma pan-methylome. They then applied unbiased dimensional reduction algorithms, such as principal component analysis (PCA), and selected the regions most highly correlated with genomically-determined tumour fraction or the subtype of interest. The methylation markers identified by this approach markers can be used as cancer-specific methylation signatures in methods of the invention for high sensitivity and accurate tracking of tumour DNA in subjects with, for example, suspected or confirmed untreated or treated prostate cancer and/or for subtyping prostate cancer patients.

Furthermore, due to the large number of regions that the inventors have found to highly correlate with prostate and prostate cancer specific DNA methylation patterns and that show the greatest variance when compared to non-cancer plasma DNA in age-matched men, the inventors have been able to develop methods that are applicable to, for example, low-pass whole genome bisulfite sequencing data, and thus will be cost-effective and clinically scalable methods for detecting, screening, monitoring, staging, classification, selecting treatment for, ascertaining whether treatment is working in, and/or prognostication of prostate cancer.

Additionally, due to the methylation markers of the signatures of the present invention being based on variance compared to non-cancer plasma DNA in age-matched men, the signatures can be used in the methods described here to provide increased sensitivity and specificity for determining the level of prostate cancer fraction in a cfDNA sample, and in particular to detect significantly lower levels than is possible using genomic screening of cfDNA. Also, as methylation markers are not affected by clonal hematopoiesis in older populations (i.e. the formation of a genetically distinct subpopulation of blood cells), which can introduce false positives in genomic alternation-based tests, the methods of the present invention are applicable to subjects of all ages. Furthermore, as the methods of the invention determine methylation levels at multiple different methylation markers of the signatures of the present invention, the methods are not biased by inter-patient differences and genomic changes that could occur in normal cells and that could introduce a false positive result in the case of genomic testing.

Surprisingly, and due to the innovative workflow of the present invention, the methylation signatures of the present invention include methylation markers that are specific to either normal prostate tissue or prostate cancer tissue. The approach can be adapted and applied to other tumour types to identify circulating tumour-specific methylation signatures that can be used to accurately detect a tumour at earlier stages and quantitate tumour fraction. Also surprisingly, the signature found by the inventors did not include genes whose methylation status has been previously reported as diagnostic of prostate cancer such as, GSTP1, APC, RASSF1 and HOXD3 (Massie, C. E, et al, J Steroid Biochem Mol Biol 166, 1-15 (2017)). Although not wishing to be bound by theory, the present inventors postulate that this finding could be explained by highly variable methylation levels at the genomic regions of the signature in non-cancer plasma DNA compared to cancer plasma DNA. The inventors therefore understand that, in view of the signatures being found by the innovative workflow of the present invention, only the most stably methylated regions in non-cancer plasma DNA are identified as discriminators between non-cancer plasma DNA and cancer plasma DNA and are included in the signatures.

The present invention finds particular utility in risk stratification of men diagnosed with localised prostate cancer. Men with prostate cancer DNA detected in plasma using methods of the present invention can be staged, classified, and/or offered additional treatment with the aim of maximising cure whilst minimising over-treatment of men who do not require it. Furthermore, the methods of the invention can be used to identify patients with poorer prognosis so that a more intensive primary treatment can be selected, i.e. patients with a high tumour fraction level in the plasma, or who have an aggressive subtype of cancer. The methods can also be used for monitoring whether a treatment for prostate cancer is working or not, and for selecting further treatment, if necessary. Also, the half-life of Plasma DNA is approximately 1 hour so changes can be seen within days when a cancer is responding/not responding. Thus testing, after start of treatment (for example days or weeks after start of treatment) could be used to identify men for whom treatment is ineffective and to guide a change to a more effective alternative, potentially improving outcomes and minimising unnecessary side-effects.

Currently PSA testing is used to determine bio-chemical progression, and whole-body MRI scanning/PSMA testing for detecting metastases. PSA testing has come under much scrutiny for its reliability and overdiagnosis. Imaging modalities cannot detect metastatic disease as early as a ctDNA test. Imaging can only detect lesions >0.5-1 cm, i.e. 1 million cells or more. On the other hand, it is possible detect DNA from a few 100 tumour cells in circulation. The methods of the present invention can therefore complement or replace imaging for more accurate detecting, screening, monitoring, staging, classification and prognostication of prostate cancer, and in particular metastatic prostate cancer.

Furthermore, the methods and approaches employed by the present inventors to find the signatures described herein can be used in methods to find further signatures useful for detecting, screening, monitoring, staging, classification, selecting treatment for, ascertaining whether treatment is working in, and/or prognostication of other solid cancers in a sample obtained from a subject, wherein the sample comprises circulating free DNA (cfDNA).

DNA cytosine methylation, also called DNA methylation or CpG methylation, plays an important part in multiple biological processes by interacting with specific methyl-CpG binding proteins or specific methyl-CpG binding domains (MBDs), a key messenger to other transcriptional regulators which result in histone modification, chromatin re-arrangement, and differential gene expressions (Ballestar, E. & Esteller, M. Biochem Cell Biol 83, 374-384 (2005); Nakayama, T. et al. Lab Invest 80, 1789-1796 (2000)). Some DNA methylation is believed to remain constant in tumour clones, and have the unique inheritance, while some methylation consequences may be later events and result in more malignant form of cancer (Beltran, H. et al. Nat Med 22, 298-305 (2016)). Therefore, it has been hypothesized that DNA methylation signatures could be an important indicator for both early carcinogenesis and advanced tumour progression.

Methods of the Invention to Determine the Level of Prostate Cancer Fraction in a cfDNA Sample

The present invention provides a method for detecting, screening, monitoring, staging, classification, selecting treatment for, ascertaining whether treatment is working in, and/or prognostication of prostate cancer in a sample obtained from a subject, wherein the sample comprises circulating free DNA (cfDNA), the method comprising:
characterizing the methylome sequence of a plurality of cfDNA molecules in the sample, wherein the methylome sequence of a cfDNA molecule is the DNA sequence and the methylation profile of the molecule;
determining the average methylation ratio at 10 or more genomic regions, each genomic region being selected from the group consisting of:
   a 100 to 200 bp region comprising or having a genomic location defined in Tables 1 to 4, and
   a 2 to 99 bp region within a genomic location defined in Tables 1 to 4 and comprising at least one CpG locus,
and wherein each of the genomic regions is covered by at least one sequence read of at least one characterized methylome sequence;
calculating a methylation score using the average methylation ratio for each of the genomic regions;
analyzing the methylation score to determine the level of prostate cancer fraction in the cfDNA sample.

Tables 1 to 4 are provided below. The genomic locations have been separated into 4 tables based on whether a region including, having, or within the genomic location is a hypermethylated region (i.e. indicative of cancer when there in an increase in methylation level for the region) or a hypomethylated region (i.e. indicative of cancer when there is a decrease in methylation level for the region) when used in the method, and a region including, having, or within the genomic location is indicative of a methylation pattern specific to prostate tissue or is indicative of a methylation pattern specific to prostate cancer. The genomic locations of Tables 1 (and Table 1b) to 4 are locations with reference to hg19.

TABLE 1
Hypermethylated region, prostate tissue specific genomic locations

| Chromosome | start | end | gene |
|---|---|---|---|
| 1 | 24649401 | 24649501 | GRHL3 |
| 1 | 36043651 | 36043751 | TFAP2E |
| 1 | 45274001 | 45274101 | BTBD19 |
| 1 | 45274051 | 45274151 | BTBD19 |
| 1 | 47909951 | 47910051 | n/a |
| 1 | 64937351 | 64937451 | CACHD1 |
| 1 | 67600301 | 67600401 | C1orf141 |
| 1 | 67600451 | 67600551 | C1orf141 |
| 1 | 68154601 | 68154701 | n/a |
| 1 | 119526151 | 119526251 | TBX15 |
| 1 | 119526201 | 119526301 | TBX15 |
| 1 | 119526251 | 119526351 | TBX15 |
| 1 | 119526301 | 119526401 | TBX15 |
| 1 | 119526351 | 119526451 | TBX15 |
| 1 | 119526401 | 119526501 | TBX15 |
| 1 | 119531601 | 119531701 | TBX15 |
| 1 | 119531651 | 119531751 | TBX15 |
| 1 | 119531701 | 119531801 | TBX15 |
| 1 | 119532751 | 119532851 | TBX15 |
| 1 | 119532801 | 119532901 | TBX15 |
| 1 | 119532851 | 119532951 | TBX15 |
| 1 | 235147651 | 235147751 | n/a |
| 1 | 235147701 | 235147801 | n/a |
| 10 | 22765851 | 22765951 | n/a |
| 10 | 22765901 | 22766001 | n/a |
| 10 | 29096801 | 29096901 | LINC01517 |
| 10 | 29096851 | 29096951 | LINC01517 |
| 10 | 31423451 | 31423551 | n/a |
| 10 | 31423501 | 31423601 | n/a |
| 10 | 31423551 | 31423651 | n/a |
| 10 | 101280651 | 101280751 | n/a |
| 10 | 101280701 | 101280801 | n/a |
| 10 | 126336751 | 126336851 | FAM53B |
| 10 | 126336801 | 126336901 | FAM53B |
| 11 | 2920151 | 2920251 | SLC22A18AS |
| 11 | 46367051 | 46367151 | DGKZ |
| 11 | 46367101 | 46367201 | DGKZ |
| 11 | 47939651 | 47939751 | n/a |
| 11 | 47939701 | 47939801 | n/a |
| 11 | 47939751 | 47939851 | n/a |
| 11 | 63973901 | 63974001 | FERMT3 |
| 11 | 111809551 | 111809651 | DIXDC1 |
| 12 | 19389701 | 19389801 | PLEKHA5 |
| 12 | 19389751 | 19389851 | PLEKHA5 |
| 12 | 45443801 | 45443901 | DBX2 |
| 12 | 54441001 | 54441101 | HOXC4 |
| 12 | 54441051 | 54441151 | HOXC4 |
| 12 | 81471601 | 81471701 | ACSS3 |
| 12 | 81471651 | 81471751 | ACSS3 |
| 12 | 90150551 | 90150651 | n/a |
| 12 | 90150601 | 90150701 | n/a |
| 12 | 95941801 | 95941901 | USP44 |
| 12 | 116997001 | 116997101 | MAP1LC3B2 |
| 12 | 116997051 | 116997151 | MAP1LC3B2 |
| 12 | 116997101 | 116997201 | MAP1LC3B2 |
| 13 | 95357651 | 95357751 | LOC101927248 |
| 13 | 95357701 | 95357801 | LOC101927248 |
| 13 | 99959551 | 99959651 | GPR183 |
| 13 | 99959601 | 99959701 | GPR183 |
| 14 | 24808701 | 24808801 | RIPK3 |
| 14 | 37124151 | 37124251 | n/a |
| 14 | 37124201 | 37124301 | n/a |
| 14 | 60973301 | 60973401 | n/a |
| 14 | 60973351 | 60973451 | n/a |
| 14 | 60976851 | 60976951 | SIX6 |
| 14 | 60976901 | 60977001 | SIX6 |
| 14 | 60977001 | 60977101 | SIX6 |
| 14 | 60977051 | 60977151 | SIX6 |
| 14 | 61109951 | 61110051 | n/a |
| 15 | 42227251 | 42227351 | EHD4 |
| 15 | 96909701 | 96909801 | n/a |
| 17 | 46667051 | 46667151 | HOXB-AS3 |
| 17 | 46673851 | 46673951 | HOXB-AS3 |
| 17 | 46673901 | 46674001 | HOXB-AS3 |
| 17 | 59534551 | 59534651 | TBX4 |
| 17 | 59534601 | 59534701 | TBX4 |
| 17 | 59534651 | 59534751 | TBX4 |
| 17 | 59534701 | 59534801 | TBX4 |
| 17 | 75471401 | 75471501 | 40057 |
| 17 | 80944051 | 80944151 | B3GNTL1 |
| 18 | 12307251 | 12307351 | TUBB6 |
| 19 | 16394401 | 16394501 | n/a |
| 19 | 16394451 | 16394551 | n/a |
| 19 | 18508551 | 18508651 | LRRC25 |
| 19 | 35396351 | 35396451 | n/a |
| 19 | 41316751 | 41316851 | n/a |
| 19 | 41316801 | 41316901 | n/a |
| 19 | 46526251 | 46526351 | PGLYRP1 |
| 19 | 46917001 | 46917101 | CCDC8 |
| 19 | 46917051 | 46917151 | CCDC8 |
| 19 | 53039001 | 53039101 | ZNF808 |
| 19 | 55592451 | 55592551 | EPS8L1 |
| 19 | 58728401 | 58728501 | n/a |
| 19 | 58728451 | 58728551 | n/a |
| 2 | 8379951 | 8380051 | LINC00299 |
| 2 | 8380001 | 8380101 | LINC00299 |
| 2 | 26521751 | 26521851 | n/a |
| 2 | 26521801 | 26521901 | n/a |
| 2 | 45465301 | 45465401 | LINC01121 |
| 2 | 46613551 | 46613651 | EPAS1 |
| 2 | 54900801 | 54900901 | n/a |
| 2 | 54900901 | 54901001 | n/a |
| 2 | 54900951 | 54901051 | n/a |
| 2 | 54901001 | 54901101 | n/a |
| 2 | 63282251 | 63282351 | OTX1 |
| 2 | 63282651 | 63282751 | OTX1 |
| 2 | 63283851 | 63283951 | OTX1 |
| 2 | 63283901 | 63284001 | OTX1 |
| 2 | 71116551 | 71116651 | LINC01143 |
| 2 | 71116601 | 71116701 | LINC01143 |
| 2 | 71116651 | 71116751 | LINC01143 |
| 2 | 71116701 | 71116801 | LINC01143 |
| 2 | 71126251 | 71126351 | n/a |
| 2 | 71131551 | 71131651 | VAX2 |
| 2 | 71131601 | 71131701 | VAX2 |
| 2 | 71134851 | 71134951 | VAX2 |
| 2 | 172945251 | 172945351 | METAP1D |
| 2 | 176964051 | 176964151 | HOXD12 |
| 2 | 177012551 | 177012651 | n/a |
| 2 | 177012601 | 177012701 | n/a |
| 2 | 177012651 | 177012751 | n/a |
| 2 | 177012701 | 177012801 | n/a |
| 2 | 201450501 | 201450601 | AOX1 |
| 2 | 201450551 | 201450651 | AOX1 |
| 2 | 201450601 | 201450701 | AOX1 |
| 2 | 201450651 | 201450751 | AOX1 |
| 2 | 206551401 | 206551501 | NRP2 |
| 2 | 206551451 | 206551551 | NRP2 |
| 2 | 228324851 | 228324951 | n/a |
| 2 | 228324901 | 228325001 | n/a |
| 2 | 228324951 | 228325051 | n/a |
| 2 | 228325001 | 228325101 | n/a |
| 2 | 238777551 | 238777651 | RAMP1 |
| 2 | 242908101 | 242908201 | LINC01237 |
| 21 | 37802451 | 37802551 | n/a |
| 21 | 37802501 | 37802601 | n/a |
| 21 | 37802551 | 37802651 | n/a |
| 3 | 33701201 | 33701301 | CLASP2 |
| 3 | 46448851 | 46448951 | CCRL2 |
| 3 | 46448901 | 46449001 | CCRL2 |
| 3 | 127453801 | 127453901 | MGLL |
| 3 | 127453851 | 127453951 | MGLL |
| 3 | 167742601 | 167742701 | GOLIM4 |
| 3 | 170746251 | 170746351 | n/a |
| 4 | 20256801 | 20256901 | SLIT2 |
| 4 | 20256851 | 20256951 | SLIT2 |
| 4 | 54959951 | 54960051 | n/a |
| 4 | 54960001 | 54960101 | n/a |
| 4 | 54975201 | 54975301 | n/a |
| 4 | 54975251 | 54975351 | n/a |
| 4 | 74809851 | 74809951 | n/a |
| 4 | 75230551 | 75230651 | EREG |
| 4 | 75230601 | 75230701 | EREG |
| 4 | 81107201 | 81107301 | PRDM8 |
| 4 | 87281351 | 87281451 | MAPK10 |
| 4 | 87281401 | 87281501 | MAPK10 |
| 4 | 108814501 | 108814601 | LOC101929595 |
| 4 | 108814551 | 108814651 | SGMS2 |
| 4 | 188917101 | 188917201 | ZFP42 |
| 5 | 297251 | 297351 | PDCD6 |
| 5 | 1608551 | 1608651 | LOC728613 |
| 5 | 1608601 | 1608701 | LOC728613 |
| 5 | 72676801 | 72676901 | n/a |
| 5 | 87439351 | 87439451 | n/a |
| 5 | 87439401 | 87439501 | n/a |
| 5 | 134735451 | 134735551 | H2AFY |
| 5 | 134880301 | 134880401 | n/a |
| 5 | 140800801 | 140800901 | PCDHGA11 |
| 5 | 170735101 | 170735201 | n/a |
| 5 | 170735251 | 170735351 | TLX3 |
| 5 | 172673051 | 172673151 | n/a |
| 6 | 6901051 | 6901151 | n/a |
| 6 | 10887701 | 10887801 | SYCP2L |
| 6 | 26088151 | 26088251 | HFE |
| 6 | 27107251 | 27107351 | HIST1H2BK |
| 6 | 27107301 | 27107401 | HIST1H4I |
| 6 | 27107651 | 27107751 | HIST1H4I |
| 6 | 27107701 | 27107801 | HIST1H2BK |
| 6 | 27107751 | 27107851 | HIST1H4I |
| 6 | 27858251 | 27858351 | HIST1H3J |
| 6 | 27858301 | 27858401 | HIST1H3J |
| 6 | 27858551 | 27858651 | HIST1H3J |
| 6 | 139795501 | 139795601 | LINC01625 |
| 6 | 147235051 | 147235151 | STXBP5-AS1 |
| 7 | 27289101 | 27289201 | n/a |
| 7 | 38361201 | 38361301 | n/a |
| 7 | 45066651 | 45066751 | CCM2 |
| 7 | 45066701 | 45066801 | CCM2 |
| 7 | 73132051 | 73132151 | STX1A |
| 7 | 73132101 | 73132201 | STX1A |
| 7 | 116140351 | 116140451 | CAV2 |
| 7 | 129423101 | 129423201 | n/a |
| 7 | 129425301 | 129425401 | n/a |
| 7 | 129425351 | 129425451 | n/a |
| 7 | 149112151 | 149112251 | n/a |
| 8 | 55066251 | 55066351 | n/a |
| 8 | 55066301 | 55066401 | n/a |
| 9 | 971451 | 971551 | n/a |
| 9 | 22005201 | 22005301 | CDKN2B |
| 9 | 22005251 | 22005351 | CDKN2B-AS1 |
| 9 | 22005301 | 22005401 | CDKN2B-AS1 |
| 9 | 22005501 | 22005601 | CDKN2B-AS1 |
| 9 | 22005551 | 22005651 | CDKN2B-AS1 |
| 9 | 22005601 | 22005701 | CDKN2B |
| 9 | 112810301 | 112810401 | PALM2-AKAP2 |
| 9 | 126775151 | 126775251 | LHX2 |
| 9 | 135462201 | 135462301 | BARHL1 |
| 9 | 139129901 | 139130001 | QSOX2 |

TABLE 2
Hypermethylated region, prostate cancer specific genomic locations

| Chromosome | start | end | gene |
|---|---|---|---|
| 1 | 12404851 | 12404951 | VPS13D |
| 1 | 19278651 | 19278751 | IFFO2 |
| 1 | 27944951 | 27945051 | FGR |
| 1 | 27945001 | 27945101 | FGR |
| 1 | 28218201 | 28218301 | RPA2 |
| 1 | 54562051 | 54562151 | TCEANC2 |
| 1 | 54562101 | 54562201 | TCEANC2 |
| 1 | 54562151 | 54562251 | TCEANC2 |
| 1 | 65399401 | 65399501 | JAK1 |
| 1 | 65399451 | 65399551 | JAK1 |
| 1 | 66839051 | 66839151 | PDE4B |
| 1 | 66839101 | 66839201 | PDE4B |
| 1 | 68154551 | 68154651 | n/a |
| 1 | 117046951 | 117047051 | n/a |
| 1 | 117047001 | 117047101 | n/a |
| 1 | 117058401 | 117058501 | CD58 |
| 1 | 117058451 | 117058551 | CD58 |
| 1 | 150971801 | 150971901 | FAM63A |
| 1 | 154376251 | 154376351 | n/a |
| 1 | 154376301 | 154376401 | n/a |
| 1 | 155506001 | 155506101 | ASH1L |
| 1 | 156462151 | 156462251 | MEF2D |
| 1 | 156509751 | 156509851 | IQGAP3 |
| 1 | 156509801 | 156509901 | IQGAP3 |
| 1 | 181031851 | 181031951 | n/a |
| 1 | 202130701 | 202130801 | PTPN7 |
| 1 | 207103601 | 207103701 | PIGR |
| 1 | 207103651 | 207103751 | PIGR |
| 1 | 217313801 | 217313901 | n/a |
| 10 | 497351 | 497451 | DIP2C |
| 10 | 22766101 | 22766201 | n/a |
| 10 | 22936901 | 22937001 | PIP4K2A |
| 10 | 22936951 | 22937051 | PIP4K2A |
| 10 | 88632601 | 88632701 | BMPR1A |
| 10 | 88632651 | 88632751 | BMPR1A |
| 10 | 94450801 | 94450901 | HHEX |
| 10 | 94450851 | 94450951 | HHEX |
| 10 | 94450901 | 94451001 | HHEX |
| 10 | 94450951 | 94451051 | HHEX |
| 10 | 94451001 | 94451101 | HHEX |
| 11 | 31817851 | 31817951 | PAX6 |
| 11 | 62455001 | 62455101 | LRRN4CL |
| 11 | 70211351 | 70211451 | PPFIA1 |
| 11 | 70211401 | 70211501 | PPFIA1 |
| 11 | 70211451 | 70211551 | PPFIA1 |
| 11 | 70211501 | 70211601 | PPFIA1 |
| 11 | 70248651 | 70248751 | CTTN |
| 11 | 70248701 | 70248801 | CTTN |
| 12 | 1642601 | 1642701 | n/a |
| 12 | 6665301 | 6665401 | IFFO1 |
| 12 | 6665351 | 6665451 | IFFO1 |
| 12 | 6665401 | 6665501 | IFFO1 |
| 12 | 7060151 | 7060251 | PTPN6 |
| 12 | 7060201 | 7060301 | PTPN6 |
| 12 | 7062051 | 7062151 | PTPN6 |
| 12 | 7062101 | 7062201 | PTPN6 |
| 12 | 7062151 | 7062251 | PTPN6 |
| 12 | 47610151 | 47610251 | PCED1B-AS1 |
| 12 | 47610201 | 47610301 | PCED1B |
| 12 | 109899001 | 109899101 | KCTD10 |
| 12 | 109899051 | 109899151 | KCTD10 |
| 12 | 111536901 | 111537001 | CUX2 |
| 12 | 111536951 | 111537051 | CUX2 |
| 12 | 115135751 | 115135851 | n/a |
| 12 | 123707851 | 123707951 | MPHOSPH9 |
| 13 | 113437951 | 113438051 | ATP11A |
| 13 | 113438001 | 113438101 | ATP11A |
| 13 | 113438051 | 113438151 | ATP11A |
| 14 | 37124901 | 37125001 | n/a |
| 14 | 37124951 | 37125051 | n/a |
| 14 | 37125001 | 37125101 | n/a |
| 14 | 37125801 | 37125901 | PAX9 |
| 14 | 37125851 | 37125951 | PAX9 |
| 14 | 37125901 | 37126001 | PAX9 |
| 14 | 37126051 | 37126151 | PAX9 |
| 14 | 38725601 | 38725701 | CLEC14A |
| 14 | 38725651 | 38725751 | CLEC14A |
| 14 | 95237351 | 95237451 | GSC |
| 14 | 95237401 | 95237501 | GSC |
| 15 | 86098551 | 86098651 | AKAP13 |
| 15 | 86098601 | 86098701 | AKAP13 |
| 15 | 96887101 | 96887201 | n/a |
| 15 | 101777751 | 101777851 | CHSY1 |
| 15 | 101777801 | 101777901 | CHSY1 |
| 15 | 101991451 | 101991551 | PCSK6 |
| 16 | 2737251 | 2737351 | KCTD5 |
| 16 | 2737301 | 2737401 | KCTD5 |
| 16 | 29675101 | 29675201 | SPN |
| 16 | 88038901 | 88039001 | BANP |
| 16 | 88866601 | 88866701 | n/a |
| 17 | 41799001 | 41799101 | n/a |
| 17 | 41799051 | 41799151 | n/a |
| 17 | 43242651 | 43242751 | HEXIM2 |
| 17 | 55533301 | 55533401 | MSI2 |
| 17 | 55533351 | 55533451 | MSI2 |
| 17 | 55562901 | 55563001 | MSI2 |
| 17 | 55562951 | 55563051 | MSI2 |
| 17 | 56407101 | 56407201 | BZRAP1-AS1 |
| 17 | 59532801 | 59532901 | TBX4 |
| 17 | 59532851 | 59532951 | TBX4 |
| 17 | 59536601 | 59536701 | TBX4 |
| 17 | 70715551 | 70715651 | SLC39A11 |
| 17 | 72776151 | 72776251 | TMEM104 |
| 17 | 72776201 | 72776301 | TMEM104 |
| 17 | 78724151 | 78724251 | RPTOR |
| 17 | 79422501 | 79422601 | BAHCC1 |
| 17 | 79422551 | 79422651 | BAHCC1 |
| 17 | 79422601 | 79422701 | BAHCC1 |
| 17 | 80740751 | 80740851 | TBCD |
| 17 | 81039651 | 81039751 | METRNL |
| 17 | 81039701 | 81039801 | METRNL |
| 19 | 2776001 | 2776101 | SGTA |
| 19 | 31843301 | 31843401 | n/a |
| 19 | 33162801 | 33162901 | ANKRD27 |
| 19 | 33162851 | 33162951 | ANKRD27 |
| 19 | 33162901 | 33163001 | ANKRD27 |
| 2 | 3246151 | 3246251 | TSSC1 |
| 2 | 3246201 | 3246301 | TSSC1 |
| 2 | 10687901 | 10688001 | n/a |
| 2 | 10687951 | 10688051 | n/a |
| 2 | 10688251 | 10688351 | n/a |
| 2 | 10688601 | 10688701 | n/a |
| 2 | 10688651 | 10688751 | n/a |
| 2 | 11674501 | 11674601 | GREB1 |
| 2 | 11674551 | 11674651 | GREB1 |
| 2 | 27298301 | 27298401 | n/a |
| 2 | 30489401 | 30489501 | n/a |
| 2 | 36776251 | 36776351 | CRIM1 |
| 2 | 36776301 | 36776401 | CRIM1 |
| 2 | 55339151 | 55339251 | n/a |
| 2 | 63279651 | 63279751 | OTX1 |
| 2 | 71132301 | 71132401 | VAX2 |
| 2 | 106415051 | 106415151 | NCK2 |
| 2 | 106415101 | 106415201 | NCK2 |
| 2 | 111875851 | 111875951 | n/a |
| 2 | 111875901 | 111876001 | n/a |
| 2 | 171569251 | 171569351 | LINC01124 |
| 2 | 172945201 | 172945301 | METAP1D |
| 2 | 172974151 | 172974251 | DLX2-AS1 |
| 2 | 172974201 | 172974301 | DLX2-AS1 |
| 2 | 198063601 | 198063701 | ANKRD44 |
| 2 | 198063651 | 198063751 | ANKRD44 |
| 2 | 202126301 | 202126401 | CASP8 |
| 2 | 202126351 | 202126451 | CASP8 |
| 2 | 204571201 | 204571301 | CD28 |
| 2 | 204571301 | 204571401 | CD28 |
| 2 | 206004801 | 206004901 | PARD3B |
| 2 | 206004851 | 206004951 | PARD3B |
| 2 | 232186801 | 232186901 | ARMC9 |
| 2 | 232186851 | 232186951 | ARMC9 |
| 2 | 232186901 | 232187001 | ARMC9 |
| 2 | 232186951 | 232187051 | ARMC9 |
| 2 | 236774051 | 236774151 | AGAP1 |
| 2 | 237623801 | 237623901 | n/a |
| 2 | 241504751 | 241504851 | n/a |
| 2 | 242048301 | 242048401 | PASK |
| 2 | 242048351 | 242048451 | PASK |
| 2 | 242785201 | 242785301 | n/a |
| 2 | 242908201 | 242908301 | LINC01237 |
| 20 | 31123201 | 31123301 | NOL4L |
| 20 | 31123251 | 31123351 | NOL4L |
| 20 | 39127001 | 39127101 | n/a |
| 22 | 23923201 | 23923301 | IGLL1 |
| 22 | 45575251 | 45575351 | NUP50 |
| 22 | 50618551 | 50618651 | PANX2 |
| 22 | 50618601 | 50618701 | PANX2 |
| 3 | 32993101 | 32993201 | CCR4 |
| 3 | 32993151 | 32993251 | CCR4 |
| 3 | 46448551 | 46448651 | CCRL2 |
| 3 | 46448751 | 46448851 | CCRL2 |
| 3 | 46448801 | 46448901 | LOC102724297 |
| 3 | 53700101 | 53700201 | CACNA1D |
| 3 | 72227101 | 72227201 | n/a |
| 3 | 72227251 | 72227351 | n/a |
| 3 | 73620851 | 73620951 | PDZRN3 |
| 3 | 121796551 | 121796651 | CD86 |
| 3 | 123063401 | 123063501 | ADCY5 |
| 3 | 160475201 | 160475301 | PPM1L |
| 3 | 160475251 | 160475351 | PPM1L |
| 3 | 167659101 | 167659201 | n/a |
| 3 | 167659151 | 167659251 | n/a |
| 3 | 177397701 | 177397801 | LINC00578 |
| 3 | 177397751 | 177397851 | LINC00578 |
| 3 | 184504551 | 184504651 | n/a |
| 3 | 190363701 | 190363801 | IL1RAP |
| 3 | 194868651 | 194868751 | XXYLT1-AS2 |
| 3 | 194868701 | 194868801 | XXYLT1-AS2 |
| 3 | 194868751 | 194868851 | XXYLT1-AS2 |
| 4 | 1221751 | 1221851 | CTBP1 |
| 4 | 1221801 | 1221901 | CTBP1 |
| 4 | 1742401 | 1742501 | TACC3 |
| 4 | 13544651 | 13544751 | NKX3-2 |
| 4 | 13544701 | 13544801 | NKX3-2 |
| 4 | 53862451 | 53862551 | SCFD2 |
| 4 | 53862501 | 53862601 | SCFD2 |
| 4 | 57522951 | 57523051 | HOPX |
| 4 | 74713351 | 74713451 | n/a |
| 4 | 77226301 | 77226401 | STBD1 |
| 4 | 77226351 | 77226451 | STBD1 |
| 4 | 101438751 | 101438851 | EMCN |
| 4 | 101438801 | 101438901 | EMCN |
| 4 | 183795701 | 183795801 | n/a |
| 4 | 183795751 | 183795851 | n/a |
| 4 | 184320501 | 184320601 | n/a |
| 4 | 186559651 | 186559751 | SORBS2 |
| 5 | 1107151 | 1107251 | SLC12A7 |
| 5 | 10445501 | 10445601 | ROPN1L |
| 5 | 10445551 | 10445651 | ROPN1L |
| 5 | 14331751 | 14331851 | TRIO |
| 5 | 14331801 | 14331901 | TRIO |
| 5 | 14676401 | 14676501 | OTULIN |
| 5 | 31470851 | 31470951 | DROSHA |
| 5 | 32734901 | 32735001 | NPR3 |
| 5 | 32734951 | 32735051 | NPR3 |
| 5 | 37834151 | 37834251 | GDNF |
| 5 | 80050751 | 80050851 | MSH3 |
| 5 | 80050801 | 80050901 | MSH3 |
| 5 | 92931551 | 92931651 | MIR548AO |
| 5 | 92931901 | 92932001 | MIR548AO |
| 5 | 134826301 | 134826401 | n/a |
| 5 | 134826351 | 134826451 | n/a |
| 5 | 135266751 | 135266851 | FBXL21 |
| 5 | 135266801 | 135266901 | FBXL21 |
| 5 | 138714601 | 138714701 | SLC23A1 |
| 5 | 138714651 | 138714751 | SLC23A1 |
| 5 | 150593601 | 150593701 | CCDC69 |
| 5 | 150593651 | 150593751 | CCDC69 |
| 5 | 176758401 | 176758501 | n/a |
| 5 | 176758451 | 176758551 | n/a |
| 5 | 179344551 | 179344651 | n/a |
| 5 | 179344601 | 179344701 | n/a |
| 6 | 2733751 | 2733851 | MYLK4 |
| 6 | 10393751 | 10393851 | n/a |
| 6 | 10425551 | 10425651 | n/a |
| 6 | 34252751 | 34252851 | n/a |
| 6 | 34252801 | 34252901 | n/a |
| 6 | 36209701 | 36209801 | n/a |
| 6 | 41394751 | 41394851 | n/a |
| 6 | 41394801 | 41394901 | n/a |
| 6 | 41394851 | 41394951 | n/a |
| 6 | 45296051 | 45296151 | RUNX2 |
| 6 | 45296101 | 45296201 | SUPT3H |
| 6 | 135516851 | 135516951 | MYB |
| 6 | 135516901 | 135517001 | MYB |
| 6 | 157184151 | 157184251 | ARID1B |
| 6 | 168107101 | 168107201 | n/a |
| 6 | 170531301 | 170531401 | n/a |
| 6 | 170531351 | 170531451 | n/a |
| 7 | 5518851 | 5518951 | FBXL18 |
| 7 | 5518901 | 5519001 | FBXL18 |
| 7 | 5518951 | 5519051 | FBXL18 |
| 7 | 6475951 | 6476051 | DAGLB |
| 7 | 6476001 | 6476101 | DAGLB |
| 7 | 6476051 | 6476151 | DAGLB |
| 7 | 6476101 | 6476201 | DAGLB |
| 7 | 27281401 | 27281501 | EVX1 |
| 7 | 27289151 | 27289251 | n/a |
| 7 | 75957101 | 75957201 | YWHAG |
| 7 | 96631701 | 96631801 | DLX6-AS1 |
| 7 | 96631751 | 96631851 | DLX6-AS1 |
| 7 | 96650001 | 96650101 | DLX5 |
| 7 | 96650051 | 96650151 | DLX5 |
| 7 | 105662751 | 105662851 | CDHR3 |
| 7 | 128579851 | 128579951 | IRF5 |
| 7 | 129411001 | 129411101 | MIR182 |
| 7 | 129411051 | 129411151 | MIR182 |
| 7 | 129411101 | 129411201 | MIR182 |
| 7 | 129411151 | 129411251 | MIR182 |
| 8 | 76318451 | 76318551 | n/a |
| 8 | 99950851 | 99950951 | STK3 |
| 8 | 99950901 | 99951001 | STK3 |
| 8 | 102149801 | 102149901 | n/a |
| 8 | 117487001 | 117487101 | n/a |
| 8 | 134072501 | 134072601 | SLA |
| 8 | 140945801 | 140945901 | TRAPPC9 |
| 8 | 141584651 | 141584751 | AGO2 |
| 8 | 141584701 | 141584801 | AGO2 |
| 8 | 144408451 | 144408551 | TOP1MT |
| 8 | 144408501 | 144408601 | TOP1MT |
| 8 | 144408551 | 144408651 | TOP1MT |
| 9 | 91006601 | 91006701 | SPIN1 |
| 9 | 91006651 | 91006751 | SPIN1 |
| 9 | 91006701 | 91006801 | SPIN1 |
| 9 | 91006751 | 91006851 | SPIN1 |
| 9 | 96080251 | 96080351 | WNK2 |
| 9 | 96080301 | 96080401 | WNK2 |
| 9 | 96080351 | 96080451 | WNK2 |
| 9 | 98790151 | 98790251 | n/a |
| 9 | 101876601 | 101876701 | TGFBR1 |
| 9 | 101876651 | 101876751 | TGFBR1 |
| 9 | 110399201 | 110399301 | n/a |
| 9 | 110399251 | 110399351 | n/a |
| 9 | 124045101 | 124045201 | GSN |
| 9 | 125796751 | 125796851 | RABGAP1 |
| 9 | 125796801 | 125796901 | GPR21 |
| 9 | 125797101 | 125797201 | GPR21 |
| 9 | 125797151 | 125797251 | RABGAP1 |
| 9 | 132650501 | 132650601 | FNBP1 |
| 9 | 132650551 | 132650651 | FNBP1 |
| 9 | 132650601 | 132650701 | FNBP1 |
| 9 | 132650651 | 132650751 | FNBP1 |
| 9 | 134151501 | 134151601 | FAM78A |
| 9 | 140586151 | 140586251 | EHMT1 |
| 9 | 140586201 | 140586301 | EHMT1 |

TABLE 3
Hypomethylated region, prostate tissue specific genomic locations

| Chromosome | start | end | gene |
|---|---|---|---|
| 1 | 2839151 | 2839251 | n/a |
| 1 | 2876451 | 2876551 | n/a |
| 1 | 2876501 | 2876601 | n/a |
| 1 | 2876551 | 2876651 | n/a |
| 1 | 43637151 | 43637251 | EBNA1BP2 |
| 1 | 95172951 | 95173051 | LINC01057 |
| 1 | 95173001 | 95173101 | LINC01057 |
| 1 | 95173051 | 95173151 | LINC01057 |
| 1 | 110676801 | 110676901 | n/a |
| 1 | 155904851 | 155904951 | KIAA0907 |
| 1 | 158465951 | 158466051 | n/a |
| 1 | 160079651 | 160079751 | n/a |
| 1 | 162527401 | 162527501 | n/a |
| 1 | 162527451 | 162527551 | n/a |
| 1 | 169696601 | 169696701 | SELE |
| 1 | 175490501 | 175490601 | TNR |
| 1 | 203829801 | 203829901 | SNRPE |
| 1 | 204165251 | 204165351 | KISS1 |
| 1 | 204349851 | 204349951 | n/a |
| 1 | 248153651 | 248153751 | OR2L1P |
| 10 | 6779901 | 6780001 | n/a |
| 10 | 6779951 | 6780051 | n/a |
| 10 | 126713301 | 126713401 | CTBP2 |
| 10 | 126713351 | 126713451 | CTBP2 |
| 11 | 19681701 | 19681801 | n/aV2 |
| 11 | 19681751 | 19681851 | n/aV2 |
| 11 | 27536001 | 27536101 | BDNF-AS |
| 11 | 27536051 | 27536151 | MIR8087 |
| 11 | 57519401 | 57519501 | BTBD18 |
| 11 | 60680451 | 60680551 | TMEM109 |
| 11 | 67615951 | 67616051 | n/a |
| 11 | 76371951 | 76372051 | LRRC32 |
| 11 | 78900901 | 78901001 | TENM4 |
| 11 | 78900951 | 78901051 | TENM4 |
| 11 | 88019001 | 88019101 | n/a |
| 11 | 88019051 | 88019151 | n/a |
| 11 | 128737201 | 128737301 | KCNJ1 |
| 11 | 132912251 | 132912351 | OPCML |
| 11 | 133445651 | 133445751 | n/a |
| 12 | 1702101 | 1702201 | FBXL14 |
| 12 | 4029951 | 4030051 | n/a |
| 12 | 4030001 | 4030101 | n/a |
| 12 | 4030051 | 4030151 | n/a |
| 12 | 5156351 | 5156451 | n/a |
| 12 | 8438301 | 8438401 | n/a |
| 12 | 8438351 | 8438451 | n/a |
| 12 | 8438401 | 8438501 | n/a |
| 12 | 130711351 | 130711451 | n/a |
| 12 | 131941401 | 131941501 | n/a |
| 12 | 132848201 | 132848301 | GALNT9 |
| 12 | 132848251 | 132848351 | GALNT9 |
| 12 | 132848301 | 132848401 | GALNT9 |
| 13 | 112906801 | 112906901 | n/a |
| 14 | 52219001 | 52219101 | n/a |
| 14 | 52219051 | 52219151 | n/a |
| 14 | 52219101 | 52219201 | n/a |
| 14 | 59296551 | 59296651 | LINC01500 |
| 14 | 59296601 | 59296701 | LINC01500 |
| 14 | 59296651 | 59296751 | LINC01500 |
| 14 | 93412701 | 93412801 | ITPK1 |
| 14 | 97497051 | 97497151 | n/a |
| 14 | 100046451 | 100046551 | CCDC85C |
| 14 | 104742051 | 104742151 | n/a |
| 14 | 104742101 | 104742201 | n/a |
| 14 | 104742151 | 104742251 | n/a |
| 14 | 104889001 | 104889101 | n/a |
| 14 | 104889051 | 104889151 | n/a |
| 15 | 22799001 | 22799101 | n/a |
| 15 | 22799051 | 22799151 | n/a |
| 15 | 28051101 | 28051201 | OCA2 |
| 16 | 23988951 | 23989051 | PRKCB |
| 16 | 86457501 | 86457601 | n/a |
| 16 | 88218201 | 88218301 | n/a |
| 17 | 39472101 | 39472201 | KRTAP17-1 |
| 17 | 66951501 | 66951601 | ABCA8 |
| 17 | 79694451 | 79694551 | n/a |
| 17 | 79694501 | 79694601 | n/a |
| 19 | 15901151 | 15901251 | n/a |
| 19 | 16178551 | 16178651 | TPM4 |
| 19 | 54177201 | 54177301 | MIR498 |
| 19 | 54177251 | 54177351 | MIR498 |
| 19 | 54778401 | 54778501 | LILRB2 |
| 19 | 55104551 | 55104651 | LILRA1 |
| 19 | 55104601 | 55104701 | LILRA1 |
| 2 | 879401 | 879501 | n/a |
| 2 | 879451 | 879551 | n/a |
| 2 | 879501 | 879601 | n/a |
| 2 | 2581201 | 2581301 | n/a |
| 2 | 2581251 | 2581351 | n/a |
| 2 | 2581301 | 2581401 | n/a |
| 2 | 59477151 | 59477251 | LOC101927285 |
| 2 | 74153201 | 74153301 | DGUOK |
| 2 | 100426901 | 100427001 | AFF3 |
| 2 | 100426951 | 100427051 | AFF3 |
| 2 | 107456801 | 107456901 | ST6GAL2 |
| 2 | 147788651 | 147788751 | n/a |
| 2 | 208795601 | 208795701 | PLEKHM3 |
| 2 | 208795651 | 208795751 | PLEKHM3 |
| 2 | 208795701 | 208795801 | PLEKHM3 |
| 2 | 232455551 | 232455651 | n/a |
| 2 | 232455601 | 232455701 | n/a |
| 20 | 1975251 | 1975351 | PDYN |
| 20 | 1975301 | 1975401 | PDYN |
| 20 | 1975351 | 1975451 | PDYN |
| 20 | 1975401 | 1975501 | PDYN |
| 20 | 19866651 | 19866751 | RIN2 |
| 20 | 19866701 | 19866801 | RIN2 |
| 20 | 62111301 | 62111401 | n/a |
| 21 | 43735451 | 43735551 | TFF3 |
| 21 | 43735501 | 43735601 | TFF3 |
| 21 | 43735551 | 43735651 | TFF3 |
| 21 | 44375551 | 44375651 | n/a |
| 22 | 24979501 | 24979601 | GGT1 |
| 22 | 24979551 | 24979651 | GGT1 |
| 22 | 24979601 | 24979701 | GGT1 |
| 22 | 49020401 | 49020501 | FAM19A5 |
| 22 | 49800101 | 49800201 | n/a |
| 22 | 49800151 | 49800251 | n/a |
| 22 | 50481601 | 50481701 | n/a |
| 3 | 29494901 | 29495001 | RBMS3 |
| 3 | 29494951 | 29495051 | RBMS3 |
| 3 | 33757901 | 33758001 | CLASP2 |
| 3 | 36360601 | 36360701 | n/a |
| 3 | 36360651 | 36360751 | n/a |
| 4 | 1047001 | 1047101 | n/a |
| 4 | 1047051 | 1047151 | n/a |
| 4 | 3895101 | 3895201 | n/a |
| 4 | 3895151 | 3895251 | n/a |
| 4 | 5368251 | 5368351 | STK32B |
| 4 | 5368301 | 5368401 | STK32B |
| 4 | 5526701 | 5526801 | LINC01587 |
| 4 | 9104401 | 9104501 | n/a |
| 4 | 9104451 | 9104551 | n/a |
| 4 | 9104551 | 9104651 | n/a |
| 4 | 16708251 | 16708351 | LDB2 |
| 4 | 16708301 | 16708401 | LDB2 |
| 4 | 79971101 | 79971201 | LINC01088 |
| 4 | 79971151 | 79971251 | LINC01088 |
| 4 | 79971201 | 79971301 | LINC01088 |
| 4 | 100576551 | 100576651 | n/a |
| 4 | 100576601 | 100576701 | n/a |
| 4 | 120502101 | 120502201 | PDE5A |
| 4 | 190283101 | 190283201 | n/a |
| 5 | 759101 | 759201 | n/a |
| 5 | 3188351 | 3188451 | n/a |
| 5 | 19531501 | 19531601 | CDH18 |
| 5 | 19531551 | 19531651 | CDH18 |
| 5 | 171808201 | 171808301 | SH3PXD2B |
| 5 | 171808251 | 171808351 | SH3PXD2B |
| 5 | 178594601 | 178594701 | ADAMTS2 |
| 6 | 87830101 | 87830201 | n/a |
| 6 | 87830151 | 87830251 | n/a |
| 6 | 133689901 | 133690001 | EYA4 |
| 6 | 152804701 | 152804801 | SYNE1 |
| 6 | 152804751 | 152804851 | SYNE1 |
| 6 | 159872201 | 159872301 | n/a |
| 6 | 159872251 | 159872351 | n/a |
| 7 | 39056301 | 39056401 | POU6F2 |
| 7 | 39056351 | 39056451 | POU6F2 |
| 7 | 39056401 | 39056501 | POU6F2 |
| 7 | 158059651 | 158059751 | PTPRN2 |
| 7 | 158059701 | 158059801 | PTPRN2 |
| 7 | 158059901 | 158060001 | PTPRN2 |
| 8 | 49984651 | 49984751 | C8orf22 |
| 8 | 49984701 | 49984801 | C8orf22 |
| 8 | 49984751 | 49984851 | C8orf22 |
| 8 | 52754401 | 52754501 | PCMTD1 |
| 8 | 105988201 | 105988301 | n/a |
| 8 | 119073651 | 119073751 | EXT1 |
| 8 | 119073701 | 119073801 | EXT1 |
| 8 | 120779851 | 120779951 | TAF2 |
| 8 | 130365251 | 130365351 | CCDC26 |
| 8 | 130365301 | 130365401 | CCDC26 |
| 8 | 133573401 | 133573501 | HPYR1 |
| 8 | 139124351 | 139124451 | n/a |
| 8 | 139124401 | 139124501 | n/a |
| 8 | 139124451 | 139124551 | n/a |
| 8 | 139784451 | 139784551 | COL22A1 |
| 8 | 142289701 | 142289801 | n/a |
| 8 | 142289751 | 142289851 | n/a |
| 8 | 142289801 | 142289901 | n/a |
| 8 | 142289851 | 142289951 | n/a |
| 9 | 5756751 | 5756851 | RIC1 |
| 9 | 38437251 | 38437351 | n/a |
| 9 | 38437301 | 38437401 | n/a |
| 9 | 92291201 | 92291301 | UNQ6494 |
| 9 | 92291251 | 92291351 | UNQ6494 |
| 9 | 128307501 | 128307601 | MAPKAP1 |
| 9 | 128307551 | 128307651 | MAPKAP1 |
| 9 | 138192201 | 138192301 | n/a |
| 9 | 138192251 | 138192351 | n/a |
| X | 47662501 | 47662601 | n/a |
| X | 47662551 | 47662651 | n/a |
| X | 52683851 | 52683951 | SSX7 |

TABLE 4
Hypomethylated region, prostate cancer specific genomic locations

| Chromosome | start | end | gene |
|---|---|---|---|
| 1 | 2013951 | 2014051 | PRKCZ |
| 1 | 4079101 | 4079201 | n/a |
| 1 | 143907551 | 143907651 | FAM72C |
| 1 | 148903551 | 148903651 | NBPF25P |
| 1 | 152648651 | 152648751 | LCE2C |
| 1 | 152648701 | 152648801 | LCE2C |
| 1 | 153174651 | 153174751 | n/a |
| 1 | 153174701 | 153174801 | n/a |
| 1 | 153174751 | 153174851 | n/a |
| 1 | 153175201 | 153175301 | LELP1 |
| 1 | 153175251 | 153175351 | LELP1 |
| 1 | 153283751 | 153283851 | PGLYRP3 |
| 1 | 153283801 | 153283901 | PGLYRP3 |
| 1 | 153352051 | 153352151 | n/a |
| 1 | 153352101 | 153352201 | n/a |
| 1 | 153353201 | 153353301 | n/a |
| 1 | 153389951 | 153390051 | S100A7A |
| 1 | 158465801 | 158465901 | n/a |
| 1 | 158465851 | 158465951 | n/a |
| 1 | 159236001 | 159236101 | n/a |
| 1 | 159236051 | 159236151 | n/a |
| 1 | 175490551 | 175490651 | TNR |
| 1 | 175490601 | 175490701 | TNR |
| 1 | 182021701 | 182021801 | n/a |
| 1 | 182021751 | 182021851 | n/a |
| 1 | 209105801 | 209105901 | n/a |
| 1 | 248308901 | 248309001 | OR2M5 |
| 1 | 248366251 | 248366351 | OR2M3 |
| 10 | 2699351 | 2699451 | n/a |
| 10 | 6664951 | 6665051 | LOC101928150 |
| 10 | 6665001 | 6665101 | LOC101928150 |
| 10 | 6807101 | 6807201 | n/a |
| 10 | 7567801 | 7567901 | n/a |
| 10 | 7567851 | 7567951 | n/a |
| 10 | 26226851 | 26226951 | MYO3A |
| 10 | 26226901 | 26227001 | MYO3A |
| 10 | 26226951 | 26227051 | MYO3A |
| 11 | 5957801 | 5957901 | n/a |
| 11 | 6865801 | 6865901 | n/a |
| 11 | 7961051 | 7961151 | OR10A3 |
| 11 | 7961101 | 7961201 | OR10A3 |
| 11 | 22219001 | 22219101 | ANO5 |
| 11 | 50220151 | 50220251 | n/a |
| 11 | 55579301 | 55579401 | OR5L1 |
| 11 | 59949051 | 59949151 | MS4A6A |
| 11 | 121762851 | 121762951 | n/a |
| 11 | 121762901 | 121763001 | n/a |
| 11 | 121986951 | 121987051 | MIR100HG |
| 11 | 122100851 | 122100951 | n/a |
| 11 | 123900601 | 123900701 | OR10G8 |
| 12 | 3053501 | 3053601 | n/a |
| 12 | 4361001 | 4361101 | CCND2-AS1 |
| 12 | 4361051 | 4361151 | CCND2-AS1 |
| 12 | 4361101 | 4361201 | CCND2-AS1 |
| 12 | 124397801 | 124397901 | DNAH10 |
| 12 | 124397851 | 124397951 | DNAH10 |
| 12 | 127348201 | 127348301 | n/a |
| 12 | 127348251 | 127348351 | n/a |
| 12 | 127348301 | 127348401 | n/a |
| 12 | 127944451 | 127944551 | n/a |
| 12 | 127980601 | 127980701 | n/a |
| 12 | 127980651 | 127980751 | n/a |
| 12 | 128869901 | 128870001 | TMEM132C |
| 12 | 129595351 | 129595451 | TMEM132D |
| 12 | 130411151 | 130411251 | n/a |
| 12 | 130411201 | 130411301 | n/a |
| 12 | 130411251 | 130411351 | n/a |
| 12 | 130494601 | 130494701 | n/a |
| 12 | 130591301 | 130591401 | n/a |
| 12 | 130683451 | 130683551 | n/a |
| 12 | 130750301 | 130750401 | n/a |
| 12 | 131402501 | 131402601 | n/a |
| 12 | 131402551 | 131402651 | n/a |
| 12 | 131418151 | 131418251 | n/a |
| 12 | 131512551 | 131512651 | ADGRD1 |
| 12 | 131512601 | 131512701 | ADGRD1 |
| 12 | 131769201 | 131769301 | n/a |
| 12 | 131769251 | 131769351 | n/a |
| 12 | 131862601 | 131862701 | n/a |
| 12 | 131941451 | 131941551 | n/a |
| 12 | 132102101 | 132102201 | n/a |
| 12 | 132142001 | 132142101 | n/a |
| 12 | 132142051 | 132142151 | n/a |
| 12 | 132663851 | 132663951 | n/a |
| 12 | 132663901 | 132664001 | n/a |
| 14 | 22315001 | 22315101 | n/a |
| 14 | 47669951 | 47670051 | MDGA2 |
| 14 | 47670001 | 47670101 | MDGA2 |
| 14 | 47670051 | 47670151 | MDGA2 |
| 14 | 47670101 | 47670201 | MDGA2 |
| 14 | 47670151 | 47670251 | MDGA2 |
| 14 | 47670201 | 47670301 | MDGA2 |
| 14 | 47670251 | 47670351 | MDGA2 |
| 14 | 97497101 | 97497201 | n/a |
| 14 | 97853651 | 97853751 | n/a |
| 14 | 97853701 | 97853801 | n/a |
| 14 | 97924251 | 97924351 | LOC101929241 |
| 14 | 97924301 | 97924401 | LOC101929241 |
| 14 | 97924401 | 97924501 | LOC101929241 |
| 14 | 97924451 | 97924551 | LOC101929241 |
| 14 | 97924501 | 97924601 | LOC101929241 |
| 14 | 98101651 | 98101751 | LOC100129345 |
| 14 | 98101701 | 98101801 | LOC100129345 |
| 14 | 99181901 | 99182001 | C14orf177 |
| 14 | 101495751 | 101495851 | MIR494 |
| 14 | 101495801 | 101495901 | MIR494 |
| 15 | 95287801 | 95287901 | n/a |
| 15 | 95287851 | 95287951 | n/a |
| 15 | 95287901 | 95288001 | n/a |
| 15 | 98646401 | 98646501 | n/a |
| 16 | 8337501 | 8337601 | n/a |
| 16 | 8337551 | 8337651 | n/a |
| 16 | 9855201 | 9855301 | GRIN2A |
| 16 | 9855251 | 9855351 | GRIN2A |
| 16 | 9857601 | 9857701 | GRIN2A |
| 16 | 10206551 | 10206651 | GRIN2A |
| 16 | 10271751 | 10271851 | GRIN2A |
| 16 | 10272701 | 10272801 | GRIN2A |
| 16 | 10272751 | 10272851 | GRIN2A |
| 16 | 23938951 | 23939051 | PRKCB |
| 16 | 23939001 | 23939101 | PRKCB |
| 16 | 23939051 | 23939151 | PRKCB |
| 16 | 24151201 | 24151301 | PRKCB |
| 16 | 24151251 | 24151351 | PRKCB |
| 16 | 24266251 | 24266351 | CACNG3 |
| 16 | 24266301 | 24266401 | CACNG3 |
| 16 | 29322201 | 29322301 | SNX29P2 |
| 16 | 29322251 | 29322351 | SNX29P2 |
| 16 | 32488401 | 32488501 | n/a |
| 16 | 46391151 | 46391251 | n/a |
| 16 | 65102901 | 65103001 | CDH11 |
| 16 | 86327651 | 86327751 | n/a |
| 16 | 86421751 | 86421851 | n/a |
| 16 | 86666101 | 86666201 | n/a |
| 16 | 87645651 | 87645751 | JPH3 |
| 17 | 3030101 | 3030201 | OR1G1 |
| 17 | 3030151 | 3030251 | OR1G1 |
| 17 | 21911401 | 21911501 | FLI36000 |
| 17 | 21911451 | 21911551 | FLI36000 |
| 17 | 22016951 | 22017051 | n/a |
| 17 | 22017001 | 22017101 | n/a |
| 17 | 22023751 | 22023851 | MTRNR2L1 |
| 17 | 77386201 | 77386301 | RBFOX3 |
| 17 | 77386351 | 77386451 | RBFOX3 |
| 17 | 77386401 | 77386501 | RBFOX3 |
| 17 | 77390001 | 77390101 | RBFOX3 |
| 18 | 5392951 | 5393051 | EPB41L3 |
| 18 | 5393001 | 5393101 | EPB41L3 |
| 18 | 11153851 | 11153951 | n/a |
| 18 | 11153901 | 11154001 | n/a |
| 19 | 2715051 | 2715151 | DIRAS1 |
| 19 | 15067451 | 15067551 | SLC1A6 |
| 19 | 29281801 | 29281901 | n/a |
| 19 | 29281851 | 29281951 | n/a |
| 19 | 29281901 | 29282001 | n/a |
| 19 | 43271101 | 43271201 | n/a |
| 19 | 54568251 | 54568351 | n/a |
| 19 | 54903901 | 54904001 | n/a |
| 19 | 54903951 | 54904051 | n/a |
| 19 | 55036651 | 55036751 | n/a |
| 19 | 55042251 | 55042351 | n/a |
| 19 | 55042301 | 55042401 | n/a |
| 19 | 55692651 | 55692751 | PTPRH |
| 19 | 55692701 | 55692801 | PTPRH |
| 19 | 56274351 | 56274451 | RFPL4A |
| 19 | 56274401 | 56274501 | RFPL4A |
| 19 | 56346551 | 56346651 | NLRP11 |
| 19 | 56346601 | 56346701 | NLRP11 |
| 19 | 57646101 | 57646201 | ZIM3 |
| 2 | 3633351 | 3633451 | n/a |
| 2 | 3633401 | 3633501 | n/a |
| 2 | 44513801 | 44513901 | SLC3A1 |
| 2 | 44513851 | 44513951 | SLC3A1 |
| 2 | 59470151 | 59470251 | LOC101927285 |
| 2 | 59470201 | 59470301 | LOC101927285 |
| 2 | 60880201 | 60880301 | n/a |
| 2 | 89215101 | 89215201 | n/a |
| 2 | 89215151 | 89215251 | n/a |
| 2 | 91910551 | 91910651 | n/a |
| 2 | 91910601 | 91910701 | n/a |
| 2 | 91936151 | 91936251 | n/a |
| 2 | 117006251 | 117006351 | n/a |
| 2 | 119134001 | 119134101 | n/a |
| 2 | 119134051 | 119134151 | n/a |
| 2 | 119471301 | 119471401 | n/a |
| 2 | 127401201 | 127401301 | n/a |
| 2 | 127529551 | 127529651 | n/a |
| 2 | 127529601 | 127529701 | n/a |
| 2 | 203636951 | 203637051 | n/a |
| 2 | 228336101 | 228336201 | MIR5703 |
| 2 | 242190801 | 242190901 | HDLBP |
| 20 | 5282851 | 5282951 | PROKR2 |
| 20 | 5282901 | 5283001 | PROKR2 |
| 20 | 5284401 | 5284501 | PROKR2 |
| 20 | 5450901 | 5451001 | LOC643406 |
| 20 | 44876301 | 44876401 | CDH22 |
| 20 | 59543701 | 59543801 | n/a |
| 20 | 59543751 | 59543851 | n/a |
| 20 | 59888501 | 59888601 | CDH4 |
| 20 | 59888551 | 59888651 | CDH4 |
| 20 | 59888601 | 59888701 | CDH4 |
| 20 | 61715801 | 61715901 | LOC63930 |
| 20 | 61754951 | 61755051 | n/a |
| 20 | 61755001 | 61755101 | n/a |
| 22 | 17073551 | 17073651 | CCT8L2 |
| 22 | 22902051 | 22902151 | PRAME |
| 22 | 49635751 | 49635851 | n/a |
| 22 | 50482001 | 50482101 | n/a |
| 3 | 13860351 | 13860451 | WNT7A |
| 3 | 38835151 | 38835251 | SCN10A |
| 3 | 38835201 | 38835301 | SCN10A |
| 3 | 38835251 | 38835351 | SCN10A |
| 3 | 46245401 | 46245501 | CCR1 |
| 3 | 100690851 | 100690951 | ABI3BP |
| 3 | 100690901 | 100691001 | ABI3BP |
| 3 | 192769601 | 192769701 | n/a |
| 3 | 192960401 | 192960501 | MGC2889 |
| 3 | 192960451 | 192960551 | MGC2889 |
| 3 | 192960551 | 192960651 | MGC2889 |
| 3 | 192973501 | 192973601 | HRASLS |
| 3 | 193096401 | 193096501 | ATP13A5 |
| 3 | 193097751 | 193097851 | n/a |
| 4 | 9453051 | 9453151 | n/a |
| 4 | 9523701 | 9523801 | n/a |
| 4 | 157059251 | 157059351 | n/a |
| 4 | 157059301 | 157059401 | n/a |
| 4 | 157059351 | 157059451 | n/a |
| 4 | 190462551 | 190462651 | n/a |
| 4 | 190751051 | 190751151 | n/a |
| 5 | 471201 | 471301 | PP7080 |
| 5 | 2866801 | 2866901 | n/a |
| 5 | 3002401 | 3002501 | n/a |
| 5 | 3002451 | 3002551 | n/a |
| 5 | 3339501 | 3339601 | n/a |
| 5 | 3454101 | 3454201 | LINC01019 |
| 5 | 3454151 | 3454251 | LINC01019 |
| 5 | 4116251 | 4116351 | n/a |
| 5 | 5492351 | 5492451 | n/a |
| 5 | 152949001 | 152949101 | GRIA1 |
| 5 | 153039151 | 153039251 | GRIA1 |
| 5 | 153039201 | 153039301 | GRIA1 |
| 6 | 133932801 | 133932901 | TARID |
| 6 | 133932851 | 133932951 | TARID |
| 6 | 153066801 | 153066901 | n/a |
| 6 | 154330951 | 154331051 | OPRM1 |
| 6 | 155777801 | 155777901 | NOX3 |
| 7 | 57218251 | 57218351 | n/a |
| 7 | 57247701 | 57247801 | GUSBP10 |
| 7 | 57324901 | 57325001 | n/a |
| 7 | 57509951 | 57510051 | ZNF716 |
| 7 | 57510151 | 57510251 | ZNF716 |
| 7 | 57510201 | 57510301 | ZNF716 |
| 7 | 57714301 | 57714401 | n/a |
| 7 | 127256601 | 127256701 | PAX4 |
| 7 | 144967601 | 144967701 | n/a |
| 8 | 32732251 | 32732351 | n/a |
| 8 | 56106751 | 56106851 | XKR4 |
| 8 | 56361951 | 56362051 | SBF1P1 |
| 8 | 64314301 | 64314401 | n/a |
| 8 | 67085751 | 67085851 | TRIM55 |
| 8 | 73849101 | 73849201 | KCNB2 |
| 8 | 73849151 | 73849251 | KCNB2 |
| 8 | 105988151 | 105988251 | n/a |
| 8 | 107139701 | 107139801 | n/a |
| 8 | 107139751 | 107139851 | n/a |
| 8 | 111906551 | 111906651 | n/a |
| 8 | 114390751 | 114390851 | CSMD3 |
| 8 | 139784401 | 139784501 | COL22A1 |
| 8 | 140624701 | 140624801 | KCNK9 |
| 9 | 27949501 | 27949601 | LINGO2 |
| X | 150944951 | 150945051 | n/a |
| X | 150945001 | 150945101 | n/a |

In Tables 1 to 4, where the gene indicated is “n/a” this means that the genomic location defined in the table is a non-coding region of DNA or not within the location of a known gene. In certain embodiments, the set of genomic locations listed in Table 1 does not include the genomic locations listed in Table 1b below:

TABLE 1b
Genomic locations that may be excluded from Table 1

| Chromosome | start | end | gene |
|---|---|---|---|
| 2 | 26521751 | 26521851 | n/a |
| 2 | 63282651 | 63282751 | OTX1 |
| 2 | 63283901 | 63284001 | OTX1 |
| 2 | 201450501 | 201450601 | AOX1 |
| 2 | 201450551 | 201450651 | AOX1 |
| 2 | 201450601 | 201450701 | AOX1 |
| 2 | 201450651 | 201450751 | AOX1 |
| 3 | 33701201 | 33701301 | CLASP2 |
| 3 | 170746251 | 170746351 | n/a |
| 4 | 20256801 | 20256901 | SLIT2 |
| 4 | 54959951 | 54960051 | n/a |
| 4 | 54960001 | 54960101 | n/a |
| 4 | 74809851 | 74809951 | n/a |
| 4 | 87281351 | 87281451 | MAPK10 |
| 4 | 87281401 | 87281501 | MAPK10 |
| 5 | 134880301 | 134880401 | n/a |
| 5 | 170735101 | 170735201 | n/a |
| 5 | 172673051 | 172673151 | n/a |
| 6 | 27858551 | 27858651 | HIST1H3J |
| 6 | 139795501 | 139795601 | LINC01625 |
| 7 | 129425301 | 129425401 | n/a |
| 9 | 971451 | 971551 | n/a |
| 12 | 81471601 | 81471701 | ACSS3 |
| 12 | 81471651 | 81471751 | ACSS3 |
| 12 | 95941801 | 95941901 | USP44 |
| 17 | 80944051 | 80944151 | B3GNTL1 |
| 19 | 46917001 | 46917101 | CCDC8 |
| 19 | 46917051 | 46917151 | CCDC8 |
| 19 | 55592451 | 55592551 | EPS8L1 |

The method is for detecting, screening, monitoring, staging, classification, selecting treatment for, ascertaining whether treatment is working in, and/or prognostication of prostate cancer. The prostate cancer may be any type of prostate cancer. Suitably, it may be acinar adenocarcinoma prostate cancer, ductal adenocarcinoma prostate cancer, transitional cell cancer of the prostate, squamous cell cancer of the prostate, or small cell prostate cancer. For example, it may be acinar adenocarcinoma prostate cancer or ductal adenocarcinoma prostate cancer. Alternatively, or additionally, the prostate cancer may be castration sensitive prostate cancer or castration resistant prostate cancer. Alternatively, or additionally, the prostate cancer may be metastatic prostate cancer, or it may be non-metastatic prostate cancer. In certain embodiments, it may be metastatic prostate cancer. In certain embodiments, the prostate cancer may be metastatic castration resistant prostate cancer or non-metastatic castration resistant prostate cancer. For example, it may be metastatic castration resistant prostate cancer.

The method is especially suitable for the detecting, screening, monitoring, staging, classification, selecting treatment for, ascertaining whether treatment is working in, and/or prognostication of metastatic prostate cancer.

The method is also especially suitable for the detecting, screening, monitoring, staging, classification, selecting treatment for, ascertaining whether treatment is working in, and/or prognostication of castration resistant prostate cancer prostate cancer.

The sample is a sample that comprises cfDNA. The sample may suitably be a blood sample, a plasma sample, or a urine sample. Preferably, the sample is a blood sample or a plasma sample. More preferably, the sample is a plasma sample.

The method may further comprise isolating the cfDNA from the sample. cfDNA can be isolated from the sample using a variety of techniques known in the art. For example, DNA (e.g., cfDNA) can be isolated by a column-based approach and/or a bead-based approach. In some embodiments, DNA (e.g., cfDNA) is isolated by means of a column-based approach, for example using a commercially available kit such as QIAamp circulating nucleic acid kit (Qiagen qiagen.com/ch/products/discovery-and-translational-research/dna-rna-purification/dna-purification/cell-free-dna/qiaamp-circulating-nucleic-acid-kit/#orderinginformation). In some embodiments, DNA (e.g., cfDNA) is isolated by means of a bead-based approach, for example an automated cf-DNA extraction system using a commercially available kit such as Maxwell RSC ccfDNA Plasma Kit (Promega (https://www.promega.co.uk/resources/protocols/technical-manuals/101/maxwell-rsc-ccfdna-plasma-kit-protocol/)).

The isolated cfDNA may be amplified before analysis. Thus the method may further comprise amplification of the isolated cfDNA. Amplification techniques are known to those of ordinary skill in the art and include, but are not limited to, cloning, polymerase chain reaction (PCR), polymerase chain reaction of specific alleles (PASA), polymerase chain ligation, nested polymerase chain reaction, and so forth.

The method comprises characterizing the methylome sequence of a plurality of cfDNA molecules in the sample, wherein the methylome sequence of a cfDNA molecule is the DNA sequence and the methylation profile of the molecule. The methylome sequence of a cfDNA molecule may be characterised by using methylation aware sequencing, by genome sequencing followed by methylation profiling, or by targeted approaches that capture specific DNA sequences (for example using DNA probes). Examples of methylation aware sequencing include bisulfite sequencing, bisulfite-free methylation-aware sequencing, methylation arrays (for example methylation microarrays), enzymatic methylation sequencing, methylation-sensitive restriction enzyme digestion, methylation-specific PCR, methylation aware PCR based assays, methylation-dependent DNA precipitation, and methylated DNA binding proteins/peptides. In certain embodiments, the methylome sequence of a plurality of cfDNA molecules in the sample is characterised using bisulfite sequencing, methylation microarrays, enzymatic methylation sequencing, bisulfite-free methylation-aware sequencing, or methylation aware PCR based assays.

Examples of targeted approaches that capture specific DNA sequences (for example using DNA probes) include cell-free methylated DNA immunoprecipitation and high-throughput sequencing (cfMeDIP-seq), methylation-dependent DNA precipitation, and methylated DNA binding proteins/peptides.

Bisulfite sequencing may comprise massive parallel sequencing with bisulfite conversion, for example treating the DNA molecule with sodium bisulfite and performing sequencing of the treated DNA molecule. Methylation assay sequencing may comprise treating the DNA molecule with sodium bisulfite, whole genome amplification, and hybridisation to a methylation-specific probe or a non-methylation probe, for example attached to a bead or chip.

Enzymatic methylation sequencing may comprise enzymatic treatment of the DNA molecule to convert methylated cytosine sites, followed by sequencing of the treated DNA. For example enzymatic methylation sequencing may comprise enzymatic treatment of the DNA molecule to convert methylated cytosine sites into a form protected from deamination, followed by deamination to convert unprotected cytosine to uracils, and sequencing of the treated DNA. An example of an enzymatic methylation sequencing kit includes NEBNext® Enzymatic Methyl-seq Kit (https://www.neb.com/products/e7120-nebnext-enzymatic-methyl-seq-kit#).

Examples of methylation aware PCR based assays include digital droplet PCR and qPCR (quantitative PCR).

An example of bisulfite-free methylation-aware sequencing is Oxford Nanopore seqeuencing (Oxford Nanopore Technologies, https://nanoporetech.com/))

In certain embodiments, the methylome sequence of a plurality of cfDNA molecules in the sample is characterised using whole genome bisulfite sequencing, for example low pass whole genome bisulfite sequencing. In another embodiment, the methylome sequence of a plurality of cfDNA molecules in the sample is characterised using reduced representation bisulfite treatments. In certain embodiments, the methylome sequence of a plurality of cfDNA molecules in the sample is characterised using methylation arrays, for example methylation microarrays, such as an Illumina Methylation Assay.

A variety of genome sequencing procedures are known in the art and may be used to practice the methods disclosed herein. For example, Sanger sequencing, Polony sequencing, 454 pyrosequencing, Combinatorial probe anchor synthesis, SOLiD sequencing, Ion Torrent semiconductor sequencing, DNA nanoball sequencing, Heliscope single molecule sequencing, Single molecule real time (SMRT) sequencing, Nanopore DNA sequencing, Microfluidic Sanger sequencing and Illumina dye sequencing.

A plurality of cfDNA molecules may be, for example, at least 100, at least 1000, at least 10,000, at least 50,000, at least 100,000, at least 500,000, at least 1,000,000 (10⁶), at least 5,000,000 (5×10⁶), at least 10,000,000 (10⁷), at least 100,000,000 (10⁸), or at least 1,000,000,000 (10⁹). Preferably, a plurality of cfDNA molecules may be, for example, at least 10,000, at least 50,000, at least 100,000, at least 500,000, at least 1,000,000 (10⁶), at least 5,000,000 (5×10⁶), at least 10,000,000 (10⁷), at least 100,000,000 (10⁸), or at least 1,000,000,000 (10⁹). More preferably, a plurality of cfDNA molecules may be, for example, at least 100,000, at least 500,000, at least 1,000,000 (10⁶), at least 5,000,000 (5×10⁶), at least 10,000,000 (10⁷), at least 100,000,000 (10⁸), or at least 1,000,000,000 (10⁹).

The method may further comprise aligning the methylome sequences with a reference genome for the subject, for example by aligning the methylome sequences with hg38, hg19, hg18, hg17 or hg16. The alignment can, for example, be carried out using a variety of techniques known in the art. For example, a DNA sequence alignment tool, (e.g., BSMAP (PMID: 19635165), Bismark (PMID: 21493656), gemBS (PMID: 30137223), Arioc (PMID: 29554207), BS-Seeker2 (PMID: 24206606), MethylCoder (PMID: 21724594) or BatMeth2 (PMID: 30669962)) can be used to align the reads to the reference genome (for example hg38, hg19, hg18, hg17 or hg16).

The genomic location assigned to each methylome sequence in the alignment is based on the reference genome adopted. The genomic locations listed in Tables 1, 1b, 2 to 9 disclosed herein correspond to reference genome hg19. The corresponding locations in a different reference genome can be found using public available tools known in the art. An example of these tools is LiftOver (http://genome.ucsc.edu/).

In certain embodiments, the method comprises removing duplications of reads of the same DNA molecule (i.e. duplications of reads of the same cfDNA molecule). In this step, sequence reads having exactly the same sequence and start and end base pairs (i.e. the same unclipped alignment start and unclipped alignment end of the sequence) are removed, as they are likely to be duplicate sequence reads of the same sequence (i.e. duplicate of reads of the same cfDNA molecule). For example, PCR duplications can be removed as part of the aligning step, such as using Picard tools v2.1.0 (http://broadinstitute.github.io/picard).

The method comprises determining the average methylation ratio at 10 or more of the genomic regions for which the average methylation ratio has been determined, each genomic region being selected from the group consisting of:
a 100 to 200 bp region comprising or having a genomic location defined in Tables 1 to 4, and
a 2 to 99 bp region within a genomic location defined in Tables 1 to 4 and comprising at least one CpG locus,
   and wherein each of the genomic regions is covered by at least one sequence read of at least one characterized methylome sequence.

In certain embodiments, each genomic region for which the average methylation ratio has been determined is covered by at least one sequence read of at least two characterized methylome sequences, for example at least one sequence read of at least 3, 4, 5, 6, 7, 8, 9, 10, 15, 20, 25, 50, 100, 1000, 10,000 characterized methylome sequences. Preferably each genomic region is covered by at least one sequence read of at least two characterized methylome sequences, for example at least one sequence read of at least 3, 4, 5, 6, 7, 8, 9, 10, 15, 20, 25, 50, 100, or 1000 characterized methylome sequences. In certain preferred embodiments, each genomic region is covered by at least one sequence read of at least 10 characterized methylome sequences, for example at least one sequence read of at least 10, at least 15, at least 20, at least 25, at least 50, at least 100, or at least 1000 characterized methylome sequences.

In certain embodiments, each genomic region for which the average methylation ratio has been determined is covered by at least 2 sequence reads, for example at least 2, 3, 4, 5, 6, 7, 8, 9, 10, 12, 15, 20, 25, 50, 100, 200, 300, 400, 500, 1000, or 10,000 sequence reads. Preferably, each genomic region is covered by at least 5 sequence reads, for example at least 6, 7, 8, 9, 10, 12, 15, 20, 25, 50, 100, 200, 300, 400, 500, 1000, or 10,000 sequence reads. More preferably, each genomic region is covered by at least 10 sequence reads, for example at least 12, 15, 20, 25, 50, 100, 200, 300, 400, 500, 1000, or 10,000 sequence reads.

In embodiments wherein each genomic region for which the average methylation ratio has been determined is covered by at least 2 sequence reads (for example at least 2, 3, 4, 5, 6, 7, 8, 9, 10, 12, 15, 20, 25, 50, 100, 200, 300, 400, 500, 1000, or 10,000 sequence reads) preferably each sequence read or the majority of the sequence reads (for example at least 50%, 60%, 70%, 80% or 90% of the sequence reads) are from different characterized methylome sequences. More preferably, each sequence read or at least 60%, 70%, 80% or 90% of the sequence reads are from different characterized methylome sequences.

In certain embodiments the method comprises determining the average methylation ratio at 12 or more genomic regions, for example 15 or more genomic regions, 20 or more genomic regions, 25 or more genomic regions, 30 or more genomic regions, 50 or more genomic regions, 75 or more genomic regions, 100 or more genomic regions, 125 or more genomic regions, 150 or more genomic regions, 200 or more genomic regions, 300 or more genomic regions, 400 or more genomic regions, 500 or more genomic regions, 600 or more genomic regions, 700 or more genomic regions, 800 or more genomic regions, 900 or more genomic regions, or 1000 genomic regions. Each genomic region may be selected from the group consisting of:
a 100 to 200 bp region comprising or having a genomic location defined in Tables 1 to 4, and
a 2 to 99 bp region within a genomic location defined in Tables 1 to 4 and comprising at least one CpG locus.

The genomic regions are preferably each different from each other. In certain preferred embodiments, the method comprises determining the average methylation ratio at 100 or more genomic regions, 125 or more genomic regions, 150 or more genomic regions, 200 or more genomic regions, 300 or more genomic regions, 400 or more genomic regions, 500 or more genomic regions, 600 or more genomic regions, 700 or more genomic regions, 800 or more genomic regions, 900 or more genomic regions, or 1000 genomic regions. Each genomic region may be selected from the group consisting of:
a 100 to 200 bp region comprising or having a genomic location defined in Tables 1 to 4, and
a 2 to 99 bp region within a genomic location defined in Tables 1 to 4 and comprising at least one CpG locus.

In certain preferred embodiments, the method comprises determining the average methylation ratio at 500 or more genomic regions, 600 or more genomic regions, 700 or more genomic regions, 800 or more genomic regions, 900 or more genomic regions, or 1000 genomic regions. Each genomic region may be selected from the group consisting of:
a 100 to 200 bp region comprising or having a genomic location defined in Tables 1 to 4, and
a 2 to 99 bp region within a genomic location defined in Tables 1 to 4 and comprising at least one CpG locus.

In certain preferred embodiments, the method comprises determining the average methylation ratio at 800 or more genomic regions, 900 or more genomic regions, or 1000 genomic regions. Each genomic region may be selected from the group consisting of:
a 100 to 200 bp region comprising or having a genomic location defined in Tables 1 to 4, and
a 2 to 99 bp region within a genomic location defined in Tables 1 to 4 and comprising at least one CpG locus.

In one embodiment, each genomic region is selected from the group consisting of:
a 100 to 200 bp region comprising or having a genomic location defined in Tables 3 and 4, and
a 2 to 99 bp region within a genomic location defined in Tables 3 and 4 and comprising at least one CpG locus.

In such embodiments, preferably the method comprises determining the average methylation ratio at 12 or more genomic regions, for example 15 or more genomic regions, 20 or more genomic regions, 25 or more genomic regions, 30 or more genomic regions, 50 or more genomic regions, 75 or more genomic regions, 100 or more genomic regions, 125 or more genomic regions, 150 or more genomic regions, 200 or more genomic regions, 300 or more genomic regions, or 400 or more genomic regions. For example, the method comprises determining the average methylation ratio at 100 or more genomic regions.

In certain embodiments, each genomic region is selected from the group consisting of:
a 100 to 200 bp region comprising or having a genomic location defined in Tables 1 and 3, and a 2 to 99 bp region within a genomic location defined in Tables 1 and 3 and comprising at least one CpG locus. More suitably, each genomic region is selected from the group consisting of: a 100 to 150 bp region comprising or having a genomic location defined in Tables 1 and 3, and 10 to 99 bp region within a genomic location defined in Tables 1 and 3 and comprising at least one CpG locus. More suitably, each genomic region is selected from the group consisting of: a 100 to 120 bp region comprising or having a genomic location defined in Tables 1 and 3, and 50 to 99 bp region within a genomic location defined in Tables 1 and 3 and comprising at least one CpG locus. More suitably, each genomic region is selected from the group consisting of: a 100 to 120 bp region comprising or having a genomic location defined in Tables 1 and 3, and 80 to 99 bp region within a genomic location defined in Tables 1 and 3 and comprising at least one CpG locus. For example, each genomic region is selected from a 100 bp region having a genomic location defined in Tables 1 and 3.

In such embodiments, preferably the method comprises determining the average methylation ratio at 12 or more genomic regions, for example 15 or more genomic regions, 20 or more genomic regions, 25 or more genomic regions, 30 or more genomic regions, 50 or more genomic regions, 75 or more genomic regions, 100 or more genomic regions, 125 or more genomic regions, 150 or more genomic regions, 200 or more genomic regions, 300 or more genomic regions, or 400 or more genomic regions. For example, the method comprises determining the average methylation ratio at 100 or more genomic regions.

In certain embodiments, each genomic region is selected from the group consisting of:
a 100 to 200 bp region comprising or having a genomic location defined in Tables 2 and 4, and a 2 to 99 bp region within a genomic location defined in Tables 2 and 4 and comprising at least one CpG locus. More suitably, each genomic region is selected from the group consisting of: a 100 to 150 bp region comprising or having a genomic location defined in Tables 2 and 4, and 10 to 99 bp region within a genomic location defined in Tables 2 and 4 and comprising at least one CpG locus. More suitably, each genomic region is selected from the group consisting of: a 100 to 120 bp region comprising or having a genomic location defined in Tables 2 and 4, and 50 to 99 bp region within a genomic location defined in Tables 2 and 4 and comprising at least one CpG locus. More suitably, each genomic region is selected from the group consisting of: a 100 to 120 bp region comprising or having a genomic location defined in Tables 2 and 4, and 80 to 99 bp region within a genomic location defined in Tables 2 and 4 and comprising at least one CpG locus. For example, each genomic region is selected from a 100 bp region having a genomic location defined in Tables 2 and 4.

In such embodiments, preferably the method comprises determining the average methylation ratio at 12 or more genomic regions, for example 15 or more genomic regions, 20 or more genomic regions, 25 or more genomic regions, 30 or more genomic regions, 50 or more genomic regions, 75 or more genomic regions, 100 or more genomic regions, 125 or more genomic regions, 150 or more genomic regions, 200 or more genomic regions, 300 or more genomic regions, or 400 or more genomic regions. For example, the method comprises determining the average methylation ratio at 100 or more genomic regions.

In certain preferred embodiments, each genomic region is selected from the group consisting of:
a 100 to 200 bp region comprising or having a genomic location defined in Tables 1 and 2, and a 2 to 99 bp region within a genomic location defined in Tables 1 and 2 and comprising at least one CpG locus. More suitably, each genomic region is selected from the group consisting of: a 100 to 150 bp region comprising or having a genomic location defined in Tables 1 and 2, and 10 to 99 bp region within a genomic location defined in Tables 1 and 2 and comprising at least one CpG locus. More suitably, each genomic region is selected from the group consisting of: a 100 to 120 bp region comprising or having a genomic location defined in Tables 1 and 2, and 50 to 99 bp region within a genomic location defined in Tables 1 and 2 and comprising at least one CpG locus. More suitably, each genomic region is selected from the group consisting of: a 100 to 120 bp region comprising or having a genomic location defined in Tables 1 and 2, and 80 to 99 bp region within a genomic location defined in Tables 1 and 2 and comprising at least one CpG locus. For example, each genomic region is selected from a 100 bp region having a genomic location defined in Tables 1 and 2.

In such preferred embodiments, preferably the method comprises determining the average methylation ratio at 12 or more genomic regions, for example 15 or more genomic regions, 20 or more genomic regions, 25 or more genomic regions, 30 or more genomic regions, 50 or more genomic regions, 75 or more genomic regions, 100 or more genomic regions, 125 or more genomic regions, 150 or more genomic regions, 200 or more genomic regions, 300 or more genomic regions, or 400 or more genomic regions. For example, the method comprises determining the average methylation ratio at 100 or more genomic regions.

In certain embodiments, each genomic region is selected from the group consisting of:
a 100 to 200 bp region comprising or having a genomic location defined in Tables 3 and 4, and a 2 to 99 bp region within a genomic location defined in Tables 3 and 4 and comprising at least one CpG locus. More suitably, each genomic region is selected from the group consisting of: a 100 to 150 bp region comprising or having a genomic location defined in Tables 3 and 4, and 10 to 99 bp region within a genomic location defined in Tables 3 and 4 and comprising at least one CpG locus. More suitably, each genomic region is selected from the group consisting of: a 100 to 120 bp region comprising or having a genomic location defined in Tables 3 and 4, and 50 to 99 bp region within a genomic location defined in Tables 3 and 4 and comprising at least one CpG locus. More suitably, each genomic region is selected from the group consisting of: a 100 to 120 bp region comprising or having a genomic location defined in Tables 3 and 4, and 80 to 99 bp region within a genomic location defined in Tables 3 and 4 and comprising at least one CpG locus. For example, each genomic region is selected from a 100 bp region having a genomic location defined in Tables 3 and 4.

In such embodiments, preferably the method comprises determining the average methylation ratio at 12 or more genomic regions, for example 15 or more genomic regions, 20 or more genomic regions, 25 or more genomic regions, 30 or more genomic regions, 50 or more genomic regions, 75 or more genomic regions, 100 or more genomic regions, 125 or more genomic regions, 150 or more genomic regions, 200 or more genomic regions, 300 or more genomic regions, or 400 or more genomic regions. For example, the method comprises determining the average methylation ratio at 100 or more genomic regions.

In one preferred embodiment, each genomic region is selected from the group consisting of:
a 100 to 200 bp region comprising or having a genomic location defined in Table 5, and a 2 to 99 bp region within a genomic location defined in Table 5 and comprising at least one CpG locus. More suitably, each genomic region is selected from the group consisting of: a 100 to 150 bp region comprising or having a genomic location defined in Table 5, and 10 to 99 bp region within a genomic location defined in Table 5 and comprising at least one CpG locus. More suitably, each genomic region is selected from the group consisting of: a 100 to 120 bp region comprising or having a genomic location defined in Table 5, and 50 to 99 bp region within a genomic location defined in Table 5 and comprising at least one CpG locus. More suitably, each genomic region is selected from the group consisting of: a 100 to 120 bp region comprising or having a genomic location defined in Table 5, and 80 to 99 bp region within a genomic location defined in Table 5 and comprising at least one CpG locus. For example, each genomic region is selected from a 100 bp region having a genomic location defined in Table 5.

TABLE 5
A preferred subset of hypermethylated and hypomethylated
region genomic locations (The genomic locations
are locations with reference to hg19)

| | | | Hyper- or |
|---|---|---|---|
| | | | hypomethylated |
| Chromosome | start | end | region |
| chr4 | 9104451 | 9104550 | hypo |
| chr12 | 54441001 | 54441100 | hyper |
| chr1 | 153174701 | 153174800 | hypo |
| chr4 | 9104401 | 9104500 | hypo |
| chr1 | 248308901 | 248309000 | hypo |
| chr12 | 4030001 | 4030100 | hypo |
| chr2 | 91936151 | 91936250 | hypo |
| chr2 | 198063601 | 198063700 | hyper |
| chr10 | 6779901 | 6780000 | hypo |
| chr19 | 56346551 | 56346650 | hypo |
| chr14 | 47670001 | 47670100 | hypo |
| chr17 | 77386351 | 77386450 | hypo |
| chr8 | 105988201 | 105988300 | hypo |
| chr2 | 54901001 | 54901100 | hyper |
| chr14 | 97924301 | 97924400 | hypo |
| chr14 | 95237401 | 95237500 | hyper |
| chr17 | 79422551 | 79422650 | hyper |
| chr14 | 97924251 | 97924350 | hypo |
| chr1 | 119526251 | 119526350 | hyper |
| chr14 | 37125801 | 37125900 | hyper |
| chr2 | 177012701 | 177012800 | hyper |
| chr14 | 47670201 | 47670300 | hypo |
| chr17 | 3030101 | 3030200 | hypo |
| chr4 | 77226351 | 77226450 | hyper |
| chr3 | 38835251 | 38835350 | hypo |
| chr5 | 87439351 | 87439450 | hyper |
| chr9 | 22005201 | 22005300 | hyper |
| chr2 | 198063651 | 198063750 | hyper |
| chr12 | 131512601 | 131512700 | hypo |
| chr2 | 879451 | 879550 | hypo |
| chr5 | 87439401 | 87439500 | hyper |
| chr1 | 204165251 | 204165350 | hypo |
| chr9 | 132650551 | 132650650 | hyper |
| chr20 | 1975351 | 1975450 | hypo |
| chr17 | 79422601 | 79422700 | hyper |
| chr9 | 110399201 | 110399300 | hyper |
| chr6 | 170531301 | 170531400 | hyper |
| chr9 | 132650601 | 132650700 | hyper |
| chr7 | 45066701 | 45066800 | hyper |
| chr8 | 139124351 | 139124450 | hypo |
| chr1 | 207103601 | 207103700 | hyper |
| chr8 | 99950901 | 99951000 | hyper |
| chr8 | 99950851 | 99950950 | hyper |
| chr7 | 45066651 | 45066750 | hyper |
| chr9 | 38437301 | 38437400 | hypo |
| chr12 | 4361001 | 4361100 | hypo |
| chr17 | 72776151 | 72776250 | hyper |
| chr12 | 4361051 | 4361150 | hypo |
| chr2 | 204571201 | 204571300 | hyper |
| chr1 | 162527451 | 162527550 | hypo |
| chr1 | 207103651 | 207103750 | hyper |
| chr4 | 108814501 | 108814600 | hyper |
| chr14 | 37125851 | 37125950 | hyper |
| chr8 | 139124401 | 139124500 | hypo |
| chr4 | 77226301 | 77226400 | hyper |
| chr20 | 1975301 | 1975400 | hypo |
| chr2 | 232186901 | 232187000 | hyper |
| chr20 | 5282901 | 5283000 | hypo |
| chr20 | 1975401 | 1975500 | hypo |
| chr6 | 152804751 | 152804850 | hypo |
| chr19 | 55042751 | 55042850 | hypo |
| chr12 | 132102101 | 132102200 | hypo |
| chr17 | 77386401 | 77386500 | hypo |
| chr14 | 47670051 | 47670150 | hypo |
| chr9 | 140586201 | 140586300 | hyper |
| chr5 | 179344551 | 179344650 | hyper |
| chr1 | 143907501 | 143907600 | hypo |
| chr1 | 143907451 | 143907550 | hypo |
| chr1 | 119526201 | 119526300 | hyper |
| chr6 | 152804701 | 152804800 | hypo |
| chr2 | 228324901 | 228325000 | hyper |
| chr19 | 55042801 | 55042900 | hypo |
| chr3 | 160475151 | 160475250 | hyper |
| chr1 | 182021751 | 182021850 | hypo |
| chr1 | 182021701 | 182021800 | hypo |
| chr8 | 111906551 | 111906650 | hypo |
| chr6 | 170531351 | 170531450 | hyper |
| chr2 | 232186851 | 232186950 | hyper |
| chr8 | 130365301 | 130365400 | hypo |
| chr2 | 117006251 | 117006350 | hypo |
| chr3 | 194868701 | 194868800 | hyper |
| chr18 | 11153901 | 11154000 | hypo |
| chr18 | 11153851 | 11153950 | hypo |
| chr1 | 175490551 | 175490650 | hypo |
| chr3 | 160475201 | 160475300 | hyper |
| chr19 | 2776001 | 2776100 | hyper |
| chr3 | 193096401 | 193096500 | hypo |
| chr2 | 228324851 | 228324950 | hyper |
| chr8 | 120779851 | 120779950 | hypo |
| chr12 | 131512551 | 131512650 | hypo |
| chr9 | 125796751 | 125796850 | hyper |
| chr3 | 194868651 | 194868750 | hyper |
| chr10 | 7567801 | 7567900 | hypo |
| chr1 | 175490601 | 175490700 | hypo |
| chr1 | 68154601 | 68154700 | hyper |
| chr17 | 3030151 | 3030250 | hypo |
| chr2 | 91910601 | 91910700 | hypo |
| chr2 | 91910551 | 91910650 | hypo |
| chr14 | 97924451 | 97924550 | hypo |
| chr9 | 22005551 | 22005650 | hyper |
| chr11 | 121986951 | 121987050 | hypo |
| chr14 | 97497051 | 97497150 | hypo |
| chr1 | 95172951 | 95173050 | hypo |
| chr3 | 38835201 | 38835300 | hypo |
| chr14 | 37124151 | 37124250 | hyper |
| chr4 | 13544651 | 13544750 | hyper |
| chrX | 150944951 | 150945050 | hypo |
| chr3 | 46448751 | 46448850 | hyper |
| chr1 | 248153651 | 248153750 | hypo |
| chr20 | 19866701 | 19866800 | hypo |
| chr2 | 3633351 | 3633450 | hypo |
| chr14 | 104742101 | 104742200 | hypo |
| chr20 | 5450901 | 5451000 | hypo |
| chr1 | 153175251 | 153175350 | hypo |
| chr9 | 22005501 | 22005600 | hyper |
| chr12 | 116997101 | 116997200 | hyper |
| chr15 | 98646401 | 98646500 | hypo |
| chr12 | 130494601 | 130494700 | hypo |
| chr4 | 120502101 | 120502200 | hypo |
| chr7 | 5518851 | 5518950 | hyper |
| chr17 | 55562951 | 55563050 | hyper |
| chr7 | 57510201 | 57510300 | hypo |
| chr5 | 3002401 | 3002500 | hypo |
| chr3 | 100690901 | 100691000 | hypo |
| chr3 | 100690851 | 100690950 | hypo |
| chr14 | 97924501 | 97924600 | hypo |
| chr2 | 206551451 | 206551550 | hyper |
| chr1 | 2876551 | 2876650 | hypo |
| chr12 | 4030051 | 4030150 | hypo |
| chr12 | 132663901 | 132664000 | hypo |
| chr1 | 153174651 | 153174750 | hypo |
| chr6 | 34252801 | 34252900 | hyper |
| chr2 | 177012651 | 177012750 | hyper |
| chr6 | 45296101 | 45296200 | hyper |
| chr12 | 8438301 | 8438400 | hypo |
| chr2 | 177012551 | 177012650 | hyper |
| chr1 | 2876501 | 2876600 | hypo |
| chr3 | 194868751 | 194868850 | hyper |
| chr7 | 6476051 | 6476150 | hyper |
| chr3 | 127453801 | 127453900 | hyper |
| chr3 | 127453851 | 127453950 | hyper |
| chr12 | 7062101 | 7062200 | hyper |
| chr14 | 59296601 | 59296700 | hypo |
| chr9 | 91006701 | 91006800 | hyper |
| chr9 | 110399251 | 110399350 | hyper |
| chr2 | 71116551 | 71116650 | hyper |
| chr3 | 72227251 | 72227350 | hyper |
| chr2 | 60880201 | 60880300 | hypo |
| chr7 | 129411101 | 129411200 | hyper |
| chr12 | 111536901 | 111537000 | hyper |
| chr17 | 55562901 | 55563000 | hyper |
| chr4 | 101438801 | 101438900 | hyper |
| chr17 | 21911401 | 21911500 | hypo |
| chr11 | 47939651 | 47939750 | hyper |
| chr2 | 54900951 | 54901050 | hyper |
| chr14 | 59296651 | 59296750 | hypo |
| chr16 | 10206551 | 10206650 | hypo |
| chr1 | 143907551 | 143907650 | hypo |
| chr14 | 47669951 | 47670050 | hypo |
| chr19 | 33162851 | 33162950 | hyper |
| chr14 | 93412701 | 93412800 | hypo |
| chr12 | 130711351 | 130711450 | hypo |
| chr2 | 100426951 | 100427050 | hypo |
| chr2 | 100426901 | 100427000 | hypo |
| chr9 | 22005601 | 22005700 | hyper |
| chr2 | 2581301 | 2581400 | hypo |
| chr17 | 59534651 | 59534750 | hyper |
| chr10 | 6779951 | 6780050 | hypo |
| chr5 | 176758401 | 176758500 | hyper |
| chr9 | 96080351 | 96080450 | hyper |
| chr7 | 129411151 | 129411250 | hyper |
| chr17 | 79422501 | 79422600 | hyper |
| chr15 | 86098601 | 86098700 | hyper |
| chr22 | 50618601 | 50618700 | hyper |
| chr19 | 55104601 | 55104700 | hypo |
| chr10 | 94450951 | 94451050 | hyper |
| chr14 | 47670101 | 47670200 | hypo |
| chr8 | 130365251 | 130365350 | hypo |
| chr1 | 2876451 | 2876550 | hypo |
| chr1 | 204165301 | 204165400 | hypo |
| chr2 | 172974201 | 172974300 | hyper |
| chr2 | 172974151 | 172974250 | hyper |
| chr17 | 72776201 | 72776300 | hyper |
| chr19 | 55036701 | 55036800 | hypo |
| chr1 | 95173001 | 95173100 | hypo |
| chr12 | 4361101 | 4361200 | hypo |
| chr7 | 5518901 | 5519000 | hyper |
| chr12 | 6665301 | 6665400 | hyper |
| chr1 | 169696601 | 169696700 | hypo |
| chr12 | 132142051 | 132142150 | hypo |
| chr12 | 132142001 | 132142100 | hypo |
| chr8 | 56361951 | 56362050 | hypo |
| chr16 | 23988951 | 23989050 | hypo |
| chr9 | 91006751 | 91006850 | hyper |
| chr2 | 228324951 | 228325050 | hyper |
| chr5 | 134826351 | 134826450 | hyper |
| chr2 | 879501 | 879600 | hypo |
| chr4 | 53862451 | 53862550 | hyper |
| chr14 | 37124201 | 37124300 | hyper |
| chr10 | 6664951 | 6665050 | hypo |
| chr8 | 56106801 | 56106900 | hypo |
| chr8 | 142289801 | 142289900 | hypo |
| chr14 | 104742051 | 104742150 | hypo |
| chr5 | 5492401 | 5492500 | hypo |
| chr20 | 31123201 | 31123300 | hyper |
| chr2 | 89215151 | 89215250 | hypo |
| chr2 | 89215101 | 89215200 | hypo |
| chr2 | 232186951 | 232187050 | hyper |
| chr5 | 10445501 | 10445600 | hyper |
| chr3 | 177397701 | 177397800 | hyper |
| chr11 | 47939701 | 47939800 | hyper |
| chr6 | 34252751 | 34252850 | hyper |
| chr19 | 57646101 | 57646200 | hypo |
| chr4 | 74809851 | 74809950 | hyper |
| chr19 | 33162801 | 33162900 | hyper |
| chr1 | 64937351 | 64937450 | hyper |
| chr1 | 68154551 | 68154650 | hyper |
| chr2 | 172945201 | 172945300 | hyper |
| chr17 | 22023751 | 22023850 | hypo |
| chr1 | 65399451 | 65399550 | hyper |
| chr19 | 46526251 | 46526350 | hyper |
| chr2 | 171569151 | 171569250 | hyper |
| chr10 | 31423501 | 31423600 | hyper |
| chr14 | 37125901 | 37126000 | hyper |
| chr11 | 57519401 | 57519500 | hypo |
| chr16 | 23939051 | 23939150 | hypo |
| chr19 | 29281851 | 29281950 | hypo |
| chr19 | 29281801 | 29281900 | hypo |
| chr10 | 94450901 | 94451000 | hyper |
| chr1l | 6865801 | 6865900 | hypo |
| chr9 | 140586151 | 140586250 | hyper |
| chr6 | 41394801 | 41394900 | hyper |
| chr4 | 108814551 | 108814650 | hyper |
| chrX | 150945001 | 150945100 | hypo |
| chr19 | 18508551 | 18508650 | hyper |
| chr9 | 96080301 | 96080400 | hyper |
| chr14 | 95237351 | 95237450 | hyper |
| chr17 | 59532801 | 59532900 | hyper |
| chr20 | 5282851 | 5282950 | hypo |
| chr8 | 142289851 | 142289950 | hypo |
| chr5 | 72676801 | 72676900 | hyper |
| chr17 | 22017001 | 22017100 | hypo |
| chr2 | 71126251 | 71126350 | hyper |
| chr2 | 59477151 | 59477250 | hypo |
| chr7 | 149112151 | 149112250 | hyper |
| chr1 | 4079101 | 4079200 | hypo |
| chr17 | 78724151 | 78724250 | hyper |
| chr14 | 60976901 | 60977000 | hyper |
| chr9 | 5756751 | 5756850 | hypo |
| chr22 | 17073551 | 17073650 | hypo |

In such embodiments, preferably the method comprises determining the average methylation ratio at 10 or more genomic regions, 12 or more genomic regions, for example 15 or more genomic regions, 20 or more genomic regions, 25 or more genomic regions, 30 or more genomic regions, 50 or more genomic regions, 75 or more genomic regions, 100 or more genomic regions, 125 or more genomic regions, 150 or more genomic regions, 200 or more genomic regions, or 250 genomic regions. For example, the method comprises determining the average methylation ratio at 10 or more genomic regions, 50 or more genomic regions or 100 or more genomic regions.

In another preferred embodiments, each genomic region is selected from the group consisting of:
a 100 to 200 bp region comprising or having a genomic location defined in Table 6, and a 2 to 99 bp region within a genomic location defined in Table 6 and comprising at least one CpG locus. More suitably, each genomic region is selected from the group consisting of: a 100 to 150 bp region comprising or having a genomic location defined in Table 6, and 10 to 99 bp region within a genomic location defined in Table 6 and comprising at least one CpG locus. More suitably, each genomic region is selected from the group consisting of: a 100 to 120 bp region comprising or having a genomic location defined in Table 6, and 50 to 99 bp region within a genomic location defined in Table 6 and comprising at least one CpG locus. More suitably, each genomic region is selected from the group consisting of: a 100 to 120 bp region comprising or having a genomic location defined in Table 6, and 80 to 99 bp region within a genomic location defined in Table 6 and comprising at least one CpG locus. For example, each genomic region is selected from a 100 bp region having a genomic location defined in Table 6.

TABLE 6
A preferred subset of hypomethylated region genomic locations
(The genomic locations are locations with reference to hg19)

| | | | Hyper- or |
|---|---|---|---|
| | | | hypomethylated |
| Chromosome | start | end | region |
| chr4 | 9104451 | 9104550 | hypo |
| chr1 | 153174701 | 153174800 | hypo |
| chr4 | 9104401 | 9104500 | hypo |
| chr1 | 248308901 | 248309000 | hypo |
| chr12 | 4030001 | 4030100 | hypo |
| chr2 | 91936151 | 91936250 | hypo |
| chr10 | 6779901 | 6780000 | hypo |
| chr19 | 56346551 | 56346650 | hypo |
| chr14 | 47670001 | 47670100 | hypo |
| chr17 | 77386351 | 77386450 | hypo |
| chr8 | 105988201 | 105988300 | hypo |
| chr14 | 97924301 | 97924400 | hypo |
| chr14 | 97924251 | 97924350 | hypo |
| chr14 | 47670201 | 47670300 | hypo |
| chr17 | 3030101 | 3030200 | hypo |
| chr3 | 38835251 | 38835350 | hypo |
| chr12 | 131512601 | 131512700 | hypo |
| chr2 | 879451 | 879550 | hypo |
| chr1 | 204165251 | 204165350 | hypo |
| chr20 | 1975351 | 1975450 | hypo |
| chr8 | 139124351 | 139124450 | hypo |
| chr9 | 38437301 | 38437400 | hypo |
| chr12 | 4361001 | 4361100 | hypo |
| chr12 | 4361051 | 4361150 | hypo |
| chr1 | 162527451 | 162527550 | hypo |
| chr8 | 139124401 | 139124500 | hypo |
| chr20 | 1975301 | 1975400 | hypo |
| chr20 | 5282901 | 5283000 | hypo |
| chr20 | 1975401 | 1975500 | hypo |
| chr6 | 152804751 | 152804850 | hypo |
| chr19 | 55042751 | 55042850 | hypo |
| chr12 | 132102101 | 132102200 | hypo |
| chr17 | 77386401 | 77386500 | hypo |
| chr14 | 47670051 | 47670150 | hypo |
| chr1 | 143907501 | 143907600 | hypo |
| chr1 | 143907451 | 143907550 | hypo |
| chr6 | 152804701 | 152804800 | hypo |
| chr19 | 55042801 | 55042900 | hypo |
| chr1 | 182021751 | 182021850 | hypo |
| chr1 | 182021701 | 182021800 | hypo |
| chr8 | 111906551 | 111906650 | hypo |
| chr8 | 130365301 | 130365400 | hypo |
| chr2 | 117006251 | 117006350 | hypo |
| chr18 | 11153901 | 11154000 | hypo |
| chr18 | 11153851 | 11153950 | hypo |
| chr1 | 175490551 | 175490650 | hypo |
| chr3 | 193096401 | 193096500 | hypo |
| chr8 | 120779851 | 120779950 | hypo |
| chr12 | 131512551 | 131512650 | hypo |
| chr10 | 7567801 | 7567900 | hypo |
| chr1 | 175490601 | 175490700 | hypo |
| chr17 | 3030151 | 3030250 | hypo |
| chr2 | 91910601 | 91910700 | hypo |
| chr2 | 91910551 | 91910650 | hypo |
| chr14 | 97924451 | 97924550 | hypo |
| chr11 | 121986951 | 121987050 | hypo |
| chr14 | 97497051 | 97497150 | hypo |
| chr1 | 95172951 | 95173050 | hypo |
| chr3 | 38835201 | 38835300 | hypo |
| chrX | 150944951 | 150945050 | hypo |
| chr1 | 248153651 | 248153750 | hypo |
| chr20 | 19866701 | 19866800 | hypo |
| chr2 | 3633351 | 3633450 | hypo |
| chr14 | 104742101 | 104742200 | hypo |
| chr20 | 5450901 | 5451000 | hypo |
| chr1 | 153175251 | 153175350 | hypo |
| chr15 | 98646401 | 98646500 | hypo |
| chr12 | 130494601 | 130494700 | hypo |
| chr4 | 120502101 | 120502200 | hypo |
| chr7 | 57510201 | 57510300 | hypo |
| chr5 | 3002401 | 3002500 | hypo |
| chr3 | 100690901 | 100691000 | hypo |
| chr3 | 100690851 | 100690950 | hypo |
| chr14 | 97924501 | 97924600 | hypo |
| chr1 | 2876551 | 2876650 | hypo |
| chr12 | 4030051 | 4030150 | hypo |
| chr12 | 132663901 | 132664000 | hypo |
| chr1 | 153174651 | 153174750 | hypo |
| chr12 | 8438301 | 8438400 | hypo |
| chr1 | 2876501 | 2876600 | hypo |
| chr14 | 59296601 | 59296700 | hypo |
| chr2 | 60880201 | 60880300 | hypo |
| chr17 | 21911401 | 21911500 | hypo |
| chr14 | 59296651 | 59296750 | hypo |
| chr16 | 10206551 | 10206650 | hypo |
| chr1 | 143907551 | 143907650 | hypo |
| chr14 | 47669951 | 47670050 | hypo |
| chr14 | 93412701 | 93412800 | hypo |
| chr12 | 130711351 | 130711450 | hypo |
| chr2 | 100426951 | 100427050 | hypo |
| chr2 | 100426901 | 100427000 | hypo |
| chr2 | 2581301 | 2581400 | hypo |
| chr10 | 6779951 | 6780050 | hypo |
| chr19 | 55104601 | 55104700 | hypo |
| chr14 | 47670101 | 47670200 | hypo |
| chr8 | 130365251 | 130365350 | hypo |
| chr1 | 2876451 | 2876550 | hypo |
| chr1 | 204165301 | 204165400 | hypo |
| chr19 | 55036701 | 55036800 | hypo |
| chr1 | 95173001 | 95173100 | hypo |
| chr12 | 4361101 | 4361200 | hypo |
| chr1 | 169696601 | 169696700 | hypo |
| chr12 | 132142051 | 132142150 | hypo |
| chr12 | 132142001 | 132142100 | hypo |
| chr8 | 56361951 | 56362050 | hypo |
| chr16 | 23988951 | 23989050 | hypo |
| chr2 | 879501 | 879600 | hypo |
| chr10 | 6664951 | 6665050 | hypo |
| chr8 | 56106801 | 56106900 | hypo |
| chr8 | 142289801 | 142289900 | hypo |
| chr14 | 104742051 | 104742150 | hypo |
| chr5 | 5492401 | 5492500 | hypo |
| chr2 | 89215151 | 89215250 | hypo |
| chr2 | 89215101 | 89215200 | hypo |
| chr19 | 57646101 | 57646200 | hypo |
| chr17 | 22023751 | 22023850 | hypo |
| chr11 | 57519401 | 57519500 | hypo |
| chr16 | 23939051 | 23939150 | hypo |
| chr19 | 29281851 | 29281950 | hypo |
| chr19 | 29281801 | 29281900 | hypo |
| chr11 | 6865801 | 6865900 | hypo |
| chrX | 150945001 | 150945100 | hypo |
| chr20 | 5282851 | 5282950 | hypo |
| chr8 | 142289851 | 142289950 | hypo |
| chr17 | 22017001 | 22017100 | hypo |
| chr2 | 59477151 | 59477250 | hypo |
| chr1 | 4079101 | 4079200 | hypo |
| chr9 | 5756751 | 5756850 | hypo |
| chr22 | 17073551 | 17073650 | hypo |
| chr22 | 24979551 | 24979650 | hypo |
| chr11 | 7961101 | 7961200 | hypo |
| chr11 | 7961051 | 7961150 | hypo |
| chr5 | 19531551 | 19531650 | hypo |
| chr1 | 175490501 | 175490600 | hypo |
| chr5 | 19531501 | 19531600 | hypo |
| chr21 | 44375551 | 44375650 | hypo |
| chr7 | 39056351 | 39056450 | hypo |
| chr14 | 47670251 | 47670350 | hypo |
| chr1 | 148903551 | 148903650 | hypo |
| chr3 | 192960551 | 192960650 | hypo |
| chr19 | 55042301 | 55042400 | hypo |
| chr14 | 104742151 | 104742250 | hypo |
| chr4 | 157059301 | 157059400 | hypo |
| chr3 | 33757901 | 33758000 | hypo |
| chr4 | 3895151 | 3895250 | hypo |
| chr14 | 97924401 | 97924500 | hypo |
| chr7 | 39056301 | 39056400 | hypo |
| chr2 | 242190801 | 242190900 | hypo |
| chr19 | 55042251 | 55042350 | hypo |
| chr6 | 159872251 | 159872350 | hypo |

In such embodiments, preferably the method comprises determining the average methylation ratio at 10 or more genomic regions, at 12 or more genomic regions, for example at 15 or more genomic regions, 20 or more genomic regions, 25 or more genomic regions, 30 or more genomic regions, 50 or more genomic regions, 75 or more genomic regions, 100 or more genomic regions, 125 or more genomic regions, or 150 genomic regions. For example, the method comprises determining the average methylation ratio at 10 or more genomic regions, 50 or more genomic regions or 100 or more genomic regions.

In another preferred embodiment, each genomic region is selected from the group consisting of:

a 100 to 200 bp region comprising or having a genomic location defined in Table 7, and a 2 to 99 bp region within a genomic location defined in Table 7 and comprising at least one CpG locus. More suitably, each genomic region is selected from the group consisting of: a 100 to 150 bp region comprising or having a genomic location defined in Table 7, and 10 to 99 bp region within a genomic location defined in Table 7 and comprising at least one CpG locus. More suitably, each genomic region is selected from the group consisting of: a 100 to 120 bp region comprising or having a genomic location defined in Table 7, and 50 to 99 bp region within a genomic location defined in Table 7 and comprising at least one CpG locus. More suitably, each genomic region is selected from the group consisting of: a 100 to 120 bp region comprising or having a genomic location defined in Table 7, and 80 to 99 bp region within a genomic location defined in Table 7 and comprising at least one CpG locus. For example, each genomic region is selected from a 100 bp region having a genomic location defined in Table 7.

TABLE 7
A preferred subset of hypermethylated and hypomethylated
region genomic locations (The genomic locations
are locations with reference to hg19)

| | | | Hyper- or |
|---|---|---|---|
| | | | hypomethylated |
| Chromosome | start | end | region |
| chr4 | 9104451 | 9104550 | hypo |
| chr12 | 54441001 | 54441100 | hyper |
| chr1 | 153174701 | 153174800 | hypo |
| chr4 | 9104401 | 9104500 | hypo |
| chr1 | 248308901 | 248309000 | hypo |
| chr12 | 4030001 | 4030100 | hypo |
| chr2 | 91936151 | 91936250 | hypo |
| chr2 | 198063601 | 198063700 | hyper |
| chr10 | 6779901 | 6780000 | hypo |
| chr19 | 56346551 | 56346650 | hypo |
| chr14 | 47670001 | 47670100 | hypo |
| chr17 | 77386351 | 77386450 | hypo |
| chr8 | 105988201 | 105988300 | hypo |
| chr2 | 54901001 | 54901100 | hyper |
| chr14 | 97924301 | 97924400 | hypo |
| chr14 | 95237401 | 95237500 | hyper |
| chr17 | 79422551 | 79422650 | hyper |
| chr14 | 97924251 | 97924350 | hypo |
| chr1 | 119526251 | 119526350 | hyper |
| chr14 | 37125801 | 37125900 | hyper |
| chr2 | 177012701 | 177012800 | hyper |
| chr14 | 47670201 | 47670300 | hypo |
| chr17 | 3030101 | 3030200 | hypo |
| chr4 | 77226351 | 77226450 | hyper |
| chr3 | 38835251 | 38835350 | hypo |
| chr5 | 87439351 | 87439450 | hyper |
| chr9 | 22005201 | 22005300 | hyper |
| chr2 | 198063651 | 198063750 | hyper |
| chr12 | 131512601 | 131512700 | hypo |
| chr2 | 879451 | 879550 | hypo |
| chr5 | 87439401 | 87439500 | hyper |
| chr1 | 204165251 | 204165350 | hypo |
| chr9 | 132650551 | 132650650 | hyper |
| chr20 | 1975351 | 1975450 | hypo |
| chr17 | 79422601 | 79422700 | hyper |
| chr9 | 110399201 | 110399300 | hyper |
| chr6 | 170531301 | 170531400 | hyper |
| chr9 | 132650601 | 132650700 | hyper |
| chr7 | 45066701 | 45066800 | hyper |
| chr8 | 139124351 | 139124450 | hypo |
| chr1 | 207103601 | 207103700 | hyper |
| chr8 | 99950901 | 99951000 | hyper |
| chr8 | 99950851 | 99950950 | hyper |
| chr7 | 45066651 | 45066750 | hyper |
| chr9 | 38437301 | 38437400 | hypo |
| chr12 | 4361001 | 4361100 | hypo |
| chr17 | 72776151 | 72776250 | hyper |
| chr12 | 4361051 | 4361150 | hypo |
| chr2 | 204571201 | 204571300 | hyper |
| chr1 | 162527451 | 162527550 | hypo |
| chr1 | 207103651 | 207103750 | hyper |
| chr4 | 108814501 | 108814600 | hyper |
| chr14 | 37125851 | 37125950 | hyper |
| chr8 | 139124401 | 139124500 | hypo |
| chr4 | 77226301 | 77226400 | hyper |
| chr20 | 1975301 | 1975400 | hypo |
| chr2 | 232186901 | 232187000 | hyper |
| chr20 | 5282901 | 5283000 | hypo |
| chr20 | 1975401 | 1975500 | hypo |
| chr6 | 152804751 | 152804850 | hypo |
| chr19 | 55042751 | 55042850 | hypo |
| chr12 | 132102101 | 132102200 | hypo |
| chr17 | 77386401 | 77386500 | hypo |
| chr14 | 47670051 | 47670150 | hypo |
| chr9 | 140586201 | 140586300 | hyper |
| chr5 | 179344551 | 179344650 | hyper |
| chr1 | 143907501 | 143907600 | hypo |
| chr1 | 143907451 | 143907550 | hypo |
| chr1 | 119526201 | 119526300 | hyper |
| chr6 | 152804701 | 152804800 | hypo |
| chr2 | 228324901 | 228325000 | hyper |
| chr19 | 55042801 | 55042900 | hypo |
| chr3 | 160475151 | 160475250 | hyper |
| chr1 | 182021751 | 182021850 | hypo |
| chr1 | 182021701 | 182021800 | hypo |
| chr8 | 111906551 | 111906650 | hypo |
| chr6 | 170531351 | 170531450 | hyper |
| chr2 | 232186851 | 232186950 | hyper |
| chr8 | 130365301 | 130365400 | hypo |
| chr2 | 117006251 | 117006350 | hypo |
| chr3 | 194868701 | 194868800 | hyper |
| chr18 | 11153901 | 11154000 | hypo |
| chr18 | 11153851 | 11153950 | hypo |
| chr1 | 175490551 | 175490650 | hypo |
| chr3 | 160475201 | 160475300 | hyper |
| chr19 | 2776001 | 2776100 | hyper |
| chr3 | 193096401 | 193096500 | hypo |
| chr2 | 228324851 | 228324950 | hyper |
| chr8 | 120779851 | 120779950 | hypo |
| chr12 | 131512551 | 131512650 | hypo |
| chr9 | 125796751 | 125796850 | hyper |
| chr3 | 194868651 | 194868750 | hyper |
| chr10 | 7567801 | 7567900 | hypo |
| chr1 | 175490601 | 175490700 | hypo |
| chr1 | 68154601 | 68154700 | hyper |
| chr17 | 3030151 | 3030250 | hypo |
| chr2 | 91910601 | 91910700 | hypo |
| chr2 | 91910551 | 91910650 | hypo |
| chr14 | 97924451 | 97924550 | hypo |
| chr9 | 22005551 | 22005650 | hyper |

In such embodiments, preferably the method comprises determining the average methylation ratio at 10 or more genomic regions, at 12 or more genomic regions, for example 15 or more genomic regions, 20 or more genomic regions, 25 or more genomic regions, 30 or more genomic regions, 50 or more genomic regions, 75 or more genomic regions, or 100 genomic regions. For example, the method comprises determining the average methylation ratio at 10 or more genomic regions, 50 or more genomic regions or 100 genomic regions.

In certain preferred embodiments, at least 25% of the genomic regions comprise, have or are within a genomic location defined in Tables 1 and/or 2. For example, at least 25% of the genomic regions comprise or have a genomic location defined in Tables 1 and/or 2.

In certain preferred embodiments, at least 30%, at least 40%, at least 50%, at least 60%, at least 70%, at least 80%, at least 90%, at least 95%, or all of the genomic regions comprise, have or are within a genomic location defined in Tables 1 and/or 2. For example, at least 30%, at least 40%, at least 50%, at least 60%, at least 70%, at least 80%, at least 90%, at least 95%, or all of the genomic regions comprise or have a genomic location defined in Tables 1 and/or 2.

In certain embodiments, at least 25% of the genomic regions comprise, have or are within a genomic location defined in Tables 3 and/or 4. For example, at least 25% of the genomic regions comprise or have a genomic location defined in Tables 3 and/or 4.

In certain embodiments, at least 30%, at least 40%, at least 50%, at least 60%, at least 70%, at least 80%, at least 90%, at least 95%, or all of the genomic regions comprise, have or are within a genomic location defined in Tables 3 and/or 4. For example, at least 30%, at least 40%, at least 50%, at least 60%, at least 70%, at least 80%, at least 90%, at least 95%, or all of the genomic regions comprise or have a genomic location defined in Tables 3 and/or 4.

In certain embodiments, at least 25% of the genomic regions comprise, have or are within a genomic location defined in Tables 1 and/or 3. For example, at least 25% of the genomic regions comprise or have a genomic location defined in Tables 1 and/or 3.

In certain embodiments, at least 30%, at least 40%, at least 50%, at least 60%, at least 70%, at least 80%, at least 90%, at least 95%, or all of the genomic regions comprise, have or are within a genomic location defined in Tables 1 and/or 3. For example, at least 30%, at least 40%, at least 50%, at least 60%, at least 70%, at least 80%, at least 90%, at least 95%, or all of the genomic regions comprise or have a genomic location defined in Tables 1 and/or 3.

In certain embodiments, at least 25% of the genomic regions comprise, have or are within a genomic location defined in Tables 2 and/or 4. For example, at least 25% of the genomic regions comprise or have a genomic location defined in Tables 2 and/or 4.

In certain embodiments, at least 30%, at least 40%, at least 50%, at least 60%, at least 70%, at least 80%, at least 90%, at least 95%, or all of the genomic regions comprise, have or are within a genomic location defined in Tables 3 and/or 4. For example, at least 30%, at least 40%, at least 50%, at least 60%, at least 70%, at least 80%, at least 90%, at least 95%, or all of the genomic regions comprise or have a genomic location defined in Tables 3 and/or 4.

In certain preferred embodiments, determining the average methylation ratio for a genomic region comprises calculating the sum of the methylation ratios of all CpGs within the genomic region and dividing the sum by the number of CpGs within the genomic region. In such embodiments, the average methylation ratio may also be referred to as the mean methylation ratio. For the avoidance of doubt, if a genomic region has only one CpG locus, the average methylation ratio for the genomic region is the same as the methylation ratio for the single CpG locus in the genomic region.

The method of the present invention comprises calculating a methylation score using the average methylation ratio for each genomic region for which the average methylation ratio has been determined.

In certain embodiments, calculating a methylation score using the average methylation ratio for each genomic region comprises:
determining the median or the mean of the average methylation ratios for all genomic regions (i.e. all genomic regions for which an average methylation ratio has been determined in the method); or
determining the median or the mean of the average methylation ratios for a first group of genomic regions to obtain a first methylation score and/or determining the median or the mean of the average methylation ratios for second group of genomic regions to obtain a second methylation score; or
comparing the average methylation ratio at each genomic region to a reference methylation ratio for each genomic region to determine a methylation ratio score for each genomic region.

In one preferred embodiment, calculating a methylation score using the average methylation ratio for each genomic region comprises:
determining the median of the average methylation ratios for all genomic regions for which the average methylation ratio has been determined; or
determining the median of the average methylation ratios for a first group of genomic regions to obtain a first methylation score and/or determining the median of the average methylation ratios for second group of genomic regions to obtain a second methylation score; or
comparing the average methylation ratio at each genomic region to a reference methylation ratio for each genomic region to determine a methylation ratio score for each genomic region.

In one preferred embodiment, calculating a methylation score using the average methylation ratio for each genomic region comprises:
determining the median of the average methylation ratios for all genomic regions for which the average methylation ratio has been determined; or
determining the median of the average methylation ratios for a first group of genomic regions to obtain a first methylation score and/or determining the median of the average methylation ratios for second group of genomic regions to obtain a second methylation score.

In one preferred embodiment, calculating a methylation score using the average methylation ratio for each genomic region comprises
determining the median of the average methylation ratios for a first group of genomic regions to obtain a first methylation score and/or determining the median of the average methylation ratios for second group of genomic regions to obtain a second methylation score.

In very preferred embodiments wherein calculating a methylation score using the average methylation ratio for each genomic region comprises determining the median (or the mean) of the average methylation ratios for a first group of genomic regions to obtain a first methylation score and/or determining the median (or the mean) of the average methylation ratios for second group of genomic regions to obtain a second methylation score, the first group of genomic regions are all of the hypermethylated genomic regions (i.e. all hypermethylated genomic regions for which an average methylation ratio has been determined in the method, i.e. selected from those comprising, having or within a genomic location defined in Table 1 or 2), and the second group of genomic regions are all of the hypomethylated genomic regions (i.e. all hypomethylated genomic regions for which an average methylation ratio has been determined in the method, i.e. selected from those comprising, having or within a genomic location defined in Table 3 or 4, or Table 6).

In another embodiment, the first group of genomic regions are all of the genomic regions (for which the average methylation ratio has been determined) having a methylation pattern specific to prostate tissue (i.e. selected from those comprising, having or within a genomic location defined in Table 1 or 3), and the second group of genomic regions are all of the genomic regions (for which the average methylation ratio has been determined) having a methylation pattern specific to prostate cancer (i.e. selected from those comprising, having or within a genomic location defined in Table 2 or 4).

In one preferred embodiment, calculating a methylation score using the average methylation ratio for each genomic region comprises
determining the median of the average methylation ratios for all of the hypermethylated genomic regions (i.e. all hypermethylated genomic regions for which an average methylation ratio has been determined in the method, i.e. selected from those comprising, having or within a genomic location defined in Table 1 or 2) to obtain a first methylation score and determining the median of the average methylation ratios for all of the hypomethylated genomic regions (i.e. all hypomethylated genomic regions for which an average methylation ratio has been determined in the method, i.e. selected from those comprising, having or within a genomic location defined in Table 3 or 4, or Table 6) to obtain a second methylation score.

In one preferred embodiment, calculating a methylation score using the average methylation ratio for each genomic region comprises
determining the median of the average methylation ratios for all of the hypermethylated genomic regions (i.e. all hypermethylated genomic regions for which an average methylation ratio has been determined in the method, i.e. selected from those those comprising, having or within a genomic location defined in Table 1 or 2) to obtain a first methylation score.

In one preferred embodiment, calculating a methylation score using the average methylation ratio for each genomic region comprises
determining the median of the average methylation ratios for all of the hypomethylated genomic regions (i.e. all hypomethylated genomic regions for which an average methylation ratio has been determined in the method, i.e. selected from those those comprising, having or within a genomic location defined in Table 3 or 4, or Table 6) to obtain a second methylation score.

In one embodiment, calculating a methylation score using the average methylation ratio for each genomic region comprises comparing the average methylation ratio at each genomic region to a reference methylation ratio for each genomic region to determine a methylation ratio score for each genomic region. In such embodiments, preferably the reference methylation ratio is the average methylation ratio for the same genomic region in or covered by:
a cfDNA sample from a healthy subject, for example a healthy age-matched subject;
a tissue sample from a healthy subject, for example a prostate tissue sample from a healthy subject;
a cancer biopsy sample from a cancer patient, for example a prostate cancer biopsy sample from a prostate cancer patient;
a cancer cell line sample, for example a prostate cancer cell line sample from a prostate cancer cell line;
a sample of white blood cells from a subject, for example the subject or a healthy subject;
a cfDNA sample from a different subject having prostate cancer, wherein the level of prostate cancer fraction in the cfDNA sample from the different subject is known (preferably multiple cfDNA samples (for example at least 2, 3, 4, 5, 10, 20, 40, 50 or 100 samples) each from a different subject having prostate cancer, wherein the level of prostate cancer fraction in each cfDNA sample from the different subjects is known, and preferably wherein each cfDNA sample has a different level of prostate cancer fraction);
a characterized methylome sequence of a white blood cell;
a characterized methylome sequence of a prostate cancer cell line;
a characterized methylome sequence of a cancerous prostate cell; and/or
a characterized methylome sequence of a non-cancerous prostate cell.

In one preferred embodiment, the reference methylation ratio is the average methylation ratio for the same genomic region in or covered by
a cfDNA sample from a healthy subject, for example a healthy age-matched subject;
a sample of white blood cells from a subject, for example the subject or a healthy subject; and/or
a characterized methylome sequence of a white blood cell.

The method of the present invention comprises analyzing the methylation ratio scores to determine the level of prostate cancer fraction in the cfDNA sample. For example, no level (for example no detectable level) of prostate cancer fraction in the cfDNA sample may be determined. Alternatively, a level of cancer fraction in the cfDNA sample may be determined. The minimum percentage level of prostate cancer fraction in the cfDNA sample that may be determined may be 0.01% of cancer fraction in the cfDNA sample. In certain embodiments, the minimum percentage level of prostate cancer fraction in the cfDNA sample that may be determined may be 0.02%, 0.03%, 0.04%, 0.06%, 0.07%, 0.08%, 0.05%, 0.09%, 0.1%, 0.2%, 0.3%, 0.4%, 0.5%, 0.6%, 0.7%, 0.8%, 0.9%, or 1% of cancer fraction in the cfDNA sample. For example, the minimum percentage level of prostate cancer fraction in the cfDNA sample that may be determined may be 0.01%, 0.05%, 0.1% or 0.5% of cancer fraction in the cfDNA sample. Preferably, the minimum percentage level of prostate cancer fraction in the cfDNA is 0.01%.

The method comprises analyzing the methylation score to determine the level of prostate cancer fraction in the cfDNA sample.

Preferably, analyzing the methylation score to determine the level of prostate cancer fraction in the cfDNA sample comprises comparing the methylation score to one or more reference methylation scores. For example, the method may comprise comparing the methylation score to one reference methylation scores. In certain embodiments, the method comprises comparing the methylation score to two or more reference methylation scores, for example 2, 3, 4, 5, 6, 8, 9, 10, 12, 15, 20, 30, 50, 100, 200, 300, 400, 500 or 1000 reference methylation scores. In certain embodiments, the method comprises comparing the methylation score to 5 or more reference methylation scores, for example 10 or more, 15 or more, 20 or more, 30, or more 50, or more 100, or more 200, or more 300, or more 400, or more 500 or 1000 or more reference methylation scores.

In embodiments wherein the method comprises comparing the methylation score to two or more reference methylation scores, the reference methylation scores may come from different types of reference samples and/or reference methylomes (for example a cfDNA sample from a healthy subject and a cancer cell line sample) and/or the same type of reference samples or reference methylomes but from different sources (for example, two or more cfDNA samples each from a different a healthy subject).

A reference methylation score is a methylation score calculated for the same genomic regions (for example, calculated using the average methylation ratio for the same genomic regions) in a reference sample or reference methylome. A reference sample or reference methylome may be selected from the group consisting of:

a cfDNA sample from a healthy subject, for example a healthy age-matched subject;
a tissue sample from a healthy subject, for example a prostate tissue sample from a healthy subject;
a cancer biopsy sample from a cancer patient, for example a prostate cancer biopsy sample from a prostate cancer patient;
a cancer cell line sample, for example a prostate cancer cell line sample from a prostate cancer cell line;
a sample of white blood cells from a subject, for example the subject or a healthy subject;
a cfDNA sample from a different subject having prostate cancer, wherein the level of prostate cancer fraction in the cfDNA sample from the different subject is known (preferably multiple cfDNA samples (for example at least 2, 3, 4, 5, 10, 20, 40, 50 or 100 samples) each from a different subject having prostate cancer, wherein the level of prostate cancer fraction in each cfDNA sample from the different subjects is known, and preferably wherein each cfDNA sample has a different level of prostate cancer fraction);
a characterized methylome sequence of a white blood cell;
a characterized methylome sequence of a prostate cancer cell line;
a characterized methylome sequence of a cancerous prostate cell; and/or
a characterized methylome sequence of a non-cancerous prostate cell.

A reference sample or reference methylome may be one that can be used to represent a sample having 0% tumour fraction, for example a reference sample or reference methylome selected from one or more of the following

a cfDNA sample from a healthy subject, for example a healthy age-matched subject;
a sample of white blood cells from a subject, for example the subject or a healthy subject; and/or
a characterized methylome sequence of a white blood cell.

A reference sample or reference methylome may be one that can be used to represent a sample having 100% tumour fraction, for example a reference sample or reference methylome selected from one or more of the following

a cancer biopsy sample from a cancer patient, for example a prostate cancer biopsy sample from a prostate cancer patient;
a cancer cell line sample, for example a prostate cancer cell line sample from a prostate cancer cell line;
a characterized methylome sequence of a prostate cancer cell line; and/or
a characterized methylome sequence of a cancerous prostate cell.

A reference sample or reference methylome may be one that can be used to represent a sample having 10 to 90% tumour fraction, for example one or more cfDNA samples from different subjects having prostate cancer, wherein the level of prostate cancer fraction in each cfDNA sample from the different subjects is/are known. A level of prostate cancer fraction in each cfDNA sample can be determined by looking at genomic markers.

Preferably, analyzing the methylation score to determine the level of prostate cancer fraction in the cfDNA sample comprises comparing the methylation score to one or more reference methylation scores that can be used to represent a sample having 100% tumour fraction, and can be used to represent a sample having 0% tumour fraction, and optionally can be used to represent a sample having 10-90% tumour fraction. For example, analyzing the methylation score to determine the level of prostate cancer fraction in the cfDNA sample comprises:

comparing the methylation score to one or more reference methylation scores for a reference sample or reference methylome selected from the group consisting of:
a cfDNA sample from a healthy subject, for example a healthy age-matched subject,
a sample of white blood cells from a subject, for example the subject or a healthy subject, and/or
a characterized methylome sequence of a white blood cell;
   and
   comparing the methylation score to one or more reference methylation scores for a reference sample or reference methylome selected from the group consisting of:
a cancer biopsy sample from a cancer patient, for example a prostate cancer biopsy sample from a prostate cancer patient;
a cancer cell line sample, for example a prostate cancer cell line sample from a prostate cancer cell line;
a characterized methylome sequence of a prostate cancer cell line; and/or
a characterized methylome sequence of a cancerous prostate cell.
   and optionally comparing the methylation score to one or more reference methylation scores for one or more cfDNA samples from different subjects having prostate cancer, wherein the level of prostate cancer fraction in each cfDNA sample from the different subjects is/are known.

Preferably, the reference methylation score for a reference sample or reference methylome that a methylation ratio score is compared to is calculated in the same way as the methylation score for the sample obtained from the subject (i.e. the sample that the method of the invention is being carried out in respect of). For example, if the methylation ratio for the selected genomic regions of the sample obtained from the subject is calculated by determining the median (or the mean) of the average methylation ratios for a first group of genomic regions to obtain a first methylation score and/or determining the median (or the mean) of the average methylation ratios for second group of genomic regions to obtain a second methylation score, the reference methylation score for a reference sample or reference methylome is calculated by determining the median (or the mean) of the average methylation ratios for the same first group of genomic regions to obtain a first reference methylation score and/or determining the median (or the mean) of the average methylation ratios for the same second group of genomic regions to obtain a second reference methylation score.

Or, for example, if the methylation ratio for the selected genomic regions of the sample obtained from the subject is calculated by determining the median (or the mean) of the average methylation ratios for all genomic regions, the reference methylation score for a reference sample or reference methylome is calculated by determining the median (or the mean) of the average methylation ratios for the same genomic regions.

In embodiments wherein the method comprises comparing the average methylation ratio at each genomic region to a reference methylation ratio for each genomic region to determine a methylation ratio score for each genomic region, analyzing the methylation ratio scores to determine the level of prostate cancer fraction in the cfDNA sample may comprise determining how many methylation ratio scores are indicative of prostate cancer fraction in the cfDNA sample.

In certain embodiments, analyzing the methylation score to determine the level of prostate cancer fraction in the cfDNA sample comprises using a mathematical model, such as a linear regression model or another linear model (for example, a general linear model, a heteroscedastic model, a generalised linear model, or a hierarchical linear model).

In certain embodiments, analyzing the methylation score to determine the level of prostate cancer fraction in the cfDNA sample comprises using a mathematical model that compares the methylation score for the sample to reference methylation scores that can be used to represent a sample having 100% tumour fraction, and can be used to represent a sample having 0% tumour fraction, and optionally can be used to represent a sample having 10-90% tumour fraction. For example, the method comprises using mathematical model that compares the methylation score for the sample to reference methylation scores for a cfDNA sample from a healthy subject, for example a healthy age-matched subject (0% tumour fraction) and/or a characterized methylome sequence of a white blood cell (0% tumour fraction) and/or a sample of white blood cells from a subject, for example the subject or a healthy subject, (0% tumour fraction) and/or a characterized methylome sequence of a prostate cancer cell line (100% tumour fraction) and/or a prostate cancer biopsy sample from a prostate cancer patient (100% tumour fraction) and/or one or more cfDNA samples (for example at least 2, 3, 4, 5, 10, 20, 40, 50, 100, 200, 300 or 500 samples) each from a different subject having prostate cancer, wherein the level of prostate cancer fraction in each cfDNA sample from the different subjects is known, and preferably wherein each cfDNA sample has a different level of prostate cancer fraction (10-90% tumour fraction).

In one embodiment, the method comprises using mathematical model that compares the methylation score for the sample to reference methylation scores for a cfDNA sample from a healthy subject, for example a healthy age-matched subject (0% tumour fraction) and/or a characterized methylome sequence of a prostate cancer cell line (100% tumour fraction) and/or a prostate cancer biopsy sample from a prostate cancer patient (100% tumour fraction) and/or one or more cfDNA samples (for example at least 2, 3, 4, 5, 10, 20, 40, 50, 100, 200, 300 or 500 samples) each from a different subject having prostate cancer, wherein the level of prostate cancer fraction in each cfDNA sample from the different subjects is known, and preferably wherein each cfDNA sample has a different level of prostate cancer fraction (10-90% tumour fraction).

The method may further comprise measuring the level of prostate-specific antigen (PSA) in a sample of blood from the subject. It may also comprise determining if the subject has an abnormal level of PSA in the blood (for example a level of PSA in the blood of at least 4.0 ng/mL). An abnormal level of PSA in the blood may be, for example, a level of PSA in the blood of at least 4.0 ng/mL). A normal level of PSA in the blood may, for example, be a level of PSA in the blood of 4.0 ng/mL or less.

In one preferred embodiment, the method is for screening, monitoring, and/or prognostication of prostate cancer, wherein prostate cancer with a poor prognosis is predicted when a level of prostate cancer is determined, for example a detectable level of prostate cancer, for example a percentage level of prostate cancer fraction of at least 0.01%. For example, a prostate cancer with a poor prognosis is predicted when at least 0.01% prostate cancer fraction is determined, or for example, at least 0.02%, at least 0.03%, at least 0.04%, at least 0.05%, at least 0.1%, at least 0.5% or at least 1% prostate cancer fraction is determined.

In some instances, a “poor” prognosis refers to a low likelihood that a subject will likely respond favorably to a drug or set of drugs, is in complete or partial remission, or there is a decrease and/or a stop in the progression of prostate cancer. In some instances, a “poor” prognosis refers to a survival of a subject that is expected to be from less than 5 years to less than 1 month. In some instances, a “poor” prognosis refers to a survival of a subject in which the survival of the subject upon treatment is expected to be from less than 5 years to less than 1 month.

In one preferred embodiment, the method is for detection of prostate cancer, wherein prostate cancer is detected when a level of prostate cancer is determined, for example a detectable level of prostate cancer, for example a percentage level of prostate cancer fraction of at least 0.01%, or for example, at least 0.02%, at least 0.03%, at least 0.04%, at least 0.05%, at least 0.1%, at least 0.5% or at least 1% prostate cancer fraction.

In one preferred embodiment, the method is for screening, monitoring, and/or prognostication of prostate cancer, wherein prostate cancer with a poor prognosis is predicted when a level of prostate cancer is determined, for example a detectable level of prostate cancer, for example a percentage level of prostate cancer fraction of at least 0.01%, for example at least 0.01% prostate cancer fraction, or for example, at least 0.02%, at least 0.03%, at least 0.04%, at least 0.05%, at least 0.1%, at least 0.5% or at least 1% prostate cancer fraction.

In one preferred embodiment, the method is for detecting, screening and/or prognostication of metastatic prostate cancer, wherein metastatic prostate cancer is predicted when a level of prostate cancer is determined, for example a detectable level of prostate cancer, for example a percentage level of prostate cancer fraction of at least 0.01%, or for example, at least 0.02%, at least 0.03%, at least 0.04%, at least 0.05%, at least 0.1%, at least 0.5% or at least 1% prostate cancer fraction.

In one preferred embodiment, the method is for selecting treatment of prostate cancer or ascertaining whether treatment is working in prostate cancer, wherein a new treatment is selected when a level of prostate cancer is determined, for example a detectable level of prostate cancer, for example a percentage level of prostate cancer fraction of at least 0.01%, or for example, at least 0.02%, at least 0.03%, at least 0.04%, at least 0.05%, at least 0.1%, at least 0.5% or at least 1% prostate cancer fraction.

In one preferred embodiment, the method is for ascertaining whether treatment of prostate cancer is working, wherein it is determined that the treatment is not working when a level of prostate cancer is determined, for example a detectable level of prostate cancer, for example a percentage level of prostate cancer fraction of at least 0.01%, or for example, at least 0.02%, at least 0.03%, at least 0.04%, at least 0.05%, at least 0.1%, at least 0.5% or at least 1% prostate cancer fraction.

The method may further comprise repeating the method on second sample obtained from the subject after the subject has undergone a treatment for prostate cancer, wherein the second sample comprises cfDNA, and comparing the level of prostate cancer fraction in each sample. Preferably, the second sample is of the same type as the first sample, for example if the first sample is a plasma sample then the second sample is a plasma sample. The invention may further comprise repeating the method on a third, and optionally a 4^{th}, 5^{th}, 6^{th} 7^{th}, 8^{th}, 9^{th} and/or 10^{th}, sample obtained from the subject after the subject has undergone a treatment for prostate cancer, wherein the third, and optionally the 4^{th}, 5^{th}, 6^{th}, 7^{th}, 8^{th}, 9^{th} and/or 10^{th}, sample comprises circulating free DNA (cfDNA), and comparing the level of prostate cancer fraction in each sample. Preferably, all samples are of the same type as the first sample, for example if the first sample is a plasma sample the all other samples are plasma samples.

In one preferred embodiment, the method is for monitoring of prostate cancer, wherein the method comprises repeating the method on a second sample obtained from the subject after the subject has undergone a treatment for prostate cancer, wherein the second sample comprises cfDNA, and comparing the level of prostate cancer fraction in each sample.

In one preferred embodiment, the method is for selecting treatment of prostate cancer, comprising repeating the method on a second sample obtained from the subject after the subject has undergone a treatment for prostate cancer, wherein the second sample comprises cfDNA, and comparing the level of prostate cancer fraction in each sample, wherein a new treatment is selected if the level of prostate cancer is increased in the second sample, for example an increase of at least 0.01%.

In one preferred embodiment, the method is for ascertaining whether treatment of prostate cancer is working, comprising repeating the method on a second sample obtained from the subject after the subject has undergone a treatment for prostate cancer, wherein the second sample comprises cfDNA, wherein it is determined that the treatment is not working if the level of prostate cancer is increased in the second sample, for example an increase of at least 0.01%.

In one preferred embodiment, the method is for prognostication of prostate cancer, comprising repeating the method on a second sample obtained from the subject after the subject has undergone a treatment for prostate cancer, wherein the second sample comprises cfDNA, wherein it is determined that the prognosis is poor if the level of prostate cancer is increased in the second sample, for example an increase of at least 0.01%. In one preferred embodiment, the method is for prognostication of prostate cancer, comprising repeating the method on a second sample obtained from the subject after the subject has undergone a treatment for prostate cancer, wherein the second sample comprises cfDNA, wherein it is determined that the prognosis is good if the level of prostate cancer is decreased in the second sample, for example a decrease of at least 0.01%. In some instances, a “good” prognosis refers to the likelihood that a subject will likely respond favorably to a drug or set of drugs, leading to a complete or partial remission, or a decrease and/or a stop in the progression of prostate cancer. In some instances, a “good” prognosis refers to the survival of a subject of from at least 1 month to at least 90 years. In some instances, a “good” prognosis refers to the survival of a subject in which the survival of the subject upon treatment is from at least 1 month to at least 90 years.

In certain preferred embodiments, the method of present invention comprises the additional step of obtaining a biological sample from a subject.

The methods of the invention can be used with the kits, methods of treatment, therapeutic agents for the treatment of prostate cancer, methods of determining one or more suitable therapeutic agents for the treatment of prostate cancer, methods for determining a treatment regimen, computerized (or computer implemented) methods, computer-assisted methods, computer products and/or computer implemented software described herein. Embodiments and preferred embodiments for the methods of the invention are equally applicable to the kits, methods of treatment, therapeutic agents for the treatment of prostate cancer, methods of determining one or more suitable therapeutic agents for the treatment of prostate cancer, methods for determining a treatment regimen, computerized (or computer implemented) methods, computer-assisted methods, computer products and/or computer implemented software described herein.

Methods of the Invention to Determine Whether a Sample Comprises cfDNA Derived from a Prostate Cancer Subtype

The present invention also provides a method for detecting, screening, monitoring, staging, classification, selecting treatment for, ascertaining whether treatment is working in, and/or prognostication of prostate cancer in a sample obtained from a subject, wherein the sample comprises cfDNA, the method comprising:
characterizing the methylome sequence of a plurality of cfDNA molecules in the sample, wherein the methylome sequence of a cfDNA molecule is the DNA sequence and the methylation profile of the molecule;
determining the average methylation ratio at 10 or more genomic regions, each genomic region being selected from the group consisting of:
a 100 to 200 bp region comprising or having a genomic location defined in Table 8, and
a 2 to 99 bp region within a genomic location defined in Table 8 and comprising at least one CpG locus,
and wherein each of the genomic regions is covered by at least one sequence read of at least one characterized methylome sequence;
calculating a methylation score using the average methylation ratio for each of the genomic regions;
analyzing the methylation score to determine whether the sample comprises cfDNA derived from a prostate cancer subtype.

Tables 8 is provided below. The genomic locations of Table 8 are locations with reference to hg19.

| Chromosome | start | end | gene |
|---|---|---|---|
| chr12 | 52240301 | 52240400 | n/a |
| chr8 | 143535751 | 143535850 | n/a |
| chr17 | 81036151 | 81036250 | n/a |
| chr8 | 143535801 | 143535900 | n/a |
| chr5 | 142005201 | 142005300 | FGF1 |
| chr17 | 81036101 | 81036200 | n/a |
| chr12 | 52240351 | 52240450 | n/a |
| chr19 | 47736001 | 47736100 | BBC3 |
| chr10 | 3480051 | 3480150 | LOC105376360 |
| chr14 | 101123351 | 101123450 | LINC00523 |
| chr8 | 144303301 | 144303400 | n/a |
| chr7 | 95155001 | 95155100 | ASB4 |
| chr8 | 143535501 | 143535600 | n/a |
| chr15 | 41219401 | 41219500 | n/a |
| chr15 | 41219451 | 41219550 | n/a |
| chr7 | 1251201 | 1251300 | n/a |
| chr8 | 143535851 | 143535950 | n/a |
| chr2 | 189191651 | 189191750 | GULP1 |
| chr8 | 144303251 | 144303350 | n/a |
| chr8 | 143535601 | 143535700 | n/a |
| chr3 | 23782851 | 23782950 | n/a |
| chr1 | 1936451 | 1936550 | n/a |
| chr7 | 158800951 | 158801050 | LINC00689 |
| chr12 | 322251 | 322350 | SLC6A12 |
| chr1 | 15655951 | 15656050 | FHAD1 |
| chr8 | 143535701 | 143535800 | n/a |
| chr20 | 36037701 | 36037800 | n/a |
| chr20 | 36037751 | 36037850 | n/a |
| chr17 | 7083051 | 7083150 | ASGR1 |
| chr7 | 5319551 | 5319650 | n/a |
| chr17 | 7083001 | 7083100 | ASGR1 |
| chr10 | 131650451 | 131650550 | EBF3 |
| chr1 | 1936501 | 1936600 | n/a |
| chr19 | 35818801 | 35818900 | n/a |
| chr10 | 3479951 | 3480050 | LOC105376360 |
| chr4 | 1160801 | 1160900 | SPON2 |
| chr19 | 47735751 | 47735850 | BBC3 |
| chr10 | 3494301 | 3494400 | LOC105376360 |
| chr17 | 78982051 | 78982150 | n/a |
| chr10 | 4331801 | 4331900 | n/a |
| chr1 | 1920801 | 1920900 | CFAP74 |
| chr9 | 132482351 | 132482450 | PRRX2 |
| chr8 | 1923051 | 1923150 | KBTBD11 |
| chr16 | 1159851 | 1159950 | n/a |
| chr2 | 189191701 | 189191800 | GULP1 |
| chr1 | 200707101 | 200707200 | n/a |
| chr20 | 48124151 | 48124250 | PTGIS |
| chr19 | 35818851 | 35818950 | n/a |
| chr10 | 131650701 | 131650800 | EBF3 |
| chr10 | 3379051 | 3379150 | LOC105376360 |
| chr10 | 3449001 | 3449100 | LOC105376360 |
| chr12 | 107297051 | 107297150 | n/a |
| chr19 | 35981501 | 35981600 | KRTDAP |
| chr13 | 106063151 | 106063250 | n/a |
| chr5 | 2207051 | 2207150 | n/a |
| chr8 | 54164751 | 54164850 | OPRK1 |
| chr3 | 129326701 | 129326800 | n/a |
| chr1 | 223435701 | 223435800 | SUSD4 |
| chr2 | 11294551 | 11294650 | PQLC3 |
| chr17 | 25798951 | 25799050 | KSR1 |
| chr22 | 37215901 | 37216000 | PVALB |
| chr11 | 45392501 | 45392600 | LOC399886 |
| chr11 | 45392551 | 45392650 | LOC399886 |
| chr17 | 35277351 | 35277450 | n/a |
| chr9 | 89410901 | 89411000 | n/a |
| chr9 | 89410951 | 89411050 | n/a |
| chr8 | 103572851 | 103572950 | ODF1 |
| chr6 | 168629801 | 168629900 | n/a |
| chr3 | 129326651 | 129326750 | n/a |
| chr1 | 204655151 | 204655250 | LRRN2 |
| chr1 | 204655201 | 204655300 | LRRN2 |
| chr1 | 88108801 | 88108900 | n/a |
| chr10 | 4386801 | 4386900 | n/a |
| chr2 | 11294501 | 11294600 | PQLC3 |
| chr16 | 49530551 | 49530650 | ZNF423 |
| chr16 | 49530601 | 49530700 | ZNF423 |
| chr7 | 95155051 | 95155150 | ASB4 |
| chr10 | 73324401 | 73324500 | CDH23 |
| chr5 | 150538351 | 150538450 | ANXA6 |
| chr7 | 1388201 | 1388300 | n/a |
| chr3 | 186170701 | 186170800 | n/a |
| chr8 | 1923101 | 1923200 | KBTBD11 |
| chr8 | 54164651 | 54164750 | OPRK1 |
| chr16 | 1316401 | 1316500 | n/a |
| chr10 | 4386851 | 4386950 | n/a |
| chr4 | 1535701 | 1535800 | n/a |
| chr8 | 144213001 | 144213100 | n/a |
| chr10 | 131650651 | 131650750 | EBF3 |
| chr10 | 3480001 | 3480100 | LOC105376360 |
| chr3 | 64305701 | 64305800 | n/a |
| chr3 | 64305751 | 64305850 | n/a |
| chr1 | 1936551 | 1936650 | n/a |
| chr10 | 3480101 | 3480200 | LOC105376360 |
| chr10 | 3277051 | 3277150 | n/a |
| chr4 | 24796601 | 24796700 | SOD3 |
| chr3 | 46622551 | 46622650 | TDGF1 |
| chr14 | 104688501 | 104688600 | n/a |
| chr1 | 55504701 | 55504800 | PCSK9 |
| chr22 | 37215951 | 37216050 | PVALB |
| chr1 | 172291651 | 172291750 | DNM3 |
| chr1 | 2527501 | 2527600 | MMEL1 |
| chr15 | 27210251 | 27210350 | n/a |
| chr8 | 54164601 | 54164700 | OPRK1 |
| chr7 | 3019151 | 3019250 | CARD11 |
| chr11 | 71010451 | 71010550 | n/a |
| chr19 | 35981451 | 35981550 | KRTDAP |
| chr16 | 876151 | 876250 | n/a |
| chr8 | 1923001 | 1923100 | KBTBD11 |
| chr7 | 1251251 | 1251350 | n/a |
| chr1 | 38606051 | 38606150 | n/a |
| chr10 | 131650501 | 131650600 | EBF3 |
| chr4 | 140201651 | 140201750 | MGARP |
| chr14 | 105052601 | 105052700 | C14orf180 |
| chr10 | 3378851 | 3378950 | LOC105376360 |
| chr14 | 106095451 | 106095550 | n/a |
| chr12 | 6933201 | 6933300 | GPR162 |
| chr8 | 54164801 | 54164900 | OPRK1 |
| chr13 | 106063101 | 106063200 | n/a |
| chr10 | 94448551 | 94448650 | n/a |
| chr8 | 54164701 | 54164800 | OPRK1 |
| chr17 | 79459401 | 79459500 | n/a |
| chr7 | 158818151 | 158818250 | LINC00689 |
| chr6 | 25727351 | 25727450 | HIST1H2AA |
| chr5 | 1010951 | 1011050 | NKD2 |
| chr1 | 2424651 | 2424750 | PLCH2 |
| chr3 | 128724951 | 128725050 | EFCC1 |
| chr12 | 322951 | 323050 | SLC6A12 |
| chr10 | 3591201 | 3591300 | LOC105376360 |
| chr10 | 3591251 | 3591350 | LOC105376360 |
| chr1 | 2424701 | 2424800 | PLCH2 |
| chr7 | 1687001 | 1687100 | n/a |
| chr17 | 27396901 | 27397000 | n/a |
| chr4 | 7252451 | 7252550 | SORCS2 |
| chr10 | 134610401 | 134610500 | n/a |
| chr7 | 1388151 | 1388250 | n/a |
| chr5 | 2207001 | 2207100 | n/a |
| chr6 | 37503051 | 37503150 | LOC100505530 |
| chr10 | 131752851 | 131752950 | EBF3 |
| chr8 | 143546801 | 143546900 | ADGRB1 |
| chr15 | 102094651 | 102094750 | n/a |
| chr14 | 101128351 | 101128450 | LINC00523 |
| chr3 | 64338501 | 64338600 | n/a |
| chr3 | 64338551 | 64338650 | n/a |
| chr2 | 209271151 | 209271250 | PTH2R |
| chr1 | 15655901 | 15656000 | FHAD1 |
| chr16 | 29267801 | 29267900 | n/a |
| chr12 | 107297101 | 107297200 | n/a |
| chr22 | 43621801 | 43621900 | SCUBE1 |
| chr10 | 5406551 | 5406650 | UCN3 |
| chr17 | 79109751 | 79109850 | AATK |
| chr14 | 105052451 | 105052550 | C14orf180 |
| chr3 | 55931401 | 55931500 | ERC2 |
| chr3 | 55931451 | 55931550 | ERC2 |
| chr16 | 1316351 | 1316450 | n/a |
| chr10 | 3708501 | 3708600 | LOC105376360 |
| chr16 | 57317701 | 57317800 | PLLP |
| chr10 | 118084351 | 118084450 | CCDC172 |
| chr10 | 3572301 | 3572400 | LOC105376360 |
| chr1 | 3507101 | 3507200 | MEGF6 |
| chr8 | 700101 | 700200 | ERICH1-AS1 |
| chr9 | 6716301 | 6716400 | n/a |
| chr6 | 112132901 | 112133000 | FYN |
| chr8 | 143535651 | 143535750 | n/a |
| chr14 | 103691501 | 103691600 | n/a |
| chr4 | 1564101 | 1564200 | n/a |
| chr12 | 322151 | 322250 | SLC6A12 |
| chr12 | 322201 | 322300 | SLC6A12 |
| chr7 | 1686751 | 1686850 | n/a |
| chr3 | 128725001 | 128725100 | EFCC1 |
| chr10 | 4414951 | 4415050 | n/a |
| chr14 | 105052551 | 105052650 | C14orf180 |
| chr9 | 129282651 | 129282750 | n/a |
| chr9 | 129282701 | 129282800 | n/a |
| chr5 | 137225301 | 137225400 | PKD2L2 |
| chr1 | 7569301 | 7569400 | CAMTA1 |
| chr12 | 44858051 | 44858150 | n/a |
| chr20 | 43377501 | 43377600 | KCNK15 |
| chr20 | 43377551 | 43377650 | KCNK15 |
| chr1 | 1974801 | 1974900 | n/a |
| chr16 | 89009501 | 89009600 | CBFA2T3 |
| chr3 | 72704651 | 72704750 | n/a |
| chr14 | 70037601 | 70037700 | CCDC177 |
| chr6 | 25727301 | 25727400 | HIST1H2AA |
| chr15 | 27210301 | 27210400 | n/a |
| chr15 | 62543151 | 62543250 | n/a |
| chr10 | 3300501 | 3300600 | n/a |
| chr7 | 99067201 | 99067300 | n/a |
| chr6 | 168617401 | 168617500 | n/a |
| chr1 | 210501351 | 210501450 | HHAT |
| chr5 | 1207401 | 1207500 | SLC6A19 |
| chr10 | 131650401 | 131650500 | EBF3 |
| chr17 | 35277401 | 35277500 | n/a |
| chr5 | 173097501 | 173097600 | n/a |
| chr5 | 173097551 | 173097650 | n/a |
| chr17 | 76522851 | 76522950 | DNAH17 |
| chr4 | 3288751 | 3288850 | n/a |
| chr19 | 49528551 | 49528650 | CGB |
| chr19 | 49528601 | 49528700 | CGB |
| chr10 | 130844201 | 130844300 | n/a |
| chr1 | 172291701 | 172291800 | DNM3 |
| chr2 | 209271101 | 209271200 | PTH2R |
| chr1 | 6531301 | 6531400 | PLEKHG5 |
| chr22 | 40051501 | 40051600 | CACNA1I |
| chr16 | 876201 | 876300 | n/a |
| chr17 | 25798651 | 25798750 | KSR1 |
| chr17 | 25798701 | 25798800 | KSR1 |
| chr14 | 106174351 | 106174450 | n/a |
| chr16 | 22776051 | 22776150 | MIR548D2 |
| chr14 | 106174301 | 106174400 | n/a |
| chr2 | 3697501 | 3697600 | n/a |
| chr16 | 29267751 | 29267850 | n/a |
| chr7 | 1459051 | 1459150 | n/a |
| chr9 | 122734551 | 122734650 | n/a |
| chr10 | 4386751 | 4386850 | n/a |
| chr6 | 37527301 | 37527400 | n/a |
| chr17 | 21278901 | 21279000 | KCNJ12 |
| chr1 | 3347951 | 3348050 | PRDM16 |
| chr8 | 1707251 | 1707350 | n/a |
| chr10 | 3708451 | 3708550 | LOC105376360 |
| chr1 | 223435751 | 223435850 | SUSD4 |
| chr4 | 24796551 | 24796650 | SOD3 |
| chr6 | 45500901 | 45501000 | RUNX2 |
| chr1 | 38513551 | 38513650 | n/a |
| chr10 | 135054951 | 135055050 | VENTX |
| chr10 | 103326701 | 103326800 | n/a |
| chr16 | 4673901 | 4674000 | MGRN1 |
| chr19 | 44146901 | 44147000 | n/a |
| chr7 | 1686701 | 1686800 | n/a |
| chr3 | 14862901 | 14863000 | FGD5 |
| chr16 | 1159801 | 1159900 | n/a |
| chr1 | 210612301 | 210612400 | HHAT |
| chr8 | 142452401 | 142452500 | MROH5 |
| chr15 | 99088101 | 99088200 | n/a |
| chr21 | 43547751 | 43547850 | UMODL1 |
| chr10 | 130959651 | 130959750 | n/a |
| chr1 | 1974751 | 1974850 | n/a |
| chr20 | 61162201 | 61162300 | MIR133A2 |
| chr12 | 52238951 | 52239050 | n/a |
| chr12 | 52239001 | 52239100 | n/a |
| chr7 | 1251151 | 1251250 | n/a |
| chr19 | 17138801 | 17138900 | n/a |
| chr19 | 17138851 | 17138950 | n/a |
| chr15 | 68699651 | 68699750 | ITGA11 |
| chr10 | 3797401 | 3797500 | n/a |
| chr10 | 3797451 | 3797550 | n/a |
| chr5 | 149683251 | 149683350 | ARSI |
| chr5 | 149683301 | 149683400 | ARSI |
| chr2 | 159705601 | 159705700 | n/a |
| chr1 | 2424601 | 2424700 | PLCH2 |
| chr14 | 103691451 | 103691550 | n/a |
| chr5 | 1010901 | 1011000 | NKD2 |
| chr12 | 133178951 | 133179050 | n/a |
| chr12 | 107297251 | 107297350 | n/a |
| chr12 | 107297301 | 107297400 | n/a |
| chr22 | 43827801 | 43827900 | MPPED1 |
| chr11 | 72974051 | 72974150 | n/a |
| chr10 | 135054901 | 135055000 | VENTX |
| chr14 | 101128401 | 101128500 | LINC00523 |
| chr9 | 132482401 | 132482500 | PRRX2 |
| chr17 | 60214601 | 60214700 | n/a |
| chr16 | 57317651 | 57317750 | PLLP |
| chr5 | 162997901 | 162998000 | n/a |
| chr9 | 140127301 | 140127400 | SLC34A3 |
| chr17 | 78982001 | 78982100 | n/a |
| chr10 | 131650551 | 131650650 | EBF3 |
| chr20 | 61979401 | 61979500 | CHRNA4 |
| chr14 | 106095501 | 106095600 | n/a |
| chr3 | 72704701 | 72704800 | n/a |
| chr1 | 14220301 | 14220400 | n/a |
| chr5 | 2207101 | 2207200 | n/a |
| chr9 | 137660401 | 137660500 | COL5A1 |
| chr11 | 64739801 | 64739900 | C11orf85 |
| chr7 | 1329401 | 1329500 | n/a |
| chr13 | 106063051 | 106063150 | n/a |
| chr4 | 1535651 | 1535750 | n/a |
| chr17 | 14206951 | 14207050 | HS3ST3B1 |
| chr16 | 22776101 | 22776200 | MIR548D2 |
| chr4 | 6575801 | 6575900 | MAN2B2 |
| chr1 | 200707051 | 200707150 | n/a |
| chr14 | 103569401 | 103569500 | EXOC3L4 |
| chr1 | 7408801 | 7408900 | CAMTA1 |
| chr1 | 1920751 | 1920850 | CFAP74 |
| chr16 | 876101 | 876200 | n/a |
| chr16 | 474251 | 474350 | n/a |
| chr4 | 3288901 | 3289000 | n/a |
| chr1 | 3534401 | 3534500 | n/a |
| chr7 | 4678651 | 4678750 | n/a |
| chr19 | 36004901 | 36005000 | DMKN |
| chr5 | 131350101 | 131350200 | n/a |
| chr6 | 134350851 | 134350950 | SLC2A12 |
| chr9 | 132383101 | 132383200 | NTMT1 |
| chr10 | 131744351 | 131744450 | EBF3 |
| chr1 | 64197451 | 64197550 | n/a |
| chr20 | 61979301 | 61979400 | CHRNA4 |
| chr20 | 44934651 | 44934750 | CDH22 |
| chr1 | 9341951 | 9342050 | n/a |
| chr10 | 94448501 | 94448600 | n/a |
| chr4 | 3288851 | 3288950 | n/a |
| chr12 | 118312351 | 118312450 | KSR2 |
| chr20 | 21483901 | 21484000 | n/a |
| chr7 | 1329351 | 1329450 | n/a |
| chr3 | 185420301 | 185420400 | IGF2BP2 |
| chr3 | 185420351 | 185420450 | IGF2BP2 |
| chr10 | 131650751 | 131650850 | EBF3 |
| chr16 | 14380701 | 14380800 | n/a |
| chr11 | 57364951 | 57365050 | SERPING1 |
| chr17 | 25583251 | 25583350 | n/a |
| chr15 | 62543101 | 62543200 | n/a |
| chr19 | 47735951 | 47736050 | BBC3 |
| chr14 | 104639751 | 104639850 | KIF26A |
| chr5 | 1856051 | 1856150 | LOC101929034 |
| chr20 | 44934701 | 44934800 | CDH22 |
| chr10 | 134610451 | 134610550 | n/a |
| chr21 | 47398651 | 47398750 | n/a |
| chr10 | 3343101 | 3343200 | n/a |
| chr7 | 3019101 | 3019200 | CARD11 |
| chr21 | 44494701 | 44494800 | CBS |
| chr16 | 89009451 | 89009550 | CBFA2T3 |
| chr17 | 79109801 | 79109900 | AATK |
| chr9 | 139587851 | 139587950 | n/a |
| chr1 | 2527451 | 2527550 | MMEL1 |
| chr21 | 46973201 | 46973300 | n/a |
| chr2 | 202753101 | 202753200 | CDK15 |
| chr1 | 157140751 | 157140850 | n/a |
| chr5 | 2207151 | 2207250 | n/a |
| chr1 | 1097301 | 1097400 | n/a |
| chr17 | 63134151 | 63134250 | RGS9 |
| chr9 | 136500151 | 136500250 | n/a |
| chr3 | 194097051 | 194097150 | n/a |
| chr3 | 129326751 | 129326850 | n/a |
| chr7 | 2728901 | 2729000 | AMZ1 |
| chr5 | 137225001 | 137225100 | PKD2L2 |
| chr15 | 102094601 | 102094700 | n/a |
| chr10 | 4230551 | 4230650 | n/a |
| chr5 | 2205701 | 2205800 | n/a |
| chr16 | 14380651 | 14380750 | n/a |
| chr1 | 25298701 | 25298800 | n/a |
| chr11 | 1102501 | 1102600 | MUC2 |
| chr11 | 14994301 | 14994400 | CALCA |
| chr11 | 14994351 | 14994450 | CALCA |
| chr14 | 106438051 | 106438150 | ADAM6 |
| chr22 | 43829751 | 43829850 | MPPED1 |
| chr8 | 22018451 | 22018550 | SFTPC |
| chr21 | 34351051 | 34351150 | n/a |
| chr10 | 3544651 | 3544750 | LOC105376360 |
| chr11 | 60482451 | 60482550 | MS4A8 |
| chr11 | 2190101 | 2190200 | TH |
| chr20 | 4705201 | 4705300 | PRND |
| chr17 | 1811301 | 1811400 | n/a |
| chr5 | 141993201 | 141993300 | FGF1 |
| chr14 | 23290301 | 23290400 | n/a |
| chr17 | 60214651 | 60214750 | n/a |
| chr4 | 140201601 | 140201700 | MGARP |
| chr20 | 61979451 | 61979550 | CHRNA4 |
| chr11 | 64739751 | 64739850 | C11orf85 |
| chr16 | 1111151 | 1111250 | n/a |
| chr4 | 3288801 | 3288900 | n/a |
| chr1 | 38513601 | 38513700 | n/a |
| chr7 | 73466051 | 73466150 | ELN |
| chr16 | 24697401 | 24697500 | n/a |
| chr16 | 85201801 | 85201900 | n/a |
| chr9 | 137859601 | 137859700 | n/a |
| chr1 | 1936751 | 1936850 | n/a |
| chr1 | 22975601 | 22975700 | n/a |
| chr1 | 22975651 | 22975750 | n/a |
| chr5 | 1207451 | 1207550 | SLC6A19 |
| chr4 | 3865101 | 3865200 | n/a |
| chr21 | 46799851 | 46799950 | n/a |
| chr3 | 13058851 | 13058950 | IQSEC1 |
| chr1 | 7130401 | 7130500 | CAMTA1 |
| chr14 | 104852051 | 104852150 | n/a |
| chr5 | 1923501 | 1923600 | n/a |
| chr16 | 2863801 | 2863900 | n/a |
| chr11 | 120592101 | 120592200 | GRIK4 |
| chr11 | 120592151 | 120592250 | GRIK4 |
| chr1 | 17022551 | 17022650 | ESPNP |
| chr11 | 128796401 | 128796500 | n/a |
| chr15 | 75019301 | 75019400 | n/a |
| chr2 | 3697451 | 3697550 | n/a |
| chr11 | 120590051 | 120590150 | GRIK4 |
| chr11 | 120590101 | 120590200 | GRIK4 |
| chr12 | 49366151 | 49366250 | WNT10B |
| chr10 | 131650351 | 131650450 | EBF3 |
| chr8 | 144472051 | 144472150 | n/a |
| chr5 | 493301 | 493400 | SLC9A3 |
| chr1 | 234039901 | 234040000 | SLC35F3 |
| chr4 | 1564151 | 1564250 | n/a |
| chr14 | 103691301 | 103691400 | n/a |
| chr8 | 142452451 | 142452550 | MROH5 |
| chr7 | 1329451 | 1329550 | n/a |
| chr22 | 43805251 | 43805350 | n/a |
| chr22 | 43805301 | 43805400 | n/a |
| chr22 | 37771301 | 37771400 | ELFN2 |
| chr3 | 194090601 | 194090700 | LRRC15 |
| chr8 | 125249851 | 125249950 | LOC101927588 |
| chr7 | 2728851 | 2728950 | AMZ1 |
| chr7 | 1388251 | 1388350 | n/a |
| chr6 | 168629951 | 168630050 | n/a |
| chr19 | 36004951 | 36005050 | DMKN |
| chr11 | 63996751 | 63996850 | DNAJC4 |
| chr20 | 4705251 | 4705350 | PRND |
| chr3 | 196515551 | 196515650 | PAK2 |
| chr3 | 196515601 | 196515700 | PAK2 |
| chr17 | 65527651 | 65527750 | PITPNC1 |
| chr20 | 23969801 | 23969900 | GGTLC1 |
| chr7 | 23471801 | 23471900 | IGF2BP3 |
| chr6 | 134350801 | 134350900 | SLC2A12 |
| chr2 | 121279851 | 121279950 | n/a |
| chr4 | 184244751 | 184244850 | n/a |
| chr12 | 124607901 | 124608000 | ZNF664 - |
| | | | FAM101A |
| chr15 | 68699601 | 68699700 | ITGA11 |
| chr2 | 242151551 | 242151650 | ANO7 |
| chr5 | 2205751 | 2205850 | n/a |
| chr5 | 172924801 | 172924900 | n/a |
| chr5 | 137225351 | 137225450 | PKD2L2 |
| chr5 | 493251 | 493350 | SLC9A3 |
| chr8 | 144367251 | 144367350 | n/a |
| chr19 | 554951 | 555050 | n/a |
| chr12 | 1675901 | 1676000 | FBXL14 |
| chr5 | 74532301 | 74532400 | ANKRD31 |
| chr15 | 78186501 | 78186600 | n/a |
| chr16 | 24697451 | 24697550 | n/a |
| chr9 | 137859551 | 137859650 | n/a |
| chr1 | 21913451 | 21913550 | n/a |
| chr4 | 1537251 | 1537350 | n/a |
| chr11 | 69706801 | 69706900 | n/a |
| chr22 | 37771251 | 37771350 | ELFN2 |
| chr10 | 3526751 | 3526850 | LOC105376360 |
| chr2 | 219487501 | 219487600 | PLCD4 |
| chr2 | 219487551 | 219487650 | PLCD4 |
| chr16 | 876251 | 876350 | n/a |
| chr14 | 104639801 | 104639900 | KIF26A |
| chr8 | 700151 | 700250 | ERICH1-AS1 |
| chr6 | 18990551 | 18990650 | n/a |
| chr20 | 1164951 | 1165050 | TMEM74B |
| chr4 | 26493401 | 26493500 | n/a |
| chr6 | 168617451 | 168617550 | n/a |
| chr1 | 7408851 | 7408950 | CAMTA1 |
| chr10 | 131650301 | 131650400 | EBF3 |
| chr22 | 37771201 | 37771300 | ELFN2 |
| chr16 | 474301 | 474400 | n/a |
| chr17 | 66288801 | 66288900 | ARSG |
| chr21 | 41027851 | 41027950 | B3GALT5 |
| chr10 | 131706751 | 131706850 | EBF3 |
| chr7 | 1748001 | 1748100 | ELFN1 |
| chr12 | 52238601 | 52238700 | n/a |
| chr12 | 52238651 | 52238750 | n/a |
| chr7 | 158828251 | 158828350 | VIPR2 |
| chr5 | 137225401 | 137225500 | PKD2L2 |
| chr21 | 43547801 | 43547900 | UMODL1 |
| chr1 | 57718951 | 57719050 | DAB1 |
| chr1 | 57719001 | 57719100 | DAB1 |
| chr15 | 99974801 | 99974900 | n/a |
| chr14 | 104688551 | 104688650 | n/a |
| chr16 | 14380751 | 14380850 | n/a |
| chr21 | 44494751 | 44494850 | CBS |
| chr9 | 89411001 | 89411100 | n/a |
| chr19 | 14313651 | 14313750 | ADGRL1 |
| chr17 | 74237601 | 74237700 | n/a |
| chr19 | 3821051 | 3821150 | MIR1268A |
| chr3 | 66139601 | 66139700 | SLC25A26 |
| chr10 | 4482651 | 4482750 | n/a |
| chr10 | 3602701 | 3602800 | LOC105376360 |
| chr10 | 3602751 | 3602850 | LOC105376360 |
| chr15 | 29825301 | 29825400 | FAM189A1 |
| chr20 | 61979351 | 61979450 | CHRNA4 |
| chr12 | 322901 | 323000 | SLC6A12 |
| chr7 | 73466001 | 73466100 | ELN |
| chr17 | 79109701 | 79109800 | AATK |
| chr10 | 5407001 | 5407100 | UCN3 |
| chr11 | 67462801 | 67462900 | n/a |
| chr7 | 45188151 | 45188250 | n/a |
| chr1 | 87994651 | 87994750 | n/a |
| chr11 | 64780701 | 64780800 | ARL2 |
| chr7 | 73790751 | 73790850 | CLIP2 |
| chr5 | 532951 | 533050 | n/a |
| chr2 | 242797901 | 242798000 | PDCD1 |
| chr15 | 23894801 | 23894900 | n/a |
| chr15 | 23894751 | 23894850 | n/a |
| chr5 | 2206751 | 2206850 | n/a |
| chr7 | 1407501 | 1407600 | n/a |
| chr20 | 23970051 | 23970150 | GGTLC1 |
| chr19 | 554851 | 554950 | n/a |
| chr5 | 2205951 | 2206050 | n/a |
| chr15 | 101807351 | 101807450 | n/a |
| chr4 | 1160751 | 1160850 | SPON2 |
| chr14 | 104768501 | 104768600 | n/a |
| chr9 | 6716351 | 6716450 | n/a |
| chr2 | 66743751 | 66743850 | MEIS1 |
| chr17 | 25798401 | 25798500 | KSR1 |
| chr11 | 102216851 | 102216950 | BIRC2 |
| chr10 | 4358501 | 4358600 | n/a |
| chr12 | 116008051 | 116008150 | n/a |
| chr14 | 70476801 | 70476900 | SMOC1 |
| chr9 | 139587901 | 139588000 | n/a |
| chr7 | 131831551 | 131831650 | PLXNA4 |
| chr5 | 141993251 | 141993350 | FGF1 |
| chr3 | 194097101 | 194097200 | n/a |
| chr16 | 88963701 | 88963800 | CBFA2T3 |
| chr15 | 29037851 | 29037950 | PDCD6IPP2 |
| chr6 | 134350901 | 134351000 | SLC2A12 |
| chr8 | 143546851 | 143546950 | ADGRB1 |
| chr9 | 129387201 | 129387300 | LMX1B |
| chr14 | 104617951 | 104618050 | KIF26A |
| chr4 | 3288401 | 3288500 | n/a |
| chr8 | 81963301 | 81963400 | PAG1 |
| chr8 | 81963351 | 81963450 | PAG1 |
| chr3 | 126080301 | 126080400 | n/a |
| chr9 | 136567001 | 136567100 | SARDH |
| chr7 | 1329001 | 1329100 | n/a |
| chr6 | 37014501 | 37014600 | n/a |
| chr6 | 37014551 | 37014650 | n/a |
| chr10 | 3544601 | 3544700 | LOC105376360 |
| chr4 | 3776451 | 3776550 | n/a |
| chr11 | 72980801 | 72980900 | P2RY6 |
| chr14 | 76877951 | 76878050 | ESRRB |
| chr11 | 120044501 | 120044600 | n/a |
| chr2 | 159705351 | 159705450 | n/a |
| chr12 | 86230751 | 86230850 | RASSF9 |
| chr12 | 86230801 | 86230900 | RASSF9 |
| chr14 | 94406501 | 94406600 | ASB2 |
| chr14 | 106438101 | 106438200 | ADAM6 |
| chr7 | 29186301 | 29186400 | CPVL |
| chr16 | 29242051 | 29242150 | n/a |
| chr4 | 187071151 | 187071250 | FAM149A |
| chr19 | 40032701 | 40032800 | n/a |
| chr17 | 77536201 | 77536300 | n/a |
| chr3 | 97542301 | 97542400 | CRYBG3 |
| chr6 | 25761601 | 25761700 | SLC17A4 |
| chr1 | 9342001 | 9342100 | n/a |
| chr17 | 60828151 | 60828250 | Mar-10 |
| chr19 | 5455301 | 5455400 | ZNRF4 |
| chr7 | 44279601 | 44279700 | CAMK2B |
| chr14 | 106174251 | 106174350 | n/a |
| chr1 | 156831151 | 156831250 | NTRK1 |
| chr5 | 150538301 | 150538400 | ANXA6 |
| chr2 | 239695751 | 239695850 | n/a |
| chr21 | 46816651 | 46816750 | n/a |
| chr5 | 162997951 | 162998050 | n/a |
| chr10 | 3457351 | 3457450 | LOC105376360 |
| chr1 | 7539101 | 7539200 | CAMTA1 |
| chr7 | 1137351 | 1137450 | C7orf50 |
| chr5 | 180597551 | 180597650 | n/a |
| chr12 | 52240401 | 52240500 | n/a |
| chr2 | 71099251 | 71099350 | n/a |
| chr11 | 62100751 | 62100850 | n/a |
| chr14 | 101928001 | 101928100 | n/a |
| chr14 | 94463751 | 94463850 | LINC00521 |
| chr14 | 94463801 | 94463900 | LINC00521 |
| chr14 | 101123451 | 101123550 | LINC00523 |
| chr7 | 3488801 | 3488900 | SDK1 |
| chr5 | 132944101 | 132944200 | FSTL4 |
| chr10 | 131034801 | 131034900 | n/a |
| chr1 | 38517201 | 38517300 | n/a |
| chr20 | 62004751 | 62004850 | n/a |
| chr5 | 1217751 | 1217850 | SLC6A19 |
| chr15 | 60919451 | 60919550 | RORA-AS1 |
| chr16 | 88963651 | 88963750 | CBFA2T3 |
| chr2 | 159705401 | 159705500 | n/a |
| chr9 | 135033201 | 135033300 | n/a |
| chr17 | 7082951 | 7083050 | ASGR1 |
| chr19 | 18902651 | 18902750 | COMP |
| chr19 | 18902701 | 18902800 | COMP |
| chr1 | 6531151 | 6531250 | PLEKHG5 |
| chr1 | 1084501 | 1084600 | n/a |
| chr1 | 1084551 | 1084650 | n/a |
| chr10 | 3708401 | 3708500 | LOC105376360 |
| chr10 | 131691251 | 131691350 | EBF3 |
| chr5 | 2205901 | 2206000 | n/a |
| chr13 | 113807851 | 113807950 | n/a |
| chr7 | 127881551 | 127881650 | LEP |
| chr5 | 2335601 | 2335700 | n/a |
| chr21 | 42219751 | 42219850 | DSCAM |
| chr10 | 130959601 | 130959700 | n/a |
| chr10 | 4697351 | 4697450 | LINC00704 |
| chr10 | 4697401 | 4697500 | LINC00705 |
| chr7 | 1407301 | 1407400 | n/a |
| chr5 | 137224951 | 137225050 | PKD2L2 |
| chr1 | 226756401 | 226756500 | C1orf95 |
| chr1 | 226756451 | 226756550 | C1orf95 |
| chr1 | 200143101 | 200143200 | NR5A2 |
| chr11 | 67219501 | 67219600 | CABP4 |
| chr6 | 168629851 | 168629950 | n/a |
| chr17 | 14207001 | 14207100 | HS3ST3B1 |
| chr4 | 74847801 | 74847900 | PF4 |
| chr11 | 67619801 | 67619900 | n/a |
| chr9 | 138171701 | 138171800 | n/a |
| chr2 | 54560551 | 54560650 | C2orf73 |
| chr1 | 15655851 | 15655950 | FHAD1 |
| chr22 | 32750851 | 32750950 | RFPL3 |
| chr1 | 156828651 | 156828750 | INSRR |
| chr14 | 103691351 | 103691450 | n/a |
| chr2 | 27938101 | 27938200 | n/a |
| chr10 | 118084301 | 118084400 | CCDC172 |
| chr16 | 85198551 | 85198650 | n/a |
| chr22 | 37499451 | 37499550 | TMPRSS6 |
| chr3 | 139258301 | 139258400 | RBP1 |
| chr22 | 50457151 | 50457250 | n/a |
| chr11 | 75222401 | 75222500 | GDPD5 |
| chr6 | 169351351 | 169351450 | n/a |
| chr5 | 532901 | 533000 | n/a |
| chr14 | 93154751 | 93154850 | RIN3 |
| chr14 | 104623601 | 104623700 | KIF26A |
| chr11 | 63996801 | 63996900 | DNAJC4 |
| chr6 | 112132951 | 112133050 | FYN |
| chr4 | 3691301 | 3691400 | n/a |
| chr7 | 4870201 | 4870300 | RADIL |
| chr15 | 66543901 | 66544000 | MEGF11 |
| chr14 | 105105101 | 105105200 | n/a |
| chr7 | 564251 | 564350 | HRAT92 |
| chr1 | 14220251 | 14220350 | n/a |
| chr16 | 1316151 | 1316250 | n/a |
| chr1 | 21044901 | 21045000 | KIF17 |
| chr3 | 169540251 | 169540350 | LRRIQ4 |
| chr1 | 64197401 | 64197500 | n/a |
| chr1 | 231761601 | 231761700 | DISC1 |
| chr3 | 54353651 | 54353750 | CACNA2D3 |
| chr10 | 3500151 | 3500250 | LOC105376360 |
| chr1 | 23521351 | 23521450 | HTR1D |
| chr9 | 139925801 | 139925900 | C9orf139 |
| chr8 | 1644901 | 1645000 | DLGAP2 |
| chr8 | 1644951 | 1645050 | DLGAP2 |
| chr5 | 150538401 | 150538500 | ANXA6 |
| chr19 | 47735701 | 47735800 | BBC3 |
| chr1 | 22889251 | 22889350 | EPHA8 |
| chr14 | 106229551 | 106229650 | n/a |
| chr22 | 43621751 | 43621850 | SCUBE1 |
| chr14 | 89881701 | 89881800 | FOXN3 |
| chr20 | 30618851 | 30618950 | CCM2L |
| chr3 | 14595751 | 14595850 | n/a |
| chr16 | 84336251 | 84336350 | WFDC1 |
| chr17 | 26795251 | 26795350 | n/a |
| chr14 | 104770801 | 104770900 | n/a |
| chr11 | 102216901 | 102217000 | BIRC2 |
| chr9 | 122734601 | 122734700 | n/a |
| chr3 | 169540101 | 169540200 | LRRIQ4 |
| chr16 | 14380601 | 14380700 | n/a |
| chr21 | 46420501 | 46420600 | LINC00162 |
| chr11 | 68781901 | 68782000 | MRGPRF-AS1 |
| chr16 | 22776001 | 22776100 | MIR548D2 |
| chr7 | 30718001 | 30718100 | CRHR2 |
| chr5 | 137225251 | 137225350 | PKD2L2 |
| chr4 | 3690751 | 3690850 | n/a |
| chr10 | 4194451 | 4194550 | n/a |
| chr1 | 205913951 | 205914050 | n/a |
| chr5 | 114514651 | 114514750 | TRIM36 |
| chr17 | 75789551 | 75789650 | n/a |
| chr9 | 33448251 | 33448350 | AQP3 |
| chr11 | 4843051 | 4843150 | OR51F2 |
| chr17 | 41739251 | 41739350 | MEOX1 |
| chr16 | 1295551 | 1295650 | n/a |
| chr2 | 159705551 | 159705650 | n/a |
| chr4 | 7652101 | 7652200 | SORCS2 |
| chr10 | 134662251 | 134662350 | CFAP46 |
| chr7 | 1329301 | 1329400 | n/a |
| chr12 | 47219951 | 47220050 | SLC38A4 |
| chr10 | 13039651 | 13039750 | CCDC3 |
| chr1 | 226791451 | 226791550 | C1orf95 |
| chr8 | 143261951 | 143262050 | n/a |
| chr17 | 81036051 | 81036150 | n/a |
| chr10 | 28971201 | 28971300 | BAMBI |
| chr17 | 34996051 | 34996150 | n/a |
| chr14 | 105052501 | 105052600 | C14orf180 |
| chr7 | 44279651 | 44279750 | CAMK2B |
| chr7 | 3018401 | 3018500 | CARD11 |
| chr10 | 131650601 | 131650700 | EBF3 |
| chr17 | 1811351 | 1811450 | n/a |
| chr21 | 47399551 | 47399650 | n/a |
| chr2 | 121279801 | 121279900 | n/a |
| chr10 | 3568801 | 3568900 | LOC105376360 |
| chr19 | 15585451 | 15585550 | PGLYRP2 |
| chr8 | 42009151 | 42009250 | n/a |
| chr11 | 2293051 | 2293150 | ASCL2 |
| chr10 | 3250701 | 3250800 | n/a |
| chr2 | 86037151 | 86037250 | n/a |
| chr1 | 1936601 | 1936700 | n/a |
| chr7 | 3018601 | 3018700 | CARD11 |
| chr17 | 78456401 | 78456500 | n/a |
| chr10 | 134303901 | 134304000 | n/a |
| chr8 | 144303201 | 144303300 | n/a |
| chr13 | 28562501 | 28562600 | URAD |
| chr13 | 28562551 | 28562650 | URAD |
| chr9 | 132482451 | 132482550 | PRRX2 |
| chr1 | 48360401 | 48360500 | TRABD2B |
| chr1 | 48360451 | 48360550 | TRABD2B |
| chr14 | 100625001 | 100625100 | DEGS2 |
| chr5 | 180597601 | 180597700 | n/a |
| chr14 | 70348401 | 70348500 | SMOC1 |
| chr14 | 70348451 | 70348550 | SMOC1 |
| chr11 | 62100701 | 62100800 | n/a |
| chr9 | 136567051 | 136567150 | SARDH |
| chr14 | 37075451 | 37075550 | n/a |
| chr10 | 4194501 | 4194600 | n/a |
| chr21 | 46799901 | 46800000 | n/a |
| chr16 | 57916851 | 57916950 | CNGB1 |
| chr10 | 3343001 | 3343100 | n/a |
| chr10 | 1602501 | 1602600 | ADARB2 |
| chr1 | 226791351 | 226791450 | C1orf95 |
| chr6 | 41435651 | 41435750 | n/a |
| chr2 | 26788701 | 26788800 | C2orf70 |
| chr20 | 62004701 | 62004800 | n/a |
| chr7 | 24328551 | 24328650 | NPY |
| chr19 | 1505901 | 1506000 | ADAMTSL5 |
| chr9 | 34588501 | 34588600 | CNTFR |
| chr10 | 3343051 | 3343150 | n/a |
| chr9 | 132383301 | 132383400 | NTMT1 |
| chr1 | 205913901 | 205914000 | n/a |
| chr2 | 242797851 | 242797950 | PDCD1 |
| chr9 | 132383351 | 132383450 | NTMT1 |
| chr4 | 8158251 | 8158350 | ABLIM2 |
| chr10 | 3281051 | 3281150 | n/a |
| chr15 | 62358751 | 62358850 | C2CD4A |
| chr15 | 33437351 | 33437450 | FMN1 |
| chr15 | 78114851 | 78114950 | n/a |
| chr7 | 99987501 | 99987600 | PILRA |
| chr4 | 1504551 | 1504650 | n/a |
| chr5 | 140710351 | 140710450 | PCDHGA1 |
| chr6 | 33561351 | 33561450 | LINC00336 |
| chr6 | 33561401 | 33561500 | LINC00336 |
| chr3 | 169540301 | 169540400 | LRRIQ4 |
| chr8 | 143570901 | 143571000 | ADGRB1 |
| chr14 | 101123301 | 101123400 | LINC00523 |
| chr15 | 99088051 | 99088150 | n/a |
| chr19 | 36195351 | 36195450 | ZBTB32 |
| chr16 | 67336051 | 67336150 | KCTD19 |
| chr1 | 63798301 | 63798400 | n/a |
| chr1 | 63798351 | 63798450 | n/a |
| chr7 | 36013301 | 36013400 | n/a |
| chr5 | 2204551 | 2204650 | n/a |
| chr3 | 139258251 | 139258350 | RBP1 |
| chr11 | 67462851 | 67462950 | n/a |
| chr19 | 36195401 | 36195500 | ZBTB32 |
| chr17 | 1202251 | 1202350 | TUSC5 |
| chr16 | 281351 | 281450 | n/a |
| chr15 | 75019351 | 75019450 | n/a |
| chr10 | 4446051 | 4446150 | LINC00703 |
| chr17 | 60214551 | 60214650 | n/a |
| chr1 | 200175551 | 200175650 | n/a |
| chr1 | 154843201 | 154843300 | KCNN3 |
| chr7 | 1747951 | 1748050 | ELFN1 |
| chr16 | 29242101 | 29242200 | n/a |
| chr8 | 143868151 | 143868250 | LY6D |
| chr4 | 3752251 | 3752350 | n/a |
| chr6 | 130992701 | 130992800 | n/a |
| chr7 | 1684601 | 1684700 | n/a |
| chr11 | 2210201 | 2210300 | n/a |
| chr17 | 79109601 | 79109700 | AATK |
| chr14 | 103569351 | 103569450 | EXOC3L4 |
| chr8 | 136510551 | 136510650 | KHDRBS3 |
| chr7 | 1358201 | 1358300 | n/a |
| chr10 | 3373301 | 3373400 | LOC105376360 |
| chr6 | 46455901 | 46456000 | RCAN2 |
| chr6 | 46455951 | 46456050 | RCAN2 |
| chr5 | 73969151 | 73969250 | HEXB |
| chr1 | 203525601 | 203525700 | n/a |
| chr22 | 37771351 | 37771450 | ELFN2 |
| chr19 | 17571601 | 17571700 | NXNL1 |
| chr2 | 202753251 | 202753350 | CDK15 |
| chr13 | 50703451 | 50703550 | DLEU1 |
| chr3 | 185866551 | 185866650 | DGKG |
| chr12 | 116008101 | 116008200 | n/a |
| chr11 | 62100801 | 62100900 | n/a |
| chr4 | 3690901 | 3691000 | n/a |
| chr9 | 140127251 | 140127350 | SLC34A3 |
| chr7 | 3018451 | 3018550 | CARD11 |
| chr7 | 99987601 | 99987700 | PILRA |
| chr5 | 2537751 | 2537850 | n/a |
| chr16 | 30034801 | 30034900 | C16orf92 |
| chr22 | 37500701 | 37500800 | TMPRSS6 |
| chr9 | 132315801 | 132315900 | n/a |
| chr10 | 2978801 | 2978900 | n/a |
| chr1 | 61408051 | 61408150 | NFIA-AS2 |
| chr11 | 62100651 | 62100750 | n/a |
| chr17 | 66288751 | 66288850 | ARSG |
| chr7 | 2959101 | 2959200 | CARD11 |
| chr22 | 25160851 | 25160950 | PIWIL3 |
| chr20 | 23970101 | 23970200 | GGTLC1 |
| chr4 | 1537551 | 1537650 | n/a |
| chr2 | 27938151 | 27938250 | n/a |
| chr1 | 226791401 | 226791500 | C1orf95 |
| chr14 | 104768451 | 104768550 | n/a |
| chr10 | 3250751 | 3250850 | n/a |
| chr1 | 218537401 | 218537500 | TGFB2 |
| chr1 | 229480101 | 229480200 | n/a |
| chr7 | 30029851 | 30029950 | SCRN1 |
| chr7 | 30029901 | 30030000 | SCRN1 |
| chr16 | 2863851 | 2863950 | n/a |
| chr3 | 64225051 | 64225150 | n/a |
| chr3 | 64225101 | 64225200 | n/a |
| chr22 | 25160451 | 25160550 | PIWIL3 |
| chr14 | 65289701 | 65289800 | SPTB |
| chr7 | 4843901 | 4844000 | RADIL |
| chr16 | 90115051 | 90115150 | URAHP |
| chr16 | 90115101 | 90115200 | URAHP |
| chr19 | 3030301 | 3030400 | TLE2 |
| chr4 | 3677601 | 3677700 | LOC100133461 |
| chr5 | 140710501 | 140710600 | PCDHGA1 |
| chr2 | 242797751 | 242797850 | PDCD1 |
| chr14 | 93154701 | 93154800 | RIN3 |
| chr15 | 29611951 | 29612050 | FAM189A1 |
| chr14 | 106208351 | 106208450 | n/a |
| chr11 | 120561251 | 120561350 | GRIK4 |
| chr17 | 27396951 | 27397050 | n/a |
| chr6 | 17988951 | 17989050 | n/a |
| chr19 | 45720101 | 45720200 | EXOC3L2 |
| chr10 | 4296351 | 4296450 | n/a |
| chr4 | 187729101 | 187729200 | n/a |
| chr4 | 187729151 | 187729250 | n/a |
| chr1 | 94270151 | 94270250 | BCAR3 |
| chr3 | 127173651 | 127173750 | n/a |
| chr16 | 84336301 | 84336400 | WFDC1 |
| chr7 | 89747951 | 89748050 | DPY19L2P4 |
| chr2 | 239048601 | 239048700 | KLHL30 |
| chr5 | 1010851 | 1010950 | NKD2 |
| chr1 | 87994701 | 87994800 | n/a |
| chr19 | 51538151 | 51538250 | KLK12 |
| chr17 | 41739201 | 41739300 | MEOX1 |
| chr10 | 112834851 | 112834950 | n/a |
| chr19 | 41062001 | 41062100 | SPTBN4 |
| chr16 | 281401 | 281500 | n/a |
| chr7 | 99987551 | 99987650 | PILRA |
| chr10 | 3313151 | 3313250 | n/a |
| chr20 | 61371501 | 61371600 | NTSR1 |
| chr22 | 26877601 | 26877700 | HPS4 |
| chr22 | 26877651 | 26877750 | HPS4 |
| chr22 | 18508301 | 18508400 | MICAL3 |
| chr16 | 3142651 | 3142750 | ZSCAN10 |
| chr6 | 170585851 | 170585950 | LOC285804 |
| chr9 | 122800851 | 122800950 | n/a |
| chr12 | 299701 | 299800 | SLC6A12 |
| chr15 | 33437301 | 33437400 | FMN1 |
| chr10 | 4378551 | 4378650 | n/a |
| chr10 | 4378601 | 4378700 | n/a |
| chr12 | 111137051 | 111137150 | n/a |
| chr7 | 2728751 | 2728850 | AMZ1 |
| chr11 | 72980851 | 72980950 | P2RY6 |
| chr19 | 3030251 | 3030350 | TLE2 |
| chr15 | 29825351 | 29825450 | FAM189A1 |
| chr1 | 210612251 | 210612350 | HHAT |
| chr16 | 88880801 | 88880900 | GALNS |
| chr15 | 60919401 | 60919500 | RORA |
| chr7 | 1137301 | 1137400 | C7orf50 |
| chr5 | 180597651 | 180597750 | n/a |
| chr2 | 42077601 | 42077700 | n/a |
| chr10 | 134610351 | 134610450 | n/a |
| chr14 | 104852001 | 104852100 | n/a |
| chr8 | 144854651 | 144854750 | n/a |
| chr10 | 94448451 | 94448550 | n/a |
| chr1 | 15685251 | 15685350 | FHAD1 |
| chr13 | 28563651 | 28563750 | URAD |
| chr6 | 25727151 | 25727250 | HIST1H2AA |
| chr17 | 75848751 | 75848850 | n/a |
| chr5 | 137225101 | 137225200 | PKD2L2 |
| chr19 | 56914751 | 56914850 | ZNF583 |
| chr7 | 23471751 | 23471850 | IGF2BP3 |
| chr14 | 104627851 | 104627950 | KIF26A |
| chr1 | 4794901 | 4795000 | AJAP1 |
| chr19 | 46651201 | 46651300 | IGFL2 |
| chr17 | 21278851 | 21278950 | KCNJ12 |
| chr12 | 58736301 | 58736400 | n/a |
| chr5 | 73969201 | 73969300 | HEXB |
| chr17 | 77644501 | 77644600 | n/a |
| chr12 | 322601 | 322700 | SLC6A12 |
| chr2 | 189191601 | 189191700 | GULP1 |
| chr1 | 14220201 | 14220300 | n/a |
| chr6 | 168629901 | 168630000 | n/a |
| chr1 | 861751 | 861850 | SAMD11 |
| chr7 | 3018351 | 3018450 | CARD11 |
| chr7 | 2728801 | 2728900 | AMZ1 |
| chr12 | 116944101 | 116944200 | n/a |
| chr7 | 89747901 | 89748000 | STEAP2-AS1 |
| chr6 | 168630001 | 168630100 | n/a |
| chr16 | 29242001 | 29242100 | n/a |
| chr7 | 1329051 | 1329150 | n/a |
| chr5 | 170743851 | 170743950 | n/a |
| chr1 | 65362451 | 65362550 | JAK1 |
| chr7 | 1407351 | 1407450 | n/a |
| chr10 | 4358551 | 4358650 | n/a |
| chr11 | 92806401 | 92806500 | n/a |
| chr14 | 101123501 | 101123600 | LINC00523 |
| chr8 | 914451 | 914550 | ERICH1-AS1 |
| chr7 | 1407251 | 1407350 | n/a |
| chr2 | 113379951 | 113380050 | n/a |
| chr14 | 100631751 | 100631850 | n/a |
| chr12 | 44858001 | 44858100 | n/a |
| chr14 | 104865801 | 104865900 | n/a |
| chr8 | 94508451 | 94508550 | LINC00535 |
| chr6 | 25727251 | 25727350 | HIST1H2AA |
| chr19 | 4566501 | 4566600 | n/a |
| chr21 | 44724701 | 44724800 | n/a |
| chr7 | 158800601 | 158800700 | LINC00689 |
| chr9 | 138109251 | 138109350 | n/a |
| chr11 | 69706751 | 69706850 | n/a |
| chr6 | 25727001 | 25727100 | HIST1H2BA |
| chr9 | 137731801 | 137731900 | COL5A1 |
| chr19 | 56914801 | 56914900 | ZNF583 |
| chr14 | 23290351 | 23290450 | n/a |
| chr5 | 137225151 | 137225250 | PKD2L2 |
| chr10 | 3300451 | 3300550 | n/a |
| chr10 | 130959701 | 130959800 | n/a |
| chr17 | 27347151 | 27347250 | n/a |
| chr4 | 1535601 | 1535700 | n/a |
| chr10 | 34496301 | 34496400 | PARD3 |
| chr3 | 14595851 | 14595950 | n/a |
| chr7 | 3018301 | 3018400 | CARD11 |
| chr6 | 168533451 | 168533550 | n/a |
| chr16 | 1198651 | 1198750 | n/a |
| chr11 | 2293201 | 2293300 | n/a |
| chr14 | 105044951 | 105045050 | C14orf180 |
| chr11 | 2293251 | 2293350 | n/a |
| chr10 | 131357151 | 131357250 | MGMT |
| chr5 | 497501 | 497600 | SLC9A3 |
| chr2 | 242797801 | 242797900 | PDCD1 |
| chr1 | 1920701 | 1920800 | CFAP74 |
| chr14 | 106320501 | 106320600 | n/a |
| chr14 | 105045001 | 105045100 | C14orf180 |
| chr3 | 185788701 | 185788800 | ETV5 |
| chr14 | 94451401 | 94451500 | n/a |
| chr11 | 118042701 | 118042800 | SCN2B |
| chr7 | 1266101 | 1266200 | n/a |
| chr1 | 2527401 | 2527500 | MMEL1 |
| chr6 | 17988901 | 17989000 | n/a |
| chr5 | 10653251 | 10653350 | ANKRD33B |
| chr5 | 10653301 | 10653400 | ANKRD33B |
| chr16 | 1198601 | 1198700 | n/a |
| chr5 | 140710451 | 140710550 | PCDHGA1 |
| chr14 | 104617901 | 104618000 | KIF26A |
| chr15 | 100016301 | 100016400 | n/a |
| chr1 | 33391451 | 33391550 | n/a |
| chr5 | 137225201 | 137225300 | PKD2L2 |
| chr3 | 97542151 | 97542250 | CRYBG3 |
| chr6 | 156954501 | 156954600 | n/a |
| chr11 | 2293301 | 2293400 | n/a |
| chr3 | 13058901 | 13059000 | IQSEC1 |
| chr17 | 74581301 | 74581400 | ST6GALNAC2 |
| chr12 | 107297151 | 107297250 | n/a |
| chr1 | 7602001 | 7602100 | CAMTA1 |
| chr14 | 104768351 | 104768450 | n/a |
| chr1 | 121260701 | 121260800 | EMBP1 |
| chr7 | 1686651 | 1686750 | n/a |
| chr14 | 100624951 | 100625050 | DEGS2 |
| chr7 | 72788001 | 72788100 | n/a |
| chr5 | 2205801 | 2205900 | n/a |
| chr17 | 74581401 | 74581500 | ST6GALNAC2 |
| chr10 | 134610301 | 134610400 | n/a |
| chr19 | 554901 | 555000 | n/a |
| chr21 | 46816601 | 46816700 | n/a |
| chr10 | 4230501 | 4230600 | n/a |
| chr7 | 1251301 | 1251400 | n/a |
| chr22 | 19744001 | 19744100 | TBX1 |
| chr8 | 143545151 | 143545250 | ADGRB1 |
| chr19 | 45003801 | 45003900 | ZNF180 |
| chr7 | 2959151 | 2959250 | CARD11 |
| chr3 | 169540351 | 169540450 | LRRIQ4 |
| chr2 | 209271201 | 209271300 | PTH2R |
| chr13 | 31620351 | 31620450 | n/a |
| chr1 | 200003301 | 200003400 | NR5A2 |
| chr11 | 67462751 | 67462850 | n/a |
| chr20 | 47278501 | 47278600 | PREX1 |
| chr22 | 37499801 | 37499900 | TMPRSS6 |
| chr7 | 73465951 | 73466050 | ELN |
| chr19 | 17571551 | 17571650 | NXNL1 |
| chr1 | 1936801 | 1936900 | n/a |
| chr11 | 2206101 | 2206200 | n/a |
| chr14 | 100631801 | 100631900 | n/a |
| chr2 | 75136551 | 75136650 | LINC01291 |
| chr10 | 12543351 | 12543450 | CAMK1D |
| chr4 | 3677551 | 3677650 | LOC100133461 |
| chr22 | 19744051 | 19744150 | TBX1 |
| chr14 | 106208401 | 106208500 | n/a |
| chr14 | 105044901 | 105045000 | n/a |
| chr22 | 37500651 | 37500750 | TMPRSS6 |
| chr6 | 168630051 | 168630150 | n/a |
| chr4 | 1537601 | 1537700 | n/a |
| chr7 | 104897151 | 104897250 | SRPK2 |
| chr14 | 106174201 | 106174300 | n/a |
| chr21 | 42219701 | 42219800 | DSCAM |
| chr10 | 79270701 | 79270800 | KCNMA1 |
| chr14 | 104623551 | 104623650 | KIF26A |
| chr1 | 7601951 | 7602050 | CAMTA1 |
| chr2 | 121279901 | 121280000 | n/a |
| chr7 | 120967801 | 120967900 | WNT16 |
| chr7 | 120967851 | 120967950 | WNT16 |
| chr7 | 65970101 | 65970200 | n/a |
| chr16 | 474201 | 474300 | n/a |
| chr1 | 1957751 | 1957850 | GABRD |
| chr1 | 3534351 | 3534450 | n/a |
| chr5 | 173738051 | 173738150 | n/a |
| chr11 | 120764501 | 120764600 | LOC101929227 |
| chr9 | 122800901 | 122801000 | n/a |
| chr9 | 129387151 | 129387250 | LMX1B |
| chr6 | 18990501 | 18990600 | n/a |
| chr3 | 72704601 | 72704700 | n/a |
| chr10* | 26502051 | 26502150 | n/a |
| chr5* | 111090051 | 111090150 | NREP |
| chr5* | 111090101 | 111090200 | NREP |
| chr10* | 26502101 | 26502200 | n/a |
| chr15* | 67841351 | 67841450 | MAP2K5 |
| chr15* | 67841401 | 67841500 | MAP2K5 |
| chr8* | 25902201 | 25902300 | EBF2 |

All regions including, having, or within a genomic location of Table 8 are hypomethylated regions except for the 7 locations indicated with a *, which are hypermethylated regions
In Table 8, where the gene indicated is “n/a” this means that the genomic location defined in the table is a non-coding region of DNA or not within the location of a known gene.

The prostate cancer subtype is one that has an aggressive clinical course and/or androgen receptor (AR) copy number gain, for example an androgen-insensitive prostate cancer subtype. The prostate cancer subtype may be a subtype (i.e. one having an aggressive clinical course and/or androgen receptor (AR) copy number gain) of acinar adenocarcinoma prostate cancer, ductal adenocarcinoma prostate cancer, transitional cell cancer of the prostate, squamous cell cancer of the prostate, or small cell prostate cancer. For example, it may be a subtype (i.e. one having an aggressive clinical course and/or androgen receptor (AR) copy number gain) of acinar adenocarcinoma prostate cancer or ductal adenocarcinoma prostate cancer. Alternatively, or additionally, the prostate cancer may be castration sensitive prostate cancer or castration resistant prostate cancer. Alternatively, or additionally, the prostate cancer may be metastatic prostate cancer, or it may be non-metastatic prostate cancer. In certain embodiments, it may be metastatic prostate cancer. In certain embodiments, the prostate cancer may be metastatic castration resistant prostate cancer or non-metastatic castration resistant prostate cancer. For example, it may be metastatic castration resistant prostate cancer.

The method is especially suitable for the detecting, screening, monitoring, staging, classification, selecting treatment for, ascertaining whether treatment is working in, and/or prognostication of metastatic prostate cancer and/or castration resistant prostate cancer, and particularly prostate cancers subtypes that have an aggressive clinical course and androgen receptor (AR) copy number gain, for example an androgen-insensitive prostate cancer subtype.

The sample is a sample that comprises cfDNA. The sample may suitably be a blood sample, a plasma sample, or a urine sample. Preferably, the sample is a blood sample or a plasma sample. More preferably, the sample is a plasma sample.

The method may further comprise isolating the cfDNA from the sample. cfDNA can be isolated from the sample using a variety of techniques known in the art. For example, DNA (e.g., cfDNA) can be isolated by a column-based approach and/or a bead-based approach. In some embodiments, DNA (e.g., cfDNA) is isolated by means of a column-based approach, for example using a commercially available kit such as QIAamp circulating nucleic acid kit (Qiagen qiagen.com/ch/products/discovery-and-translational-research/dna-rna-purification/dna-purification/cell-free-dna/qiaamp-circulating-nucleic-acid-kit/#orderinginformation). In some embodiments, DNA (e.g., cfDNA) is isolated by means of a bead-based approach, for example an automated cf-DNA extraction system using a commercially available kit such as Maxwell RSC ccfDNA Plasma Kit (Promega (https://www.promega.co.uk/resources/protocols/technical-manuals/101/maxwell-rsc-ccfdna-plasma-kit-protocol/)).

The isolated cfDNA may be amplified before analysis. Thus the method may further comprise amplification of the isolated cfDNA. Amplification techniques are known to those of ordinary skill in the art and include, but are not limited to, cloning, polymerase chain reaction (PCR), polymerase chain reaction of specific alleles (PASA), polymerase chain ligation, nested polymerase chain reaction, and so forth.

The method comprises characterizing the methylome sequence of a plurality of cfDNA molecules in the sample, wherein the methylome sequence of a cfDNA molecule is the DNA sequence and the methylation profile of the molecule. The methylome sequence of a cfDNA molecule may be characterised by using methylation aware sequencing, by genome sequencing followed by methylation profiling, or by targeted approaches that capture specific DNA sequences (for example using DNA probes). Examples of methylation aware sequencing include bisulfite sequencing, bisulfite-free methylation-aware sequencing, methylation arrays (for example methylation microarrays), enzymatic methylation sequencing, methylation-sensitive restriction enzyme digestion, methylation-specific PCR, methylation aware PCR based assays, methylation-dependent DNA precipitation, methylated DNA binding proteins/peptides, single molecule sequences without sodium bisulfite treatment. In certain embodiments, the methylome sequence of a plurality of cfDNA molecules in the sample is characterised using bisulfite sequencing, methylation microarrays, enzymatic methylation sequencing, bisulfite-free methylation-aware sequencing, or methylation aware PCR based assays.

Examples of targeted approaches that capture specific DNA sequences (for example using DNA probes) include cell-free methylated DNA immunoprecipitation and high-throughput sequencing (cfMeDIP-seq), methylation-dependent DNA precipitation, and methylated DNA binding proteins/peptides.

Bisulfite sequencing may comprise massive parallel sequencing with bisulfite conversion, for example treating the DNA molecule with sodium bisulfite and performing sequencing of the treated DNA molecule. Methylation assay sequencing may comprise treating the DNA molecule with sodium bisulfite, whole genome amplification, and hybridisation to a methylation-specific probe or a non-methylation probe, for example attached to a bead or chip.

Enzymatic methylation sequencing may comprise enzymatic treatment of the DNA molecule to convert methylated cytosine sites, followed by sequencing of the treated DNA. For example enzymatic methylation sequencing may comprise enzymatic treatment of the DNA molecule to convert methylated cytosine sites into a form protected from deamination, followed by deamination to convert unprotected cytosine to uracils, and sequencing of the treated DNA. An example of an enzymatic methylation sequencing kit includes NEBNext® Enzymatic Methyl-seq Kit (https://www.neb.com/products/e7120-nebnext-enzymatic-methyl-seq-kit#).

Examples of methylation aware PCR based assays include digital droplet PCR and qPCR (quantitative PCR).

An example of bisulfite-free methylation-aware sequencing is Oxford Nanopore seqeuencing (Oxford Nanopore Technologies, https://nanoporetech.com/))

In certain embodiments, the methylome sequence of a plurality of cfDNA molecules in the sample is characterised using whole genome bisulfite sequencing, for example low pass whole genome bisulfite sequencing. In another embodiment, the methylome sequence of a plurality of cfDNA molecules in the sample is characterised using reduced representation bisulfite treatments. In certain embodiments, the methylome sequence of a plurality of cfDNA molecules in the sample is characterised using methylation arrays, for example methylation microarrays, such as a Illumina Methylation Assay.

A variety of genome sequencing procedures are known in the art and may be used to practice the methods disclosed herein. For example, Sanger sequencing, Polony sequencing, 454 pyrosequencing, Combinatorial probe anchor synthesis, SOLiD sequencing, Ion Torrent semiconductor sequencing, DNA nanoball sequencing, Heliscope single molecule sequencing, Single molecule real time (SMRT) sequencing, Nanopore DNA sequencing, Microfluidic Sanger sequencing and Illumina dye sequencing.

A plurality of cfDNA molecules may be, for example, at least 100, at least 1000, at least 10,000, at least 50,000, at least 100,000, at least 500,000, at least 1,000,000 (10⁶), at least 5,000,000 (5×10⁶), at least 10,000,000 (10⁷), at least 100,000,000 (10⁸), or at least 1,000,000,000 (10⁹). Preferably, a plurality of cfDNA molecules may be, for example, at least 10,000, at least 50,000, at least 100,000, at least 500,000, at least 1,000,000 (10⁶), at least 5,000,000 (5×10⁶), at least 10,000,000 (10⁷), at least 100,000,000 (10⁸), or at least 1,000,000,000 (10⁹). More preferably, a plurality of cfDNA molecules may be, for example, at least 100,000, at least 500,000, at least 1,000,000 (10⁶), at least 5,000,000 (5×10⁶), at least 10,000,000 (10⁷), at least 100,000,000 (10⁸), or at least 1,000,000,000 (10⁹).

The method may further comprise aligning the methylome sequences with a reference genome for the subject, for example by aligning the methylome sequences with hg38, hg19, hg18, hg17 or hg16. The alignment can, for example, be carried out using a variety of techniques known in the art. For example, a DNA sequence alignment tool, (e.g., BSMAP (PMID: 19635165), Bismark (PMID: 21493656), gemBS (PMID: 30137223), Arioc (PMID: 29554207), BS-Seeker2 (PMID: 24206606), MethylCoder (PMID: 21724594) or BatMeth2 (PMID: 30669962)) can be used to align the reads to the reference genome (for example hg38, hg19, hg18, hg17 or hg16).

The genomic location assigned to each methylome sequence in the alignment is based on the reference genome adopted. The genomic locations listed in Tables 1, 1b, 2 to 9 disclosed herein correspond to reference genome hg19. The corresponding locations in a different reference genome can be found using public available tools known in the art. An example of these tools is LiftOver (http://genome.ucsc.edu/).

In certain embodiments, the method comprises removing duplications of reads of the same DNA molecule (i.e. duplications of reads of the same cfDNA molecule). In this step, sequence reads having exactly the same sequence and start and end base pairs (for example the same unclipped alignment start and unclipped alignment end of the sequence) are removed, as they are likely to be duplicate sequence reads of the same sequence (i.e. duplicate of reads of the same cfDNA molecule). For example, PCR duplications can be removed as part of the aligning step, such as using Picard tools v2.1.0 (http://broadinstitute.github.io/picard).

The method comprises determining the average methylation ratio at 10 or more of the genomic regions for which the average methylation ratio has been determined, each genomic region being selected from the group consisting of:
a 100 to 200 bp region comprising or having a genomic location defined in Table 8, and
a 2 to 99 bp region within a genomic location defined in Table 8 and comprising at least one CpG locus,
   and wherein each of the genomic regions is covered by at least one sequence read of at least one characterized methylome sequence.

In one preferred embodiment, the method comprises determining the average methylation ratio at 10 or more of the genomic regions for which the average methylation ratio has been determined, each genomic region being selected from the group consisting of:

a 100 to 200 bp region comprising or having a genomic location defined in Table 9, and
a 2 to 99 bp region within a genomic location defined in Table 9 and comprising at least one CpG locus,
and wherein each of the genomic regions is covered by at least one sequence read of at least one characterized methylome sequence.

In certain embodiments, each genomic region for which the average methylation ratio has been determined is covered by at least one sequence read of at least two characterized methylome sequences, for example at least one sequence read of at least 3, 4, 5, 6, 7, 8, 9, 10, 15, 20, 25, 50, 100, 1000, 10,000 characterized methylome sequences. Preferably each genomic region is covered by at least one sequence read of at least two characterized methylome sequences, for example at least one sequence read of at least 3, 4, 5, 6, 7, 8, 9, 10, 15, 20, 25, 50, 100, or 1000 characterized methylome sequences. In certain preferred embodiments, each genomic region is covered by at least one sequence read of at least 10 characterized methylome sequences, for example at least one sequence read of at least 10, at least 15, at least 20, at least 25, at least 50, at least 100, or at least 1000 characterized methylome sequences.

In certain embodiments, each genomic region for which the average methylation ratio has been determined is covered by at least 2 sequence reads, for example at least 2, 3, 4, 5, 6, 7, 8, 9, 10, 12, 15, 20, 25, 50, 100, 200, 300, 400, 500, 1000, or 10,000 sequence reads. Preferably, each genomic region is covered by at least 5 sequence reads, for example at least 6, 7, 8, 9, 10, 12, 15, 20, 25, 50, 100, 200, 300, 400, 500, 1000, or 10,000 sequence reads. More preferably, each genomic region is covered by at least 10 sequence reads, for example at least 12, 15, 20, 25, 50, 100, 200, 300, 400, 500, 1000, or 10,000 sequence reads.

In embodiments wherein each genomic region for which the average methylation ratio has been determined is covered by at least 2 sequence reads (for example at least 2, 3, 4, 5, 6, 7, 8, 9, 10, 12, 15, 20, 25, 50, 100, 200, 300, 400, 500, 1000, or 10,000 sequence reads) preferably each sequence read or the majority of the sequence reads (for example at least 50%, 60%, 70%, 80% or 90% of the sequence reads) are from different characterized methylome sequences. More preferably, each sequence read or at least 60%, 70%, 80% or 90% of the sequence reads are from different characterized methylome sequences.

In certain embodiments the method comprises determining the average methylation ratio at 12 or more genomic regions, for example 15 or more genomic regions, 20 or more genomic regions, 25 or more genomic regions, 30 or more genomic regions, 50 or more genomic regions, 75 or more genomic regions, 100 or more genomic regions, 125 or more genomic regions, 150 or more genomic regions, 200 or more genomic regions, 300 or more genomic regions, 400 or more genomic regions, or 500 or more genomic regions. Each genomic region may be selected from the group consisting of:
a 100 to 200 bp region comprising or having a genomic location defined in Table 8, and
a 2 to 99 bp region within a genomic location defined in Table 8 and comprising at least one CpG locus.

The genomic regions are preferably each different from each other. In certain preferred embodiments, the method comprises determining the average methylation ratio at 100 or more genomic regions, 125 or more genomic regions, 150 or more genomic regions, 200 or more genomic regions, 300 or more genomic regions, 400 or more genomic regions, or 500 or more genomic regions. Each genomic region may be selected from the group consisting of:
a 100 to 200 bp region comprising or having a genomic location defined in Table 8, and
a 2 to 99 bp region within a genomic location defined in Table 8 and comprising at least one CpG locus.

In such embodiments, preferably the method comprises determining the average methylation ratio at 12 or more genomic regions, for example 15 or more genomic regions, 20 or more genomic regions, 25 or more genomic regions, 30 or more genomic regions, 50 or more genomic regions, 75 or more genomic regions, 100 or more genomic regions, 125 or more genomic regions, 150 or more genomic regions, 200 or more genomic regions, 300 or more genomic regions, 400 or more genomic regions, or 500 or more genomic regions. For example, the method comprises determining the average methylation ratio at 100 or more genomic regions.

In certain embodiments the method comprises determining the average methylation ratio at 12 or more genomic regions, for example 15 or more genomic regions, 20 or more genomic regions, 25 or more genomic regions, 30 or more genomic regions, 50 or more genomic regions, 75 or more genomic regions, 100 or more genomic regions, 125 or more genomic regions, or 150 genomic regions. Each genomic region may be selected from the group consisting of:
a 100 to 200 bp region comprising or having a genomic location defined in Table 9, and
a 2 to 99 bp region within a genomic location defined in Table 9 and comprising at least one CpG locus.

The genomic regions are preferably each different from each other.

In certain embodiments, each genomic region is selected from the group consisting of:
a 100 to 200 bp region comprising or having a genomic location defined in Table 8, and a 2 to 99 bp region within a genomic location defined in Table 8 and comprising at least one CpG locus.

More suitably, each genomic region is selected from the group consisting of: a 100 to 150 bp region comprising or having a genomic location defined in Table 8, and 10 to 99 bp region within a genomic location defined in Table 8 and comprising at least one CpG locus. More suitably, each genomic region is selected from the group consisting of: a 100 to 120 bp region comprising or having a genomic location defined in Table 8, and 50 to 99 bp region within a genomic location defined in Table 8 and comprising at least one CpG locus. More suitably, each genomic region is selected from the group consisting of: a 100 to 120 bp region comprising or having a genomic location defined in Table 8, and 80 to 99 bp region within a genomic location defined in Table 8 and comprising at least one CpG locus. For example, each genomic region is selected from a 100 bp region having a genomic location defined in Table 8.

In such embodiments, preferably the method comprises determining the average methylation ratio at 12 or more genomic regions, for example 15 or more genomic regions, 20 or more genomic regions, 25 or more genomic regions, 30 or more genomic regions, 50 or more genomic regions, 75 or more genomic regions, 100 or more genomic regions, 125 or more genomic regions, 150 or more genomic regions, 200 or more genomic regions, 300 or more genomic regions, or 400 or more genomic regions. For example, the method comprises determining the average methylation ratio at 100 or more genomic regions.

In certain embodiments, each genomic region is selected from the group consisting of:
a 100 to 200 bp region comprising or having a genomic location defined in Table 9, and a 2 to 99 bp region within a genomic location defined in Table 9 and comprising at least one CpG locus.

More suitably, each genomic region is selected from the group consisting of: a 100 to 150 bp region comprising or having a genomic location defined in Table 9, and 10 to 99 bp region within a genomic location defined in Table 9 and comprising at least one CpG locus. More suitably, each genomic region is selected from the group consisting of: a 100 to 120 bp region comprising or having a genomic location defined in Table 9, and 50 to 99 bp region within a genomic location defined in Table 9 and comprising at least one CpG locus. More suitably, each genomic region is selected from the group consisting of: a 100 to 120 bp region comprising or having a genomic location defined in Table 9, and 80 to 99 bp region within a genomic location defined in Table 9 and comprising at least one CpG locus. For example, each genomic region is selected from a 100 bp region having a genomic location defined in Table 9.

In such embodiments, preferably the method comprises determining the average methylation ratio at 12 or more genomic regions, for example 15 or more genomic regions, 20 or more genomic regions, 25 or more genomic regions, 30 or more genomic regions, 50 or more genomic regions, 75 or more genomic regions, 100 or more genomic regions, 125 or more genomic regions, or 150 genomic regions. For example, the method comprises determining the average methylation ratio at 100 or more genomic regions.

In certain preferred embodiments, determining the average methylation ratio for a genomic region comprises calculating the sum of the methylation ratios of all CpGs within the genomic region and dividing the sum by the number of CpGs within the genomic region. In such embodiments, the average methylation ratio may also be referred to as the mean methylation ratio. For the avoidance of doubt, if a genomic region has only one CpG locus, the average methylation ratio for the genomic region is the same as the methylation ratio for the single CpG locus in the genomic region.

The method of the present invention comprises calculating a methylation score using the average methylation ratio for each genomic region for which the average methylation ratio has been determined.

In certain embodiments, calculating a methylation score using the average methylation ratio for each genomic region comprises:
determining the median or the mean of the average methylation ratios for all genomic regions (i.e. all genomic regions for which an average methylation ratio has been determined in the method); or
determining the median or the mean of the average methylation ratios for a first group of genomic regions to obtain a first methylation score and/or determining the median or the mean of the average methylation ratios for second group of genomic regions to obtain a second methylation score; or
comparing the average methylation ratio at each genomic region to a reference methylation ratio for each genomic region to determine a methylation ratio score for each genomic region.

In one preferred embodiment, calculating a methylation score using the average methylation ratio for each genomic region comprises:
determining the median of the average methylation ratios for all genomic regions for which the average methylation ratio has been determined; or
determining the median of the average methylation ratios for a first group of genomic regions to obtain a first methylation score and/or determining the median of the average methylation ratios for second group of genomic regions to obtain a second methylation score; or
comparing the average methylation ratio at each genomic region to a reference methylation ratio for each genomic region to determine a methylation ratio score for each genomic region.

In one preferred embodiment, calculating a methylation score using the average methylation ratio for each genomic region comprises:
determining the median of the average methylation ratios for all genomic regions for which the average methylation ratio has been determined; or
determining the median of the average methylation ratios for a first group of genomic regions to obtain a first methylation score and/or determining the median of the average methylation ratios for second group of genomic regions to obtain a second methylation score.

In one preferred embodiment, calculating a methylation score using the average methylation ratio for each genomic region comprises
determining the median of the average methylation ratios for a first group of genomic regions to obtain a first methylation score and/or determining the median of the average methylation ratios for second group of genomic regions to obtain a second methylation score.

In embodiments wherein calculating a methylation score using the average methylation ratio for each genomic region comprises determining the median (or the mean) of the average methylation ratios for a first group of genomic regions to obtain a first methylation score and/or determining the median (or the mean) of the average methylation ratios for a second group of genomic regions to obtain a second methylation score, the first group of genomic regions are all of the hypermethylated genomic regions (i.e. all hypermethylated genomic regions for which an average methylation ratio has been determined in the method, i.e. selected from those comprising, having or within a genomic location defined in Table 8), and the second group of genomic regions are all of the hypomethylated genomic regions (i.e. all hypomethylated genomic regions for which an average methylation ratio has been determined in the method, i.e. selected from those comprising, having or within a genomic location defined in Table 8).

In one preferred embodiment, calculating a methylation score using the average methylation ratio for each genomic region comprises
determining the median of the average methylation ratios for all of the hypermethylated genomic regions (i.e. all hypermethylated genomic regions for which an average methylation ratio has been determined in the method) to obtain a first methylation score and determining the median of the average methylation ratios for all of the hypomethylated genomic regions (i.e. all hypomethylated genomic regions for which an average methylation ratio has been determined in the method to obtain a second methylation score.

In one embodiment, calculating a methylation score using the average methylation ratio for each genomic region comprises
determining the median of the average methylation ratios for all of the hypermethylated genomic regions (i.e. all hypermethylated genomic regions for which an average methylation ratio has been determined in the method) to obtain a first methylation score.

In one especially preferred embodiment, calculating a methylation score using the average methylation ratio for each genomic region comprises
determining the median of the average methylation ratios for all of the hypomethylated genomic regions (i.e. all hypomethylated genomic regions for which an average methylation ratio has been determined in the method) to obtain a second methylation score.

In one embodiment, calculating a methylation score using the average methylation ratio for each genomic region comprises comparing the average methylation ratio at each genomic region to a reference methylation ratio for each genomic region to determine a methylation ratio score for each genomic region. In such embodiments, preferably the reference methylation ratio is the average methylation ratio for the same genomic region in or covered by:
a cfDNA sample from a healthy subject, for example a healthy age-matched subject;
a tissue sample from a healthy subject, for example a prostate tissue sample from a healthy subject;
a cancer biopsy sample from a cancer patient, for example a prostate cancer biopsy sample from a prostate cancer patient, for example a prostate cancer patient with a known subtype;
a cancer cell line sample, for example a prostate cancer cell line sample from a prostate cancer cell line, for example a prostate cancer cell line of a known subtype;
a sample of white blood cells from a subject, for example the subject or a healthy subject;
a cfDNA sample from a different subject having prostate cancer, wherein preferably the sample is known to comprise cfDNA derived from the prostate cancer subtype (preferably multiple cfDNA samples (for example at least 2, 3, 4, 5, 10, 20, 40, 50, 100, 200, 300 or 500 samples) each from a different subject having prostate cancer, wherein preferably each sample is known to comprise cfDNA derived from the prostate cancer subtype, and more preferably wherein each cfDNA sample has a different level of cfDNA derived from the prostate cancer subtype);
a characterized methylome sequence of a white blood cell;
a characterized methylome sequence of a prostate cancer cell line, for example a prostate cancer cell line of a known subtype;
a characterized methylome sequence of a cancerous prostate cell, for example a cancerous prostate cell of a known subtype; and/or
a characterized methylome sequence of a non-cancerous prostate cell.

In one preferred embodiment, the reference methylation ratio is the average methylation ratio for the same genomic region in or covered by
a cfDNA sample from a different subject having prostate cancer, wherein preferably the sample is known to comprise cfDNA derived from the prostate cancer subtype (preferably multiple cfDNA samples (for example at least 2, 3, 4, 5, 10, 20, 40, 50, 100, 200, 300 or 500 samples) each from a different subject having prostate cancer, wherein preferably each sample is known to comprise cfDNA derived from the prostate cancer subtype, and more preferably wherein each cfDNA sample has a different level of cfDNA derived from the prostate cancer subtype).

In one preferred embodiment, the reference methylation ratio is the average methylation ratio for the same genomic region in or covered by
a cancer biopsy sample from a cancer patient, for example a prostate cancer biopsy sample from a prostate cancer patient, for example a prostate cancer patient with a known subtype;
a cancer cell line sample, for example a prostate cancer cell line sample from a prostate cancer cell line, for example a prostate cancer cell line of a known subtype;
a cfDNA sample from a different subject having prostate cancer, wherein preferably the sample is known to comprise cfDNA derived from the prostate cancer subtype (preferably multiple cfDNA samples (for example at least 2, 3, 4, 5, 10, 20, 40, 50, 100, 200, 300 or 500 samples) each from a different subject having prostate cancer, wherein preferably each sample is known to comprise cfDNA derived from the prostate cancer subtype, and more preferably wherein each cfDNA sample has a different level of cfDNA derived from the prostate cancer subtype);
a characterized methylome sequence of a prostate cancer cell line, for example a prostate cancer cell line of a known subtype; and/or
a characterized methylome sequence of a cancerous prostate cell, for example a cancerous prostate cell of a known subtype.

The method of the present invention comprises analyzing the methylation ratio scores to determine whether the sample comprises cfDNA derived from a prostate cancer subtype and/or determine the level of cfDNA in the sample that is derived from a prostate cancer subtype. For example, no level (for example no detectable level) of cfDNA derived from a prostate cancer subtype in the cfDNA sample may be determined. Alternatively, a level of cfDNA derived from a prostate cancer subtype in the cfDNA sample may be determined. The minimum percentage level of cfDNA derived from a prostate cancer subtype in the cfDNA sample that may be determined may be 0.01% of cfDNA derived from a prostate cancer subtype in the cfDNA sample. In certain embodiments, the minimum percentage level of cfDNA derived from a prostate cancer subtype in the cfDNA sample that may be determined may be 0.02%, 0.03%, 0.04%, 0.06%, 0.07%, 0.08%, 0.05%, 0.09%, 0.1%, 0.2%, 0.3%, 0.4%, 0.5%, 0.6%, 0.7%, 0.8%, 0.9%, 1%, 2%, 3% 4%, 5%, 10%, 15%, 20%, 30%, 40%, 50% of cfDNA derived from a prostate cancer subtype in the cfDNA sample. For example, the minimum percentage level of cfDNA derived from a prostate cancer subtype in the cfDNA sample that may be determined may be 0.01%, 0.05%, 0.1% or 0.5%. Preferably, the minimum percentage level of cfDNA derived from a prostate cancer subtype in the cfDNA is 0.01%.

The method comprises analyzing the methylation score to determine the level of cfDNA derived from a prostate cancer subtype in the cfDNA sample.

If level of cfDNA derived from a prostate cancer subtype in the cfDNA sample is determined, the subject can be classed as having the subtype. As such, analyzing the methylation score to determine whether there is a level of cfDNA derived from a prostate cancer subtype in the cfDNA sample may also be referred as analyzing the methylation score to determine whether a subject has a prostate cancer subtype.

Preferably, analyzing the methylation score to determine the level of cfDNA derived from a prostate cancer subtype in the cfDNA sample comprises comparing the methylation score to one or more reference methylation scores. For example, the method may comprise comparing the methylation score to one reference methylation scores. In certain embodiments, the method comprises comparing the methylation score to two or more reference methylation scores, for example 2, 3, 4, 5, 6, 8, 9, 10, 12, 15, 20, 30, 50, 100, 200, 300, 400, 500 or 1000 reference methylation scores. In certain embodiments, the method comprises comparing the methylation score to 5 or more reference methylation scores, for example 10 or more, 15 or more, 20 or more, 30, or more 50, or more 100, or more 200, or more 300, or more 400, or more 500 or 1000 or more reference methylation scores.

In embodiments wherein the method comprises comparing the methylation score to two or more reference methylation scores, the reference methylation scores may come from different types of reference samples and/or reference methylomes (for example a cfDNA sample from a healthy subject and a cancer cell line sample) and/or the same type of reference samples or reference methylomes but from different sources (for example, two or more cfDNA samples each from a different healthy subject).

A reference methylation score is a methylation score calculated for the same genomic regions (for example, calculated using the average methylation ratio for the same genomic regions) in a reference sample or reference methylome. A reference sample or reference methylome may be selected from the group consisting of:
a cfDNA sample from a healthy subject, for example a healthy age-matched subject;
a tissue sample from a healthy subject, for example a prostate tissue sample from a healthy subject;
a cancer biopsy sample from a cancer patient, for example a prostate cancer biopsy sample from a prostate cancer patient, for example a prostate cancer patient with a known subtype;
a cancer cell line sample, for example a prostate cancer cell line sample from a prostate cancer cell line, for example a prostate cancer cell line of a known subtype;
a sample of white blood cells from a subject, for example the subject or a healthy subject;
a cfDNA sample from a different subject having prostate cancer, wherein preferably the sample is known to comprise cfDNA derived from the prostate cancer subtype (preferably multiple cfDNA samples (for example at least 2, 3, 4, 5, 10, 20, 40, 50, 100, 200, 300 or 500 samples) each from a different subject having prostate cancer, wherein preferably each sample is known to comprise cfDNA derived from the prostate cancer subtype, and more preferably wherein each cfDNA sample has a different level of cfDNA derived from the prostate cancer subtype);
a characterized methylome sequence of a white blood cell;
a characterized methylome sequence of a prostate cancer cell line, for example a prostate cancer cell line of a known subtype;
a characterized methylome sequence of a cancerous prostate cell, for example a cancerous prostate cell of a known subtype; and/or
a characterized methylome sequence of a non-cancerous prostate cell.

A reference sample or reference methylome may be one that can be used to represent a sample having no cfDNA derived from the prostate cancer subtype (for example an undetectable level of cfDNA in the prostate cancer subtype in the cfDNA sample), for example a reference sample or reference methylome selected from one or more of the following
a cfDNA sample from a healthy subject, for example a healthy age-matched subject;
a sample of white blood cells from a subject, for example the subject or a healthy subject; and/or
a characterized methylome sequence of a white blood cell.

A reference sample or reference methylome may be one that can be used to represent a sample having 100% cfDNA derived from the prostate cancer subtype, for example a reference sample or reference methylome selected from one or more of the following
a cancer biopsy sample from a cancer patient, for example a prostate cancer biopsy sample from a prostate cancer patient, for example a prostate cancer patient with a known subtype;
a cancer cell line sample, for example a prostate cancer cell line sample from a prostate cancer cell line, for example a prostate cancer cell line of a known subtype;
a characterized methylome sequence of a prostate cancer cell line, for example a prostate cancer cell line of a known subtype;
a characterized methylome sequence of a cancerous prostate cell, for example a cancerous prostate cell of a known subtype; and/or

A reference sample or reference methylome may be one that can be used to represent a sample having 10 to 90% cfDNA derived from a prostate cancer subtype, for example one or more cfDNA samples from different subjects having prostate cancer known to have the prostate cancer subtype, wherein the level of cfDNA derived from the prostate cancer subtype in each cfDNA sample from the different subjects is/are known. A level of cfDNA derived from the prostate cancer subtype in each cfDNA sample can be determined by looking at genomic markers.

Preferably, analyzing the methylation score to determine the level of cfDNA derived from the prostate cancer subtype in the cfDNA sample comprises comparing the methylation score to one or more reference methylation scores that can be used to represent a sample having 100% cfDNA derived from the prostate cancer subtype, and can be used to represent a sample having 0% cfDNA derived from the prostate cancer subtype, and optionally can be used to represent a sample having 10-90% cfDNA derived from the prostate cancer subtype. For example, analyzing the methylation score to determine the level of cfDNA derived from a prostate cancer subtype in the cfDNA sample comprises:

comparing the methylation score to one or more reference methylation scores for a reference sample or reference methylome selected from the group consisting of:
a cfDNA sample from a healthy subject, for example a healthy age-matched subject,
a sample of white blood cells from a subject, for example the subject or a healthy subject, and/or
a characterized methylome sequence of a white blood cell;
   and
   comparing the methylation score to one or more reference methylation scores for a reference sample or reference methylome selected from the group consisting of:
a cancer biopsy sample from a cancer patient, for example a prostate cancer biopsy sample from a prostate cancer patient, for example a prostate cancer patient with a known subtype;
a cancer cell line sample, for example a prostate cancer cell line sample from a prostate cancer cell line, for example a prostate cancer cell line of a known subtype;
a characterized methylome sequence of a prostate cancer cell line, for example a prostate cancer cell line of a known subtype;
a characterized methylome sequence of a cancerous prostate cell, for example a cancerous prostate cell of a known subtype
   and optionally comparing the methylation score to one or more reference methylation scores for one or more cfDNA samples from different subjects having prostate cancer, wherein the level of cfDNA derived from the prostate cancer subtype in each cfDNA sample from the different subjects is/are known.

Preferably, the reference methylation score for a reference sample or reference methylome that a methylation ratio score methylation ratio score is compared to is calculated in the same way as the methylation score for the sample obtained from the subject (i.e. the sample that the method of the invention is being carried out in respect of). For example, if the methylation ratio for the selected genomic regions of the sample obtained from the subject is calculated by determining the median (or the mean) of the average methylation ratios for a first group of genomic regions to obtain a first methylation score and/or determining the median (or the mean) of the average methylation ratios for second group of genomic regions to obtain a second methylation score, the reference methylation score for a reference sample or reference methylome is calculated by determining the median (or the mean) of the average methylation ratios for the same first group of genomic regions to obtain a first reference methylation score and/or determining the median (or the mean) of the average methylation ratios for the same second group of genomic regions to obtain a second reference methylation score.

Or, for example, if the methylation ratio for the selected genomic regions of the sample obtained from the subject is calculated by determining the median (or the mean) of the average methylation ratios for all genomic regions, the reference methylation score for a reference sample or reference methylome is calculated by determining the median (or the mean) of the average methylation ratios for the same genomic regions.

In embodiments wherein the method comprises comparing the average methylation ratio at each genomic region to a reference methylation ratio for each genomic region to determine a methylation ratio score for each genomic region, analyzing the methylation ratio scores to determine the level of cfDNA derived from the prostate cancer subtype in the cfDNA sample may comprise determining how many methylation ratio scores are indicative of the prostate cancer subtype.

In certain embodiments, analyzing the methylation score to determine the level of cfDNA derived from the prostate cancer subtype in the cfDNA sample comprises using a mathematical model, such as a linear regression model or another linear model (for example, a general linear model, a heteroscedastic model, a generalised linear model, or a hierarchical linear model).

In certain embodiments, analyzing the methylation score to determine the level of level of cfDNA derived from the prostate cancer subtype in the cfDNA sample comprises using a mathematical model that compares the methylation score for the sample to reference methylation scores that can be used to represent a sample having 100% cfDNA derived from the prostate cancer subtype in the cfDNA, and can be used to represent a sample having 0% cfDNA derived from the prostate cancer subtype in the cfDNA, and optionally can be used to represent a sample having 10-90% cfDNA derived from the prostate cancer subtype in the cfDNA. For example, the method comprises using mathematical model that compares the methylation score for the sample to reference methylation scores for a cfDNA sample from a healthy subject, for example a healthy age-matched subject (0% cfDNA derived from the prostate cancer subtype in the cfDNA) and/or a characterized methylome sequence of a white blood cell (0% cfDNA derived from the prostate cancer subtype in the cfDNA) and/or a sample of white blood cells from a subject, for example the subject or a healthy subject, (0% cfDNA derived from the prostate cancer subtype in the cfDNA sample) and/or a characterized methylome sequence of a prostate cancer cell line (100% cfDNA derived from the prostate cancer subtype in the cfDNA sample) and/or a prostate cancer biopsy sample from a prostate cancer patient (100% cfDNA derived from the prostate cancer subtype in the cfDNA sample) and/or one or more cfDNA samples (for example at least 2, 3, 4, 5, 10, 20, 40, 50, 100, 200, 300 or 500 samples) each from a different subject having prostate cancer, wherein the level of cfDNA derived from the prostate cancer subtype in each cfDNA sample from the different subjects is known, and preferably wherein each cfDNA sample has a different level of cfDNA derived from the prostate cancer subtype (10-90% cfDNA derived from the prostate cancer subtype in the cfDNA sample).

In one embodiment, the method comprises using mathematical model that compares the methylation score for the sample to reference methylation scores for a cfDNA sample from a healthy subject, for example a healthy age-matched subject (0% cfDNA derived from the prostate cancer subtype in the cfDNA sample) and/or a characterized methylome sequence of a prostate cancer cell line (100% cfDNA derived from the prostate cancer subtype in the cfDNA sample) and/or a prostate cancer biopsy sample from a prostate cancer patient (100% cfDNA derived from the prostate cancer subtype in the cfDNA sample) and/or one or more cfDNA samples (for example at least 2, 3, 4, 5, 10, 20, 40, 50, 100, 200, 300 or 500 samples) each from a different subject having prostate cancer, wherein the level of cfDNA derived from the prostate cancer subtype in the cfDNA in each cfDNA sample from the different subjects is known, and preferably wherein each cfDNA sample has a different level of cfDNA derived from the prostate cancer subtype in the cfDNA sample (10-90% cfDNA derived from the prostate cancer subtype in the cfDNA sample).

The method may further comprise measuring the level of prostate-specific antigen (PSA) in a sample of blood from the subject. It may also comprise determining if the subject has an abnormal level of PSA in the blood (for example a level of PSA in the blood of at least 4.0 ng/mL). An abnormal level of PSA in the blood may be, for example, a level of PSA in the blood of at least 4.0 ng/mL). A normal level of PSA in the blood may, for example, be a level of PSA in the blood of 4.0 ng/mL or less.

In one preferred embodiment, the method is for screening, monitoring, and/or prognostication of prostate cancer, wherein prostate cancer with a poor prognosis is predicted when a level of cfDNA derived from the prostate cancer subtype in the cfDNA sample is determined, for example a detectable level of cfDNA derived from the prostate cancer subtype in the sample, for example a percentage level of cfDNA derived from the prostate cancer subtype in the sample of at least 0.01%. For example, a prostate cancer with a poor prognosis is predicted when at least 0.01% cfDNA derived from the prostate cancer subtype in the sample is determined, or for example, at least 0.02%, at least 0.03%, at least 0.04%, at least 0.05%, at least 0.1%, at least 0.5% or at least 1% cfDNA derived from the prostate cancer subtype in the sample is determined.

In some instances, a “poor” prognosis refers to a low likelihood that a subject will likely respond favorably to a drug or set of drugs, is in complete or partial remission, or there is a decrease and/or a stop in the progression of prostate cancer. In some instances, a “poor” prognosis refers to a survival of a subject that is expected to be from less than 5 years to less than 1 month (for example less than 3 years to less than 1 month, or less than 3 years to less than 6 months). In some instances, a “poor” prognosis refers to a survival of a subject in which the survival of the subject upon treatment is expected to be from less than 5 years to less than 1 month.

In one preferred embodiment, the method is for detection of prostate cancer, wherein the prostate cancer subtype is detected when a level of cfDNA derived from a prostate cancer subtype in the cfDNA sample is determined, for example a detectable level of cfDNA derived from the prostate cancer subtype in the cfDNA sample, for example a percentage level of cfDNA derived from the prostate cancer subtype in the cfDNA sample of at least 0.01%, or for example, at least 0.02%, at least 0.03%, at least 0.04%, at least 0.05%, at least 0.1%, at least 0.5% or at least 1% cfDNA derived from the prostate cancer subtype in the cfDNA sample.

In one preferred embodiment, the method is for screening, monitoring, and/or prognostication of prostate cancer, wherein prostate cancer with a poor prognosis is predicted when a level of cfDNA derived from the prostate cancer subtype in the cfDNA sample is determined, for example a detectable level of prostate cancer, for example a percentage level of cfDNA derived from the prostate cancer subtype in the cfDNA sample of at least 0.01%, for example at least 0.01% cfDNA derived from the prostate cancer subtype in the cfDNA sample, or for example, at least 0.02%, at least 0.03%, at least 0.04%, at least 0.05%, at least 0.1%, at least 0.5% or at least 1% cfDNA derived from the prostate cancer subtype in the cfDNA sample.

In one preferred embodiment, the method is for detecting, screening and/or prognostication of metastatic prostate cancer, wherein metastatic prostate cancer is predicted when a level of cfDNA derived from the prostate cancer subtype in the cfDNA sample is determined, for example a detectable level of cfDNA derived from the prostate cancer subtype in the cfDNA sample, for example a percentage level of cfDNA derived from the prostate cancer subtype in the cfDNA sample of at least 0.01%, or for example, at least 0.02%, at least 0.03%, at least 0.04%, at least 0.05%, at least 0.1%, at least 0.5% or at least 1% cfDNA derived from the prostate cancer subtype in the cfDNA sample.

In one preferred embodiment, the method is for selecting treatment of prostate cancer or ascertaining whether treatment is working in prostate cancer, wherein a new treatment is selected when a level of cfDNA derived from the prostate cancer subtype in the cfDNA sample is determined, for example a detectable level of cfDNA derived from the prostate cancer subtype in the cfDNA sample, for example a percentage level of cfDNA derived from the prostate cancer subtype in the cfDNA sample of at least 0.01%, or for example, at least 0.02%, at least 0.03%, at least 0.04%, at least 0.05%, at least 0.1%, at least 0.5% or at least 1% cfDNA derived from the prostate cancer subtype in the cfDNA sample.

In one preferred embodiment, the method is for ascertaining whether treatment of prostate cancer is working, wherein it is determined that the treatment is not working when a level of prostate cancer is determined, for example a detectable level of cfDNA derived from the prostate cancer subtype in the cfDNA sample, for example a percentage level of cfDNA derived from the prostate cancer subtype in the cfDNA sample of at least 0.01%, or for example, at least 0.02%, at least 0.03%, at least 0.04%, at least 0.05%, at least 0.1%, at least 0.5% or at least 1% cfDNA derived from the prostate cancer subtype in the cfDNA sample.

The method may further comprising repeating the method on second sample obtained from the subject after the subject has undergone a treatment for prostate cancer, wherein the second sample comprises cfDNA, and comparing the level of cfDNA derived from the prostate cancer subtype in each sample. Preferably, the second sample is of the same type as the first sample, for example if the first sample is a plasma sample then the second sample is a plasma sample. The invention may further comprise repeating the method on a third, and optionally a 4^{th}, 5^{th}, 6^{th}, 7^{th}, 8th, 9^{th} and/or 10^{th}, sample obtained from the subject after the subject has undergone a treatment for prostate cancer, wherein the third, and optionally the 4^{th}, 5^{th}, 6^{th}, 7^{th}, 8^{th}, 9^{th} and/or 10^{th}, sample comprises cfDNA, and comparing the level of cfDNA derived from the prostate cancer subtype in each sample. Preferably, all samples are of the same type as the first sample, for example if the first sample is a plasma sample the all other samples are plasma samples.

In one preferred embodiment, the method is for monitoring of prostate cancer, wherein the method comprises repeating the method on a second sample obtained from the subject after the subject has undergone a treatment for prostate cancer, wherein the second sample comprises cfDNA, and comparing the level of cfDNA derived from the prostate cancer subtype in each cfDNA sample.

In one preferred embodiment, the method is for selecting treatment of prostate cancer, comprising repeating the method on a second sample obtained from the subject after the subject has undergone a treatment for prostate cancer, wherein the second sample comprises cfDNA, and comparing the level of cfDNA derived from the prostate cancer subtype in each cfDNA sample, wherein a new treatment is selected if the level of prostate cancer is increased in the second sample, for example an increase of at least 0.01%.

In one preferred embodiment, the method is for ascertaining whether treatment of prostate cancer is working, comprising repeating the method on a second sample obtained from the subject after the subject has undergone a treatment for prostate cancer, wherein the second sample comprises cfDNA, wherein it is determined that the treatment is not working if the level of cfDNA derived from the prostate cancer subtype is increased in the second sample, for example an increase of at least 0.01%.

In one preferred embodiment, the method is for prognostication of prostate cancer, comprising repeating the method on second sample obtained from the subject after the subject has undergone a treatment for prostate cancer, wherein the second sample comprises cfDNA, wherein it is determined that the prognosis is poor if the level of cfDNA derived from the prostate cancer subtype is increased in the second sample, for example an increase of at least 0.01%. In one preferred embodiment, the method is for prognostication of prostate cancer, comprising repeating the method on a second sample obtained from the subject after the subject has undergone a treatment for prostate cancer, wherein the second sample comprises cfDNA, wherein it is determined that the prognosis is good if the level of cfDNA derived from the prostate cancer subtype is decreased in the second sample, for example a decrease of at least 0.01%. In some instances, a “good” prognosis refers to the likelihood that a subject will likely respond favorably to a drug or set of drugs, leading to a complete or partial remission, or a decrease and/or a stop in the progression of prostate cancer. In some instances, a “good” prognosis refers to the survival of a subject of from at least 1 month to at least 90 years. In some instances, a “good” prognosis refers to the survival of a subject in which the survival of the subject upon treatment is from at least 1 month to at least 90 years.

In certain preferred embodiments, the method of present invention comprises the additional step of obtaining a biological sample from a subject.

The methods can be used with the kits, methods of treatment, therapeutic agents for the treatment of prostate cancer, methods of determining one or more suitable therapeutic agents for the treatment of prostate cancer, methods for determining a treatment regimen, computerized (or computer implemented) methods, computer-assisted methods, computer products and/or computer implemented software described herein. Embodiments and preferred embodiments for the methods are equally applicable to the kits, methods of treatment, therapeutic agents for the treatment of prostate cancer, methods of determining one or more suitable therapeutic agents for the treatment of prostate cancer, methods for determining a treatment regimen, computerized (or computer implemented) methods, computer-assisted methods, computer products and/or computer implemented software described herein.

### Kits

A further aspect, the invention provides an in-vitro diagnostic kit for detecting, screening, monitoring, staging, classification, selecting treatment for, ascertaining whether treatment is working in, and/or prognostication of prostate cancer in a sample obtained from a subject, wherein the sample comprises cfDNA. Preferably, the kits of the invention comprise one or more reagents for detecting the presence or absence of at least 10 DNA molecules having a DNA sequence corresponding to all or part of a genomic location comprising at least one CpG locus defined in Tables 1 to 4.

In certain embodiments, the kit comprises DNA sampling reagents and, preferably, methylome analysis reagents, such as bisulfate reagents. In certain embodiments, the kit comprises DNA amplification agents, for example primers for amplification of specific DNA molecules, for example for amplification of at least 10 DNA molecules having a DNA sequence corresponding to all or part of a genomic location comprising at least one CpG locus defined in Tables 1 to 4.

In one preferred embodiment, the kit comprises instructions for use. In certain embodiments, the kit comprises instructions for detecting, screening, monitoring, staging, classification, selecting treatment for, ascertaining whether treatment is working in, and/or prognostication of prostate cancer in a sample using the kit. For example the kit comprises instructions for use which define how to determine the level of prostate cancer fraction in a sample comprising cfDNA from a subject, for example by following a method of the invention defined herein.

In one preferred embodiment, the kit comprises a computer product or a computer-executable software for detecting, screening, monitoring, staging, classification, selecting treatment for, ascertaining whether treatment is working in, and/or prognostication of prostate cancer in a sample using the kit. In certain embodiments, the computer product comprises a non-transitory computer readable medium storing a plurality of instructions that when executed control a computer system to perform a method of the invention. In certain embodiments, the computer-executable software comprises software for performing a method of the invention.

In certain embodiments the kit comprises of one or more containers and may also include sampling equipment, for example, bottles, bags (such as intravenous fluid bags), vials, syringes, and test tubes. Other components may include needles, diluents, wash reagents and buffers. Usefully, the kit may include at least one container comprising a pharmaceutically-acceptable buffer, such as phosphate-buffered saline, Ringer's solution and dextrose solution.

If a reagent is for detecting the presence or absence of a DNA molecule having a DNA sequence corresponding to all of a genomic location defined in Tables 1 to 4, the reagent is able to detect the presence of a DNA sequence having or comprising a genomic location defined in Tables 1 to 4. For example, the reagent is able to detect the presence of the a DNA sequence having a genomic location defined in Tables 1 to 4 or comprising a genomic location defined in Tables 1 to 4 and having a sequence length of 101 to 200 bp, for example having a sequence length of 101 to 180, a sequence length of 101 to 150, a sequence length of 101 to 140, a sequence length of 101 to 130, a sequence length of 101 to 120, or a sequence length of 101 to 110 bp.

If a reagent is for detecting the presence or absence of a DNA molecule having a DNA sequence corresponding to a part of a genomic location defined in Tables 1 to 4, the reagent is able to detect the presence of a DNA sequence comprising at least a 10 bp continuous sequence within a genomic location defined in Tables 1 to 4 and comprising at least one CpG locus. Preferably, if a reagent is for detecting the presence or absence of a DNA molecule having a DNA sequence corresponding to a part of a genomic location defined in Tables 1 to 4, the reagent is able to detect the presence of a DNA sequence comprising at least a 15 bp continuous sequence within a genomic location defined in Tables 1 to 4 and comprising at least one CpG locus, for example at least a 20, 25, 30, 35, 40, 45, 50, 50, 55, 60, 65, 70, 75, 80, 85, 90, 95 or 99 bp continuous sequence within a genomic location defined in Tables 1 to 4 and comprising at least one CpG locus. In certain preferred embodiments, if a reagent is for detecting the presence or absence of a DNA molecule having a DNA sequence corresponding to a part of a genomic location defined in Tables 1 to 4, the reagent is able to detect the presence of a DNA sequence comprising (or consisting of) a 20, 25, 30, 35, 40, 45, 50, 50, 55, 60, 65, 70, 75, 80, 85, 90, 95 or 99 bp continuous sequence within a genomic location defined in Tables 1 to 4 and comprising at least one CpG locus.

In certain embodiments, the kit comprises one or more reagents for detecting the presence or absence of at least 15 DNA molecules. For example, the kit comprises one or more reagents for detecting the presence or absence of 15, 20, 30, 40, 50, 75, 100, 150, 200, 250, 300, 400, 500, 600, 700, 800, 900 or 1000 DNA molecules.

In certain embodiments, the kit comprises one or more reagents for detecting the presence or absence of at least 50 DNA molecules (for example, the kit comprises one or more reagents for detecting the presence or absence of 50, 75, 100, 150, 200, 250, 300, 400, 500, 600, 700, 800, 900 or 1000 DNA molecules), at least 75 DNA molecules (for example, the kit comprises one or more reagents for detecting the presence or absence of 75, 100, 150, 200, 250, 300, 400, 500, 600, 700, 800, 900 or 1000 DNA molecules), at least 100 DNA molecules (for example, the kit comprises one or more reagents for detecting the presence or absence of 100, 150, 200, 250, 300, 400, 500, 600, 700, 800, 900 or 1000 DNA molecules), at least 150 DNA molecules (for example, the kit comprises one or more reagents for detecting the presence or absence of 150, 200, 250, 300, 400, 500, 600, 700, 800, 900 or 1000 DNA molecules), at least 250 DNA molecules (for example, the kit comprises one or more reagents for detecting the presence or absence of 250, 300, 400, 500, 600, 700, 800, 900 or 1000 DNA molecules), at least 500 DNA molecules (for example, the kit comprises one or more reagents for detecting the presence or absence of 500, 600, 700, 800, 900 or 1000 DNA molecules), at least 700 DNA molecules or at least 900 DNA molecules (for example, the kit comprises one or more reagents for detecting the presence or absence of 900 or 1000 DNA molecules).

In certain preferred embodiments, the genomic location is a location defined in Tables 1 and 2. In certain embodiments, the genomic location is a location defined in Tables 3 and 4. In certain embodiments, the genomic location is a location defined in Tables 1 and 3. In certain embodiments, the genomic location is a location defined in Tables 2 and 4.

In certain preferred embodiments, the genomic location is a location defined in Table 5. In certain preferred embodiments, the genomic location is a location defined in Table 6. In certain preferred embodiments, the genomic location is a location defined in Table 7.

In certain embodiments, the kit comprises oligonucleotides for specifically hybridizing to at least a section of the at least 10 DNA molecules having a DNA sequence corresponding to all or part of a genomic location comprising at least one CpG locus defined in Tables 1 to 4. An oligonucleotide for specifically hybridizing to at least a section of a DNA molecules may be for hybridizing to at least a 10 bp section, at least a 12 bp section, at least a 14 bp section, at least a 15 bp section, at least a 18 bp section, at least a 20 bp section of a DNA molecule, at least a 25 bp section of a DNA molecule, at least a 30 bp section of a DNA molecule or at least a 40 bp section of a DNA molecule. In certain embodiments, an oligonucleotide for specifically hybridizing to at least a section of a DNA molecule may be for hybridizing to a 10 bp section, 12 bp section, 14 bp section, 15 bp section, 18 bp section, 20 bp section, 25 bp section or 30 bp section.

An oligonucleotide for specifically hybridizing to at least a section of a DNA molecule may have a sequence of at least 12, 13, 14, 15, 16, 17, 18, 19, 20, 21, 22, 23, 24, 25, 30, 35, 40, 45, 50, 55, 60, 65, 70, 75, 80, 85, 90 or 95 bp. An oligonucleotide for specifically hybridizing to at least a section of a DNA molecule may comprise not more than 100, 90, 80, or 70 bp. An oligonucleotide for specifically hybridizing to at least a section of a DNA molecule may have a sequence of 12, 13, 14, 15, 16, 17, 18, 19, 20, 21, 22, 23, 24, 25, 30, 35, 40, 45, 50, 55, 60, 65, 70, 75, 80, 85, 90 or 95 bp. Preferably, an oligonucleotide for specifically hybridizing to at least a section of a DNA molecule may have a sequence of 15, 18, 19, 20, 21, 22, 23, 24, 25, 30, 40, 50, 60 or 70 bp. In certain embodiments, an oligonucleotide for specifically hybridizing to at least a section of a DNA molecule may have a sequence of 20 to 90 bp, for example 30 to 80 bp, 50 to 80 bp. In certain embodiments, an oligonucleotide for specifically hybridizing to at least a section of a DNA molecule may have a sequence of 55 to 95 bp. In certain embodiments, an oligonucleotide for specifically hybridizing to at least a section of a DNA molecule may have a sequence of 60 to 80 bp, for example a sequence of 70 bp.

In certain embodiments, the kit comprises oligonucleotides for specifically hybridizing to at least a section of at least 15, 20, 25, 30, 35, 40, 45, 50, 75, 100, 200, 250, 300, 400, 500, 600, 700, 800, or 900 DNA molecules corresponding to a genomic region having or comprising a genomic location defined in Tables 1 to 4. In certain embodiments, the kit comprises oligonucleotides for specifically hybridizing to at least a section of 15, 20, 25, 30, 35, 40, 45, 50, 75, 100, 200, 250, 300, 400, 500, 600, 700, 800, 900, or 1000 DNA molecules corresponding to a genomic region having a genomic location defined in Tables 1 to 4.

In the kits of the invention comprising oligonucleotides, preferably at least one of the oligonucleotides for specifically hybridizing to at least a section of the DNA molecules is an amplification primer. Even more preferably, each oligonucleotide for specifically hybridizing to at least a section of the DNA molecules is an amplification primer.

### Method of Treatment and Uses of Therapeutic Agents for the Treatment of a Subject Having Prostate Cancer

As the methods of the invention of the present invention are for detecting, screening, monitoring, staging, classification, selecting treatment for, ascertaining whether treatment is working in, and/or prognostication of prostate cancer, a method of the invention may be used in a method of treatment of a subject having prostate cancer and/or used with a therapeutic agent for use in the treatment of a subject having prostate cancer.

A therapeutic agent for the treatment of prostate cancer for use in the methods of treatment and uses of the present invention, as well as in the methods, kits, and other aspects of the present invention, is selected from the group consisting of a hormonal agent, a targeted agent, a biologic agent, an immunotherapy agent, a chemotherapy agent and a radionuclide agent.

A hormonal agent for the treatment of prostate cancer is selected from the group consisting of LHRH agonists (for example leuprolide, goserelin, triptorelin, or histrelin), LHRH antagonists (for example degarelix), androgen blockers (for example abiraterone or ketoconazole), anti-androgens (for example flutamide, bicalutamide, nilutamide, enzalutamide, apalutamide or darolutamide), estrogens and steroids (for example prednisone or dexamethasone).

A targeted agent for the treatment of prostate cancer is selected from the group consisting of poly(ADP-ribose) polymerase (PARP) inhibitors (for example olaparib, rucaparib, niraparib or talazoparib), epidermal growth factor receptor (EGFR) inhibitors (for example gefitinib, erlotinib, afatinib, brigatinib, icotinib, cetuximab, osimertinib, adavosertib, or lapatinib), and tyrosine kinase inhibitors (for example imatinib, gefitinib, erlotinib, or sunitinib).

A biologic agent for the treatment of prostate cancer is selected from the group consisting of monoclonal antibodies (for example pertuzumab, trastuzumab or solitomab), hormones (for example a hormonal agent selected from LHRH agonists (for example leuprolide, goserelin, triptorelin, or histrelin), LHRH antagonists (for example degarelix), androgen blockers (for example abiraterone or ketoconazole), anti-androgens (for example flutamide, bicalutamide, nilutamide, enzalutamide, apalutamide or darolutamide), and estrogens), interferons (for example interferons-α, -β, -γ), and interleukin-based products (for example interleukin-2).

An immunotherapy agent for the treatment of prostate cancer is selected from the group consisting of cancer vaccines (for example sipuleucel-T), T-cell therapies, monoclonal antibody therapies, immune checkpoint therapies (for example a PD-1 inhibitor (e.g. pembrolizumab, nivolumab, cemiplimab, or spartalizumab), PD-L1 inhibitors (e.g. atezolizumab, avelumab or durvalumab), or a CTLA-4 (e.g. ipilimumab)), and non-specific immunotherapies (for example interferons or inerleukins).

A chemotherapy agent for the treatment of prostate cancer is selected from the group consisting selected from docetaxel, cabazitaxel, and c-Met inhibitors (for example cabozantinib).

A radionuclide agent for the treatment of prostate cancer is selected from Radium223 and PSMA-labelled radionuclide (for example ²²⁵Ac-Labeled PSMA-617 or ¹⁷⁷Lu-Labeled PSMA-617).

A therapeutic agent for the treatment of prostate cancer may be administered in amounts indicated in the Physicians' Desk Reference (PDR) or as otherwise determined by one of ordinary skill in the art.

In certain preferred embodiments, a therapeutic agent for the treatment of prostate cancer for use in the methods of treatment and uses of the present invention, as well as in the methods, kits, and other aspects of the present invention, is a hormonal agent and optionally a chemotherapy agent and/or optionally a further hormonal agent and/or optionally a targeted agent and/or optionally a radionuclide agent and/or an immunotherapy agent. For example, a hormonal agent selected from a LHRH agonist (for example leuprolide, goserelin, triptorelin, or histrelin) and a LHRH antagonist (for example degarelix), and optionally docetaxel and/or optionally a PARP inhibitor (for example olaparib, rucaparib, niraparib or talazoparib). Or, for example, a LHRH agonist (for example leuprolide, goserelin, triptorelin, or histrelin) or a LHRH antagonist (for example degarelix), and optionally a chemotherapy agent (for example docetaxel, cabazitaxel, carboplatin) and/or optionally a further hormonal treatment (for example enzalutamide, abiraterone, darolutamide) and/or optionally a radionuclide agent (Radium223 or PSMA-labelled radionuclide) and/or optionally a PARP inhibitor (for example olaparib, rucaparib, niraparib or talazoparib) and/or an immunotherapy agent (for example nivolumab, pembroluzimab, ipilumimab, durvalumab).

In embodiments wherein the prostate cancer is castration sensitive prostate cancer, preferably the therapeutic agent is a LHRH agonist (for example leuprolide, goserelin, triptorelin, or histrelin) or a LHRH antagonist (for example degarelix) and optionally a chemotherapy agent (for example docetaxel, cabazitaxel, carboplatin) and/or optionally a further hormonal treatment (for example enzalutamide, abiraterone, darolutamide) and/or optionally a radionuclide agent (Radium223 or PSMA-labelled radionuclide (for example ²²⁵Ac-Labeled PSMA-617 or ¹⁷⁷Lu-Labeled PSMA-617)) and/or optionally a PARP inhibitor (for example olaparib, rucaparib, niraparib or talazoparib) and/or immunotherapy (for example nivolumab, pembroluzimab, ipilumimab, durvalumab).

In embodiments wherein the prostate cancer is castration resistant prostate cancer, preferably the therapeutic agent for the treatment of prostate cancer is a LHRH agonist (for example leuprolide, goserelin, triptorelin, or histrelin) or a LHRH antagonist (for example degarelix), and optionally a chemotherapy agent (for example docetaxel, cabazitaxel, carboplatin) and/or optionally a further hormonal treatment (for example enzalutamide, abiraterone, darolutamide) and/or optionally a radionuclide agent (Radium223 or a PSMA-labelled radionuclide (for example ²²⁵Ac-Labeled PSMA-617 or ¹⁷⁷Lu-Labeled PSMA-617)) and/or optionally a PARP inhibitor (for example olaparib, rucaparib, niraparib or talazoparib) and/or immunotherapy agent (for example nivolumab, pembroluzimab, ipilumimab, durvalumab).

A non-therapeutic treatment for the treatment of prostate cancer is selected from surgery and radiotherapy. A surgical treatment of prostate cancer is selected from the group consisting of radical prostatectomy, a trans-urethral resection of the prostate, and an orchidectomy. A radiotherapy treatment of prostate cancer is selected from external beam localized radiotherapy of the prostate, external beam radiotherapy of metastatic sites.

In certain embodiments, methods of treatment of the present invention comprise treating the subject using a therapeutic agent for the treatment of prostate cancer, surgery, and/or radiotherapy. In certain embodiments, methods of treatment of the present invention comprise administering to the subject an effective amount of a therapeutic agent for the treatment of prostate cancer, and/or radiotherapy, and/or performing surgery. In certain embodiments, methods of treatment of the present invention comprise starting, ceasing or altering treatment with a therapeutic agent, or initiating a non-therapeutic treatment (e.g., surgery or radiation).

The present invention provides a method for treating prostate cancer in a subject comprising a method defined herein (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein) and further comprising treating the subject using a therapeutic agent for the treatment of prostate cancer, surgery, and/or radiotherapy.

The present invention also provides a method for treating prostate cancer in a subject comprising a method defined herein (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein) and further comprising administering to the subject an effective amount of a therapeutic agent for the treatment of prostate cancer, and/or radiotherapy, and/or performing surgery.

A method of treatment of the present invention is performed before and/or after a method of the invention defined herein (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein).

Preferably, a method for treating prostate cancer of the present invention comprises administering to the subject an effective amount of a therapeutic agent for the treatment of prostate cancer surgery, and/or radiotherapy after a method of the invention defined herein, for example after the subject has been determined to have a level of prostate cancer fraction, or determined to have cfDNA derived from a prostate cancer subtype, based on a method as described herein. In another preferred embodiment, a method for treating prostate cancer of the present invention comprises administering to the subject an effective amount of a therapeutic agent for the treatment of prostate cancer after a method of the invention defined herein, for example after the subject has been determined to have a level of prostate cancer fraction, or determined to have cfDNA derived from a prostate cancer subtype, based on a method as described herein.

In one embodiment, a method for treating prostate cancer of the present invention comprises administering a therapeutic agent for the treatment of prostate cancer to the subject for at least 1 week, 2 weeks, 3 weeks, 4 weeks, 1 month, 2 months, 3 months, 6 months, 9 months, 12 months, 24 months or 36 months. A therapeutic agent for the treatment of prostate cancer may be administered, for example, daily, every second day, twice per week, weekly or monthly.

In one embodiment, a method for treating prostate cancer of the present invention comprises treating a subject using a therapeutic agent for the treatment of prostate cancer for at least 1 week, 2 weeks, 3 weeks, 4 weeks, 1 month, 2 months, 3 months, 6 months, 9 months, 12 months, 24 months or 36 months.

A therapeutic agent for the treatment of prostate cancer may be administered in amounts and at frequencies indicated in the Physicians' Desk Reference (PDR) or as otherwise determined by one of ordinary skill in the art.

In one preferred embodiment, a method for treating prostate cancer of the present invention comprises performing the method of the invention (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein) before treating the subject, and subsequently repeating the method of the invention, for example at least 1 week, at least 2 weeks, at least 3 weeks, at least 4 weeks, at least 1 month, at least 2 months, at least 3 months, at least 6 months, at least 9 months, at least 12 months, at least 24 months or at least 36 months after starting or finishing the treatment, for example after administering to the subject an effective amount of a therapeutic agent for the treatment of prostate cancer, and/or radiotherapy, and/or performing surgery.

In another preferred embodiment, a method for treating prostate cancer of the present invention comprises performing the method (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein) before treating the subject, and subsequently repeating the method, for example at least 1 week, at least 2 weeks, at least 3 weeks, at least 4 weeks, at least 1 month, at least 2 months, at least 3 months, at least 6 months, at least 9 months, at least 12 months, at least 24 months or at least 36 months after performing the first method of the invention.

In embodiments comprising repeating the method (for example, repeating the method for detecting, screening, monitoring, staging, classification, selecting treatment for, ascertaining whether treatment is working in, and/or prognostication of prostate cancer in a sample obtained from a subject, wherein the sample comprises cfDNA as defined herein), the method may be repeated once, or it may be repeated multiple times, for examples 2, 3, 4, 5, 6 or more times.

In embodiments comprising repeating the method (for example, repeating the method for detecting, screening, monitoring, staging, classification, selecting treatment, ascertaining whether treatment is working, and/or prognostication of prostate cancer in a sample obtained from a subject, wherein the sample comprises cfDNA as defined herein), after the subsequent method(s) is performed, the method may further comprise continuing to treat the subject with the therapeutic agent for the treatment of prostate cancer if the level of prostate cancer tumour fraction is the same or substantially the same in the initial and subsequent method(s) or lower in the subsequent method(s) than in the initial method.

In embodiments comprising repeating the method (for example, repeating the method for detecting, screening, monitoring, staging, classification, selecting treatment for, ascertaining whether treatment is working in, and/or prognostication of prostate cancer in a sample obtained from a subject, wherein the sample comprises cfDNA as defined herein), after the subsequent method(s) is performed, the method may further comprise:

ceasing or altering (e.g. changing the dose or frequency of the dosing) treatment with the therapeutic agent for the treatment of prostate cancer; and/or
initiating treatment with a second or further therapeutic agent for the treatment of prostate cancer; and/or
initiating a non-therapeutic agent treatment (e.g., surgery or radiation),
if the level of prostate cancer tumour fraction is substantially the same in the initial and subsequent method or higher in the subsequent method than in the initial method; or
if the sample comprises cfDNA derived from a prostate cancer subtype and/or the sample comprises a level of cfDNA derived from a prostate cancer subtype that is substantially the same in the initial and subsequent method or higher in the subsequent method than in the initial method.

The invention further provides a method of treating a subject in need of treatment with a therapeutic agent for the treatment of prostate cancer, comprising

i) performing a method of the invention (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein) to determine the level of prostate cancer tumour fraction in the subject;
ii) administering a therapeutic agent for the treatment of prostate cancer if the subject has a level of prostate cancer tumour fraction or if the sample comprises cfDNA derived from a prostate cancer subtype and/or if the sample comprises a level of cfDNA derived from a prostate cancer subtype (for example 0.01% or more, more preferably 0.02% or more, more preferably 0.03% or more, more preferably 0.04% or more, for example 0.05% or more, 0.1% or more, 0.5% or more, or 1% or more cfDNA derived from a prostate cancer subtype.

In certain embodiments, the method of treating a subject comprises administering a therapeutic agent for the treatment of prostate cancer if the subject has a detectable level of prostate cancer tumour DNA, for example 0.01% or more, 0.02% or more, 0.03% or more, 0.04% or more, 0.05% or more, 0.1% or more, 0.5% or more, or 1% or more, prostate cancer fraction.

In certain embodiments the method further comprises administering a second therapeutic agent for the treatment of prostate cancer if the subject has a level of prostate cancer fraction (for example a detectable level of prostate cancer fraction, for example 0.01% or more, 0.02% or more, 0.03% or more, 0.04% or more, 0.05% or more, 0.1% or more, 0.5% or more, or 1% or more, prostate cancer fraction). In one preferred embodiment, the method further comprises administering a second therapeutic agent for the treatment of prostate cancer if the subject has a level of prostate cancer fraction 0.01% or more, more preferably 0.02% or more, more preferably 0.03% or more, more preferably 0.04% or more, for example 0.05% or more, 0.1% or more, 0.5% or more, or 1% or more, prostate cancer fraction.

In certain embodiments, the method of treating a subject comprises

(iii) at least 1 week, at least 2 weeks, at least 3 weeks, at least 4 weeks, at least 1 month, at least 2 months, at least 3 months, at least 6 months, at least 9 months, at least 12 months, at least 24 months, or at least 36 months, after the administration of the therapeutic agent, a further sample comprising cfDNA is obtained from the subject, and the method of the invention (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein) is performed to determine the level of prostate cancer fraction in the further sample.

The invention also provides a therapeutic agent for the treatment of prostate cancer, for use in the treatment of prostate cancer, wherein

i) a method of the invention (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein) is performed to determine the level of prostate cancer prostate cancer fraction in a subject;
ii) the therapeutic agent is administered if the subject has a level of prostate cancer.

In certain embodiments, the therapeutic agent for use in the treatment of prostate cancer is one for use in a treatment that comprises administering a therapeutic agent for the treatment of prostate cancer if the subject has a detectable level of prostate cancer tumour DNA, for example 0.01% or more, 0.02% or more, 0.03% or more, 0.04% or more, 0.05% or more, 0.1% or more, 0.5% or more, or 1% or more, prostate cancer fraction.

In certain embodiments, the therapeutic agent for use in the treatment of prostate cancer is one for use in a treatment that comprises administering a second therapeutic agent for the treatment of prostate cancer if the subject has a level of prostate cancer fraction (for example a detectable level of prostate cancer fraction, for example 0.01% or more, 0.02% or more, 0.03% or more, 0.04% or more, 0.05% or more, 0.1% or more, 0.5% or more, or 1% or more, prostate cancer fraction). In one preferred embodiment, the therapeutic agent for use in the treatment of prostate cancer is one for use in a treatment that comprises administering a second therapeutic agent for the treatment of prostate cancer if the subject has a level of prostate cancer fraction 0.01% or more, more preferably 0.02% or more, more preferably 0.03% or more, more preferably 0.04% or more, for example 0.05% or more, 0.1% or more, 0.5% or more, or 1% or more, prostate cancer fraction.

In certain embodiments, the therapeutic agent for use in the treatment of prostate cancer is one for use in a treatment in which

(iii) at least 1 week, at least 2 weeks, at least 3 weeks, at least 4 weeks, at least 1 month, at least 2 months, at least 3 months, at least 6 months, at least 9 months, at least 12 months, at least 24 months, or at least 36 months, after the administration of the therapeutic agent, a further sample comprising cfDNA is obtained from the subject, and the method of the invention (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein) is performed to determine the level of prostate cancer fraction in the further sample.

### Applications of Methods of the Invention

The present invention also provides a method of determining one or more suitable therapeutic agents for the treatment of prostate cancer in a subject having prostate cancer comprising
performing a method of invention (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein, to determine the level of prostate cancer fraction in the cfDNA sample);
determining the one or more suitable therapeutic agents for the treatment of prostate cancer by reference to the level of prostate cancer, whereby one therapeutic agent is suitable for a subject with no level of prostate cancer tumour fraction or a percentage level of prostate cancer fraction of less than 0.01%, and two or more therapeutic agents are suitable for a subject with a level of prostate cancer fraction or a percentage level of prostate cancer fraction of 0.01% or more;
   or whereby a therapeutic agent selected from a first list of therapeutic agents is suitable for a subject with no level of prostate cancer fraction or a percentage level of prostate cancer fraction of less than 0.01%, and a therapeutic agent from a second list of therapeutic agents, or two or more therapeutic agents from the first list, is suitable for a subject with a level of prostate cancer fraction or a percentage level of prostate cancer fraction of 0.01% or more.

In certain embodiments, no level of prostate cancer tumour is no detectable level of prostate cancer. In certain embodiments, a level of prostate cancer tumour is a detectable level of prostate cancer, for example 0.01% or more, 0.02% or more, 0.03% or more, 0.04% or more, 0.05% or more, 0.1% or more, 0.5% or more, or 1% or more, prostate cancer fraction. In certain embodiments, a level of prostate cancer tumour is a detectable level of prostate cancer. In certain embodiments, a level of prostate cancer fraction 0.01% or more, more preferably 0.02% or more, more preferably 0.03% or more, more preferably 0.04% or more, for example 0.05% or more, 0.1% or more, 0.5% or more, or 1% or more, prostate cancer fraction.

In certain embodiments, the method of determining one or more suitable therapeutic agents for the treatment of prostate cancer for a subject having prostate cancer comprises
performing a method of invention;
determining the one or more suitable therapeutic agents for the treatment of prostate cancer by reference to the level of prostate cancer, whereby one therapeutic agent is suitable for a subject with no level of prostate cancer tumour fraction, and two or more therapeutic agents are suitable for a subject with a level of prostate cancer fraction;
   or whereby a therapeutic agent selected from a first list of therapeutic agents is suitable for a subject with no level of prostate cancer fraction, and a therapeutic agent from a second list of therapeutic agents, or two or more therapeutic agents from the first list, is suitable for a subject with a level of prostate cancer fraction.

In certain embodiments, the method of determining one or more suitable therapeutic agents for the treatment of prostate cancer for a subject having prostate cancer comprises
performing a method of invention;
determining the one or more suitable therapeutic agents for the treatment of prostate cancer by reference to the level of prostate cancer, whereby one therapeutic agent is suitable for a subject with a level of prostate cancer fraction of less than 0.01%, and two or more therapeutic agents are suitable for a subject with a level of prostate cancer fraction of 0.01% or more;
   or whereby a therapeutic agent selected from a first list of therapeutic agents is suitable for a subject with a level of prostate cancer fraction of less than 0.01%, and a therapeutic agent from a second list of therapeutic agents, or two or more therapeutic agents from the first list, is suitable for a subject with a level of prostate cancer fraction of 0.01% or more.

The present invention also provides a method of determining a suitable treatment regimen for a subject having prostate cancer comprising:
performing a method of invention (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein, to determine the level of prostate cancer fraction in the cfDNA sample);
determining the treatment regimen by reference to the level of prostate cancer fraction, whereby a standard treatment is suitable for a subject having no level of prostate cancer fraction or a level of prostate cancer fraction of less than 0.01%, and a non-standard treatment is suitable for a subject with a level of prostate cancer fraction or a level of prostate cancer fraction of 0.01% or more.

In certain embodiments, no level of prostate cancer tumour is no detectable level of prostate cancer. In certain embodiments, a percentage level of prostate cancer tumour is a detectable level of prostate cancer, for example 0.01% or more, 0.02% or more, 0.03% or more, 0.04% or more, 0.05% or more, 0.1% or more, 0.5% or more, or 1% or more, prostate cancer fraction. In certain embodiments, a level of prostate cancer tumour is a detectable level of prostate cancer. In certain embodiments, a percentage level of prostate cancer fraction 0.01% or more, more preferably 0.02% or more, more preferably 0.03% or more, more preferably 0.04% or more, for example 0.05% or more, 0.1% or more, 0.5% or more, or 1% or more, prostate cancer fraction.

In certain embodiments, a standard treatment is a treatment with a therapeutic agent for the treatment of prostate cancer, and a non-standard treatment is a treatment with two or more therapeutic agents for the treatment of prostate cancer.

In certain embodiments, a standard treatment is a treatment with a hormonal agent for the treatment of prostate cancer, and a non-standard treatment is a treatment with a hormonal agent for the treatment of prostate cancer, and a chemotherapeutic agent for the treatment of prostate cancer and/or a immunotherapy treatment of prostate cancer and/or a targeted treatment of prostate cancer and/or a biologic agent treatment of prostate cancer and/or a radionuclide agent treatment.

In certain embodiments, the method of determining a suitable treatment regimen for a subject having prostate cancer comprising
performing a method of invention;
determining the treatment regimen by reference to the level of prostate cancer fraction, whereby a standard treatment is suitable for a subject having no level of prostate cancer fraction, and a non-standard treatment is suitable for a subject with a level of prostate cancer fraction.

In certain embodiments, the method of determining a suitable treatment regimen for a subject having prostate cancer comprising
performing a method of invention;
determining the treatment regimen by reference to the level of prostate cancer fraction, whereby a standard treatment is suitable for a subject having a percentage level of prostate cancer fraction of less than 0.01%, and a non-standard treatment is suitable for a subject with a percentage level of prostate cancer fraction of 0.01% or more.

In certain embodiments, the method of determining a suitable treatment regimen for a subject having prostate cancer comprising
performing a method of invention;
determining the treatment regimen by reference to the level of prostate cancer fraction, whereby a standard treatment is suitable for a subject having a percentage level of prostate cancer fraction of less than 0.02%, and a non-standard treatment is suitable for a subject with a percentage level of prostate cancer fraction of 0.02% or more.

In certain embodiments, the method of determining a suitable treatment regimen for a subject having prostate cancer comprising
performing a method of invention;
determining the treatment regimen by reference to the level of prostate cancer fraction, whereby a standard treatment is suitable for a subject having a percentage level of prostate cancer fraction of less than 0.05%, and a non-standard treatment is suitable for a subject with a percentage level of prostate cancer fraction of 0.05% or more.

In certain embodiments, the method of determining a suitable treatment regimen for a subject having prostate cancer comprising
performing a method of invention;
determining the treatment regimen by reference to the level of prostate cancer fraction, whereby a standard treatment is suitable for a subject having a percentage level of prostate cancer fraction of less than 0.1%, and a non-standard treatment is suitable for a subject with a percentage level of prostate cancer fraction of 0.1% or more.

In certain embodiments, the method of determining a suitable treatment regimen for a subject having prostate cancer comprising
performing a method of invention;
determining the treatment regimen by reference to the level of prostate cancer fraction, whereby a standard treatment is suitable for a subject having a percentage level of prostate cancer fraction of less than 0.5%, and a non-standard treatment is suitable for a subject with a percentage level of prostate cancer fraction of 0.5% or more.

In certain embodiments, the method of determining a suitable treatment regimen for a subject having prostate cancer comprising

performing a method of invention;
determining the treatment regimen by reference to the level of prostate cancer fraction, whereby a standard treatment is suitable for a subject having a percentage level of prostate cancer fraction of less than 1%, and a non-standard treatment is suitable for a subject with a percentage level of prostate cancer fraction of 1% or more.

The present invention also provides a method of performing a method of invention (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein, to determine whether the sample comprises cfDNA derived from a prostate cancer subtype);

determining the one or more suitable therapeutic agents for the treatment of prostate cancer by reference to whether the sample comprises cfDNA derived from a prostate cancer subtype and/or the level of cfDNA in the sample that is derived from a prostate cancer subtype, whereby one therapeutic agent is suitable for a subject with a sample having no cfDNA derived from a prostate cancer subtype (for example an undetectable level of cfDNA derived from a prostate cancer subtype) or a level of cfDNA derived from a prostate cancer subtype of less than 0.01%, and two or more therapeutic agents are suitable for a subject with a level of cfDNA derived from a prostate cancer subtype (for example a percentage level of cfDNA derived from a prostate cancer subtype of at least 0.01%);
or whereby a therapeutic agent selected from a first list of therapeutic agents is suitable for a subject with a sample having no cfDNA derived from a prostate cancer subtype (for example an undetectable level of cfDNA derived from a prostate cancer subtype) or a level of cfDNA derived from a prostate cancer subtype of less than 0.01%, and a therapeutic agent from a second list of therapeutic agents, or two or more therapeutic agents from the first list, is suitable for a subject with a level of cfDNA derived from a prostate cancer subtype (for example a percentage level of cfDNA derived from a prostate cancer subtype of at least 0.01%).

In certain embodiments, no cfDNA derived from a prostate cancer subtype is no detectable cfDNA derived from a prostate cancer subtype. In certain embodiments, a percentage level of cfDNA derived from a prostate cancer subtype is a detectable level of cfDNA derived from a prostate cancer subtype, for example 0.01% or more, 0.02% or more, 0.03% or more, 0.04% or more, 0.05% or more, 0.1% or more, 0.5% or more, or 1% or more, prostate cancer fraction. In certain embodiments, a level of cfDNA derived from a prostate cancer subtype is a detectable level of prostate cancer. In certain embodiments, a percentage level of cfDNA derived from a prostate cancer subtype is 0.01% or more, more preferably 0.02% or more, more preferably 0.03% or more, more preferably 0.04% or more, for example 0.05% or more, 0.1% or more, 0.5% or more, or 1% or more, prostate cancer fraction.

The present invention also provides a method of determining a suitable treatment regimen for a subject having prostate cancer comprising:

performing a method of invention (for example, a method for detecting, screening, monitoring, staging, classification, selecting treatment for, ascertaining whether treatment is working in, and/or prognostication of prostate cancer in a sample obtained from a subject, wherein the sample comprises cfDNA as defined herein, to determine whether the sample comprises cfDNA derived from a prostate cancer subtype);
determining the treatment regimen by reference whether the sample comprises cfDNA derived from a prostate cancer subtype and/or the level of cfDNA in the sample that is derived from a prostate cancer subtype, whereby a standard treatment is suitable for a subject with a sample having no cfDNA derived from a prostate cancer subtype (for example an undetectable level of cfDNA derived from a prostate cancer subtype) or a percentage level of cfDNA derived from a prostate cancer subtype of less than 0.01%, and a non-standard treatment is suitable for a subject with a level cfDNA derived from a prostate cancer subtype (for example a detectable level of prostate cancer fraction) or a percentage level of prostate cancer fraction of at least 0.01%.

In certain embodiments, the method of determining a suitable treatment regimen for a subject having prostate cancer comprising

performing a method of invention;
determining the treatment regimen by reference whether the sample comprises cfDNA derived from a prostate cancer subtype and/or the level of cfDNA in the sample that is derived from a prostate cancer subtype, whereby a standard treatment is suitable for a subject with a sample having no cfDNA derived from a prostate cancer subtype (for example an undetectable level of cfDNA derived from a prostate cancer subtype) or a percentage level of cfDNA derived from a prostate cancer subtype of less than 0.1%, and a non-standard treatment is suitable for a subject with a level cfDNA derived from a prostate cancer subtype (for example a detectable level of prostate cancer fraction) or a percentage level of prostate cancer fraction of at least 0.1%.

In certain embodiments, the method of determining a suitable treatment regimen for a subject having prostate cancer comprising

performing a method of invention;
determining the treatment regimen by reference whether the sample comprises cfDNA derived from a prostate cancer subtype and/or the level of cfDNA in the sample that is derived from a prostate cancer subtype, whereby a standard treatment is suitable for a subject with a sample having no cfDNA derived from a prostate cancer subtype (for example an undetectable level of cfDNA derived from a prostate cancer subtype) or a percentage level of cfDNA derived from a prostate cancer subtype of less than 1%, and a non-standard treatment is suitable for a subject with a level cfDNA derived from a prostate cancer subtype (for example a detectable level of prostate cancer fraction) or a percentage level of prostate cancer fraction of at least 1%.

In certain embodiments, a standard treatment is a treatment with a therapeutic agent for the treatment of prostate cancer, and a non-standard treatment is a treatment with two or more therapeutic agents for the treatment of prostate cancer.

In certain embodiments, a standard treatment is a treatment with a hormonal agent for the treatment of prostate cancer, and a non-standard treatment is a treatment with a hormonal agent for the treatment of prostate cancer, and a chemotherapeutic agent for the treatment of prostate cancer and/or a immunotherapy treatment of prostate cancer and/or a targeted treatment of prostate cancer and/or a biologic agent treatment and/or a radionuclide agent treatment of prostate cancer.

### Computer Implemented Methods and Software

The invention also provides a computerized (or computer implemented) method and/or computer-assisted method and/or a computer product and/or a computer implemented software for performing or implementing the method defined herein, for example the method for detecting, screening, monitoring, staging, classification, selecting treatment for, ascertaining whether treatment is working in, and/or prognostication of prostate cancer in a sample obtained from a subject described herein, the methods of treatment and therapeutic agents for use described herein, the methods of determining one or more suitable therapeutic agents for the treatment of prostate cancer described herein, the methods for determining a treatment regimen described herein, and the methods of determining a solid cancer cfDNA methylome signature. A kit of the invention may comprise a computerized method and/or computer-assisted method and/or a computer product and/or a computer implemented software of the present invention.

A computerized method and/or computer-assisted method and/or a computer product and/or a computer implemented software for performing or implementing a method defined herein comprises performing one or more steps of the relevant method, or in certain embodiments, comprises performing the relevant method. A computerized (or computer implemented) method and/or computer-assisted method and/or a computer implemented software can control a computer product to execute, perform or implement one or more steps of the relevant method, or in certain embodiments, comprises performing the relevant method.

In certain embodiments, the present invention provides a computer product. A computer product of the present invention has the means for performing or implementing one or more method described herein.

In some embodiments, a computer product of the present invention comprises at least one memory containing at least one computer program or software adapted to control the operation of the computer system to perform or implement a method that includes receiving and characterizing DNA methylation data e.g., receiving and characterizing methylome sequences of a plurality of cfDNA molecules and determining the average methylation ratio at 10 or more genomic regions, and at least one processor for executing the computer program or software.

In some embodiments, a computer product of the present invention comprises a non-transitory computer readable medium storing a plurality of instructions that, when executed, control a computer system to perform one or more steps of a method described herein or comprises performing or implementing a method described herein.

In certain embodiments, a computer product is a product having a computer, where the computer comprises a computer-readable medium embodying software to operate the computer. In some cases, the computer system includes one or more general or special purpose processors and associated memory, including volatile and non-volatile memory devices. In some cases, the computer product memory stores software or computer programs for controlling the operation of the computer system to make a special purpose system according to the invention or to implement a system to perform the methods according to the invention. In some cases, the computer system includes a single or multi-core central processing unit (CPU), an ARM processor or similar computer processor for processing the data. In some cases, the CPU or microprocessor is any conventional general purpose single- or multi-chip microprocessor, a RISC or MISS processor, a Power PC processor, or an ALPHA processor. In some cases, the microprocessor is any conventional or special purpose microprocessor such as a digital signal processor or a graphics processor. The microprocessor typically has conventional address lines, conventional data lines, and one or more conventional control lines. The software or computer program may be executed on dedicated system or on a general purpose computer having, for example, a Windows, Unix, Linux or other operating system. In some instances, the system includes non-volatile memory, such as disk memory and solid state memory for storing computer programs, software and data and volatile memory, such as high speed ram for executing programs and software.

In certain embodiments, a computer product is a storage device used for storing data accessible by a computer, as well as any other means for providing access to data by a computer. Examples of a storage device-type computer-readable medium include: a magnetic hard disk; an optical disk, such as a CD-ROM and a DVD; a magnetic tape; a memory chip. Examples of a computer-readable physical storage media include any physical computer-readable storage medium, e.g., solid state memory (such as flash memory), magnetic and optical computer-readable storage media and devices, and memory that uses other persistent storage technologies. In certain embodiments, a computer product is computer readable media selected from the group consisting of RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, and magnetic disk storage.

In one preferred embodiment, the present invention provides a computerized (or computer implemented) method and/or computer-assisted method and/or a computer product and/or computerized (or computer implemented) software for detection, screening, monitoring, staging, classification and/or prognostication of prostate cancer in a sample obtained from a subject, wherein the sample comprises cfDNA, the method comprising:

receiving a data set in a computer comprising a processor and a computer readable medium, wherein the data set comprises the methylome sequence of a plurality of cfDNA molecules in the sample; and wherein the computer readable medium comprises instructions that, when executed by the processors, causes the computer to perform or implement a method of the invention.

For example, in one embodiment it causes the computer to perform or implement a method comprising the following steps:

characterize the methylome sequence of a plurality of cfDNA molecules in the sample, wherein the methylome sequence of a cfDNA molecule is the DNA sequence and the methylation profile of the molecule;
determine the average methylation ratio at 10 or more genomic regions, each genomic region being selected from the group consisting of:
a 100 to 200 bp region comprising or having a genomic location defined in Tables 1 to 4, and
a 2 to 99 bp region within a genomic location defined in Tables 1 to 4 and comprising at least one CpG locus,
and wherein each genomic region is covered by at least one sequence read of at least one characterized methylome sequence;
calculate a methylation score using the average methylation ratio for each genomic region;
analyse the methylation score to determine the level of prostate cancer fraction in the cfDNA sample.

For example, in one embodiment it causes the computer to perform or implement a method comprising the following steps:

characterize the methylome sequence of a plurality of cfDNA molecules in the sample, wherein the methylome sequence of a cfDNA molecule is the DNA sequence and the methylation profile of the molecule;
determine the average methylation ratio at 10 or more genomic regions, each genomic region being selected from the group consisting of:
a 100 to 200 bp region comprising or having a genomic location defined in Table 8, and
a 2 to 99 bp region within a genomic location defined in Table 8 and comprising at least one CpG locus,
and wherein each of the genomic regions is covered by at least one sequence read of at least one characterized methylome sequence;
calculate a methylation score using the average methylation ratio for each of the genomic regions;
analyze the methylation score to determine whether the sample comprises cfDNA derived from a prostate cancer subtype.

In one preferred embodiment, the present invention provides a computerized (or computer implemented) method and/or computer-assisted method and/or a computer product method and/or computerized (or computer implemented) software for classifying a prostate cancer patient into one or more of a plurality of treatment categories, the method comprising determining the level of prostate cancer fraction in a sample obtained from a subject, wherein the sample comprises cfDNA, the method comprising:

receiving a data set in a computer comprising a processor and a computer readable medium, wherein the data set comprises the methylome sequence of a plurality of cfDNA molecules in a sample obtained from a subject, wherein the sample comprises cfDNA;
and wherein the computer readable medium comprises instructions that, when executed by the processors, causes the computer to perform or implement a method of the invention.

For example, in one embodiment it causes the computer to perform or implement a method comprising the following steps:

characterize the methylome sequence of a plurality of cfDNA molecules in the sample, wherein the methylome sequence of a cfDNA molecule is the DNA sequence and the methylation profile of the molecule;
determine the average methylation ratio at 10 or more genomic regions, each genomic region being selected from the group consisting of:
a 100 to 200 bp region comprising or having a genomic location defined in Tables 1 to 4, and
a 2 to 99 bp region within a genomic location defined in Tables 1 to 4 and comprising at least one CpG locus,
and wherein each genomic region is covered by at least one sequence read of at least one characterized methylome sequence;
calculate a methylation score using the average methylation ratio for each genomic region;
analyse the methylation score to determine the level of prostate cancer fraction in the cfDNA sample.

For example, in one embodiment it causes the computer to perform or implement a method comprising the following steps:

characterize the methylome sequence of a plurality of cfDNA molecules in the sample, wherein the methylome sequence of a cfDNA molecule is the DNA sequence and the methylation profile of the molecule;
determine the average methylation ratio at 10 or more genomic regions, each genomic region being selected from the group consisting of:
a 100 to 200 bp region comprising or having a genomic location defined in Table 8, and
a 2 to 99 bp region within a genomic location defined in Table 8 and comprising at least one CpG locus,
and wherein each of the genomic regions is covered by at least one sequence read of at least one characterized methylome sequence;
calculate a methylation score using the average methylation ratio for each of the genomic regions;
analyze the methylation score to determine whether the sample comprises cfDNA derived from a prostate cancer subtype.

In another preferred embodiment, the present invention provides a computerized (or computer implemented) method and/or computer-assisted method and/or a computer product method and/or computerized (or computer implemented) software for classifying a prostate cancer patient into one or more of a plurality of treatment categories, the method comprising determining the subtype of prostate cancer a sample obtained from a subject, wherein the sample comprises cfDNA, the method comprising:

receiving a data set in a computer comprising a processor and a computer readable medium, wherein the data set comprises the methylome sequence of a plurality of cfDNA molecules in a sample obtained from a subject, wherein the sample comprises cfDNA;
and wherein the computer readable medium comprises instructions that, when executed by the processors, causes the computer to perform or implement a method of the invention.

For example, in one embodiment it causes the computer to perform or implement a method comprising the following steps:

characterize the methylome sequence of a plurality of cfDNA molecules in the sample, wherein the methylome sequence of a cfDNA molecule is the DNA sequence and the methylation profile of the molecule;
determine the average methylation ratio at 10 or more genomic regions, each genomic region being selected from the group consisting of:
a 100 to 200 bp region comprising or having a genomic location defined in Tables 1 to 4, and
a 2 to 99 bp region within a genomic location defined in Tables 1 to 4 and comprising at least one CpG locus,
and wherein each genomic region is covered by at least one sequence read of at least one characterized methylome sequence;
calculate a methylation score using the average methylation ratio for each genomic region;
analyse the methylation score to determine the level of prostate cancer fraction in the cfDNA sample.

For example, in one embodiment it causes the computer to perform or implement a method comprising the following steps:

characterize the methylome sequence of a plurality of cfDNA molecules in the sample, wherein the methylome sequence of a cfDNA molecule is the DNA sequence and the methylation profile of the molecule;
determine the average methylation ratio at 10 or more genomic regions, each genomic region being selected from the group consisting of:
a 100 to 200 bp region comprising or having a genomic location defined in Table 8, and
a 2 to 99 bp region within a genomic location defined in Table 8 and comprising at least one CpG locus,
and wherein each of the genomic regions is covered by at least one sequence read of at least one characterized methylome sequence;
calculate a methylation score using the average methylation ratio for each of the genomic regions;
analyze the methylation score to determine whether the sample comprises cfDNA derived from a prostate cancer subtype.

In one embodiment, the plurality of treatment categories are selected from a hormonal agent, a targeted agent, a biologic agent, an immunotherapy agent, and a chemotherapy agent.

In one embodiment, the plurality of treatment categories are selected from a treatment with a single agent (for example a hormonal agent, a targeted agent, a biologic agent, an immunotherapy agent, and a chemotherapy agent); and treatment with a combination of agents (for example, a combination of two or more agents selected from the group consisting of a hormonal agent, a targeted agent, a biologic agent, an immunotherapy agent, and a chemotherapy agent).

In one preferred embodiment, the plurality of treatment categories are selected from a treatment with a single agent (for example a hormonal agent, a targeted agent, a biologic agent, an immunotherapy agent, and a chemotherapy agent); and treatment with a combination of two, three, four of five agents (for example, a combination of two, three, four of five agents selected from the group consisting of a hormonal agent, a targeted agent, a biologic agent, an immunotherapy agent, and a chemotherapy agent).

For example, the plurality of treatment categories are selected from a hormonal agent; and a hormonal agent and a chemotherapeutic agent and/or a further hormonal agent.

In one preferred embodiment, the plurality of treatment categories are selected from a standard treatment (for example a treatment with a hormonal agent); and a non-standard treatment (for example a hormonal agent for the treatment of prostate cancer, and a chemotherapeutic agent for the treatment of prostate cancer and/or a immunotherapy treatment of prostate cancer and/or a targeted treatment of prostate cancer and/or a biologic agent treatment of prostate cancer).

In certain embodiments, a computerized (or computer implemented) method and/or computer-assisted method and/or a computer product and/or a computer implemented software described herein further comprises treating the subject for prostate cancer using a therapeutic agent for the treatment of prostate cancer;

or ceasing or altering treatment with a therapeutic agent for the treatment of prostate cancer; or initiating a non-therapeutic agent treatment for prostate cancer (for example initiation of treatment by surgery or radiation).

In another preferred embodiment, the present invention provides a computerized (or computer implemented) method and/or computer-assisted method and/or a computer product and/or computerized (or computer implemented) software for determining a solid cancer cfDNA methylome signature for use in the detecting, screening, monitoring, staging, classification, selecting treatment for, ascertaining whether treatment is working in, and/or prognostication of the solid cancer, the method comprising:

receiving a data set in a computer comprising a processor and a computer readable medium, wherein the data set comprises the methylome sequence of a plurality of cfDNA molecules in a sample from a subject known to have the solid cancer;
and wherein the computer readable medium comprises instructions that, when executed by the processors, causes the computer to to perform or implement a method comprising the following steps:
(i) characterize the methylome sequence of a plurality of cfDNA molecules in a first sample comprising cfDNA from a subject known to have the solid cancer, wherein the methylome sequence of a cfDNA molecule is the DNA sequence and the methylation profile of the molecule;
(ii) determine the respective number of characterised cfDNA molecules corresponding to a CpG locus or a genomic region of 2 to 10,000 bp (preferably 2 to 200 bp) in the first sample by aligning the methylome sequences;
(iii) determine the methylation ratio of each CpG locus and/or average methylation ratio of each genomic region of 2 to 10,000 bp (preferably 2 to 200 bp) in the first sample;
repeating steps (i) to (iii) for one or more further samples comprising cfDNA each from subjects known to have the solid cancer;
perform a variance analysis of all or a selection of the methylation ratios of the CpG loci and/or all or a selection of average methylation ratios of the genomic regions of the samples;
select a group of CpG loci and/or genomic regions associated with a feature of the samples; and
select CpG loci and/or genomic regions in the group to provide the cfDNA methylome signature.
Prostate Cancer cfDNA Methylome Signatures

The invention also provides a cfDNA methylome signature comprising a set of genomic locations defining 10 or more genomic regions.

In one embodiment, the set of genomic locations defining 10 or more genomic regions are genomic locations selected from the group consisting of:
a 100 to 200 bp (for example a 100 to 150 bp or 100 to 120 bp) genomic location comprising or having a genomic location defined in Tables 1 to 4, and
a 2 to 99 bp (for example a 20 to 99 bp or 50 to 99 bp) genomic location within a genomic location defined in Tables 1 to 4 and comprising at least one CpG locus.

In such embodiments, preferably the signature comprises a set of genomic locations defining 12 or more genomic regions, for example a set of genomic locations defining 15 or more genomic regions, a set of genomic locations defining 20 or more genomic regions, a set of genomic locations defining 25 or more genomic regions, a set of genomic locations defining 30 or more genomic regions, a set of genomic locations defining 50 or more genomic regions, a set of genomic locations defining 75 or more genomic regions, a set of genomic locations defining 100 or more genomic regions, a set of genomic locations defining 125 or more genomic regions, a set of genomic locations defining 150 or more genomic regions, a set of genomic locations defining 200 or more genomic regions, a set of genomic locations defining 300 or more genomic regions, a set of genomic locations defining 400 or more genomic regions, a set of genomic locations defining 500 or more genomic regions, a set of genomic locations defining 600 or more genomic regions, a set of genomic locations defining 700 or more genomic regions, a set of genomic locations defining 800 or more genomic regions, a set of genomic locations defining 900 or more genomic regions, or a set of genomic locations defining 1000 genomic regions.

The signature is for detecting, screening, monitoring, staging, classification, selecting treatment for, ascertaining whether treatment is working in, prognostication and/or treatment of prostate cancer. The methylation state (for example the average methylation ratio) of the genomic regions defined by the set of genomic locations of the signature may be used to indicate one or more of the following: the presence of prostate cancer cfDNA in the cfDNA sample, the level of prostate cancer fraction in the cfDNA sample, a subtype of prostate cancer (for example a genomic subtype or molecular subtype, such as castration resistant prostate cancer), if the prostate cancer is metastatic, the aggression of the prostate cancer, the prognosis of the prostate cancer, the tumour response to a treatment, the relapse of the prostate cancer, and/or the residual disease following curative treatment. The methylation state of the genomic regions defined by the set of genomic locations of the signature may be used to indicate the presence of prostate cancer cfDNA in the cfDNA sample and/or the level of prostate cancer fraction in the cfDNA sample.

In one embodiment, the set of genomic locations defining 10 or more genomic regions are genomic locations selected from the group consisting of:

a 100 to 200 bp (for example a 100 to 150 bp or 100 to 120 bp) genomic location comprising or having a genomic location defined in Tables 1 and 3, and
a 2 to 99 bp (for example a 20 to 99 bp or 50 to 99 bp) genomic location within a genomic location defined in Tables 1 and 3 and comprising at least one CpG locus. For example, the set of genomic locations defining 10 or more genomic regions are genomic locations selected from the the 100 bp genomic locations defined in Tables 1 and 3.

In such embodiments, preferably the signature comprises a set of genomic locations defining 12 or more genomic regions, for example a set of genomic locations defining 15 or more genomic regions, a set of genomic locations defining 20 or more genomic regions, a set of genomic locations defining 25 or more genomic regions, a set of genomic locations defining 30 or more genomic regions, a set of genomic locations defining 50 or more genomic regions, a set of genomic locations defining 75 or more genomic regions, a set of genomic locations defining 100 or more genomic regions, a set of genomic locations defining 125 or more genomic regions, a set of genomic locations defining 150 or more genomic regions, a set of genomic locations defining 200 or more genomic regions, a set of genomic locations defining 300 or more genomic regions, a set of genomic locations defining 400 or more genomic regions. In one embodiment, the set of genomic locations defining 10 or more genomic regions are genomic locations selected from the group consisting of:

a 100 to 200 bp (for example a 100 to 150 bp or 100 to 120 bp) genomic location comprising or having a genomic location defined in Tables 2 and 4, and
a 2 to 99 bp (for example a 20 to 99 bp or 50 to 99 bp) genomic location within a genomic location defined in Tables 2 and 4 and comprising at least one CpG locus. For example, the set of genomic locations defining 10 or more genomic regions are genomic locations selected from the 100 bp genomic locations defined in Tables 2 and 4.

In such embodiments, preferably the signature comprises a set of genomic locations defining 12 or more genomic regions, for example a set of genomic locations defining 15 or more genomic regions, a set of genomic locations defining 20 or more genomic regions, a set of genomic locations defining 25 or more genomic regions, a set of genomic locations defining 30 or more genomic regions, a set of genomic locations defining 50 or more genomic regions, a set of genomic locations defining 75 or more genomic regions, a set of genomic locations defining 100 or more genomic regions, a set of genomic locations defining 125 or more genomic regions, a set of genomic locations defining 150 or more genomic regions, a set of genomic locations defining 200 or more genomic regions, a set of genomic locations defining 300 or more genomic regions, a set of genomic locations defining 400 or more genomic regions.

In one embodiment, the set of genomic locations defining 10 or more genomic regions are genomic locations selected from the group consisting of:

a 100 to 200 bp (for example a 100 to 150 bp or 100 to 120 bp) genomic location comprising or having a genomic location defined in Tables 1 and 2, and
a 2 to 99 bp (for example a 20 to 99 bp or 50 to 99 bp) genomic location within a genomic location defined in Tables 1 and 2 and comprising at least one CpG locus. For example, the set of genomic locations defining 10 or more genomic regions are genomic locations selected from the 100 bp genomic locations defined in Tables 1 and 2.

In such embodiments, preferably the signature comprises a set of genomic locations defining 12 or more genomic regions, for example a set of genomic locations defining 15 or more genomic regions, a set of genomic locations defining 20 or more genomic regions, a set of genomic locations defining 25 or more genomic regions, a set of genomic locations defining 30 or more genomic regions, a set of genomic locations defining 50 or more genomic regions, a set of genomic locations defining 75 or more genomic regions, a set of genomic locations defining 100 or more genomic regions, a set of genomic locations defining 125 or more genomic regions, a set of genomic locations defining 150 or more genomic regions, a set of genomic locations defining 200 or more genomic regions, a set of genomic locations defining 300 or more genomic regions, a set of genomic locations defining 400 or more genomic regions, or a set of genomic locations defining 500 or more genomic regions.

In one embodiment, the set of genomic locations defining 10 or more genomic regions are genomic locations selected from the group consisting of:

a 100 to 200 bp (for example a 100 to 150 bp or 100 to 120 bp) genomic location comprising or having a genomic location defined in Tables 3 and 4, and
a 2 to 99 bp (for example a 20 to 99 bp or 50 to 99 bp) genomic location within a genomic location defined in Tables 3 and 4 and comprising at least one CpG locus. For example, the set of genomic locations defining 10 or more genomic regions are genomic locations selected from the 100 bp genomic locations defined in Tables 3 and 4.

In such embodiments, preferably the signature comprises a set of genomic locations defining 12 or more genomic regions, for example a set of genomic locations defining 15 or more genomic regions, a set of genomic locations defining 20 or more genomic regions, a set of genomic locations defining 25 or more genomic regions, a set of genomic locations defining 30 or more genomic regions, a set of genomic locations defining 50 or more genomic regions, a set of genomic locations defining 75 or more genomic regions, a set of genomic locations defining 100 or more genomic regions, a set of genomic locations defining 125 or more genomic regions, a set of genomic locations defining 150 or more genomic regions, a set of genomic locations defining 200 or more genomic regions, a set of genomic locations defining 300 or more genomic regions, a set of genomic locations defining 400 or more genomic regions.

In one embodiment, the set of genomic locations defining 10 or more genomic regions are genomic locations selected from the group consisting of:

a 100 to 200 bp (for example a 100 to 150 bp or 100 to 120 bp) genomic location comprising or having a genomic location defined in Table 5, and
a 2 to 99 bp (for example a 20 to 99 bp or 50 to 99 bp) genomic location within a genomic location defined in Table 5 and comprising at least one CpG locus. For example, the set of genomic locations defining 10 or more genomic regions are genomic locations selected from the 100 bp genomic locations defined in Table 5.

In such embodiments, preferably the signature comprises a set of genomic locations defining 12 or more genomic regions, for example a set of genomic locations defining 15 or more genomic regions, a set of genomic locations defining 20 or more genomic regions, a set of genomic locations defining 25 or more genomic regions, a set of genomic locations defining 30 or more genomic regions, a set of genomic locations defining 50 or more genomic regions, a set of genomic locations defining 75 or more genomic regions, a set of genomic locations defining 100 or more genomic regions, a set of genomic locations defining 125 or more genomic regions, a set of genomic locations defining 150 or more genomic regions.

In one embodiment, the set of genomic locations defining 10 or more genomic regions are genomic locations selected from the group consisting of:

a 100 to 200 bp (for example a 100 to 150 bp or 100 to 120 bp) genomic location comprising or having a genomic location defined in Table 6, and
a 2 to 99 bp (for example a 20 to 99 bp or 50 to 99 bp) genomic location within a genomic location defined in Table 6 and comprising at least one CpG locus. For example, the set of genomic locations defining 10 or more genomic regions are genomic locations selected from the 100 bp genomic locations defined in Table 6.

In such embodiments, preferably the signature comprises a set of genomic locations defining 12 or more genomic regions, for example a set of genomic locations defining 15 or more genomic regions, a set of genomic locations defining 20 or more genomic regions, a set of genomic locations defining 25 or more genomic regions, a set of genomic locations defining 30 or more genomic regions, a set of genomic locations defining 50 or more genomic regions, a set of genomic locations defining 75 or more genomic regions, a set of genomic locations defining 100 or more genomic regions, a set of genomic locations defining 125 or more genomic regions, a set of genomic locations defining 150 or more genomic regions.

In one embodiment, the set of genomic locations defining 10 or more genomic regions are genomic locations selected from the group consisting of:

a 100 to 200 bp (for example a 100 to 150 bp or 100 to 120 bp) genomic location comprising or having a genomic location defined in Table 7, and
a 2 to 99 bp (for example a 20 to 99 bp or 50 to 99 bp) genomic location within a genomic location defined in Table 7 and comprising at least one CpG locus. For example, the set of genomic locations defining 10 or more genomic regions are genomic locations selected from the 100 bp genomic locations defined in Table 7.

In such embodiments, preferably the signature comprises a set of genomic locations defining 12 or more genomic regions, for example a set of genomic locations defining 15 or more genomic regions, a set of genomic locations defining 20 or more genomic regions, a set of genomic locations defining 25 or more genomic regions, a set of genomic locations defining 30 or more genomic regions, a set of genomic locations defining 50 or more genomic regions, a set of genomic locations defining 75 or more genomic regions, a set of genomic locations defining 100 genomic regions.

The invention also provides a cfDNA methylome signature comprising a set of genomic locations defining 10 or more genomic regions.
a 100 to 200 bp (for example a 100 to 150 bp or 100 to 120 bp) genomic location comprising or having a genomic location defined in Table 8, and
a 2 to 99 bp (for example a 20 to 99 bp or 50 to 99 bp) genomic location within a genomic location defined in Table 8 and comprising at least one CpG locus.

In such embodiments, preferably the signature comprises a set of genomic locations defining 12 or more genomic regions, for example a set of genomic locations defining 15 or more genomic regions, a set of genomic locations defining 20 or more genomic regions, a set of genomic locations defining 25 or more genomic regions, a set of genomic locations defining 30 or more genomic regions, a set of genomic locations defining 50 or more genomic regions, a set of genomic locations defining 75 or more genomic regions, a set of genomic locations defining 100 or more genomic regions, a set of genomic locations defining 125 or more genomic regions, a set of genomic locations defining 150 or more genomic regions, a set of genomic locations defining 200 or more genomic regions, a set of genomic locations defining 300 or more genomic regions, a set of genomic locations defining 400 or more genomic regions, a set of genomic locations defining 500 or more genomic regions, a set of genomic locations defining 600 or more genomic regions, a set of genomic locations defining 700 or more genomic regions, a set of genomic locations defining 800 or more genomic regions, a set of genomic locations defining 900 or more genomic regions, or a set of genomic locations defining 1000 genomic regions.

The signature is for detecting, screening, monitoring, staging, classification, selecting treatment for, ascertaining whether treatment is working in, prognostication and/or treatment of prostate cancer.

The methylation state (for example the average methylation ratio) of the genomic regions defined by the set of genomic locations of the signature may be used to indicate one or more of the following: the presence of prostate cancer cfDNA in the cfDNA sample, a subtype of prostate cancer (for example a genomic subtype or molecular subtype, such as one that has an aggressive clinical course and/or a AR copy number gain), if the prostate cancer is metastatic, the aggression of the prostate cancer, the prognosis of the prostate cancer, the tumour response to a treatment, the relapse of the prostate cancer, and/or the residual disease following curative treatment. Preferably, the methylation state of the genomic regions defined by the set of genomic locations of the signature may be used to indicate the presence of prostate cancer cfDNA in the cfDNA sample and/or a subtype of prostate cancer, such as one that has an aggressive clinical course and/or a AR copy number gain.

In one embodiment, the set of genomic locations defining 10 or more genomic regions are genomic locations selected from the group consisting of:

a 100 to 200 bp (for example a 100 to 150 bp or 100 to 120 bp) genomic location comprising or having a genomic location defined in Table 9, and
a 2 to 99 bp (for example a 20 to 99 bp or 50 to 99 bp) genomic location within a genomic location defined in Table 9 and comprising at least one CpG locus. For example, the set of genomic locations defining 10 or more genomic regions are genomic locations selected from the 100 bp genomic locations defined in Table 9.

TABLE 9
A preferred subset of hypomethylated region genomic locations
of Table 8 (The genomic locations are locations with reference
to hg19; all regions including, having, or within the genomic
locations of table 9 are hypomethylated regions)

| Chromosome | start | end |
|---|---|---|
| chr12 | 52240301 | 52240400 |
| chr8 | 143535751 | 143535850 |
| chr17 | 81036151 | 81036250 |
| chr8 | 143535801 | 143535900 |
| chr5 | 142005201 | 142005300 |
| chr17 | 81036101 | 81036200 |
| chr12 | 52240351 | 52240450 |
| chr19 | 47736001 | 47736100 |
| chr10 | 3480051 | 3480150 |
| chr14 | 101123351 | 101123450 |
| chr8 | 144303301 | 144303400 |
| chr7 | 95155001 | 95155100 |
| chr8 | 143535501 | 143535600 |
| chr15 | 41219401 | 41219500 |
| chr15 | 41219451 | 41219550 |
| chr7 | 1251201 | 1251300 |
| chr8 | 143535851 | 143535950 |
| chr2 | 189191651 | 189191750 |
| chr8 | 144303251 | 144303350 |
| chr8 | 143535601 | 143535700 |
| chr3 | 23782851 | 23782950 |
| chr1 | 1936451 | 1936550 |
| chr7 | 158800951 | 158801050 |
| chr12 | 322251 | 322350 |
| chr1 | 15655951 | 15656050 |
| chr8 | 143535701 | 143535800 |
| chr20 | 36037701 | 36037800 |
| chr20 | 36037751 | 36037850 |
| chr17 | 7083051 | 7083150 |
| chr7 | 5319551 | 5319650 |
| chr17 | 7083001 | 7083100 |
| chr10 | 131650451 | 131650550 |
| chr1 | 1936501 | 1936600 |
| chr19 | 35818801 | 35818900 |
| chr10 | 3479951 | 3480050 |
| chr4 | 1160801 | 1160900 |
| chr19 | 47735751 | 47735850 |
| chr10 | 3494301 | 3494400 |
| chr17 | 78982051 | 78982150 |
| chr10 | 4331801 | 4331900 |
| chr1 | 1920801 | 1920900 |
| chr9 | 132482351 | 132482450 |
| chr8 | 1923051 | 1923150 |
| chr16 | 1159851 | 1159950 |
| chr2 | 189191701 | 189191800 |
| chr1 | 200707101 | 200707200 |
| chr20 | 48124151 | 48124250 |
| chr19 | 35818851 | 35818950 |
| chr10 | 131650701 | 131650800 |
| chr10 | 3379051 | 3379150 |
| chr10 | 3449001 | 3449100 |
| chr12 | 107297051 | 107297150 |
| chr19 | 35981501 | 35981600 |
| chr13 | 106063151 | 106063250 |
| chr5 | 2207051 | 2207150 |
| chr8 | 54164751 | 54164850 |
| chr3 | 129326701 | 129326800 |
| chr1 | 223435701 | 223435800 |
| chr2 | 11294551 | 11294650 |
| chr17 | 25798951 | 25799050 |
| chr22 | 37215901 | 37216000 |
| chr11 | 45392501 | 45392600 |
| chr11 | 45392551 | 45392650 |
| chr17 | 35277351 | 35277450 |
| chr9 | 89410901 | 89411000 |
| chr9 | 89410951 | 89411050 |
| chr8 | 103572851 | 103572950 |
| chr6 | 168629801 | 168629900 |
| chr3 | 129326651 | 129326750 |
| chr1 | 204655151 | 204655250 |
| chr1 | 204655201 | 204655300 |
| chr1 | 88108801 | 88108900 |
| chr10 | 4386801 | 4386900 |
| chr2 | 11294501 | 11294600 |
| chr16 | 49530551 | 49530650 |
| chr16 | 49530601 | 49530700 |
| chr7 | 95155051 | 95155150 |
| chr10 | 73324401 | 73324500 |
| chr5 | 150538351 | 150538450 |
| chr7 | 1388201 | 1388300 |
| chr3 | 186170701 | 186170800 |
| chr8 | 1923101 | 1923200 |
| chr8 | 54164651 | 54164750 |
| chr16 | 1316401 | 1316500 |
| chr10 | 4386851 | 4386950 |
| chr4 | 1535701 | 1535800 |
| chr8 | 144213001 | 144213100 |
| chr10 | 131650651 | 131650750 |
| chr10 | 3480001 | 3480100 |
| chr3 | 64305701 | 64305800 |
| chr3 | 64305751 | 64305850 |
| chr1 | 1936551 | 1936650 |
| chr10 | 3480101 | 3480200 |
| chr10 | 3277051 | 3277150 |
| chr4 | 24796601 | 24796700 |
| chr3 | 46622551 | 46622650 |
| chr14 | 104688501 | 104688600 |
| chr1 | 55504701 | 55504800 |
| chr22 | 37215951 | 37216050 |
| chr1 | 172291651 | 172291750 |
| chr1 | 2527501 | 2527600 |
| chr15 | 27210251 | 27210350 |
| chr8 | 54164601 | 54164700 |
| chr7 | 3019151 | 3019250 |
| chr11 | 71010451 | 71010550 |
| chr19 | 35981451 | 35981550 |
| chr16 | 876151 | 876250 |
| chr8 | 1923001 | 1923100 |
| chr7 | 1251251 | 1251350 |
| chr1 | 38606051 | 38606150 |
| chr10 | 131650501 | 131650600 |
| chr4 | 140201651 | 140201750 |
| chr14 | 105052601 | 105052700 |
| chr10 | 3378851 | 3378950 |
| chr14 | 106095451 | 106095550 |
| chr12 | 6933201 | 6933300 |
| chr8 | 54164801 | 54164900 |
| chr13 | 106063101 | 106063200 |
| chr10 | 94448551 | 94448650 |
| chr8 | 54164701 | 54164800 |
| chr17 | 79459401 | 79459500 |
| chr7 | 158818151 | 158818250 |
| chr6 | 25727351 | 25727450 |
| chr5 | 1010951 | 1011050 |
| chr1 | 2424651 | 2424750 |
| chr3 | 128724951 | 128725050 |
| chr12 | 322951 | 323050 |
| chr10 | 3591201 | 3591300 |
| chr10 | 3591251 | 3591350 |
| chr1 | 2424701 | 2424800 |
| chr7 | 1687001 | 1687100 |
| chr17 | 27396901 | 27397000 |
| chr4 | 7252451 | 7252550 |
| chr10 | 134610401 | 134610500 |
| chr7 | 1388151 | 1388250 |
| chr5 | 2207001 | 2207100 |
| chr6 | 37503051 | 37503150 |
| chr10 | 131752851 | 131752950 |
| chr8 | 143546801 | 143546900 |
| chr15 | 102094651 | 102094750 |
| chr14 | 101128351 | 101128450 |
| chr3 | 64338501 | 64338600 |
| chr3 | 64338551 | 64338650 |
| chr2 | 209271151 | 209271250 |
| chr1 | 15655901 | 15656000 |
| chr16 | 29267801 | 29267900 |
| chr12 | 107297101 | 107297200 |
| chr22 | 43621801 | 43621900 |
| chr10 | 5406551 | 5406650 |
| chr17 | 79109751 | 79109850 |

In such embodiments, preferably the signature comprises a set of genomic locations defining 12 or more genomic regions, for example a set of genomic locations defining 15 or more genomic regions, a set of genomic locations defining 20 or more genomic regions, a set of genomic locations defining 25 or more genomic regions, a set of genomic locations defining 30 or more genomic regions, a set of genomic locations defining 50 or more genomic regions, a set of genomic locations defining 75 or more genomic regions, a set of genomic locations defining 100 or more genomic regions, a set of genomic locations defining 125 or more genomic regions, a set of genomic locations defining 150 genomic regions.

Methods for Determining a Solid Cancer cfDNA Methylome Signature

The present invention also provides methods for determining a solid cancer cfDNA methylome signature. Suitably, such signatures are used, for example, in detecting, screening, monitoring, staging, classification, selecting treatment for, ascertaining whether treatment is working in, prognostication and/or treatment of a solid cancer. They can also suitably be used with the methods and kits for detecting, screening, monitoring, staging, classification, selecting treatment for, ascertaining whether treatment is working in, prognostication and/or treatment of a solid cancer and in methods for treatment of solid cancer.

In one embodiment, the invention provides a method for determining a solid cancer cfDNA methylome signature for use in detecting, screening, monitoring, staging, classification, selecting treatment for, ascertaining whether treatment is working in, prognostication and/or treatment of the solid cancer, the method comprising:
(i) characterizing the methylome sequence of a plurality of cfDNA molecules in a first sample comprising cfDNA from a subject known to have the solid cancer, wherein the methylome sequence of a cfDNA molecule is the DNA sequence and the methylation profile of the molecule;
(ii) determining the respective number of characterised cfDNA molecules corresponding to a CpG locus or a genomic region of 2 to 10,000 bp (preferably 2 to 200 bp) in the first sample by aligning the methylome sequences;
(iii) determining the methylation ratio of each CpG locus and/or average methylation ratio of each genomic region of 2 to 10,000 bp (preferably 2 to 200 bp) in the first sample;
repeating steps (i) to (iii) for one or more further samples comprising cfDNA each from subjects known to have the solid cancer;
performing a variance analysis of all or a selection of the methylation ratios of the CpG loci and/or all or a selection of average methylation ratios of the genomic regions of the samples;
selecting a group of CpG loci and/or genomic regions associated with a feature of the samples; and
selecting CpG loci and/or genomic regions in the group to provide the cfDNA methylome signature.

In certain embodiments the solid cancer is prostate cancer (for example acinar adenocarcinoma prostate cancer, ductal adenocarcinoma prostate cancer, transitional cell cancer of the prostate, squamous cell cancer of the prostate, or small cell prostate cancer, and particularly acinar adenocarcinoma prostate cancer or ductal adenocarcinoma prostate cancer). In certain embodiments, the solid cancer is a metastatic cancer. In certain embodiments, the solid cancer is a relapsed and/or refractory solid cancer. In certain embodiments, the solid cancer is a subtype of a solid cancer, for example a subtype of a prostate cancer, for example a prostate cancer with specific molecular characteristics and/or genetic characteristics of the cancer cells.

The first sample is a sample that comprises cfDNA. The sample may suitably be a blood sample, a plasma sample, or a urine sample. In certain embodiments, the sample is a blood sample or a plasma sample. In certain embodiments, the sample is a urine sample.

Each further sample is a sample that comprises cfDNA. Each further sample may suitably be a blood sample, a plasma sample, or a urine sample. In certain embodiments, one or more further sample(s) is/are blood sample(s) or plasma sample(s). In certain embodiments, one or more further sample(s) is/are urine sample(s). In certain embodiments, all of the further samples are of the same type, for example each further sample is a blood sample; or each further sample is a plasma sample; or each further sample is a urine sample. In certain embodiments, each further sample is a blood sample; or each further sample is a plasma sample.

In one preferred embodiment, the first sample and each further sample are all samples of the same type. For example, the first sample and each further sample are all blood samples; or the first sample and each further sample are all plasma samples; or the first sample and each further sample are all urine samples. In certain embodiments, the first sample and each further sample are all blood samples; or the first sample and each further sample are all plasma samples.

In one embodiment, the first sample comprising cfDNA is from a subject known to have or suspected of having metastatic solid cancer. For example, the sample comprising cfDNA is from a subject known to have metastatic solid cancer.

In one embodiment, the one or more further samples comprising cfDNA are each from subjects known to have or suspected of having metastatic solid cancer. For example, the one or more further samples comprising cfDNA are each from subjects known to have metastatic solid cancer.

In one embodiment, the first and each further sample comprising cfDNA are each from subjects known to have or suspected of having metastatic solid cancer. For example, the first and each further sample comprising cfDNA are each from subjects known to have metastatic solid cancer.

In one embodiment, the first sample and one or more of the further samples are from the same subject, for example the same subject but at different time points, for example before treatment, during a treatment, after a treatment, before progression, after progression, after relapse, and/or after change of the disease to metastatic cancer.

In one embodiment, the first sample and each of the further samples are from the same subject, for example the same subject but at different time points, for example before treatment, during a treatment, after a treatment, before progression, after progression, after relapse, and/or after change of the disease to metastatic cancer.

In one embodiment, the first sample and one or more of the further samples are from different subjects. The different subjects may all have the same type of the solid cancer, or may all have a different type of the solid cancer, or some may have the same and some may have a different type of the solid cancer. A type of solid cancer may be metastatic, and a different type may be non-metastatic cancer. Another type of solid cancer may be a solid cancer that responds to a certain treatment (e.g. a hormonal agent), and a solid cancer that does not respond to that treatment (e.g. a hormonal agent). For prostate cancer, different types of that solid cancer include acinar adenocarcinoma prostate cancer, ductal adenocarcinoma prostate cancer, transitional cell cancer of the prostate, squamous cell cancer of the prostate, and small cell prostate cancer. For prostate cancer, different types of that solid cancer also include castration sensitive prostate cancer and castration resistant prostate cancer.

In one embodiment, the first sample and one or more of the further samples are from different subjects. The different subjects may all have the same subtype of the solid cancer, or may all have a different subtype of the solid cancer, or some may have the same and some may have a different subtype of the solid cancer. A subtype of solid cancer may be subtype based on characteristics of the cancer cells, and in particular molecular and genetic characteristics of the cells. An example of prostate cancer subtypes include androgen sensitive prostate cancer, androgen insensitive prostate cancer, AR copy number gain, and prostate cancer with an aggressive clinical course.

In one embodiment, the first sample and one or more of the further samples have different levels of cancer fraction of cfDNA. In one embodiment, the first sample and one or more of the further samples have similar levels of cancer fraction of cfDNA. The level of cancer fraction in a cfDNA sample can be determined by, for example, using methods that estimate tumour fraction using genomic markers.

Each subject is preferably the same species, for example each subject (i.e. the first subject and each of the one or more further subjects) are human.

In certain embodiments, the method comprises the additional step of obtaining a biological sample from the first subject and/or obtaining a biological sample from one or more further subjects, for example from each of the one or more further subjects.

The method for determining a solid cancer cfDNA methylome signature may further comprise isolating the cfDNA from the first sample, and isolating the cfDNA from the one or more further samples. Methods for isolating the cfDNA from the sample described elsewhere herein may be used in the method for determining a solid cancer cfDNA methylome signature.

The method comprises characterizing the methylome sequence of a plurality of cfDNA molecules in a first sample, wherein the methylome sequence of a cfDNA molecule is the DNA sequence and the methylation profile of the molecule. The method also comprises characterizing the methylome sequence of a plurality of cfDNA molecules in each of one or more further samples, wherein the methylome sequence of a cfDNA molecule is the DNA sequence and the methylation profile of the molecule. Methods for characterizing the methylome sequence of a plurality of cfDNA molecules described elsewhere herein may be used in the method for determining a solid cancer cfDNA methylome signature.

A plurality of cfDNA molecules may be, for example, at least 100, at least 1000, at least 10,000, at least 50,000, at least 100,000, at least 500,000, at least 1,000,000 (10⁶), at least 5,000,000 (5×10⁶), at least 10,000,000 (10⁷), at least 100,000,000 (10⁸), or at least 1,000,000,000 (10⁹). Preferably, a plurality of cfDNA molecules may be, for example, at least 10,000, at least 50,000, at least 100,000, at least 500,000, at least 1,000,000 (10⁶), at least 5,000,000 (5×10⁶), at least 10,000,000 (10⁷), at least 100,000,000 (10⁸), or at least 1,000,000,000 (10⁹). More preferably, a plurality of cfDNA molecules may be, for example, at least 100,000, at least 500,000, at least 1,000,000 (10⁶), at least 5,000,000 (5×10⁶), at least 10,000,000 (10⁷), at least 100,000,000 (10⁸), or at least 1,000,000,000 (10⁹). The plurality of cfDNA molecules that are characterised for the first sample and for each of the one or more further samples may be the same or may be different.

The method comprises determining the respective number of characterised cfDNA molecules corresponding to a CpG locus or a genomic region of 2 to 10,000 bp (preferably 2 to 200 bp) by aligning the methylome sequences in the first sample. The method also comprises determining the respective number of characterised cfDNA molecules corresponding to a CpG locus or a genomic region of 2 to 10,000 bp (preferably 2 to 200 bp) by aligning the methylome sequences in each of of the one or more further samples. Aligning the methylome sequences can, for example, be carried out using a variety of techniques known in the art. For example, a DNA sequence alignment tool, (e.g., BSMAP (PMID: 19635165), Bismark (PMID: 21493656), gemBS (PMID: 30137223), Arioc (PMID: 29554207), BS-Seeker2 (PMID: 24206606), MethylCoder (PMID: 21724594) or BatMeth2 (PMID: 30669962)) can be used to align the reads. The reads may be aligned to reference genome (for example hg38, hg19, hg18, hg17 or hg16).

In certain embodiments, the method comprises removing duplications of reads of the same DNA molecule (i.e. duplications of reads of the same cfDNA molecule). In this step, sequence reads having exactly the same sequence and start and end base pairs (i.e. the same unclipped alignment start and unclipped alignment end of the sequence) are removed, as they are likely to be duplicate sequence reads of the same sequence (i.e. duplicate of reads of the same cfDNA molecule). For example, PCR duplications can be removed as part of the aligning step, such as using Picard tools v2.1.0 (http://broadinstitute.github.io/picard).

Preferably, determining the respective number of characterised cfDNA molecules corresponding to a CpG locus or a genomic region of 2 to 10,000 bp (preferably 2 to 200 bp) in the first sample comprises aligning the methylome sequences with a reference genome for the subject, for example for a human subject by aligning the methylome sequences with hg38, hg19, hg18, hg17 or hg16.

Preferably, determining the respective number of characterised cfDNA molecules corresponding to a CpG locus or a genomic region of 2 to 10,000 bp (preferably 2 to 200 bp) in the one or more further samples comprises aligning the methylome sequences for each of the one or more further samples with a reference genome for the subject, for example for a human subject by aligning the methylome sequences with hg38, hg19, hg18, hg17 or hg16.

Preferably, the methylome sequences in the first sample and the methylome sequences in each of the one or more further samples are aligned to the same reference genome, for example the methylome sequences in the first sample and the methylome sequences in each of the one or more further samples are aligned to hg38; or the methylome sequences in the first sample and the methylome sequences in each of the one or more further samples are aligned to hg19; or the methylome sequences in the first sample and the methylome sequences in each of the one or more further samples are aligned to hg18; or the methylome sequences in the first sample and the methylome sequences in each of the one or more further samples are aligned to hg17; or the methylome sequences in the first sample and the methylome sequences in each of the one or more further samples are aligned to hg16.

In certain preferred embodiments, the cfDNA molecules in the first sample and the one or more further samples may correspond to a CpG locus or a genomic region of 2 to 5000 bp. More preferably, cfDNA molecules correspond to a CpG locus or a genomic region of 2 to 5000 bp, 2 to 4000 bp, 2 to 3000 bp, 2 to 2000 bp, 2 to 1000 bp, 2 to 800 bp, 2 to 600 bp, 2 to 500 bp, 2 to 400 bp, 2 to 300 bp, or 2 to 200 bp. In one very preferred embodiment, the cfDNA molecules correspond to a CpG locus or a genomic region of 2 to 200 bp for example 10, 20, 30, 40, 50, 60, 70, 80, 90, 100, 110, 120, 130, 140, 150, 160, 170, 180, 190 or 200 bp. In another preferred embodiment, the cfDNA molecules correspond to a CpG locus or a genomic region of 10 to 150 bp, 20 to 150 bp, 50 to 150 bp, 50 to 120 bp, 80 to 120 bp, 90 to 110 bp. In one preferred embodiment, the cfDNA molecules correspond to a genomic region of 100 bp.

The method comprises determining the methylation ratio of each CpG locus or the average methylation ratio of each genomic region of 2 to 10,000 bp (preferably 2 to 200 bp) in the first sample, and determining the methylation ratio of each CpG locus or the average methylation ratio of each genomic region of (preferably 2 to 200 bp) in each of the one or more further samples.

The average methylation ratio is the average of the methylation ratios of all the CpG loci within a given genomic region, and can be calculated by determining the sum of the methylation ratios of all CpG within a given genomic region and dividing the sum by the number of CpG within the given genomic region. If a genomic region has only 1 CpG locus, the average methylation is the same as the methylation ratio for the single CpG locus in the genomic region.

The method comprises repeating steps (i) to (iii) for one or more further samples comprising cfDNA each from subjects known to have the solid cancer. As such, the method comprises:

characterizing the methylome sequence of a plurality of cfDNA molecules in each of one or more further samples comprising cfDNA each from a subject known to have the solid cancer, wherein the methylome sequence of a cfDNA molecule is the DNA sequence and the methylation profile of the molecule;
determining the respective number of characterised cfDNA molecules corresponding to a CpG locus or a genomic region of 2 to 10,000 bp (preferably 2 to 200 bp) in each of one or more further samples by aligning the methylome sequences;
determining the methylation ratio of each CpG locus or the average methylation ratio of each genomic region of 2 to 10,000 bp (preferably 2 to 200 bp) in each of one or more further samples.

Thus, for the first sample and for each of the one or more further samples, the methylation ratio of each CpG locus or the average methylation ratio of each genomic region of 2 to 10,000 bp (preferably 2 to 200 bp) in the characterised cfDNA molecules are determined.

In certain embodiments, there is one further sample. In certain embodiments there is more than one further sample.

Preferably there are 2 or more further samples, 3 or more further samples, 4 or more further samples, 5 or more further samples, 6 or more further samples, 7 or more further samples, 8 or more further samples, 9 or more further samples, 10 or more further samples, 12 or more further samples, 15 or more further samples, 20 or more further samples, 25 or more further samples, 30 or more further samples, 40 or more further samples, 50 or more further samples, 60 or more further samples, 70 or more further samples, 80 or more further samples, 90 or more further samples, 100 or more further samples, 200 or more further samples, 300 or more further samples, 400 or more further samples, 500 or more further samples or 1000 or more further samples.

In one preferred embodiment there are 5 or more further samples, 10 or more further samples, 15 or more further samples, 20 or more further samples, 25 or more further samples, 50 or more further samples, 100 or more further samples, 200 or more further samples, 300 or more further samples, 400 or more further samples, 500 or more further samples or 1000 or more further samples.

In one preferred embodiment there are 10 or more further samples, 15 or more further samples, 20 or more further samples, 25 or more further samples, 50 or more further samples, 100 or more further samples, 200 or more further samples, 300 or more further samples, 400 or more further samples, 500 or more further samples or 1000 or more further samples.

The method comprises performing a variance analysis of all or a selection of the methylation ratios of the CpG loci and/or all or a selection of average methylation ratios of the genomic regions of the samples. A variance analysis results in groupings of CpG locus and/or genomic regions associated with features of the samples.

A cfDNA sample from a subject having a solid cancer is a heterogenous mixture of cfDNA from a primary source (for example, for a blood or plasma sample the primary source of cfDNA molecules are cfDNA from white blood cells, or in a urine sample the primary source of cfDNA molecules is a mixture of cfDNA from white blood cells, immune cell and urinary tract lining cells) and cfDNA from cancer cells. cfDNA in different samples (i.e. samples from different subjects and/or from the same subject at different time points) have differences in methylation levels. The inventors have surprisingly found that very useful methylome signatures can be found by performing a variance analysis of methylation ratios of CpG loci and/or average methylation ratios of genomic regions in multiple cfDNA samples from cancer patients. As not all DNA ends up as cfDNA, in view of the method of the invention determining variance in cfDNA samples, the signatures found using the method include CpG loci and/or genomic regions that are found in cfDNA samples. Additionally, the signatures found using this method can include both cancer-specific and tissue specific methylation. Thus, signatures found using the method of the invention will be especially useful and accurate when used in methods for detecting, screening, monitoring, staging, classification, selecting treatment for, ascertaining whether treatment is working in, prognostication and/or treatment of a solid cancer in a cfDNA sample, and especially in a sample of the same type as was used to find the signature.

A selection of the methylation ratios and/or a selection of average methylation ratios may be, for example at least 95%, at least 90%, at least 80%, at least 70%, at least 60%, at least 50%, at least 40%, at least 30%, at least 20%, at least 10%, or at least 5% methylation ratios and/or average methylation ratios. A selection of the methylation ratios and/or a selection of average methylation ratios of the genomic regions of the samples may be, for example less than 95%, less than 90%, less than 80%, less than 70%, less than 60%, less than 50%, less than 40%, less than 30%, less than 20%, less than 10%, or less than 5% methylation ratios and/or average methylation ratios.

A selection of the methylation ratios and/or a selection of average methylation ratios of the genomic regions of the samples may be a selection of the methylation ratios of the CpG loci and/or a selection of average methylation ratios of the genomic regions for one or more chromosomes. For example, selection of the methylation ratios and/or a selection of average methylation ratios of the genomic regions for one or more of chromosome 1, 2, 3, 4, 5, 6, 7, 8, 9, 10, 11, 12, 13, 14, 15, 16, 17, 18, 19, 20, 21, 22, 23, X and/or Y.

A selection of the methylation ratios and/or a selection of average methylation ratios of the genomic regions of the samples may be a selection of the methylation ratios of the CpG loci and/or a selection of average methylation ratios of the genomic regions wherein all samples have at least 1 characterised cfDNA molecule covering of each of the CpG loci and/or genomic regions. For example, wherein each sample has at least 10 (for example at least 15, 20, 25, 50, 100 or 1000) characterised cfDNA molecules covering each of the CpG loci and/or genomic regions.

In one preferred embodiment, the variance analysis performed is dimensionality reduction. For example, the variance analysis performed is a principal component analysis, a logistic regression analysis, a nearest neighbour analysis, a support vector machine, a neural network model, a NMF (non-negative matrix factorisation), an ICA (independent component analysis), FA (factor analysis), surrogate variable analysis (SVA), and independent surrogate variable analysis (ISVA).

In one preferred embodiment, the variance analysis performed is a principal component analysis.

In embodiments wherein the variance analysis performed is a principal component analysis, the CpG locus and/or genomic regions associated with features of the samples are the groupings of the different principal components, such as principal component 1, principal component 2, principal component 3, principal component 4, principal component 5, principal component 6, principal component 7, principal component 8 or a higher principal component.

The variance analysis performed will group CpG loci and/or genomic regions associated with different feature of the samples.

The variance analysis (for example the dimensionality reduction) is optionally followed by feature selection methods. An optional feature selection method can be implemented using R, python languages or equivalent statistical application or software.

The method comprises selecting a group of CpG loci and/or genomic regions associated with a feature of the samples, i.e. selecting a group from all of the groups that the variance analysis results in. For example, in embodiments wherein the variance analysis performed is a principal component analysis, the selecting a group of CpG loci and/or genomic regions associated with a feature of the samples comprises selecting one of principal component 1, principal component 2, principal component 3, principal component 4, principal component 5, principal component 6, principal component 7, principal component 8 or a higher principal component.

A feature of the samples may be any feature of the samples, which are each from subjects known to have the solid cancer and which all comprise cfDNA. Examples of a feature of the samples that a group of CpG loci and/or genomic regions may be associated with include, but are not limited to, level of solid cancer fraction in the cfDNA, a type of solid cancer, a subtype of solid cancer, a prognosis, aggression of the solid cancer, and susceptibility of the solid cancer to a treatment.

In certain embodiments, the group selected is a group of CpG loci and/or genomic regions associated with a level of solid cancer fraction in the cfDNA.

In certain embodiments, the group selected is a group of CpG loci and/or genomic regions associated with a type of solid cancer, for example associated with metastatic cancer; associated with non-metastatic cancer; associated with a type of solid cancer that responds to a certain treatment (e.g. a hormonal agent); or associated with a solid cancer that does not respond to a certain treatment (e.g. a hormonal agent). For a solid cancer that is a prostate cancer, in certain embodiments the group selected is a group of CpG loci and/or genomic regions associated with a type of solid cancer, for example associated with castration resistant prostate cancer; associated with castration sensitive prostate cancer; associated with acinar adenocarcinoma prostate cancer; associated with ductal adenocarcinoma prostate cancer; associated with transitional cell cancer of the prostate; associated with squamous cell cancer of the prostate; or associated with small cell prostate cancer.

In certain embodiments, the group selected is a group of CpG loci and/or genomic regions associated with a subtype of solid cancer, for example associated with molecular characteristics of the cancer cells; and/or associated with genetic characteristics of the cancer cells. For a solid cancer that is a prostate cancer, in certain embodiments the group selected is a group of CpG loci and/or genomic regions associated with a subtype of the solid cancer, for example associated with AR copy number gain; and/or associated with an aggressive clinical course.

In certain embodiments, the group selected is a group of CpG loci and/or genomic regions associated with a prognosis, for example associated with a good prognosis (for example survival of the subject upon treatment is from at least 1 month to at least 90 years); or associated with a poor prognosis (for example survival of a subject that is expected to be from less than 5 years to less than 1 month).

In certain embodiments, the group selected is a group of CpG loci and/or genomic regions associated aggression of the solid cancer.

In certain embodiments, the group selected is a group of CpG loci and/or genomic regions associated with susceptibility of the solid cancer to a treatment. For example associated with susceptibility of the solid cancer to a treatment with one or more of the following: a hormonal agent, a targeted agent, a biologic agent, an immunotherapy agent, a chemotherapy agent and a radionuclide treatment.

The method further comprises selecting CpG loci and/or genomic regions in the group to provide the cfDNA methylome signature. This may include selecting all of the CpG loci and/or genomic regions in the group or selecting a plurality of the CpG loci and/or genomic regions in the group, for example selecting at least 95%, at least 90%, at least 80%, at least 70%, at least 60%, at least 50%, at least 40%, at least 30%, at least 20%, at least 10%, or at least 5%, or for example selecting less than 95%, less than 90%, less than 80%, less than 70%, less than 60%, less than 50%, less than 40%, less than 30%, less than 20%, less than 10%, or less than 5%.

Selecting CpG loci and/or genomic regions in the group to provide the cfDNA methylome signature may comprise selecting at least 10,000 CpG loci and/or genomic regions, at least 8000 CpG loci and/or genomic regions, at least 5000 CpG loci and/or genomic regions, at least 4000 CpG loci and/or genomic regions, at least 3000 CpG loci and/or genomic regions, at least 2000 CpG loci and/or genomic regions, at least 1000 CpG loci and/or genomic regions, at least 800 CpG loci and/or genomic regions, at least 700 CpG loci and/or genomic regions, at least 600 CpG loci and/or genomic regions, at least 500 CpG loci and/or genomic regions, at least 400 CpG loci and/or genomic regions, at least 300 CpG loci and/or genomic regions, at least 250 CpG loci and/or genomic regions, at least 200 CpG loci and/or genomic regions, at least 150 CpG loci and/or genomic regions, at least 100 CpG loci and/or genomic regions, at least 50 CpG loci and/or genomic regions or at least 10 CpG loci and/or genomic regions.

Selecting CpG loci and/or genomic regions in the group to provide the cfDNA methylome signature may comprise selecting 10,000 or fewer CpG loci and/or genomic regions, 8000 or fewer CpG loci and/or genomic regions, 5000 or fewer CpG loci and/or genomic regions, 4000 or fewer CpG loci and/or genomic regions, 3000 or fewer CpG loci and/or genomic regions, 2000 or fewer CpG loci and/or genomic regions, 1000 or fewer CpG loci and/or genomic regions, 800 or fewer CpG loci and/or genomic regions, 700 or fewer CpG loci and/or genomic regions, 600 or fewer CpG loci and/or genomic regions, 500 or fewer CpG loci and/or genomic regions, 400 or fewer CpG loci and/or genomic regions, 300 or fewer CpG loci and/or genomic regions, 250 or fewer CpG loci and/or genomic regions, 200 or fewer CpG loci and/or genomic regions, 150 or fewer CpG loci and/or genomic regions, 100 or fewer CpG loci and/or genomic regions, 50 or fewer CpG loci and/or genomic regions or 10 or fewer CpG loci and/or genomic regions.

Selecting CpG loci and/or genomic regions in the group to provide the cfDNA methylome signature may comprise selecting 10,000 CpG loci and/or genomic regions, 8000 CpG loci and/or genomic regions, 5000 CpG loci and/or genomic regions, 4000 CpG loci and/or genomic regions, 3000 CpG loci and/or genomic regions, 2000 CpG loci and/or genomic regions, 1000 CpG loci and/or genomic regions, 800 CpG loci and/or genomic regions, 700 CpG loci and/or genomic regions, 600 CpG loci and/or genomic regions, 500 CpG loci and/or genomic regions, 400 CpG loci and/or genomic regions, 300 CpG loci and/or genomic regions, 250 CpG loci and/or genomic regions, 200 CpG loci and/or genomic regions, 150 CpG loci and/or genomic regions, 100 CpG loci and/or genomic regions, 50 CpG loci and/or genomic regions or 10 CpG loci and/or genomic regions.

Preferably, the method comprises selecting at least 5 CpG loci (for example at least 8, at least 10, at least 12, at least 15, at least 20, at least 25, at least 30, at least 40, at least 50, at least 75, at least 100, at least 200, at least 300, at least 400, at least 500, at least 600, at least 700, at least 800, at least 900, at least 1000 or at least 10,000) and/or at least 5 genomic regions (for example at least 8, at least 10, at least 12, at least 15, at least 20, at least 25, at least 30, at least 40, at least 50, at least 75, at least 100, at least 200, at least 300, at least 400, at least 500, at least 600, at least 700, at least 800, at least 900, at least 1000 or at least 10,000) in the group to provide a cfDNA methylome signature.

In one embodiment, the method comprises selecting at least 5 CpG loci in the group to provide a cfDNA methylome signature, for example at least 8, at least 10, at least 12, at least 15, at least 20, at least 25, at least 30, at least 40, at least 50, at least 75, at least 100, at least 200, at least 300, at least 400, at least 500, at least 600, at least 700, at least 800, at least 900, at least 1000 or at least 10,000 CpG loci. In one preferred embodiment, the method comprises selecting at least 10 CpG loci, at least 100 CpG loci, at least 250 CpG loci, or at least 500 CpG loci in the group to provide a cfDNA methylome signature. For example the method comprises selecting 10 CpG loci, 100 CpG loci, 250 CpG loci, 500 CpG loci in the group to provide a cfDNA methylome signature.

In another embodiment, the method comprises selecting at least 5 genomic regions in the group to provide a cfDNA methylome signature, for example at least 8, at least 10, at least 12, at least 15, at least 20, at least 25, at least 30, at least 40, at least 50, at least 75, at least 100, at least 200, at least 300, at least 400, at least 500, at least 600, at least 700, at least 800, at least 900, at least 1000 or at least 10,000 genomic regions. In one preferred embodiment, the method comprises selecting at least 10 genomic regions, at least 100 genomic regions, at least 250 genomic regions, or at least 500 genomic regions in the group to provide a cfDNA methylome signature. For example the method comprises selecting 10 genomic regions, 100 genomic regions, 250 genomic regions, 500 genomic regions in the group to provide a cfDNA methylome signature.

In one preferred embodiment, selecting the CpG loci and/or genomic regions in the group to provide the cfDNA methylome signature comprises selecting the CpG loci and/or genomic regions in the group that have strong (for example high) association with the feature to provide the cfDNA methylome signature. The CpG loci and/or genomic regions with strong (for example high) association with the feature may be CpG loci and/or genomic regions that are within the top 10,000 CpG loci and/or genomic regions most correlated with the feature in the group. For example, CpG loci and/or genomic regions with strong (for example high) association with the feature are CpG loci and/or genomic regions that are within the top 8000 CpG loci and/or genomic regions most correlated with the feature in the group; CpG loci and/or genomic regions with strong (for example high) association with the feature are CpG loci and/or genomic regions that are within the top 6000 CpG loci and/or genomic regions most correlated with the feature in the group; CpG loci and/or genomic regions with strong (for example high) association with the feature are CpG loci and/or genomic regions that are within the top 5000 CpG loci and/or genomic regions most correlated with the feature in the group; CpG loci and/or genomic regions with strong (for example high) association with the feature are CpG loci and/or genomic regions that are within the top 4000 CpG loci and/or genomic regions most correlated with the feature in the group; CpG loci and/or genomic regions with strong (for example high) association with the feature are CpG loci and/or genomic regions that are within the top 3000 CpG loci and/or genomic regions most correlated with the feature in the group; CpG loci and/or genomic regions with strong (for example high) association with the feature are CpG loci and/or genomic regions that are within the top 2000 CpG loci and/or genomic regions most correlated with the feature in the group; CpG loci and/or genomic regions with strong (for example high) association with the feature are CpG loci and/or genomic regions that are within the top 1000 CpG loci and/or genomic regions most correlated with the feature in the group; CpG loci and/or genomic regions with strong (for example high) association with the feature are CpG loci and/or genomic regions that are within the top 800 CpG loci and/or genomic regions most correlated with the feature in the group; or CpG loci and/or genomic regions with strong (for example high) association with the feature are CpG loci and/or genomic regions that are within the top 500, 400, 300, 250, 200, 150, 100, 50 or 10 CpG loci and/or genomic regions most correlated with the feature in the group.

In one preferred embodiment, CpG loci and/or genomic regions correlated with the feature in the group that have strong (for example high) association with the feature are CpG loci and/or genomic regions that are within the top 1000 CpG loci and/or genomic regions most correlated with the feature in the group. More preferably, CpG loci and/or genomic regions correlated with the feature in the group that have strong (for example high) association with the feature are CpG loci and/or genomic regions that are within the top 800 CpG loci and/or genomic regions most correlated with the feature in the group; or even more preferably CpG loci and/or genomic regions most correlated with the feature in the group that have strong (for example high) association with the feature may be CpG loci and/or genomic regions that are within the top 500, 400, 300, 250, 200, 150, 100, 50 or 10 CpG loci and/or genomic regions most correlated with the feature in the group.

In one embodiment wherein the level of methylation variance is determined using a principal component analysis, selecting the CpG loci and/or genomic regions in the group comprises selecting a plurality of CpG loci and/or genomic regions of principal component 1, 2, 3, 4, 5, 6, 7 or 8, for example selecting a plurality of CpG loci and/or genomic regions of principal component 1, 2, 3, 4, 5, 6, 7 or 8 that have strong (for example high) association with the feature of principal component 1, 2, 3, 4, 5, 6, 7 or 8, for example selecting CpG loci and/or genomic regions that are within the top 10,000 CpG loci and/or genomic regions of principal component 1, 2, 3, 4, 5, 6, 7 or 8 most correlated with the feature of principal component 1, 2, 3, 4, 5, 6, 7 or 8; or selecting CpG loci and/or genomic regions that are within the top 5000 CpG loci and/or genomic regions of principal component 1, 2, 3, 4, 5, 6, 7 or 8 most correlated with the feature of principal component 1, 2, 3, 4, 5, 6, 7 or 8; selecting CpG loci and/or genomic regions that are within the top 4000 CpG loci and/or genomic regions of principal component 1, 2, 3, 4, 5, 6, 7 or 8 most correlated with the feature of principal component 1, 2, 3, 4, 5, 6, 7 or 8; selecting CpG loci and/or genomic regions that are within the top 3000 CpG loci and/or genomic regions of principal component 1, 2, 3, 4, 5, 6, 7 or 8 most correlated with the feature of principal component 1, 2, 3, 4, 5, 6, 7 or 8; selecting CpG loci and/or genomic regions that are within the top 2000 CpG loci and/or genomic regions of principal component 1, 2, 3, 4, 5, 6, 7 or 8 most correlated with the feature of principal component 1, 2, 3, 4, 5, 6, 7 or 8; selecting CpG loci and/or genomic regions that are within the top 1000 CpG loci and/or genomic regions of principal component 1, 2, 3, 4, 5, 6, 7 or 8 most correlated with the feature of principal component 1, 2, 3, 4, 5, 6, 7 or 8; or selecting CpG loci and/or genomic regions that are within the top 500, 400, 300, 250, 200, 150, 100, 50 or 10 CpG loci and/or genomic regions of principal component 1, 2, 3, 4, 5, 6, 7 or 8 most correlated with the feature of principal component 1, 2, 3, 4, 5, 6, 7 or 8.

In one embodiment wherein the level of methylation variance is determined using a principal component analysis, selecting the CpG loci and/or genomic regions in the group that that have strong (for example high) association with the feature comprises selecting a plurality of CpG loci and/or genomic regions of principal component 1 correlated with the feature of principal component 1, for example selecting CpG loci and/or genomic regions that are within the top 10,000 CpG loci and/or genomic regions of principal component 1 most correlated with the feature of principal component 1; or selecting CpG loci and/or genomic regions that are within the top 5000 CpG loci and/or genomic regions of principal component 1 most correlated with the feature of principal component 1; selecting CpG loci and/or genomic regions that are within the top 4000 CpG loci and/or genomic regions of principal component 1 most correlated with the feature of principal component 1; selecting CpG loci and/or genomic regions that are within the top 3000 CpG loci and/or genomic regions of principal component 1 most correlated with the feature of principal component 1; selecting CpG loci and/or genomic regions that are within the top 2000 CpG loci and/or genomic regions of principal component 1 most correlated with the feature of principal component 1; selecting CpG loci and/or genomic regions that are within the top 1000 CpG loci and/or genomic regions of principal component 1 most correlated with the feature of principal component 1; or selecting CpG loci and/or genomic regions that are within the top 500, 400, 300, 250, 200, 150, 100, 50 or 10 CpG loci and/or genomic regions of principal component 1 most correlated with the feature of principal component 1.

The method for determining a solid cancer cfDNA methylome signature may further comprise comparing the methylation state of each of the selected CpG loci and/or genomic regions in the first sample and in the one or more further samples with the methylation state of the same CpG locus and/or genomic region in one or more of the following:
a sample of non-cancerous tissue of origin of the solid cancer;
a sample of the solid cancer;
a cell-line of the solid cancer;
a sample of cfDNA from a subject known to have the solid cancer (for example an age-matched subject known to have the solid cancer, and for example wherein the level of cancer fraction in the cfDNA sample from the different subject is known and/or wherein the sample is known to comprise cfDNA derived from a prostate cancer subtype);
a sample of white blood cells; and/or
a sample of cfDNA from a healthy subject (for example an age-matched healthy subject); and
optionally determining if the selected CpG locus and/or genomic region are associated with methylation patterns in the tissue of origin of the solid cancer and/or the solid cancer.

A sample of non-cancerous tissue of origin of the solid cancer, sample of the solid cancer, cell-line of the solid cancer; and/or sample of white blood cells may come from the same subject as the first sample and/or the one or more further samples comprising cfDNA; and/or a sample of non-cancerous tissue of origin of the solid cancer, sample of the solid cancer, cell-line of the solid cancer, and/or sample of white blood cells may come from a different subject as the first sample and/or the one or more further samples comprising cfDNA; and/or a sample of non-cancerous tissue of origin of the solid cancer, sample of the solid cancer, cell-line of the solid cancer, and/or sample of white blood cells may come from a different subject as the first sample and each of the one or more further samples comprising cfDNA.

In embodiments where the sample is a sample of the solid cancer, a sample of non-cancerous tissue of origin of the solid cancer and/or a sample of white blood cell, preferably the sample is from the same subject as the subject of the first sample or a subject of the one or more further samples. Additionally, or alternatively, samples of the solid cancer, samples of non-cancerous tissue of origin of the solid cancerm and/or samples of white blood cell from one or more different subjects to the subject of the first sample and the subjects of the one or more further samples are compared.

If a sample is from a different subject to the subject of the first sample and/or the subjects of the one or more further samples, preferably the different sample is from a subject that is age-matched subject with the subject of the first sample and/or the subjects of the one or more further samples.

In one preferred embodiment, the method for determining a solid cancer cfDNA methylome signature further comprises comparing the methylation state of each of the selected CpG loci and/or genomic regions in the first sample and in the one or more further samples with the methylation state of the same CpG locus and/or genomic region with one or more of the following:

a sample of white blood cells from the subject; and/or
a sample cfDNA from a healthy subject.

In one embodiment, the method for determining a solid cancer cfDNA methylome signature further comprises comparing the methylation state of each of the selected CpG loci and/or genomic regions in the first sample and in the one or more further samples with the methylation state of the same CpG locus and/or genomic region with one or more of the following:

a sample of white blood cells from the subject;
a sample of the solid cancer from the subject; and/or
a sample of non-cancerous tissue of origin of the solid cancer from the subject.

In one embodiment, the method for determining a solid cancer cfDNA methylome signature further comprises comparing the methylation state of each of the selected CpG loci and/or genomic regions in the first sample and in the one or more further samples with the methylation state of the same CpG locus and/or genomic region with one or more of the following:

a sample of cfDNA from a healthy subject (for example an age-matched healthy subject); and/or
a sample of non-cancerous tissue of origin of the solid cancer from the subject from a healthy subject (for example an age-matched healthy subject).

In one embodiment, the method for determining a solid cancer cfDNA methylome signature further comprises comparing the methylation state of each of the selected CpG CpG loci and/or genomic regions in the first sample and in the one or more further samples with the methylation state of the same CpG locus and/or genomic region with one or more of the following:

a sample of the solid cancer from multiple different subjects and optionally a sample of the solid cancer from the subject;
a cell-line of the solid cancer from multiple different subjects; and/or
a sample of cfDNA from a subject known to have the solid cancer (for example an age-matched subject known to have the solid cancer, and for example wherein the level of cancer fraction in the cfDNA sample from the different subject is known and/or wherein the sample is known to comprise cfDNA derived from a prostate cancer subtype).

In one embodiment, the method for determining a solid cancer cfDNA methylome signature further comprises comparing the methylation state of each of the selected CpG loci and/or genomic regions in the first sample and in the one or more further samples with the methylation state of the same CpG locus and/or genomic region with one or more of the following:

a sample of the solid cancer from multiple different subjects and optionally a sample of the solid cancer from the subject;
cell-lines of the solid cancer from multiple different subjects;
a sample of white blood cells from the subject;
samples of white blood cells multiple different subjects; and/or
samples of non-cancerous tissue of origin of the solid cancer from multiple different subjects;
a sample of non-cancerous tissue of origin of the solid cancer from the subject; and/or
a sample of cfDNA from a subject known to have the solid cancer (for example an age-matched subject known to have the solid cancer, and for example wherein the level of cancer fraction in the cfDNA sample from the different subject is known and/or wherein the sample is known to comprise cfDNA derived from a prostate cancer subtype).

In one embodiment, the method for determining a solid cancer cfDNA methylome signature further comprises comparing the methylation state of each of the selected CpG loci and/or genomic regions in the first sample and in the one or more further samples with the methylation state of the same CpG loci and/or genomic regions in a sample of cfDNA from a subject known to have the solid cancer (for example an age-matched subject known to have the solid cancer, and for example wherein the level of cancer fraction in the cfDNA sample from the different subject is known and/or wherein the sample is known to comprise cfDNA derived from a prostate cancer subtype), and preferably multiple cfDNA samples (for example at least 2, 3, 4, 5, 10, 20, 40, 50, 100, 200 or 500 samples) each from a different subject known to have the solid cancer (for example each from a different age-matched subject known to have the solid cancer, and for example wherein the level of cancer fraction in the each cfDNA sample from the different subjects is known and/or wherein each the sample is known to comprise cfDNA derived from a prostate cancer subtype).

The method for determining a solid cancer cfDNA methylome signature may further comprise determining a reference value for each of the selected CpG loci and/or genomic regions. In certain embodiments, the reference value is based on the methylation level (e.g. the methylation ratio for a CpG locus or the average methylation ratio for a genomic region) of the same CpG locus and/or genomic region in a cfDNA sample from one or more healthy subjects. In certain embodiments, the reference value is based on the methylation level (e.g. the methylation ratio for a CpG locus or the average methylation ratio for a genomic region) of the same CpG locus and/or genomic region in one or more white blood cell samples. In certain embodiments, the reference value is based on the methylation level (e.g. the methylation ratio for a CpG locus or the average methylation ratio for a genomic region) of the same CpG locus and/or genomic region in a sample of tissue from one or more healthy subjects. In certain embodiments, the reference value is based on the methylation level (e.g. the methylation ratio for a CpG locus or the average methylation ratio for a genomic region) of the same CpG locus and/or genomic region in one or more samples of solid cancer tumour and/or one or more solid cancer cell lines. In certain embodiments, the reference value is based on the methylation level (e.g. the methylation ratio for a CpG locus or the average methylation ratio for a genomic region) of the same CpG locus and/or genomic region in a sample of cfDNA from a subject known to have the solid cancer (for example an age-matched subject known to have the solid cancer, and for example wherein the level of cancer fraction in the cfDNA sample from the different subject is known and/or wherein the sample is known to comprise cfDNA derived from a prostate cancer subtype).

In one embodiment, the reference value is based on the methylation level (e.g. the methylation ratio for a CpG locus or the average methylation ratio for a genomic region) of the same CpG locus and/or genomic region in a cfDNA sample from one or more healthy subjects. In another embodiment, the reference value is based on the methylation level (e.g. the methylation ratio for a CpG locus or the average methylation ratio for a genomic region) of the same CpG locus and/or genomic region in one or more white blood cell samples.

In certain embodiments, a reference value for each of the selected CpG loci and/or genomic regions is the average methylation ratio of the same CpG locus and/or genomic region in or covered by:

a cfDNA sample from a healthy subject, for example a healthy age-matched subject;
a tissue sample from a healthy subject, for example a prostate tissue sample from a healthy subject;
a cancer biopsy sample from a cancer patient, for example a prostate cancer biopsy sample from a prostate cancer patient;
a cancer cell line sample, for example a prostate cancer cell line sample from a prostate cancer cell line;
a sample of white blood cells from a subject, for example the subject or a healthy subject;
a characterized methylome sequence of a white blood cell;
a characterized methylome sequence of a prostate cancer cell line;
a characterized methylome sequence of a cancerous prostate cell;
a characterized methylome sequence of a non-cancerous prostate cell; or
a sample of cfDNA from a subject known to have the solid cancer (for example an age-matched subject known to have the solid cancer, and for example wherein the level of cancer fraction in the cfDNA sample from the different subject is known and/or wherein the sample is known to comprise cfDNA derived from a prostate cancer subtype).

The method for determining a solid cancer cfDNA methylome signature may further comprise determining two or more (for example 3 or more, 4 or more, 5 or more, 10 or more, 15 or more, or 20 or more) reference values for each of the selected CpG loci and/or genomic regions (for example 2, 3, 4, 5, 6, 7, 8, 9 10, 15, 20, 30, 40, 50, 100, 200, 500 or 1000 reference values for each of the selected CpG loci and/or genomic regions). The two or more reference values may be selected from the average methylation ratio of the same CpG locus and/or genomic region in or covered by one or more of the following:

a cfDNA sample from a healthy subject, for example a healthy age-matched subject;
a tissue sample from a healthy subject, for example a prostate tissue sample from a healthy subject;
a cancer biopsy sample from a cancer patient, for example a prostate cancer biopsy sample from a prostate cancer patient;
a cancer cell line sample, for example a prostate cancer cell line sample from a prostate cancer cell line;
a sample of white blood cells from a subject, for example the subject or a healthy subject;
a characterized methylome sequence of a white blood cell;
a characterized methylome sequence of a prostate cancer cell line;
a characterized methylome sequence of a cancerous prostate cell; or
a characterized methylome sequence of a non-cancerous prostate cell;
a sample of cfDNA from a subject known to have the solid cancer (for example an age-matched subject known to have the solid cancer, and for example wherein the level of cancer fraction in the cfDNA sample from the different subject is known and/or wherein the sample is known to comprise cfDNA derived from a prostate cancer subtype).

The method for determining a solid cancer cfDNA methylome signature may further comprise establishing an algorithm for detecting, screening, monitoring, staging, classification, selecting treatment for, ascertaining whether treatment is working in, prognostication and/or treatment of the solid cancer using the cfDNA methylome signature.

The algorithm may be established using, for example, a random forest classifier, a regression analysis algorithm, for example a least absolute shrinkage and selection operator (LASSO) algorithm, a Naïve Bayes classifier, a support vector machine, a perceptron learning algorithm, a decision tree, a gradient boosting tree, a neural network or k-nearest neighbour algorithm. The algorithm can be implemented using R, python languages or equivalent statistical application or software (such as STATA) by one of ordinary skill in the art.

In certain embodiments, the algorithm is for determining the presence of solid cancer in a further sample comprising DNA using the cfDNA methylome signature.

In certain embodiments, the algorithm is for determining the level of a solid cancer in a further sample comprising DNA using the cfDNA methylome signature, for example the level of solid cancer tumour fraction.

In certain embodiments, the algorithm is for determining a subtype of solid cancer in a further sample comprising DNA using the cfDNA methylome signature.

In preferred embodiments the algorithm comprises comparing the methylation status, the methylation ratio, or the average methylation ratio, for some or all of the selected CpG loci and/or genomic regions of the cfDNA methylome signature to the methylation status, the methylation ratio, or the average methylation ratio for some or all of the selected CpG loci and/or genomic regions in a further sample comprising DNA. Additionally, or alternatively, the algorithm comprises comparing the methylation status, the methylation ratio, or the average methylation ratio, for some or all of the selected CpG loci and/or genomic regions of the cfDNA methylome signature to a reference value for each CpG locus and/or genomic region.

The invention will now be illustrated in a non-limiting way by reference to the following Example.

### EXAMPLES

### Example 1: New Prostate Cancer Plasma Methylation Signatures

### Materials and Methods

### Study Design

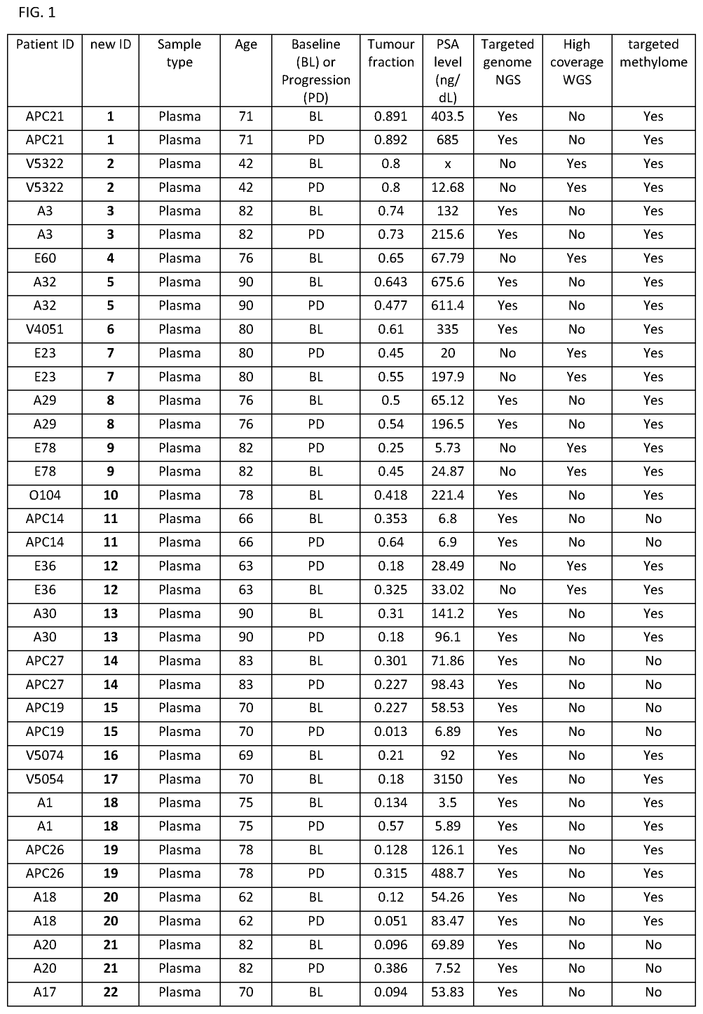

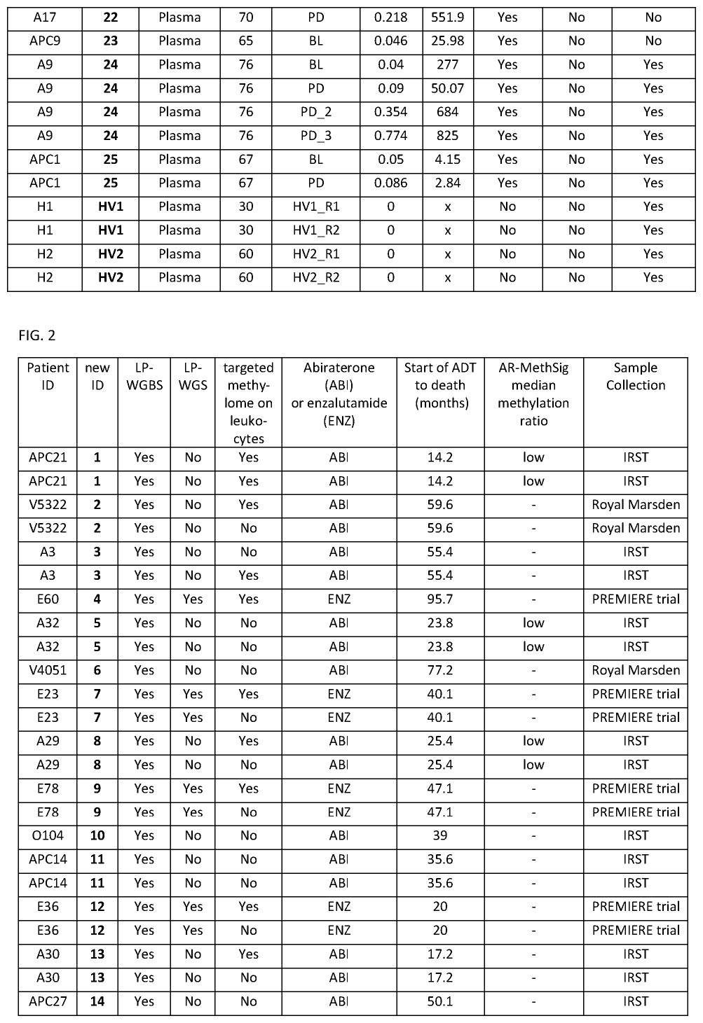

Plasma samples were collected within 30 days of treatment initiation and at progression in two biomarker studies, separately approved by the Istituto Scientifico Romagnolo per lo Studio e la Cura dei Tumori (IRST), Meldola, Italy (REC 2192/2013) and Royal Marsden, London, UK (REC 04/Q0801/6) and in the PREMIERE trial (EudraCT: 2014-003192-28, NCT02288936) that was sponsored and conducted by the Spanish Genito-Urinary oncology Group (SOGUG) (FIGS. 1 and 2). All patients provided written informed consent for these analyses.

These cohorts were described in Romanel et al. (Romanel, A., et al. Sci Transl Med 7, 312re310 (2015)) and Conteduca et al (Conteduca, V. et al., Ann Oncol 28, 1508-1516, (2017)). Briefly, patients needed to have histologically or biochemically confirmed prostate adenocarcinoma and be starting abiraterone or enzalutamide treatment for progressive mCRPC. Patients were required to receive abiraterone or enzalutamide until disease progression as defined by at least two of the following: a rise in PSA, worsening symptoms, or radiological progression defined as progression in soft-tissue lesions measured by computed tomography (CT) imaging according to modified Response Evaluation Criteria in Solid Tumors or progression on bone scanning according to criteria adapted from the Prostate Cancer Clinical Trials Working Group 2 guidelines. Patients with sufficient vials to allow both genome and methylome assessment were prioritised. Metastases were obtained at rapid warm autopsy in the Peter MacCallum warm autopsy program CASCADE (Cancer tissue Collection After Death) described by Alsop et al. (Alsop, K. et al. A, Nat Biotechnol 34, 1010-1014 (2016). (HREC 15/98, FIG. 2).

### Plasma DNA Sequencing

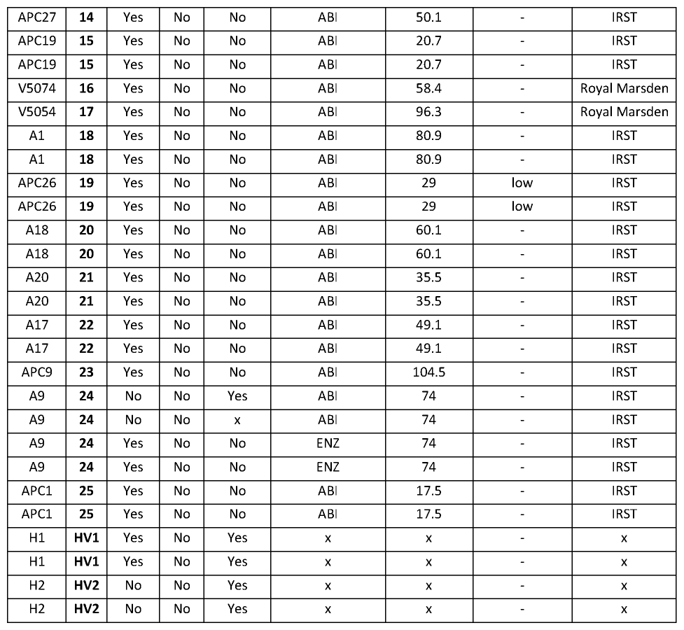

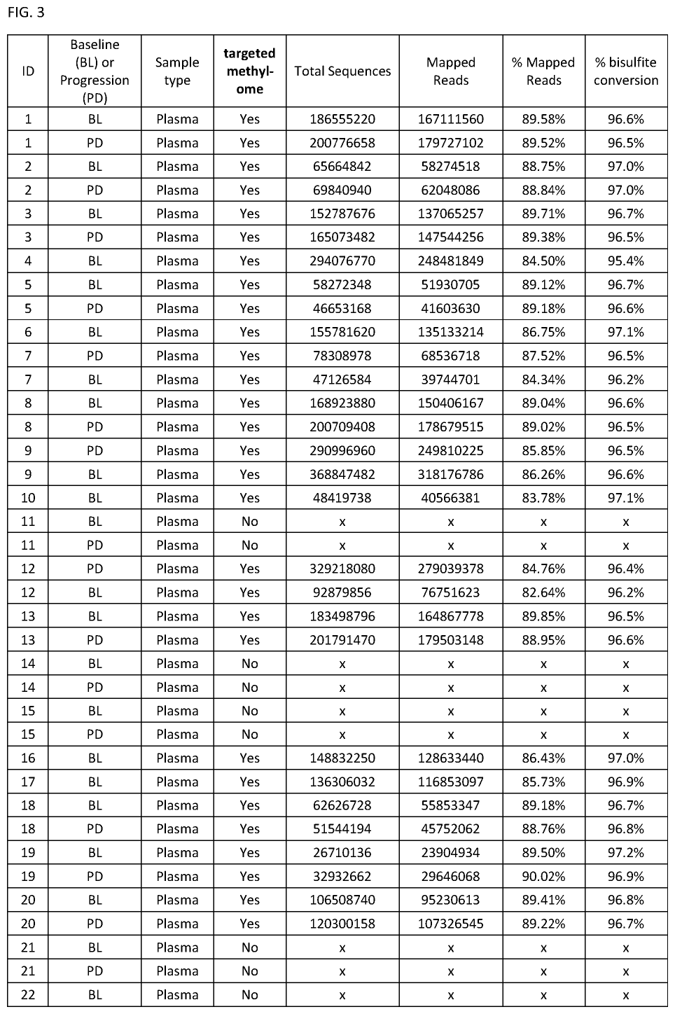

Circulating DNA (10-25 ng) was extracted from plasma using the QIAamp Circulating Nucleic Acid kit (Qiagen™) and quantified using the Quant-iT high-sensitivity Picogreen double-stranded DNA Assay Kit (Invitrogen by Thermo Fisher™). Germline DNA was extracted from white blood cells using the QIAamp DNA kit (Qiagen™). Genomic NGS was performed as described previously (Romanel, A. et al. Sci Transl Med 7, 404 312re310 (2015)). For methylation assessment, raw plasma DNA was bisulfite treated using the ZYMO™ Gold Kit as per the manufacturer's protocol. Swift Bioscience™ Methyl-Seq was used to generate libraries. CpGs were selected from prior data generated using Illumina Infinium HumanMethylation450k microarray (Roche Nimblegen™ targeted capture kit, Epi CpGiant). Probes were designed to hybridize to strands of fully methylated, partially methylated and fully unmethylated derivatives of the target as described below. Libraries were quantified by KAPA library quantification kit (Roche™) before pooling and sequencing on an Illumina™ HiSeq 2500 using paired-end 100-base pair reads. Sequencing matrices for targeted methylome and LP-WGBS are shown in FIGS. 2 and 3, and details on the pipelines for analysis of sequencing data are provided below.

### Processing of Targeted Methylation NGS Data

Data were processed using fastqc to assess quality and read through adapters were trimmed using Trimmomatic v0.36. Since DNA was bisulfite treated, reads were aligned based on three nucleotides (thymine (T), adenosine (A), guanine (G)) to the human genome (hg)19 using the BSMAP v2.90 (Xi, Y. & Li, W., BMC Bioinformatics 10, 232 (2009); Bolger, A. M., et al, Bioinformatics 30, 2114-2120 (2014)). The duplicated reads were removed with Picard tools v2.1.0 (http://broadinstitute.github.io/picard), and unaligned reads were clipped (hard-clipped) using the bamUtil 1.0.13 (Jun, G et al, Genome Res 25, 918-925 (2015)).

The CpG methylation ratio of each loci was calculated using formula (I), which takes cytosine (C) and thymidine (T) counts from all reads covering each CpG loci.

$$Methylation Ratio = \frac{C}{C + T} \qquad ( I )$$

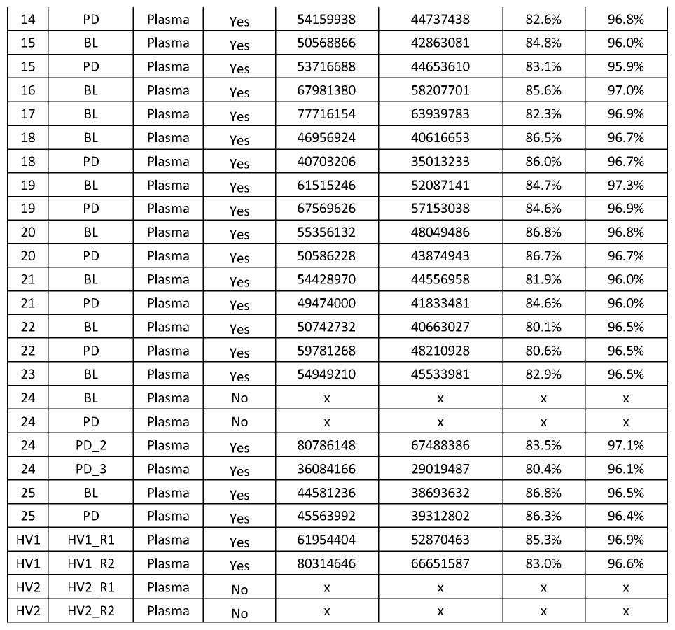

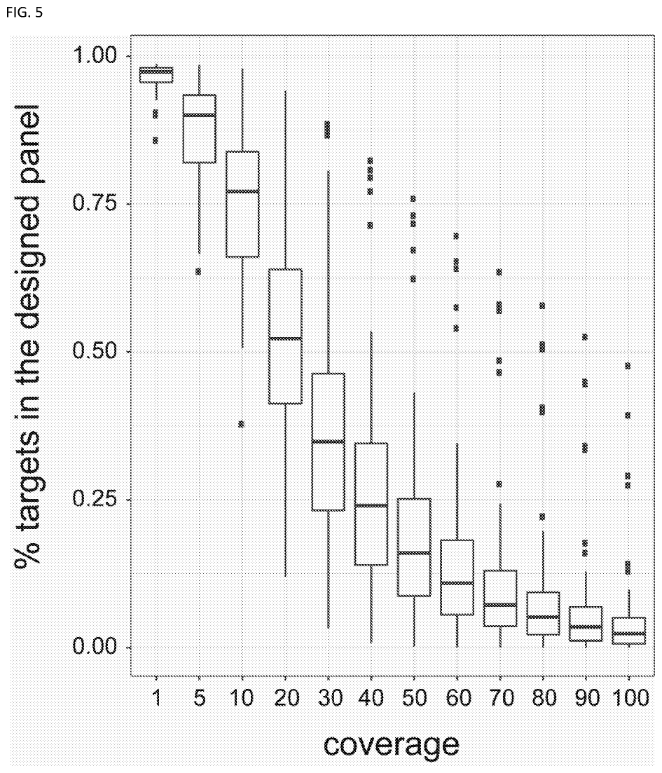

From all sites included in the predesigned capture panel (Roche Nimblegen SeqCap EpiGiant), only sites with a minimum coverage of 10 reads were considered for further analysis of CpG (FIG. 5). The methylation ratio was computed using the methylKit R package v1.6.2 (Akalin, A. et al. Genome Biol 13, R87 (2012)).

### Selection of Optimal Data Inputs for PCA

Adjacent CpG methylation levels are usually highly related, and previously studies have demonstrated high sensitivity of identifying tissue-specific methylation markers using sliding window approaches (Lehmann-Werman, R. et al. Proc Natl Acad Sci USA 113, E1826-1834 (2016); Guo, S. et al. Nat Genet 49, 635-642 (2017); Sun, K. et al. Proc Natl Acad Sci USA 112, E5503-5512 (2015)). Here adjacent CpG sites were combined into methylation segments of fixed length (the term “methylation segment” and the term “segment” as used in the examples section may also be referred to as a genomic region), and the average methylation ratio across all CpGs within the segment was calculated and used to represent the methylation ratio of the segment using methylKit R package v1.6.2 (Akalin, A. et al. Genome Biol 13, R87 (2012)). Initially 100 bp with sliding window of 50 bp were used and generated >1.47 million windows across all CpGs in the target panel. Principal component analysis (PCA) was applied using the FactoMineR v1.41 package.

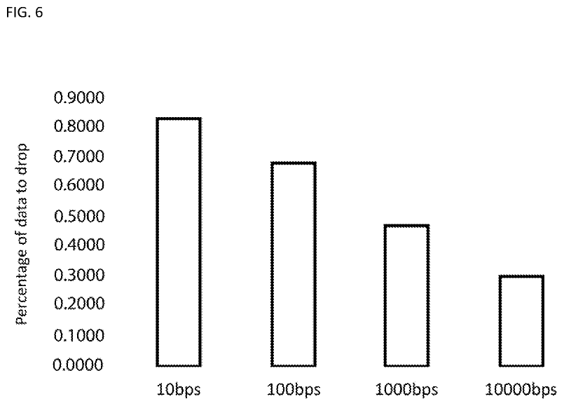

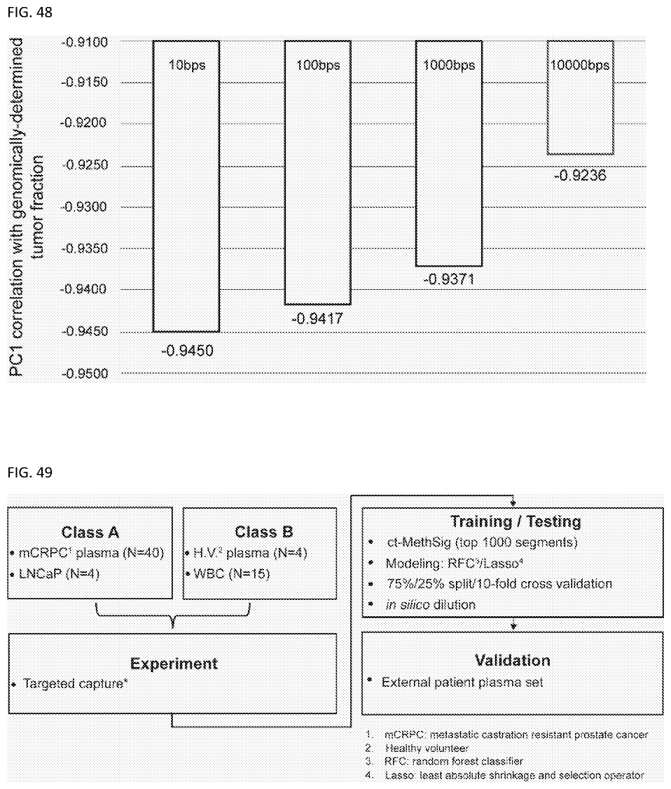

To eliminate potential biases due to the selection of segmentation length, segmentation length parameters were optimised. To do so, segments of 10 bp, 100 bp, 1000 bp and 10,000 bp were tested with sliding windows of 5 bp, 50 bp, 500 bp and 5000 bp, respectively. It was found that the smaller the window size, the more data that had to be drop when combining plasma samples due to variable inputs and sequencing coverage (FIG. 6). It was also found that the average methylation ratio of 100 bp segments with 50 bp sliding windows showed high consistency with the methylation ratio estimated at single CpG level (FIG. 7). The correlation of PC1 with genomically-determined tumour fraction was >90% regardless of window sizes (FIG. 48).

Thus, to preserve more detailed methylation information, and to guarantee successful execution in a reasonable amount of time, the setting of 100 bp segments with 50 bp sliding window was applied for the rest of the analysis. However, other segment sizes and windows could have been used.

### Principal Component Analysis of Targeted Plasma Methylome

The methylation segments for which methylation ratios available in all baseline samples (n=19) and for which the standard deviation values were in the upper two quartiles, were subjected to principal component analysis (FactorMineR R package v1.41, as described in Lê, S., Josse, J. & Husson, F. FactoMineR: An R Package for Multivariate Analysis. 2008 25, 18, doi:10.18637/jss.v025.i01 (2008).).

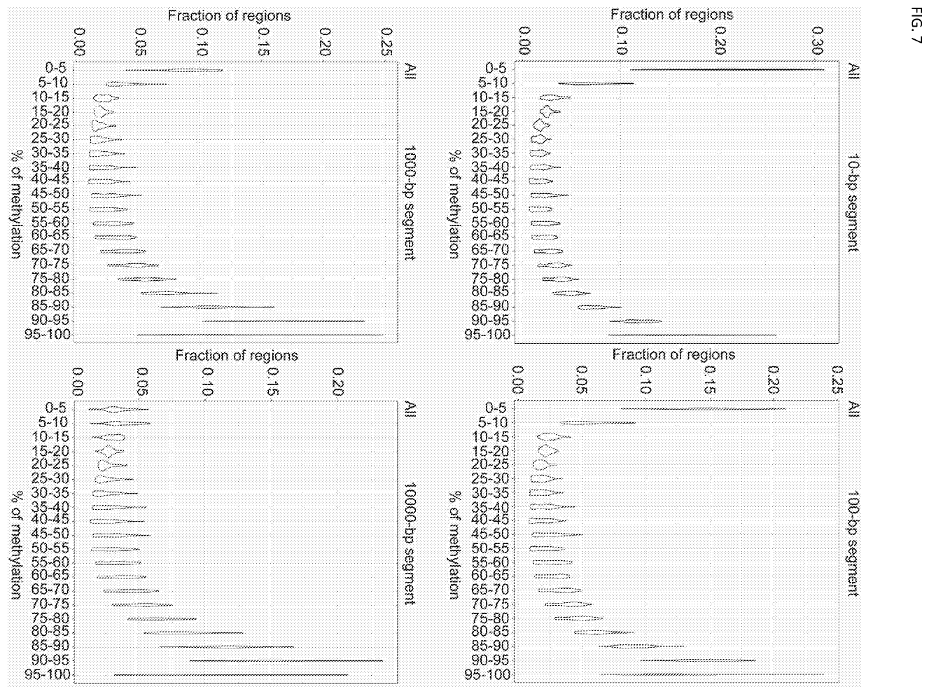

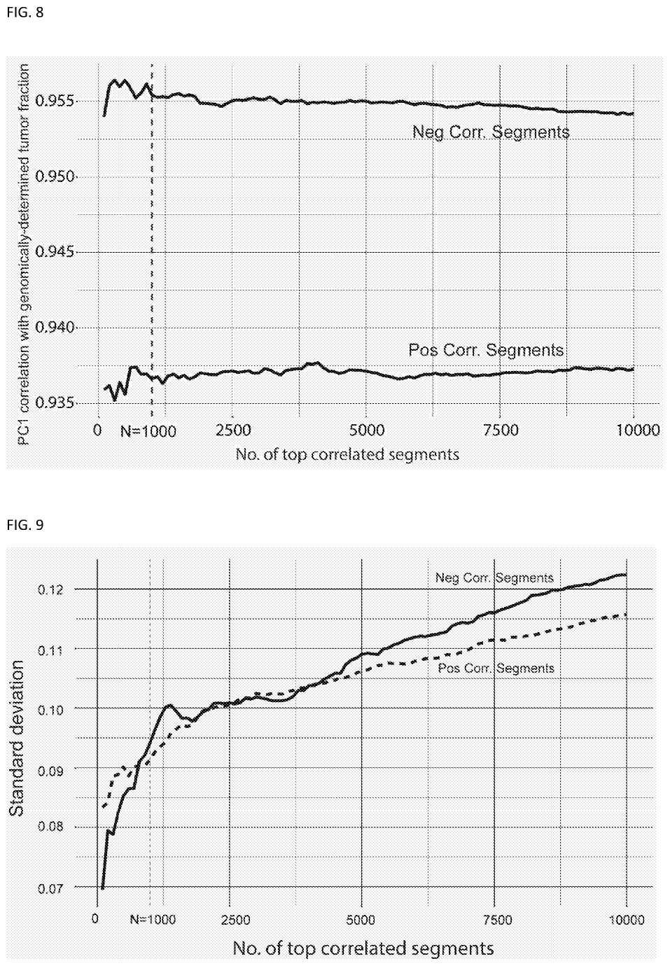

More specifically, unscaled PCA using FactoMineR (http://factominer.free.fr) (Lê, S., Josse, J. & Husson, F. FactoMineR: An R Package for Multivariate Analysis. 2008 25, 18 (2008)) was applied. The PCA model comes with the eigenvector, eigenvalues and correlation matrix comprised of correlation coefficient by each segment. The distribution of the top-K highly correlated segments was plotted based on the correlation matrix returned by PCA, and these segments were highly representative of each eigenvector (e.g., principal component 1, or PC1). To identify the optimal value K of highly correlated segments, multiple K values equal to 10, 100, 1,000, and 10,000 were tested and intra-sample variance calculated, and the correlation between the median of the average methylation ratios with genomically-determined tumour fraction was determined (FIGS. 8 and 9).

Significant principal components were determined using a permutation test as implemented in the jackstraw R package (v1.2) (https://CRAN.R-project.org/package=jackstraw). The projection of all the samples based on the PCA eigenvectors was based on the average methylation ratio of each segment (i.e. average methylation ratio of all the CpG loci within each region) used in the initial PCA for all the samples. Missing values were imputed based on the PCA method as implemented in the missMDA R package (v1.13), as described in Josse, J. & Husson, F. missMDA: A Package for Handling Missing Values in Multivariate Data Analysis. 2016 70, 31, doi:10.18637/jss.v070.i01 (2016).

### Tumour Fraction Estimation

Genomically-determined tumour fraction was determined from targeted next-generation sequencing (NGS) using CLONET as described in Romanel et al. (2015) and Prandi et al. (Prandi, D. et al. Genome Biol 15, 439 (2014)). On high-coverage targeted methylation NGS, PC1 values were calculated as described above, and the median of PC1 values extracted from healthy volunteers were set as 0%, while the median of PC1 values derived from LNCaP samples were set as 100% tumour purity. The tumour fractions of all the plasma samples were obtained with interpolation using PC1 projected values. For tumour fraction estimation based on low-passage whole genome sequencing (LP-WGS) on bisulfite-treated or non-treated plasma DNA, ichorCNA (Adalsteinsson, V. A. et al. Nat Commun 8, 1324 (2017)) was used as described below. For LP-WGBS PC1 projected values were used.

Analysis of LP-WGS by ichorCNA

LP-WGS on both bisulfite-treated and untreated plasma DNA was performed with a target 1× coverage. For each sample, reads from LP-WGS on untreated plasma DNA were aligned to the hg19 using BWA-MEM version 0.7.12-r1039 and de-duplicated using Picard tools v2.1.0. The human genome was then divided into non-overlapping bins of 1 million base pairs, and, for each sample, the de-duplicated reads were counted per bin using HMM Copy (http://compbio.bccrc.ca/software/hmmcopy/) (Ha, G. et al. Genome Res 22, 1995-2007 (2012)). Next, ichorCNA (https://github.com/broadinstitute/ichorCNA) was applied to estimate the tumour content of each sample (Adalsteinsson, V. A. et al. Nat Commun 8, 1324 (2017)). The algorithm first removed bins in the centromere regions with a flanking region of 100,000 base pairs. For all the remaining bins read counts were corrected by GC content and mappability issues. The normalised read counts were then fed into the Hidden Markov model (HMM), which is a probabilistic model assigning each bin into one possible state (hemizygous deletions (HETD, 1 copy), copy neutral (NEUT, 2 copies), copy gain (GAIN, 3 copies), amplification (AMP, 4 copies), and high-level amplification (HLAMP, 5 or more copies). Based on the copy number profile, the model estimated a ploidy and tumour content for every sample. Finally, the algorithm was initiated with ploidy values 2 and 3, and normal fraction, which is 1 minus tumour fraction of 0.0, 0.1, 0.2, 0.3, 0.4, 0.5, 0.6, 0.7, 0.8, 0.9, 0.95. The solution with maximum likelihood among all of these initial combinations was automatically assigned. The CNA status was estimated based on the log R values of each 1 Mbp region obtained by the ichorCNA analysis with fixed threshold of 0.5 (GAIN: log R≥0.5, LOSS: log R≤0.5).

### Analysis of Low Passage Whole Genome Bisulfite Sequencing (LP-WGBS)

Reads from LP-WGBS were processed as high coverage NGS. To calculate PC1 values derived from LP-WGBS, the default segmentation length of 100 bp was used and the average methylation ratio of each segment (i.e. average methylation ratio of all the CpG loci within each region) was calculated based on formula (I) to determine the methylation ratio of each loci, and then then mean of all CpG loci in a segment was calculated to arrive at the average methylation ratio for a segment. To maximize the available information obtained from the data, methylation data from higher coverage bisulfite data based regularised iterative PCA algorithm (Josse, J. & Husson, F. missMDA: A Package for Handling Missing Values in Multivariate Data Analysis. 2016 70, 31) (missMDA R package (v1.13)) was inputted, and projected on the PCA model as described above. The regularisation process with random initialisation can also circumvent the over-fitting problem, which might reduce the generalization capabilities of the findings.

### Analysis of Illumina HumanMethylation450 BeadChip Dataset

The microarray processed data were obtained from the Gene Expression Omnibus (Edgar, R., et al, Nucleic Acids Res 30, 207-210 (2002)) repository (GSE84043). From the dataset probes overlapping with PC1 segments were selected. The average methylation ratio of each segment was obtained considering the median of the 13 values of the overlapping probes. The tumour fraction estimates by different methods were obtained by the sample information published (Fraser, M. et al, Nature 541, 359-364 (2017)).

### Statistical Analysis

### Overview

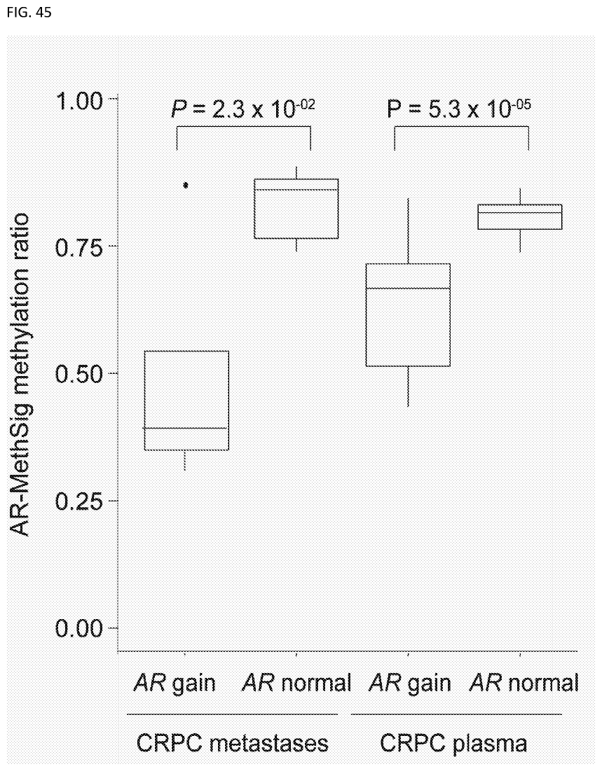

Pearson correlation was used to measure the association between two parameters (principal component values versus genomically determined tumour fraction estimation, or different approaches of tumour fraction estimations). The association between copy number status of each region and principal components was estimated using the Kruskal-Wallis test. Mann-Whitney U test was used to test significance between two groups (AR gain versus AR non-gain—see FIG. 45). Hazard ratio in overall survival analysis was calculated using the Mantel-Haenszel method. For all tests, a significance threshold of 0.05 was required unless otherwise specified.

### Correlation and Association Analysis

Correlation analyses of continuous measures were performed using the Pearson correlation method as implemented in the R v3.4.0 stats package. The association analysis between principal components and CNA of each region was performed by grouping the principal component values of each sample based on the CNA observed for the region (LOSS, NEUTRAL and GAIN). The differences in the principal component values distribution among groups was then assessed using the Kruskal-Wallis test (one-way ANOVA on ranks) as implemented in the R v3.4.0 stats package.

Methylation Ratio Difference with Kruskal-Wallis and Dunn's Test

The samples were grouped based on tissue of origin and clinical status (white blood cells, plasma healthy volunteer, plasma baseline and plasma progression). Samples were grouped by ct-MethSig and AR-MethSig, and the average methylation ratio of each 100 bp segment was estimated in each group of samples. To keep the analysis consistent, only segments present in all samples (340,467 segments) were considered. All the selected segments were split in two groups based on the overlap with the promoter region of known genes (263,262 non-promoter segments, 77,205 promoter segments). The promoter region was defined as 1k base-pair upstream and downstream of the transcription start site (TSS). The significance of the differences among each group was calculated using Kruskal-Wallis test (one-way ANOVA on ranks) as implemented in the R v3.4.0 (https://www.R-project.org (2018)) stats package. After defining the significance of the differences, the difference of the average methylation ratio across each group was assessed using the Dunn's test as implemented in FSA R package v0.8.22 (https://github.com/droglenc/FSA).

### Functional Enrichment Analysis

Functional enrichment analysis (chemical and genetic perturbations, MSigDB) was executed using the enrich R package (v0.1) based on all the MSigDB main categories (MSigDB database v6.0) (Liberzon, A. et al. Cell Syst 1, 417-425 (2015)) with a significance threshold of 0.05 on Benjamini corrected p values.

### Motif Enrichment Analysis

Motif enrichment analysis was used to identify potential transcriptomic regulators of methylation signatures (MethSig). MethSig top 1000 correlated segments were submitted to find the possible motif binding sequences over-represented as compared to the default background set (Zambelli, F., et al, Nucleic Acids Res 41, W535-543 (2013)). The pipeline (Pscan-Chip) (Zambelli, F., et al, Nucleic Acids Res 41, W535-543 (2013)) originally designed for the analysis of chromatin immunoprecipitation followed by next generation sequencing technologies was applied. The program automatically scanned 75 bp preceding and after the ‘peak’ regions that were submitted with controlled background, and know transcriptional factor binding motifs obtained from JASPAR version 2018. Local enrichment p-value was two-tailed and denoted whether the motif was over-represented in the 150-bp region compared to the genomic regions flanking them. Global enrichment denoted whether the motif binding sequence was over-represented in the region with respect to global background composed of pan-genome putative regulatory regions from various cell lines. The analysis on top 1000 highly correlated segments with PC1 (i.e. ct-MethSig) or PC3 (i.e. AR-MethSig) was performed and other randomly selected regions from the custom, targeted enrichment panel. The result of AR-MethSig was validated by an orthogonal pipeline (Heinz, S. et al. Mol Cell 38, 576-589 (2010)), and the finding was consistent to original approach as described above.

### Gaussian Mixture Model (GMM)

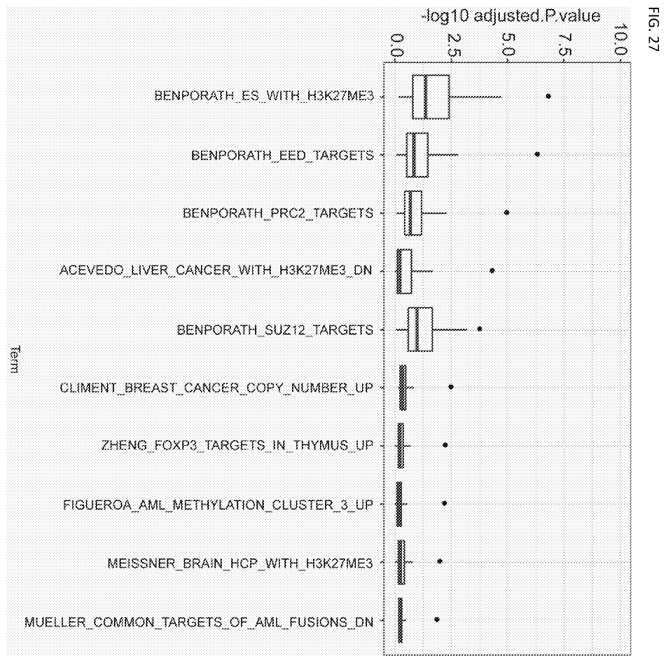

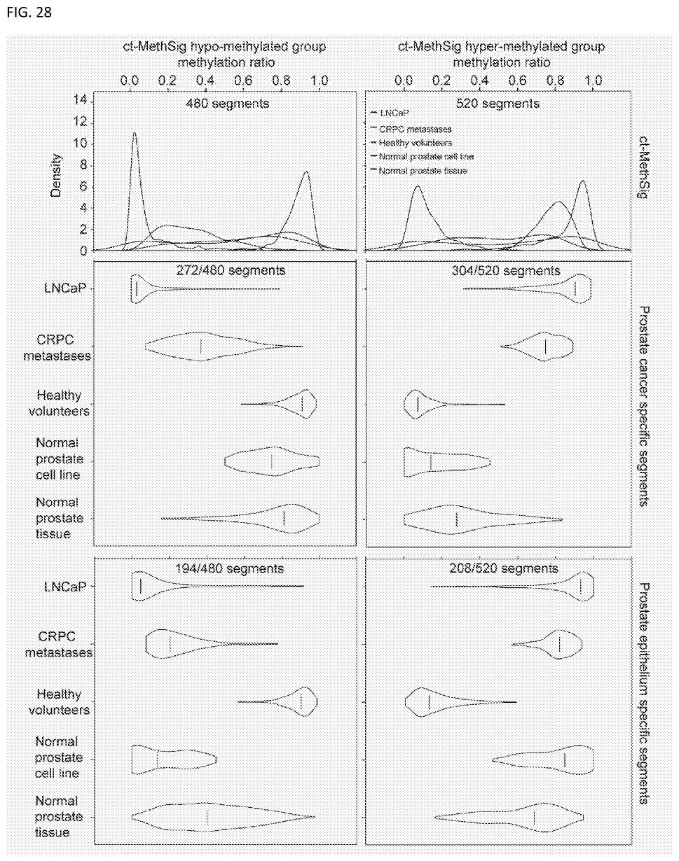

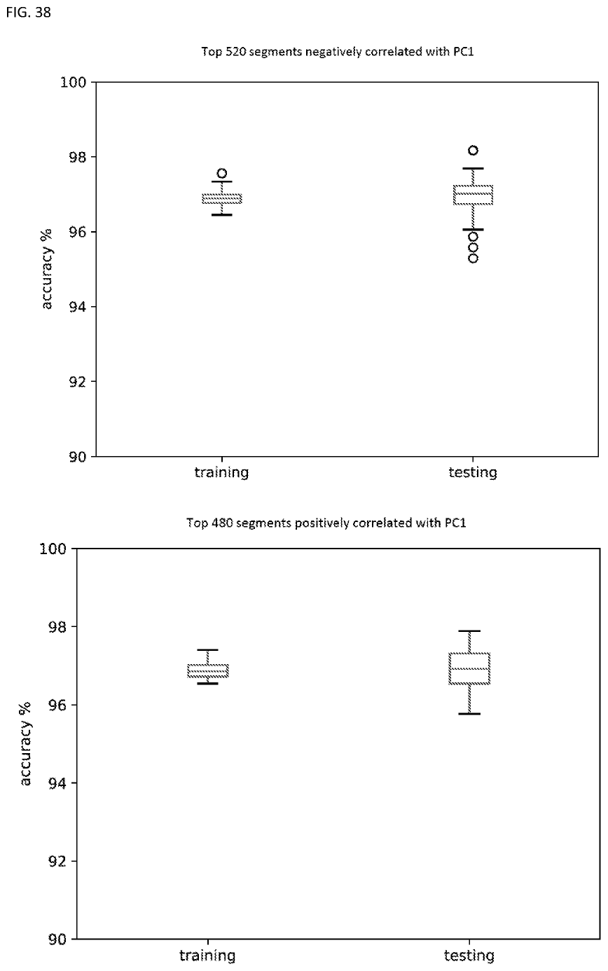

Average methylation ratios of ct-MethSig segments derived from LNCaP cell lines, and healthy volunteer plasma were extracted. To estimate the probability density function (pdf), kernel density estimation (kde) was applied, assuming a mixture of two Gaussian distributions consistent with the input dataset of normal prostate epithelium (FIG. 28). The Gaussian mixture model (see formula (II) below) applies expectation-maximization (EM) to fit the mixtures of Gaussian distributions by an iterative process (Pedregosa, F. et al. J. Mach. Learn. Res. 12, 2825-2830 (2011)). In the experiment, the model was executed with maximum iterations of 100 times and ‘k-means’ method for initialization, and it was hypothesized that there were two Gaussian distributions, each of them with its own general covariance. The Gaussian mixture model was subject to cross-validation on random split set of regions over 100 times to prove the robustness of the approach (FIG. 38). The fitted GMM (number of class=2) was then used to predict the top 1000 segments of PC1, and thus arrive at the ct-MethSig segments of prostate epithelium (PrEC) (Pidsley, R. et al. Genome Biol 17, 208 (2016)).

Gaussian mixture model: gⱼ(x)=ø_{θ}_{ⱼ}(x); where θⱼ=(μᵢ,σⱼ²)

### Results

### Results

### Interrogating the Plasma DNA Methylome in Metastatic Prostate Cancer

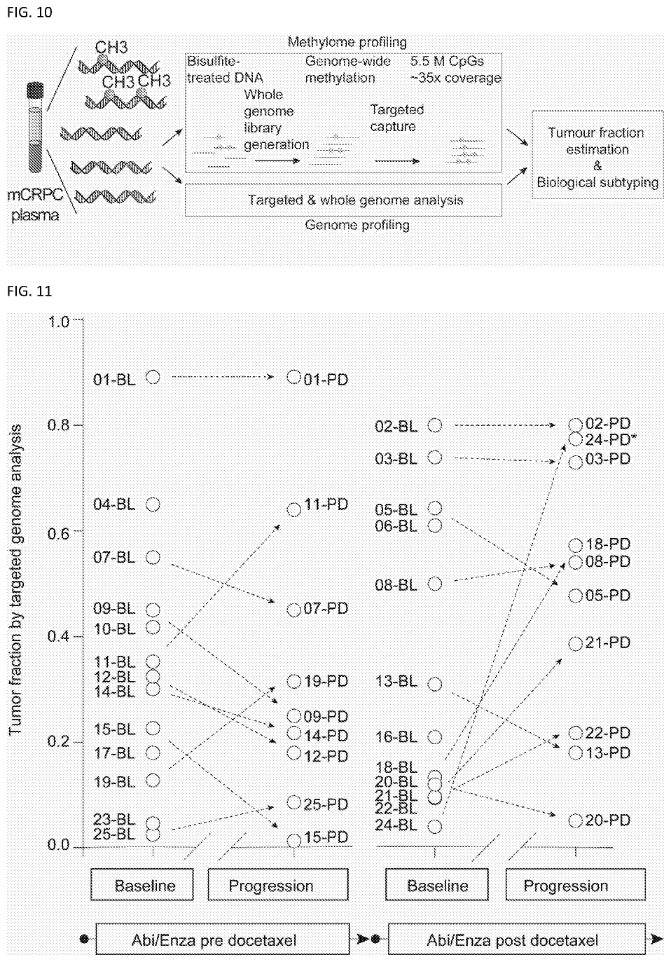

The mCRPC plasma methylome and genome were concurrently characterized (FIG. 10). Plasma DNA was subjected to either high-coverage targeted or whole genome NGS in order to determine tumour fractions and copy number status. Tumour fractions were derived using genomic information at heterozygous single-nucleotide polymorphisms (SNPs) to computationally determine the abundance of deletions involving 8p21 or 21q22, designated as prostate cancer anchor lesions that were used previously as a proxy for tumour fraction (Prandi, D. et al, Genome Biol 15, 439 (2014); Carreira, S. et al. Sci Transl Med 6, 254ra125 (2014)). Plasma has been collected up to 30 days prior to abiraterone or enzalutamide (baseline) from 25 mCRPC patients (median age: 76 years; range: 42-90) participating in prospective biomarker protocols, with a wide range of genomically-determined tumour fractions and from across the disease spectrum (docetaxel-naïve or docetaxel-treated). From the 25 patients, plasma had been collected from 19 patients at radiographic progression and four control samples were collected from two healthy, male volunteers (aged 30 and 60, FIG. 11, FIGS. 1 and 2). The median and range of genomically-determined tumour fractions in the mCRPC cohort were 0.41 (0.04-0.89) and 0.42 (0.09-0.89) for baseline and progression plasma, respectively.

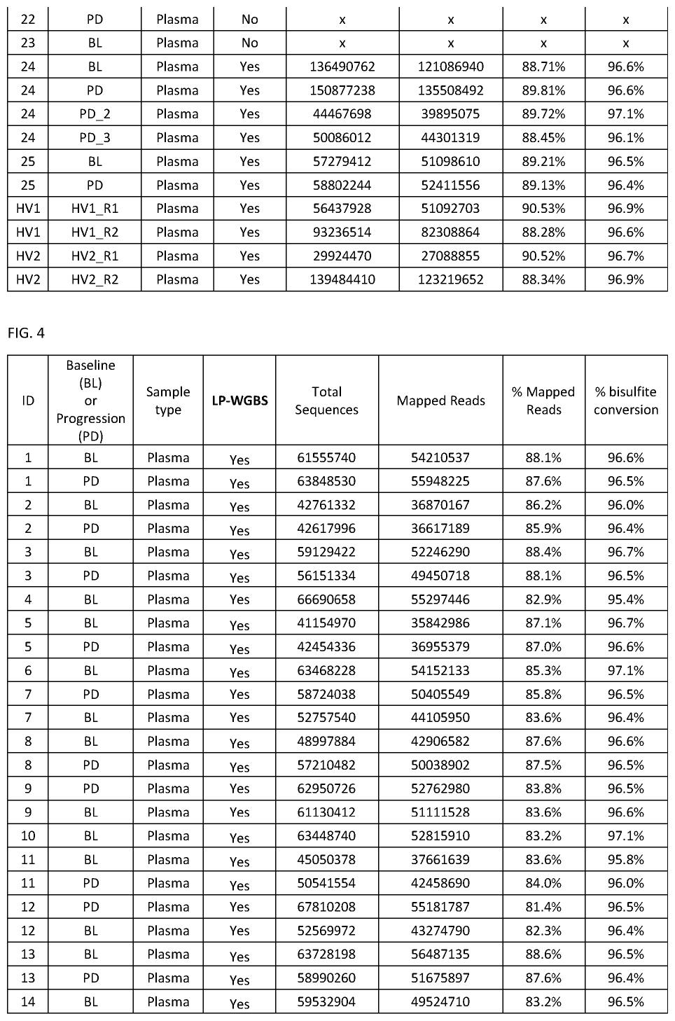

A separate aliquot of DNA was subjected to bisulfite treatment and target enrichment NGS for 5.5 million pan-genome CpG sites was performed (target coverage: ≥30×; key sequencing parameters in FIGS. 2 and 3). These CpGs were selected based on their known involvement in or proximity to regions that had been associated with disease (Roche Nimblegen™ targeted capture kit, Epi CpGiant). In total targeted capture was performed on 39 plasma samples (19 baseline, 16 progression, 4 healthy volunteer plasma samples from two individuals, FIG. 5 and FIG. 3). Low-pass whole genome bisulfite sequencing (LP-WGBS) was also performed on 46 plasma samples (24 baseline, 20 progression, two plasma samples from one healthy volunteer—FIGS. 4 and 5). Additionally, targeted bisulfite NGS on 15 white blood cell samples, including white blood cells collected prior to and 108 days after treatment with abiraterone from one patient was conducted (FIGS. 1 and 2).

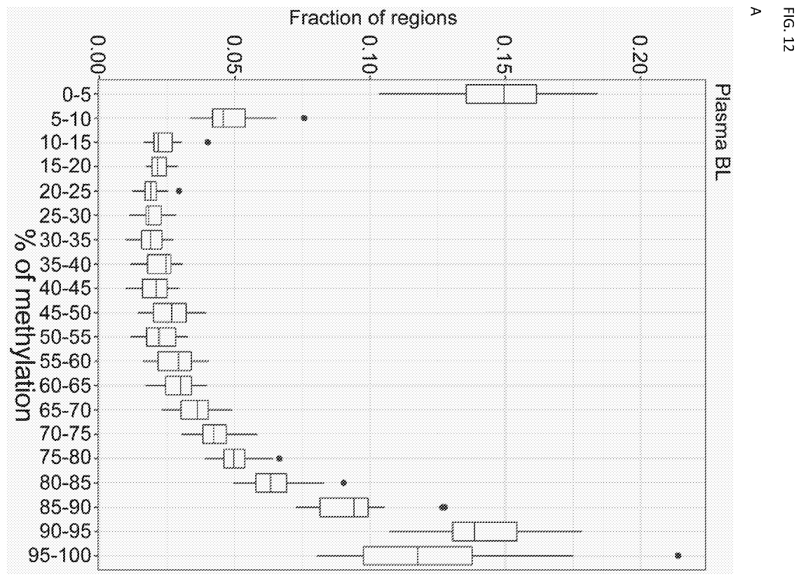

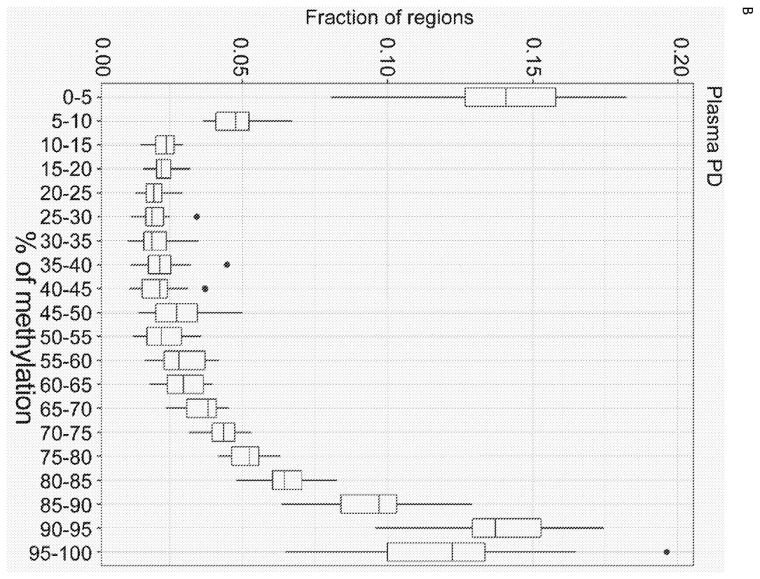

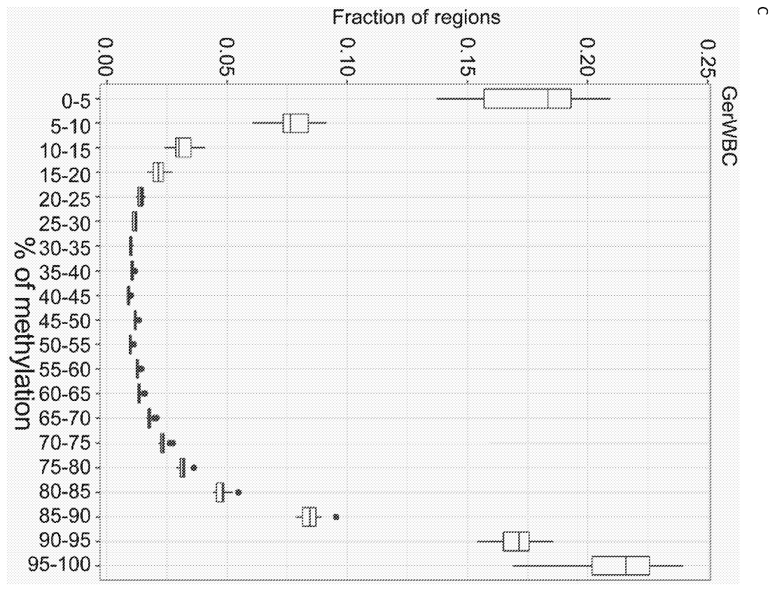

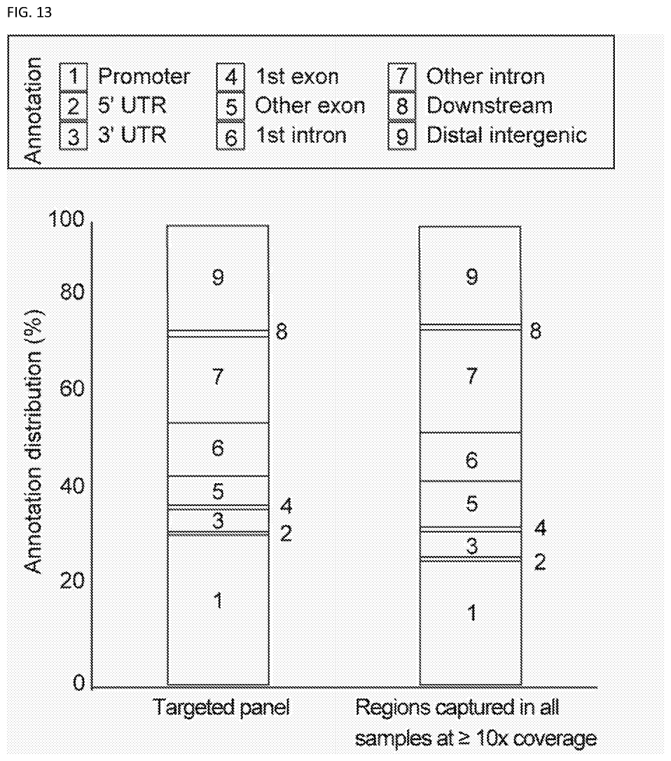

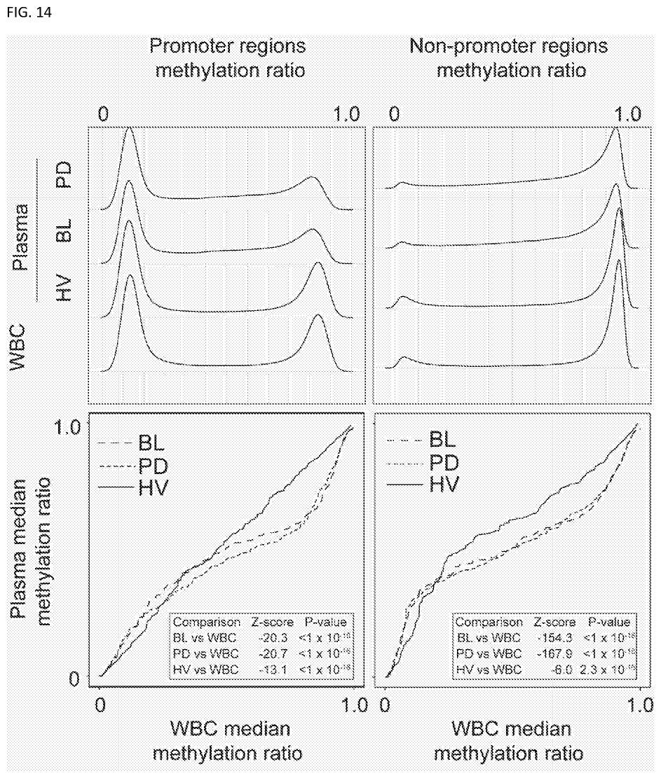

Adjacent CpG methylation patterns are usually highly correlated (Guo, S. et al. Nat Genet 49, 635-642, (2017); Lehmann-Werman, R. et al. Proc Natl Acad Sci USA 113, E1826-1834 (2016)). A 100 base-pair sliding window was applied and the data divided into 1.47 million methylation segments as described above. In keeping with prior studies on tissues, the methylation ratio distribution across all methylation segments in plasma and white blood cell samples showed a density peak for hypermethylation and hypomethylation (FIG. 12). Regions with a minimum of 10× coverage were selected. When separated by annotation category (such as promoter, exon, intron), the distribution was consistent with the targeted regions (FIG. 13) (Yu, G., et al, Bioinformatics 31, 2382-2383 (2015)). It was observed that methylation segments in promoter regions were primarily hypomethylated whilst other categories were primarily hypermethylated (FIG. 14, top panel). The average methylation ratio distribution for segments in baseline, progression plasma and healthy volunteer plasma were then compared with white blood cell DNA, and significant differences between plasma and white blood cell samples were observed (P<10⁻¹⁵, Kruskal-Wallis test). The difference was more pronounced in cancer patients' plasma samples compared to healthy volunteers' ((respectively, Z scores for promoter regions were −20.3, −19.6 and −15.6 and non-promoter regions: −157.2, −170.1 and −5.9; all P<10⁻⁹, Dunn's test, FIG. 14, bottom panel). In keeping with previous studies that the cancer genome is characterized by more hypo-methylation events, the mCRPC plasma methylome that includes a mixture of cancer and normal DNA, is globally more hypomethylated than healthy volunteer plasma.

### An Unbiased Approach Identifies Tumour Fraction as the Major Determinant of Global Plasma DNA Methylation Variance

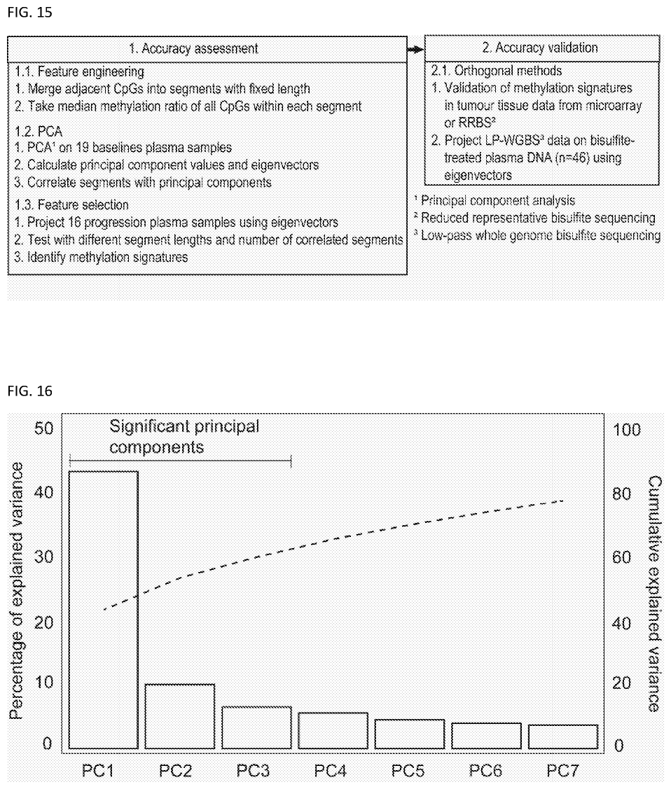

The analytical framework was applied on baseline plasma methylome (n=19) to identify methylation features associated with genomically-determined tumour fraction. To use an unbiased approach to explore the complexity of pan-genome plasma methylation changes, principal component analysis (PCA) was performed. Different parameters were experimented on and confirmed the robustness of the finding on progression, healthy volunteer plasma methylome and LNCaP cell line methylome. To expand the applicability of the approach, segments highly correlated with principal components were extracted and tested on LP-WGBS plasma methylome, and external, well-defined tissue data sets using orthogonal approaches (FIG. 15).

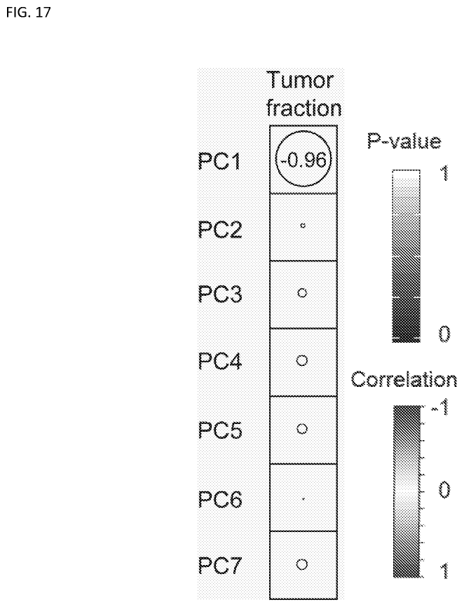

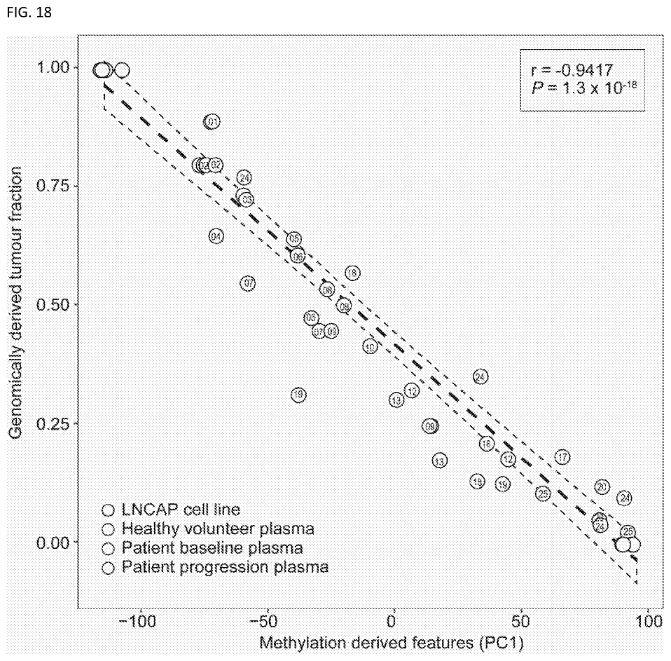

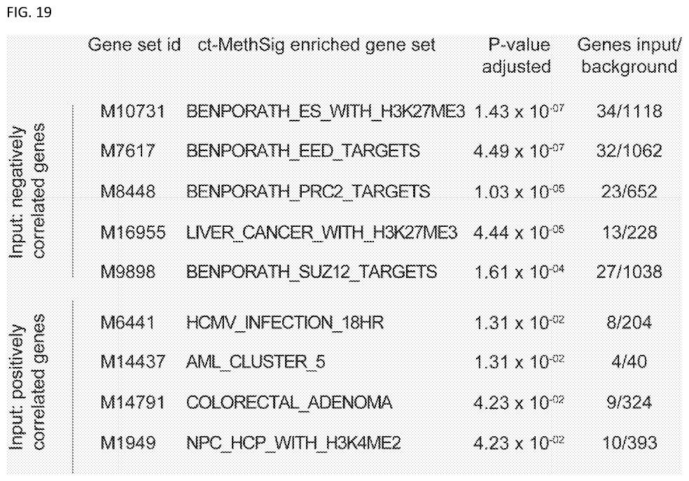

The first principal component (PC1) contributed 42% of the variance (FIG. 16) and showed a high correlation with genomically-determined tumour fraction (r=−0.96, P=1.3×10⁻¹⁰, Pearson correlation, FIG. 17). To investigate whether treatment with AR targeting agents affected the association of PC1 with tumour fraction, PCA eigenvectors were used to project the progression samples, healthy volunteer controls (“0” tumour fraction) and the LNCaP prostate cancer cell line (100% tumour, three replicates, FIG. 18). After including the projected samples, the correlation of PC1 and genomically-determined tumour fraction remained high (r=−0.94, P=1.3×10⁻¹⁸, FIG. 19).

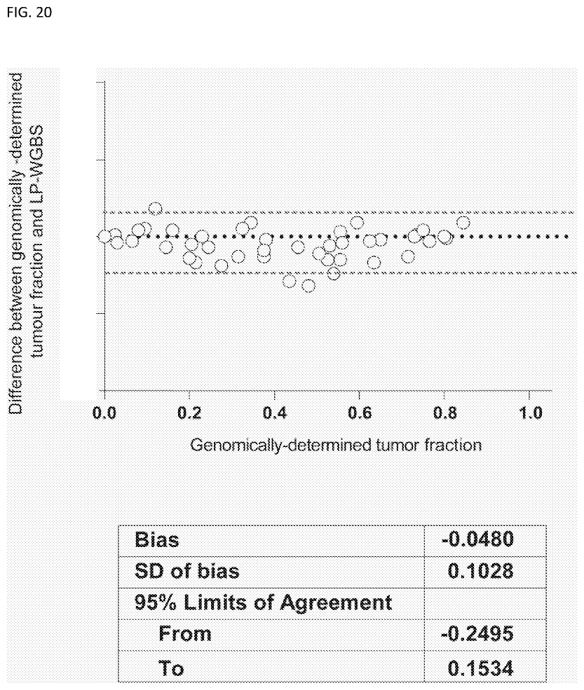

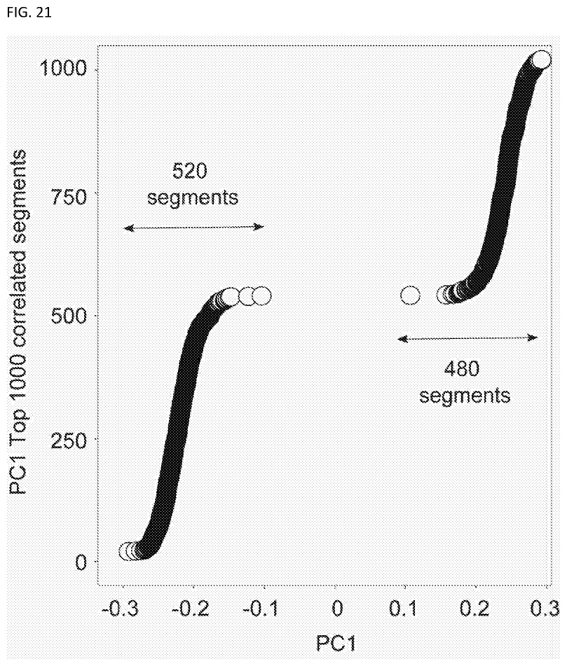

To evaluate the clinical applicability of the findings using LP-WGBS, scaled PC1 values were extracted from LP-WGBS. Applying Bland-Altman analysis, a good agreement was found between LP-WGBS derived tumour fraction estimation and estimates from high-coverage targeted NGS (95% limits of agreement: −0.25 to 0.15, bias: —0.05) introducing the opportunity for scalable and cost-efficient circulating tumour DNA detection and quantitation using LP-WGBS (FIG. 20).

### Methylation Ratio can Serve as a Proxy for Tumour Fraction

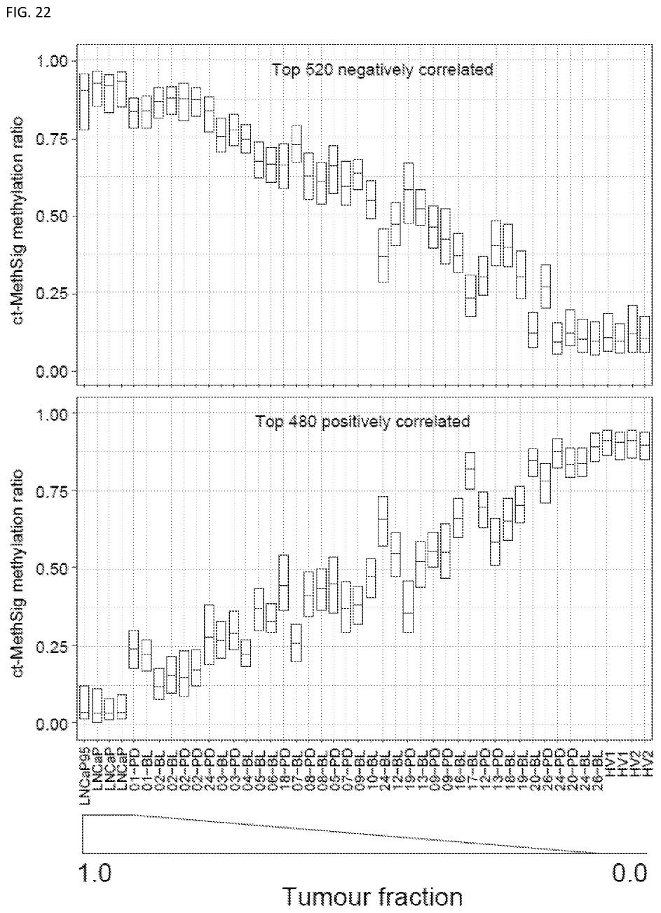

To test features identified by NGS in datasets with fewer data-points, such as methylation arrays, it was hypothesized that the median of the average methylation ratios of the segments that most strongly correlated to the component features could serve as a proxy of tumour fraction. A high correlation (r≥0.93, Pearson correlation) of the average methylation ratio of the segments with genomically-determined tumour fraction was consistently observed in both negatively (i.e. hypermethylated) and positively (i.e. hypomethylated) correlated group when including 10 to 10,000 segments. Also, the intra-sample variance of average methylation ratios of segments in the top correlated segments gradually increased when more segments were included (FIGS. 8 and 9). The 1000 segments that showed the highest correlation with principle component 1 were selected (the selected 1000 segments are referred to herein as circulating tumour methylation signature or ct-MethSig, FIG. 22). These 1000 segments are shown in Tables 1 to 4 above, grouped by their origin (prostate tissue or cancer specific) and their correlation (negative (i.e. hyper-methylated) or positive (i.e. hypo-methylated)).

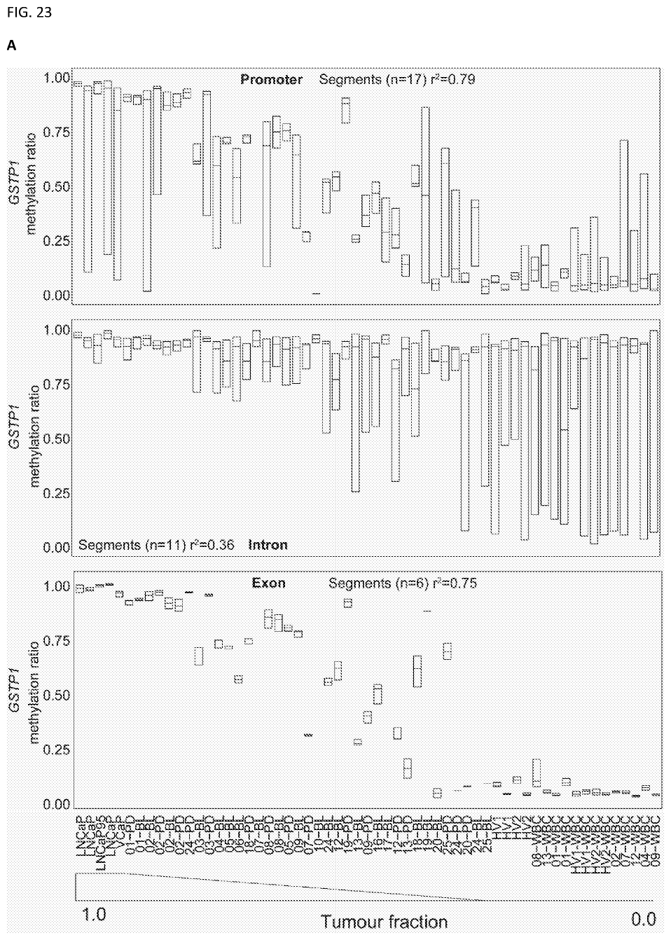

It was confirmed that the median of the average methylation ratios of the selected 1000 segments of the ctMethSig showed a high correlation with tumour fraction (520 segments in negatively (i.e. hypermethylated) correlated regions, hyper-methylated group: r=0.95, P=8.4×10⁻¹⁹; 480 segments in positively (i.e. hypomethylated) correlated regions, hypo-methylated group: r=−0.93, P=3×10⁻¹⁶, Pearson correlation, FIG. 22). It is noted that ct-MethSig did not include genes whose methylation status has been previously reported as diagnostic of prostate cancer such as, GSTP1, APC, and RASSF1 ((Massie, C. E., et al, J Steroid Biochem Mol Biol 166, 1-15) as the segments overlapping with these genes were not as strongly correlated with PC1 value as ct-MethSig (FIG. 23)).

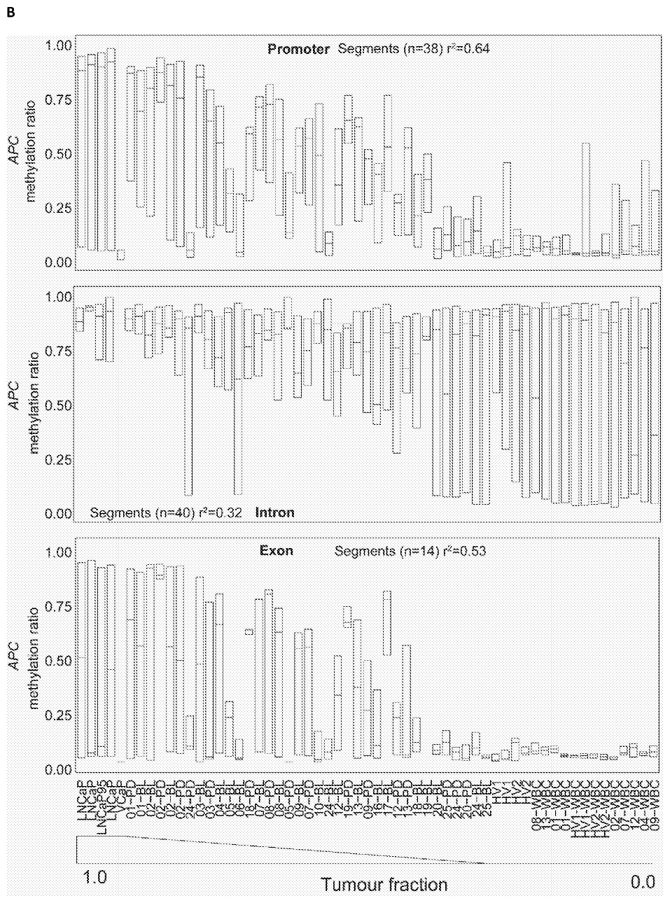

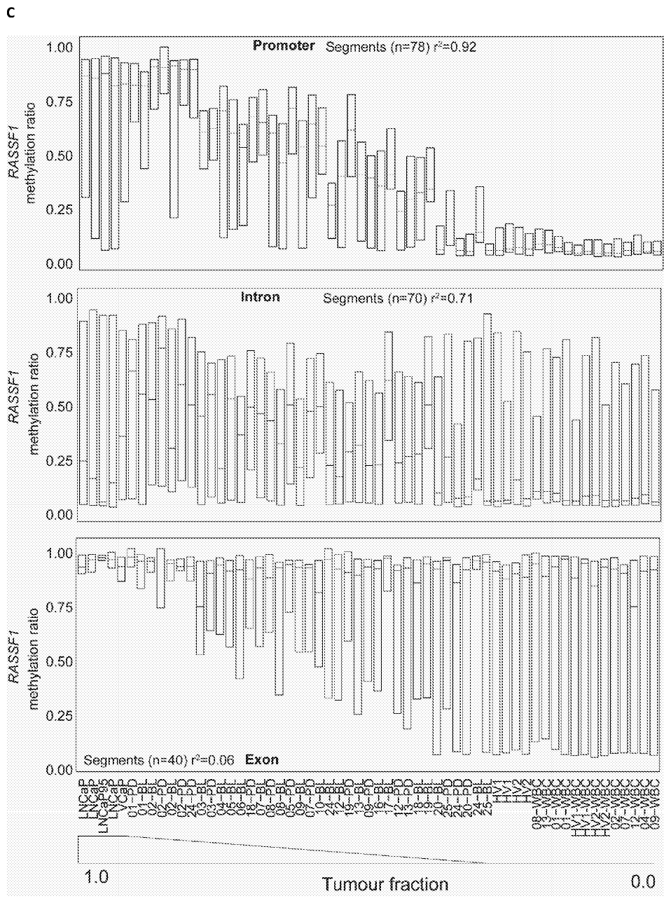

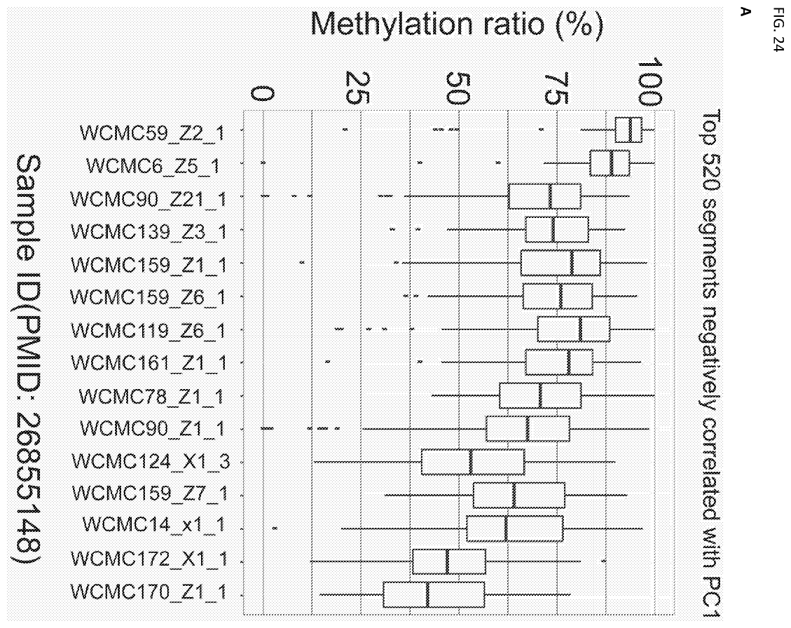

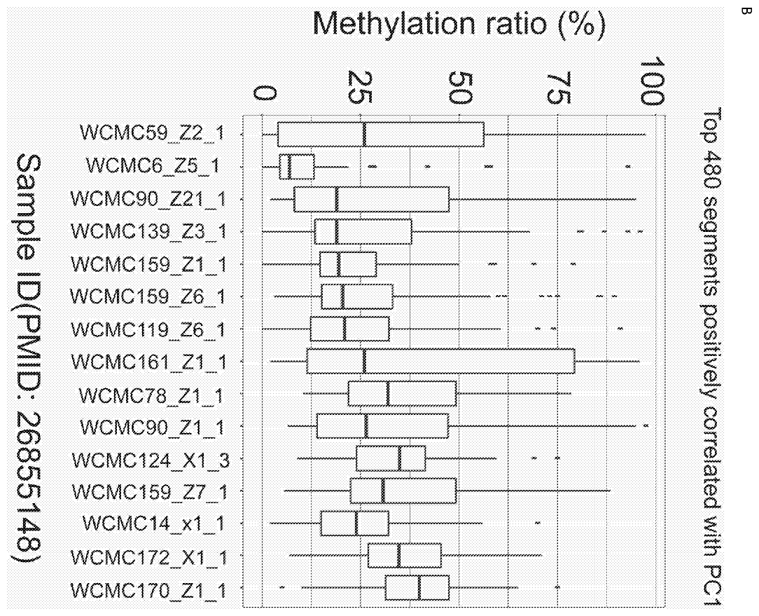

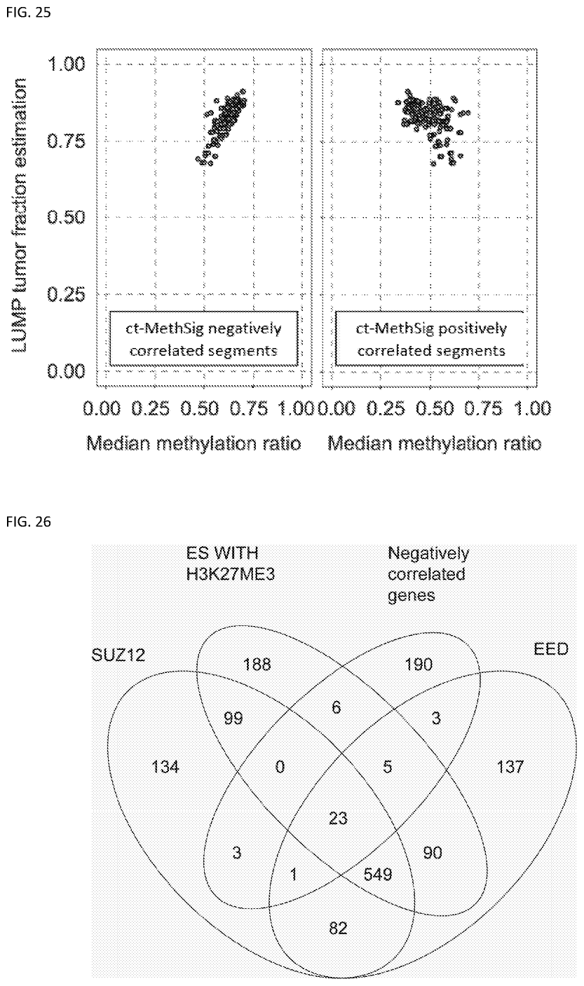

Additionally, the finding that the median of the average methylation ratios of all 1000 segments of the ctMethSig can be used as a proxy for tumour fraction was tested in published tissue data sets and confirmed a high correlation with tumour fraction both in mCRPC (Beltran, H. et al. Nat Med 22, 298-305 (2016)) (hypermethylated group: r=0.92, P<1.5×10⁻⁶; hypomethylated group: r=−0.74, P<1.4 10⁻³, Pearson correlation, FIGS. 24A and 24B), and hormone-sensitive prostate cancer (HSPC) (Fraser, M. et al. Nature 541, 359-364 (2017)) (hypermethylated group: r=0.91, P<10⁻⁶°; hypomethylated group: r=−0.61, P<10⁻¹⁷, Pearson correlation) (FIG. 25).

### Functional Enrichment Identifies Hypermethylation of Polycomb Repressor Complex 2 Targets in Circulating Prostate Cancer DNA

To study the biological processes underlying PC1, gene set enrichment analysis (GSEA) was performed on genes overlapping with ct-MethSig segments (i.e. the DNA segments of the genomic locations shown in Tables 1 to 4 above). Significant enrichment (adjusted P<10⁻⁴) was observed for targets of the polycomb repressor complex 2 (Lee, T. I. et al. Cell 125, 301-313 (2006)) (PRC2 related category in the Molecular Signature Database or MSigDB, FIG. 26). That was of particular interest as a previous mRNA profiling study showed that prostate cancer was distinguished from non-cancer prostate epithelium by down-regulation of genes that are repressed by PRC2 (Yu, J. et al. Cancer Res 67, 10657-10663 (2007)). It was noted that these PRC2 genes were only in the ct-MethSig hypermethylated group, representing an increase in methylation ratio with increasing fraction. Overall, the 520 negatively-correlated segments included 231 genes. Of these, 41 were collectively either components of PRC2-EED (Embryonic Ectoderm Development) (Cao, Q. et al. Nat Commun 5, 3127 (2014)) and SUZ12 (suppressor of zesta 12) (Hojfeldt, J. W. et al. Nat Struct Mol Biol 25, 225-232, (2018)) or H3K27ME3 (tri-methylation of lysine 27 on histone H3 protein subunit) (FIG. 26). A permutation test was performed and the result indicated that PRC2-regulated components were more enriched in ct-MethSig as compared to 1000 randomly selected genomic segments (FIG. 27). The inventors' discovery of hypermethylation in promoters upstream of these genes provides a biological explanation for their down-regulation and introduces a strategy for extending this biological difference to a liquid biopsy application (Beltran, H. et al, Nat Med 22, 298-305 (2016); Yu, J. et al, Cancer Res 67, 10657-10663 (2007)).

### The Circulating Tumour Methylation Signature Comprises Segments Specific to Either Normal or Malignant Prostate Epithelium

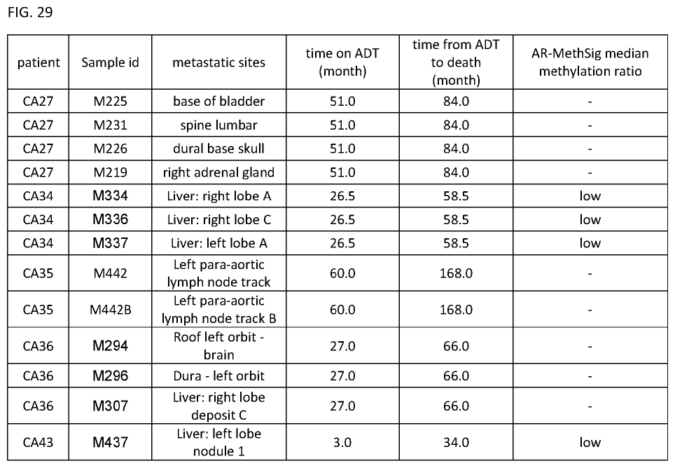

It was postulated that ct-MethSig included components that were specific to either prostate malignant or non-malignant epithelium. The kernel density estimation of the ct-MethSig average methylation ratios in whole genome bisulfite sequencing data derived from the non-malignant prostate epithelium cell line (PrEC) (Pidsley, R. et al. Genome Res 28, 625-638, (2018)) was plotted and it was observed that there was a bimodal distribution (FIG. 28). A Gaussian mixture model was adapted on the average methylation ratios of ct-MethSig segments from the prostate cancer cell line LNCaP and the two healthy volunteer plasma samples and then the fitted Gaussian distribution was used on normal prostate epithelium (PrEC). PrEC segments were identified whose average methylation ratio distribution aligned with either LNCaP or healthy volunteer plasma. It was concluded that the former segments with average methylation ratios in normal prostate epithelium similar to LNCaP were prostate epithelium-specific, while the segments with average methylation ratios similar to healthy volunteer plasma were prostate cancer-specific (FIG. 28). These findings were confirmed by showing that CRPC metastases (bone, bladder, liver and lymph nodes, described further in FIG. 29) included segments attributed to both normal and cancerous prostate epithelium whilst normal prostate (54 year-old male donor, ENCODE donor ID: ENCDO451RUA) included only segments attributable to normal prostate epithelium. As a result, ct-MethSig could be split into two components, circulating cancer-specific and normal prostate-specific signatures. Circulating cancer-specific segments are shown in Tables 2 and 4, and normal prostate-specific segments are shown in Tables 1 and 3, above.

Finally, methylation microarray data from 553 prostate cancers from TCGA and 12 CRPC adenocarcinoma from Beltran et al. (Beltran, H. et al, Nat Med 22, 298-305 (2016)) was used to show that the distribution of ctMethSig segments in localized prostate cancer and CRPC tissue includes both cancer and normal components (FIG. 30).

### Prostate Cancer Detection Using Plasma Methylome

To build a classifier for detection of prostate cancer to accurately categorise prostate cancer subjects and healthy subjects, metastatic prostate cancer plasma samples (N=44) were used as described before (FIGS. 1 and 2) plus fifteen leukocyte samples derived from patients and two healthy volunteer plasma and leukocyte samples. Patient plasma samples were labelled as class A, while the leukocyte and samples collected from healthy volunteer were labelled as class B. The steps to obtain the classifier are shown in FIG. 49.

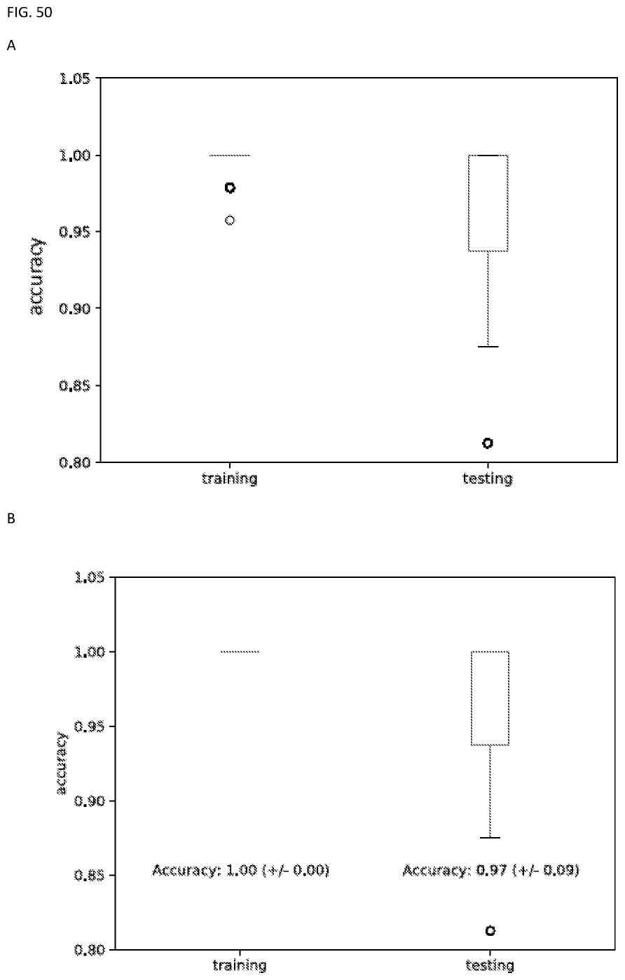

The median of the average methylation ratios of all 1000 segments of ct-MethSig across all samples were used as input for random forest classifier (RFC), a classic machine learning classification method. A RFC model was built on and fitted a number of decision trees each of which categorized a subset of samples to improve the prediction accuracy and control for overfitting. The RFC was run with 1000 times cross-validation to ensure the stability of the model. Briefly, the samples were split into two groups—a training group (plasma DNA containing prostate tumour DNA) and a testing group plasma DNA not containing prostate tumour DNA. The classification model was initially built on the training group and the classifier was tested on the testing group. The model was initially built model selecting 10 trees in one forest, and the result showed 100% accuracy (STD=1%) on training and 95% on testing (STD=11%, FIG. 50A). When the number of trees in the forest were increased to 100, the model performance slightly improved to 100% accuracy (STD=1%) on training and 100% on testing (STD=6%, FIG. 50B).

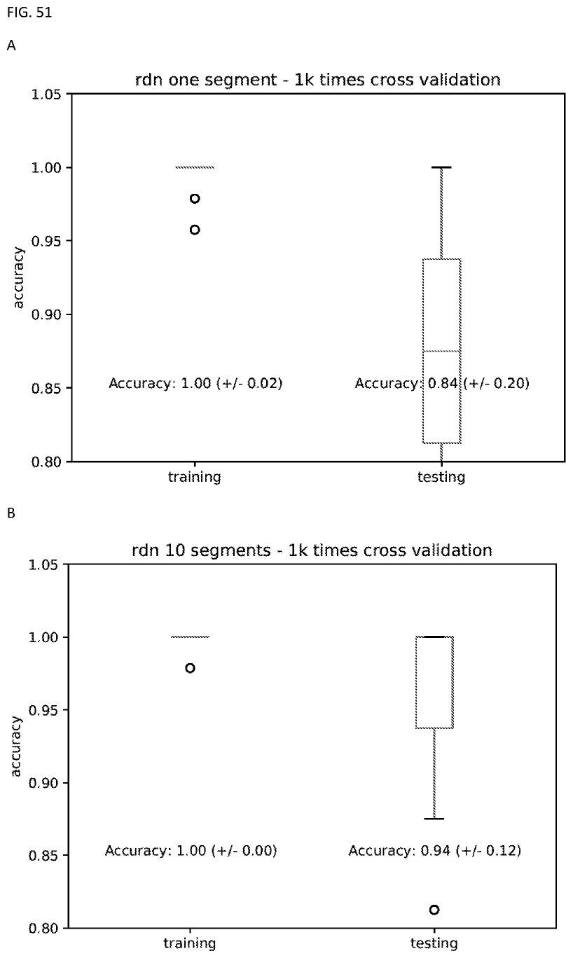

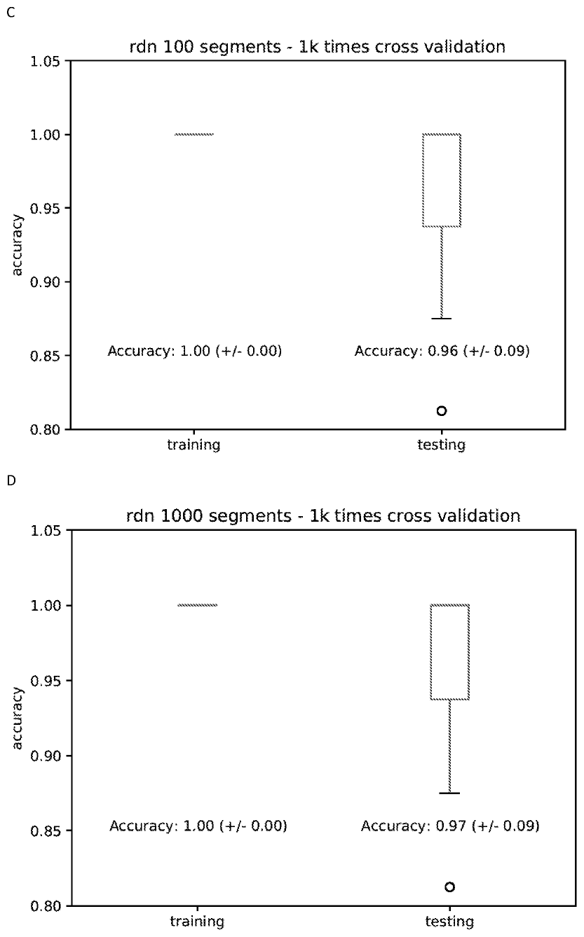

To investigate whether the randomly selected 1, 10 or 100 segments, or all 1000 segments, of ct-MethSig could construct a reliable classifier, a fixed number of segments (1, 10, and 100) were randomly selected, and these segment(s) used to build RFC (n_estimators=100) with 1000-time iteration. The results indicated that using only 1 randomly selected the testing accuracy was 84% (STD %=20%). The testing accuracy gradually improved when more segments were included (FIGS. 51A to D).

In summary, the development of a methylation based classifier was achievable and able to identify plasma samples containing circulating tumour DNA with high accuracy.

### Methylation Signatures Specific to an Individual's Cancer

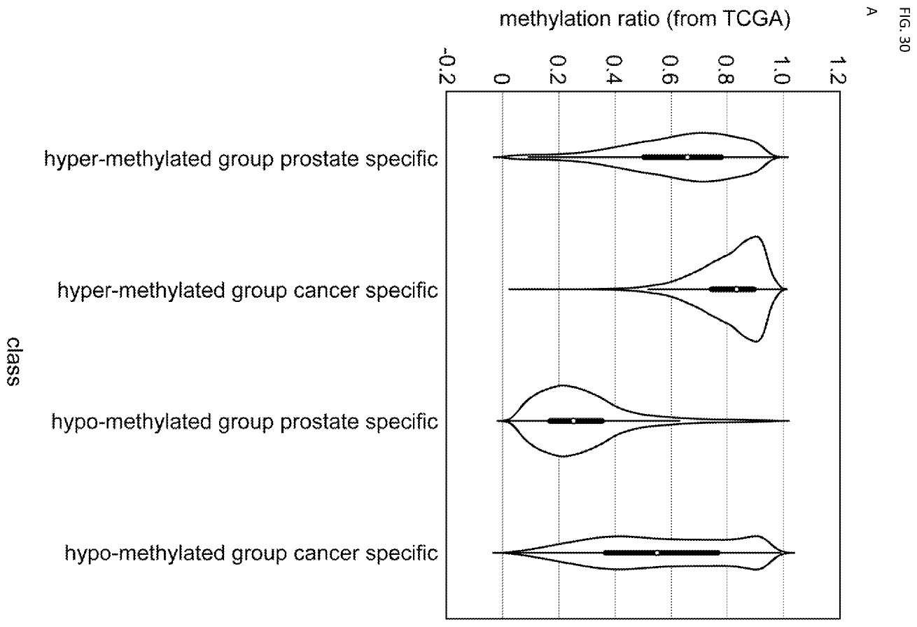

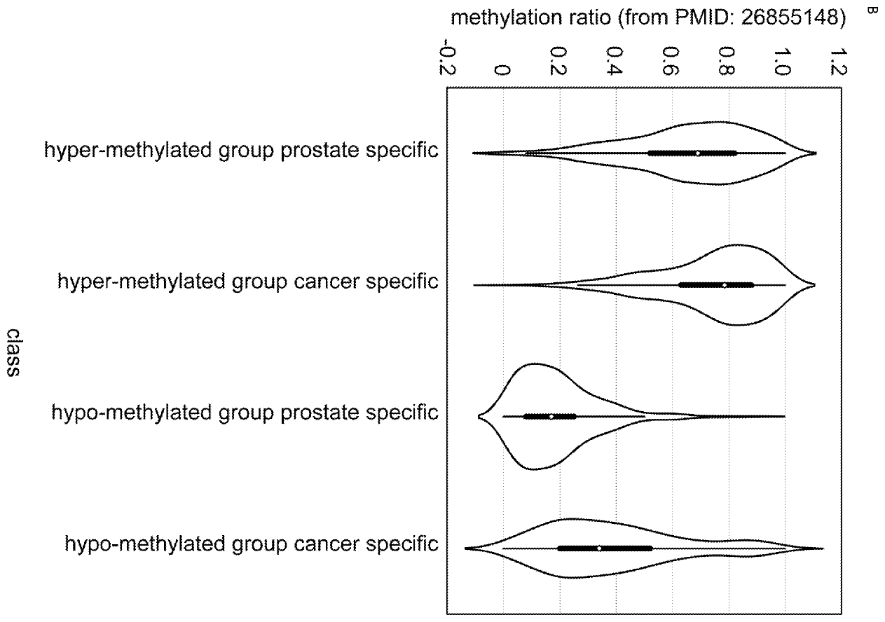

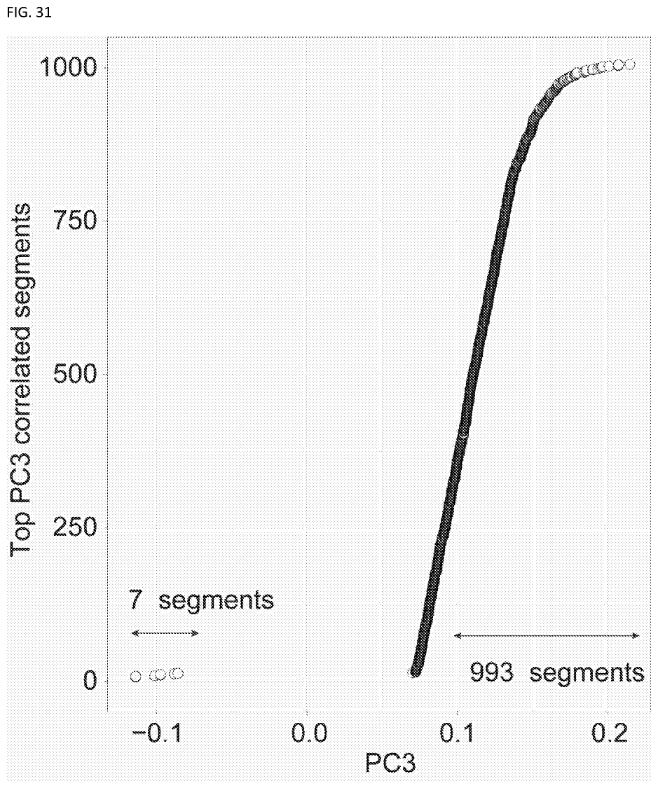

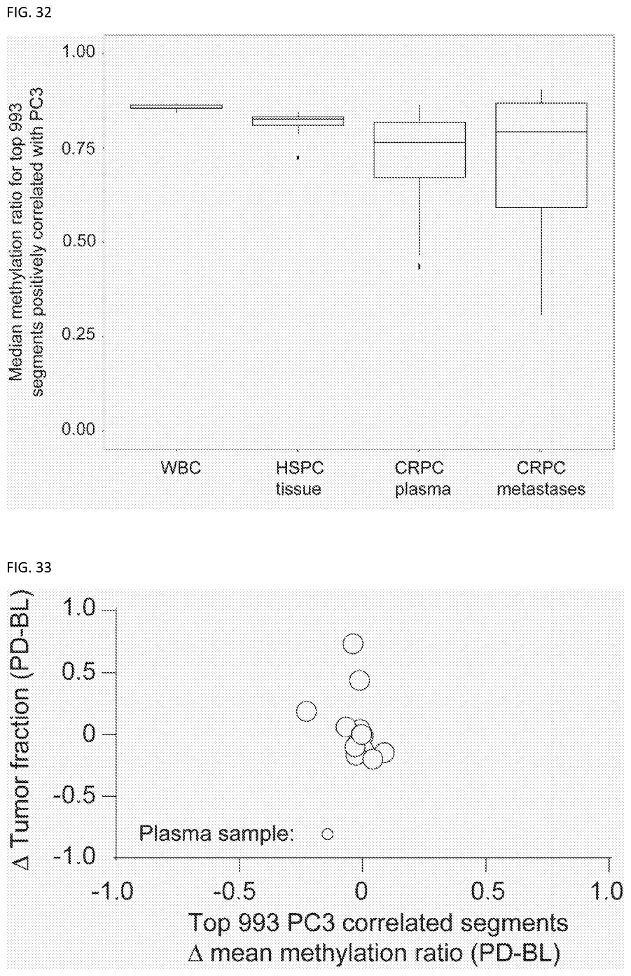

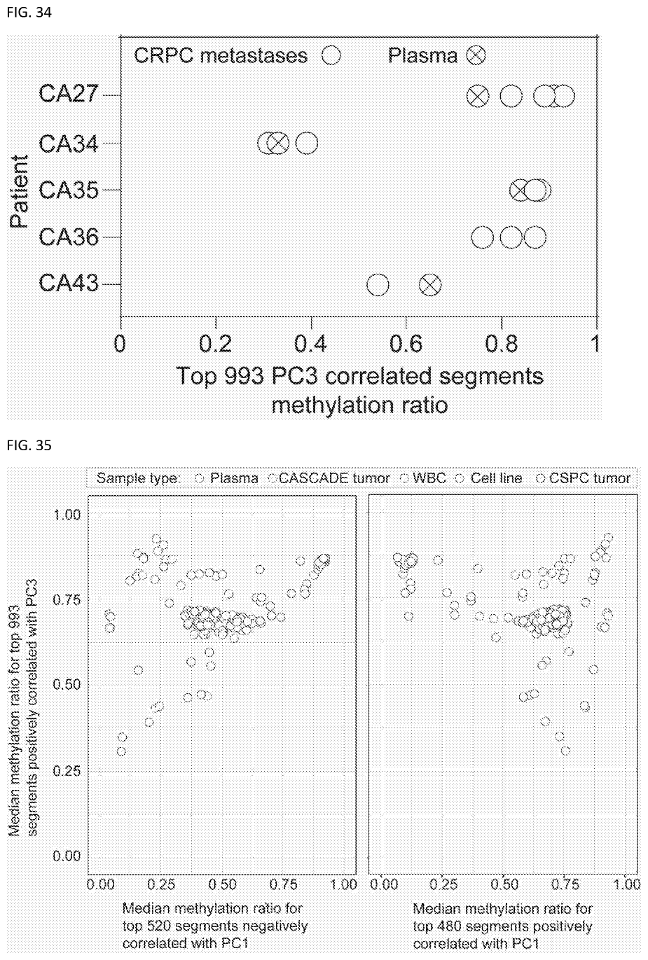

Next plasma DNA methylation changes that could potentially identify distinct methylation subtypes were investigated. The second principal component (PC2) was driven by a single patient (02) and was not investigated further. In the third principal component (PC3) a weak correlation with tumour fraction was found (r=0.01, P=0.96, Pearson correlation) (FIG. 17) and this principal component was investigated in more detail. Similar to the methodology applied to ct-MethSig, the top 1000 segments that were most correlated with this component's values were identified. In contrast to ct-MethSig, these were predominantly positively correlated (i.e. hypomethylated) (FIG. 31). Using the median of the average methylation ratios of all 1000 segments of ctMethSig, it was possible to incorporate array-based methylation data from biopsies from intermediate-risk castration-sensitive prostate cancer (CSPC) (Fraser, M. et al, Nature 541, 359-364 (2017)) and mCRPC (Beltran, H. et al, Nat Med 22, 298-305 (2016)). It was found that the median of the average methylation ratios for the 1000 segments in CRPC plasma and tumour samples presented a greater variability in contrast to CSPC or white blood cells (FIGS. 32 and 35). It was noted that, in contrast to ct-MethSig, a change in tumour fraction before and after treatment did not change the median of the average methylation ratios of the top correlated segments with principal component 3 (FIG. 33). Similarly, inter-patient differences were greater than intra-patient variability in multiple metastases and plasma harvested from the same patient at autopsy (FIGS. 34 and 29).

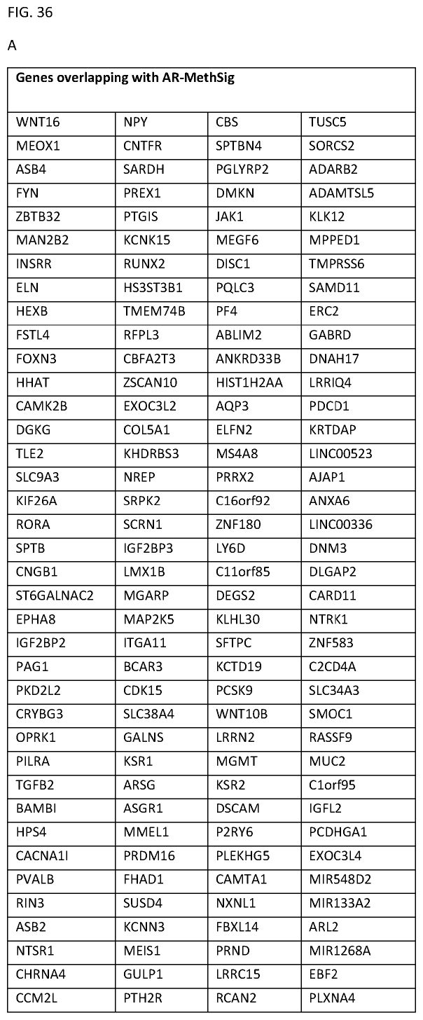

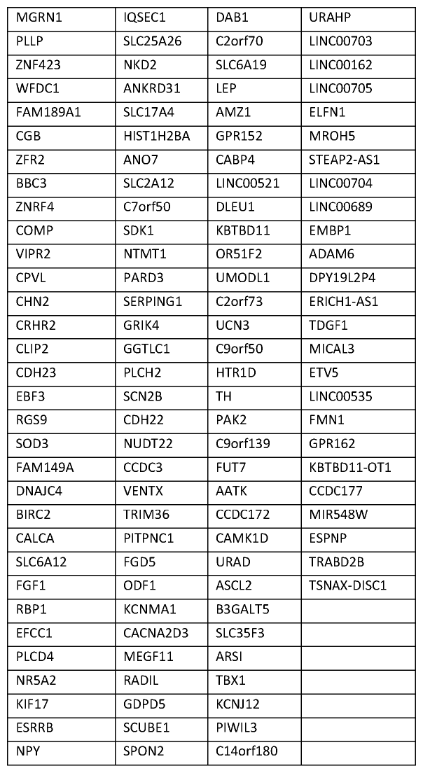

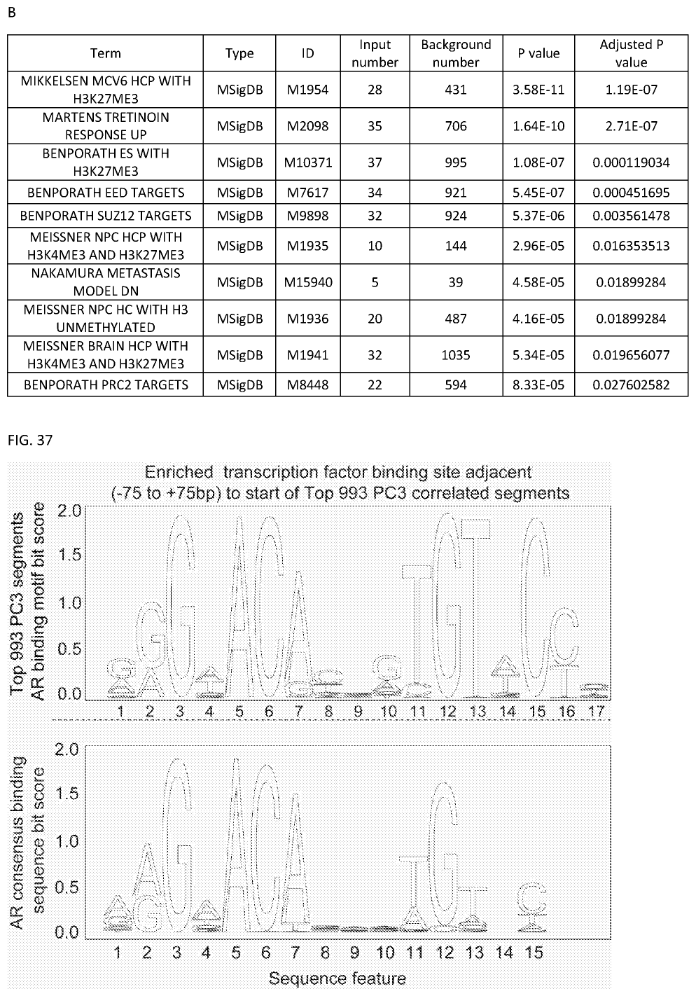

Functional enrichment analysis on the top 1000 segments of PC3 (referred to herein as AR-MethSig and the segments shown in Table 8 above) showed enrichment in histone H3 tri-methylation markers (FIGS. 36A and 36B). It was hypothesized that this methylation signature was regulated by a common transcriptional pathway. Therefore known transcriptional factor binding sites (TFBSs) adjacent to within 75 base-pairs of the start of the top 1000 segments using a protocol described previously were investigated (Zambelli, F., et al, Nucleic Acids Res 41, W535-543 (2013)). Notably, the AR binding motif was the only significantly over-represented binding site (local enrichment P=6×10⁻⁴, global enrichment P=3×10⁻¹⁶; FIGS. 37 and 38). Thus profile was denoted as AR-MethSig.

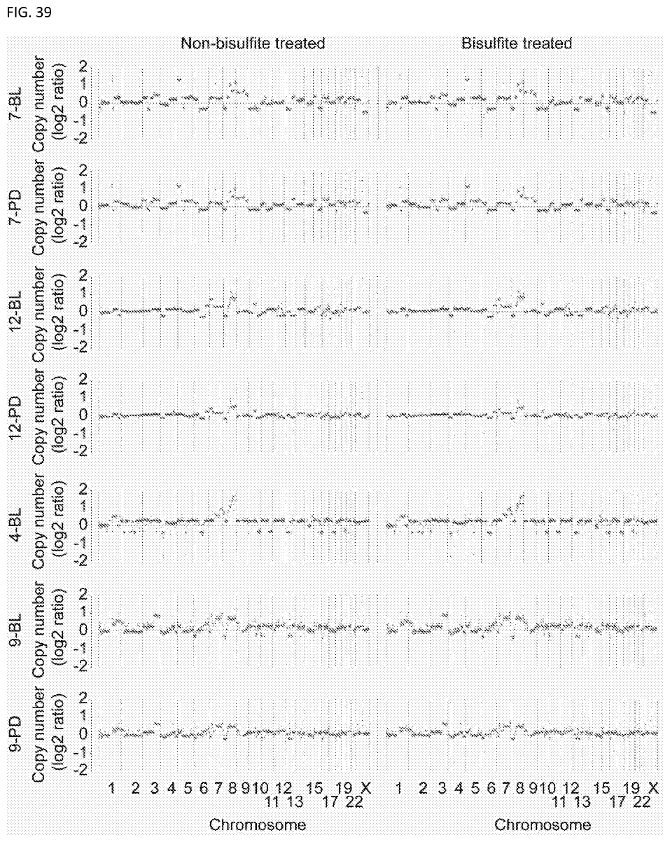

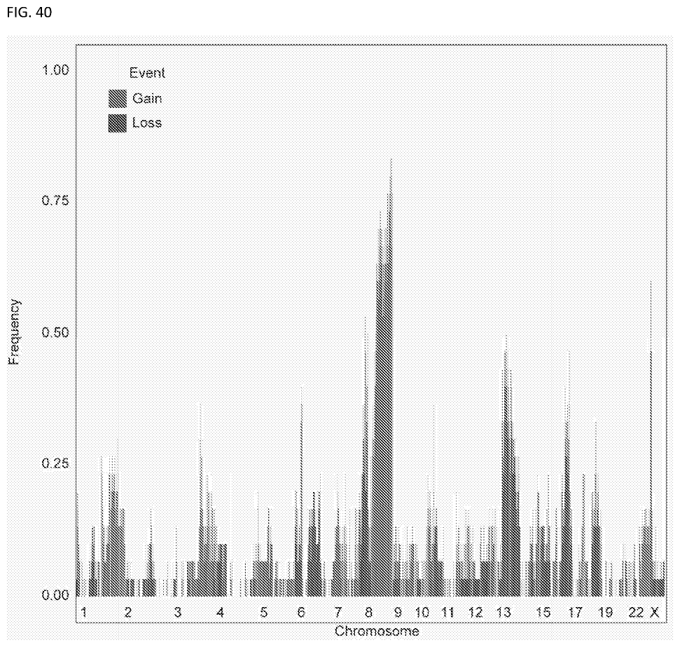

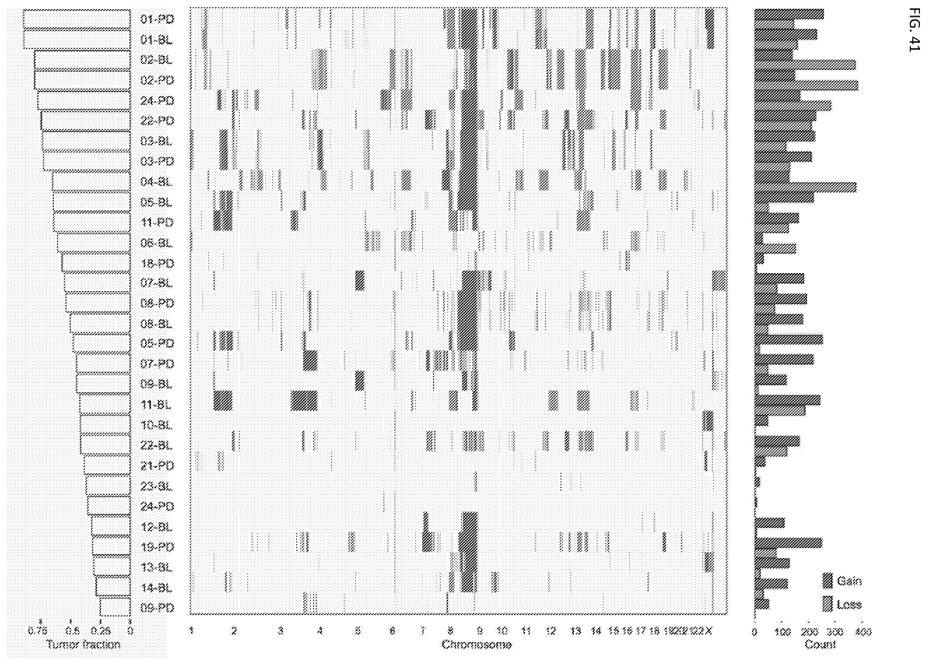

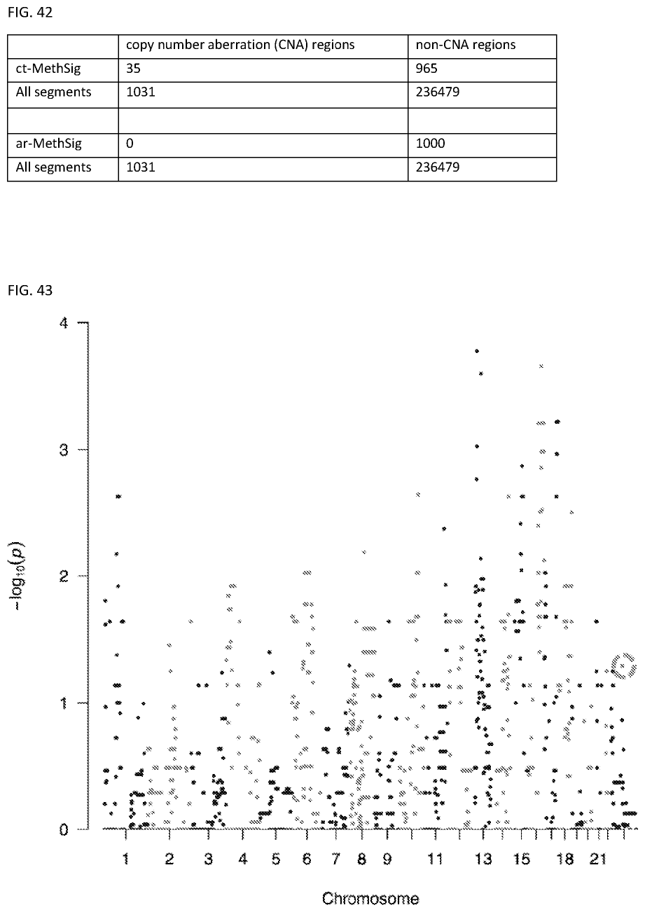

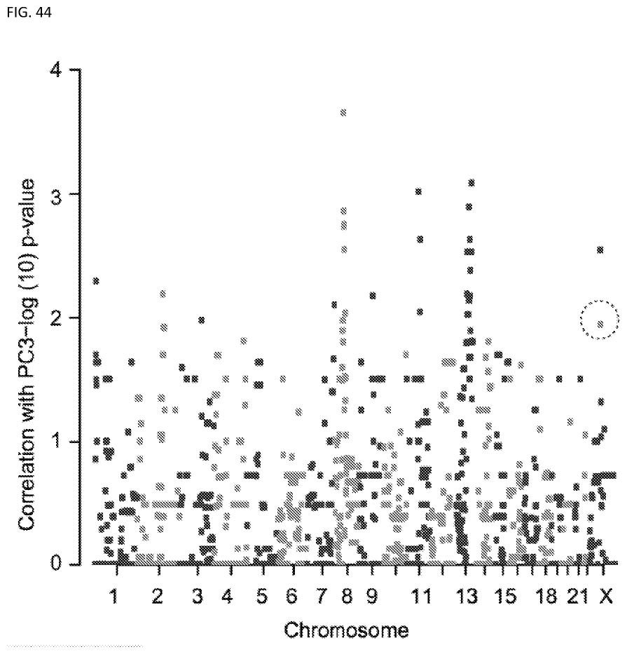

AR-MethSig hypomethylation strongly associates with AR copy number gainNext, genome-wide copy number profiles were extracted from LP-WGS and confirmed high similarity between results from the same sample with and without bisulfite treatment (FIG. 39). Using LP-WGBS from plasma samples, copy number alterations were observed at a frequency consistent with previously described studies of mCRPC tissue or plasma (Annala, M. et al, Cancer Discov 8, 444-457(2018); Robinson, D. et al. Cell 162, 454 (2015)) (for example, most commonly: 8q21-24 gain: prevalence 70%; Xq12 gain: prevalence 60%; 8p21 loss: prevalence ≥50%, FIG. 40). More copy number changes were observed with increasing PC1 values, as an increasing tumour fraction improved copy number detection (FIG. 41). It was then confirmed that ct-MethSig or AR-MethSig were not located more frequently in regions of copy number alterations (FIG. 42). To integrate genomic copy number data with specific methylation signatures, the correlation of the copy number of every segment across the genome and PC1 values were evaluated (Kruskal-Wallis test, FIG. 43). Most notably, a significant difference in principal component 3 (PC3) value distributions was identified when comparing AR copy number gain and AR non-gain samples (P=0.018, Kruskal-Wallis test, FIG. 44).

### The AR-Regulatory Methylation Signature May Identify Distinct Clinical Phenotypes

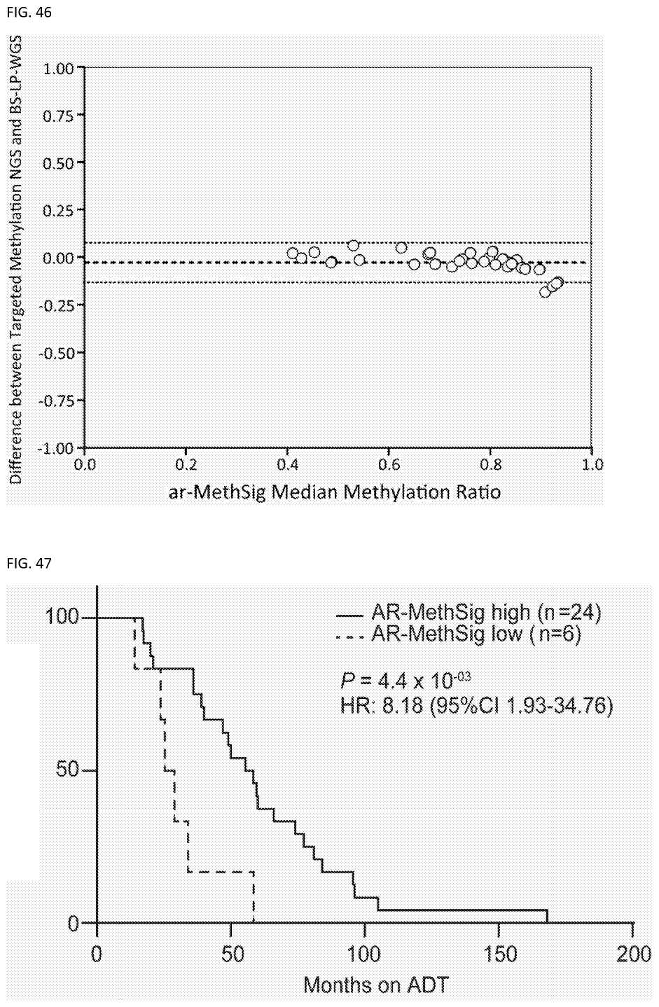

Given the association of PC3 values with AR copy number it was confirmed that patient plasma and tissue samples with AR gain had significantly lower average methylation ratios in the AR-MethSig segments (i.e. average methylation ratios in the AR-MethSig segments indicative of hypomethylation) than AR copy number normal samples (P<0.001 and P=0.023 respectively, Wilcoxon signed-rank test; FIG. 45). A high agreement was found for the median methylation ratio of AR-MethSig extracted from high-coverage targeted NGS and LP-WGBS (95% limits of agreement: −0.136 to 0.076; FIG. 46), again supporting the use of LP-WGBS that is amenable to clinical implementation for methylation-based patient stratification. No hormone-sensitive cancers harboring a low median of the average methylation ratios for the AR-MethSig (i.e. average methylation ratios in the AR-MethSig segments indicative of hypomethylation) were identified, nor did either of the two commonly studied AR-regulated prostate cancer cell lines have a low median of the average methylation ratios for the AR-MethSig (LNCaP and VCaP, FIG. 35). Evaluating the clinical relevance of AR-MethSig was of interest, and a change over time in AR-MethSig median methylation ratio was not observed, so fixed time-points over the disease independent of the time of sampling were chosen: namely time from start of androgen deprivation therapy (ADT) to death. It was observed that AR-MethSig low cancers (i.e. cancers having a median of the average methylation ratios in the AR-MethSig segments indicative of hypomethylation)) had poor clinical prognosis (HR=8.18, 95% CI=1.93-34.76, P=0.0044; Mantel-Cox log-rank test; FIG. 47).

### Discussion

In Example 1, the present inventors performed next-generation sequencing (NGS) on plasma DNA with and without bisulfite treatment from mCRPC patients receiving either abiraterone or enzalutamide in the pre- or post-chemotherapy setting. Using principal component analysis on the mCRPC plasma methylome, the inventors surprisingly found that the main contributor to methylation variance (principal component one, or PC1) was strongly correlated with genomically-determined tumour fraction (r=−0.96; P<10⁻⁸). Further the 1000 top correlated segments of the PC1, “ct-MethSig”, which are presented in Tables 1 to 4 above, revealed that these segments comprised of methylation patterns specific to either prostate cancer or prostate normal epithelium.

The inventors used a custom target-capture approach to define the methylation status of pan-genome CpG islands. By using 100 bp sliding window strategy, the inventors obtained close to 0.5 million methylation segments with 10× coverage in all of the 19 “baseline” plasma DNA samples and used them to construct a principal component analysis. Novel to the inventors' approach was the construction of their model using solely mCRPC plasma DNA that has a variable ratio of normal DNA, primarily arising from white blood cells (Moss, J. et al. Nat Commun 9, 5068, (2018)), and validating the model using tumour DNA that harbors methylation changes that are either prostate epithelium-specific or cancer-specific. The method resulted in the ct-MethSig signature, the segments of which are shown in Tables 1 to 4. These segments can be used as described herein to very accurately determine the level of prostate cancer fraction in a cfDNA sample as shown, for example, in FIG. 50. The inventors have also found that they were able to implement the signature of Tables 1 to 4 in methylation data with variable CpG coverage, including methylation microarrays or reduced representation bisulfite sequencing.

The inventors found that the ct-MethSig did not include genes whose methylation status has been previously reported as diagnostic of prostate cancer such as, GSTP1, APC, and RASSF1 (Massie, C. E, et al, J Steroid Biochem Mol Biol 166, 1-15 (2017)). Although not wishing to be bound by theory, the present inventors being that this finding could be explained by highly variable methylation levels at the genomic segments of the signature in non-cancer plasma DNA compared to cancer plasma DNA.

As well as the signature of Tables 1 to 4 derived from the PC1 found by the present inventors that can be used to determine prostate cancer fraction from a sample, the inventors also surprisingly found a signature that can be used to extract information specific to an individual's cancer. That signature was derived from an orthogonal methylation signature (principal component three (PC3)), and the segments of this signature are defined in Table 8. The inventors surprisingly found that this signature can be used to identify a sub-group of cancers characterized by a more aggressive clinical course and that is enriched for AR copy number gain. In particular, this signature showed enrichment for androgen receptor binding sequences and hypomethylation at putative AR binding sites associated with AR copy number gain. Previous studies have reported worse outcome for patients with AR gain in plasma (Romanel, A. et al. Sci Transl Med 7, 312re310, (2015); Conteduca, V. et al., Ann Oncol 28, 1508-1516, (2017)) and given the high overlap between this genomic lesion and this signature, the inventors believe that this methylation signature identifies the same phenotype. Thus the inventors surprisingly found that a methylation signature can be used to detect a gene abnormality.

Thus, in summary, the present inventors' plasma methylome investigation using their innovative workflow has led to two novel signatures that can be used in methods, kits and uses as defined herein, to very accurately quantitate tumour fraction or identify distinct biologically-relevant subtypes of mCRPC with distinct biological mechanisms and differential clinical outcomes. As such, the signatures can be used for detecting, screening, monitoring, staging, classification, selecting treatment for, ascertaining whether treatment is working in, and/or prognostication of prostate cancer in a sample obtained from a subject, wherein the sample comprises cfDNA.

### Further Aspects of the Invention are Defined in the Following Numbered Clauses

§ 1. A method for detecting, screening, monitoring, staging, classification, selecting treatment for, ascertaining whether treatment is working in, and/or prognostication of prostate cancer in a sample obtained from a subject, wherein the sample comprises circulating free DNA (cfDNA), the method comprising:
characterizing the methylome sequence of a plurality of cfDNA molecules in the sample, wherein the methylome sequence of a cfDNA molecule is the DNA sequence and the methylation profile of the molecule;
determining the average methylation ratio at 10 or more genomic regions, each genomic region being selected from the group consisting of:
   a 100 to 200 bp region comprising or having a genomic location defined in Tables 1 to 4, and
   a 2 to 99 bp region within a genomic location defined in Tables 1 to 4 and comprising at least one CpG locus,
and wherein each of the genomic regions is covered by at least one sequence read of at least one characterized methylome sequence;
calculating a methylation score using the average methylation ratio for each of the genomic regions;
analyzing the methylation score to determine the level of prostate cancer fraction in the cfDNA sample.

§ 2. The method of clause 1, wherein each of the genomic regions is covered by at least one sequence read of at least two characterized methylome sequences, for example at least one sequence read of at least 3, 4, 5, 6, 7, 8, 9, 10, 12, 15, 20, 25, 50, 100, 200, 300, 400, 500, or 1000 characterized methylome sequences.

§ 3. The method of clause 1 or 2, wherein each of the genomic regions is covered by at least 10 sequence reads, for example at least 10, 12, 15, 20, 25, 50, 100, 200, 300, 400, 500, or 1000 sequence reads, and preferably wherein each sequence read or the majority of the sequence reads (for example at least 50%, 60%, 70%, 80% or 90% of the sequence reads) are from different characterized methylome sequences.

§ 4. The method of any one of clauses 1 to 3, wherein calculating a methylation score using the average methylation ratio for each genomic region comprises:
determining the median (or the mean) of the average methylation ratios for all genomic regions for which the average methylation ratio has been determined; or
determining the median (or the mean) of the average methylation ratios for a first group of genomic regions to obtain a first methylation score and/or determining the median (or the mean) of the average methylation ratios for second group of genomic regions to obtain a second methylation score; or
comparing the average methylation ratio at each genomic region to a reference methylation ratio for each genomic region to determine a methylation ratio score for each genomic region.

§ 5. The method of clause 4, wherein the first group of genomic regions are all of the hypermethylated genomic regions for which the average methylation ratio has been determined, and the second group of genomic regions are all of the hypomethylated genomic regions for which the average methylation ratio has been determined.

§ 6. The method of any one of clauses 1 to 5, wherein analyzing the methylation score to determine the level of prostate cancer fraction in the cfDNA sample comprises comparing the methylation score to one or more reference methylation scores, wherein a reference methylation score is a methylation score calculated for the same genomic regions (for example, calculated using the average methylation ratio for the same genomic regions) in one or more of the following a cfDNA sample from a healthy subject, for example a healthy age-matched subject;
a tissue sample from a healthy subject, for example a prostate tissue sample from a healthy subject;
a cancer biopsy sample from a cancer patient, for example a prostate cancer biopsy sample from a prostate cancer patient;
a cancer cell line sample, for example a prostate cancer cell line sample from a prostate cancer cell line;
a sample of white blood cells from a subject, for example the subject or a healthy subject;
a cfDNA sample from a different subject having prostate cancer, preferably wherein the level of prostate cancer fraction in the cfDNA sample from the different subject is known (more preferably multiple cfDNA samples (for example at least 2, 3, 4, 5, 10, 20, 40, 50, 100, 200, 300 or 500 samples) each from a different subject having prostate cancer, wherein preferably the level of prostate cancer fraction in each cfDNA sample from the different subjects is known, and more preferably wherein each cfDNA sample has a different level of prostate cancer fraction);
a characterized methylome sequence of a white blood cell;
a characterized methylome sequence of a prostate cancer cell line;
a characterized methylome sequence of a cancerous prostate cell; and/or
a characterized methylome sequence of a non-cancerous prostate cell.

§ 7. The method of any one of clauses 1 to 6, wherein calculating a methylation score using the average methylation ratio for each genomic region comprises:
determining the median (or the mean) of the average methylation ratios for all genomic regions for which the average methylation ratio has been determined, and
   wherein calculating a reference methylation score using the average methylation ratio for each genomic region comprises:
determining the median (or the mean) of the average methylation ratios for all genomic regions; or
   wherein calculating a methylation score using the average methylation ratio for each genomic region comprises:
determining the median (or the mean) of the average methylation ratios for a first group of genomic regions to obtain a first methylation score and/or determining the median (or the mean) of the average methylation ratios for second group of genomic regions to obtain a second methylation score (for example wherein the first group of genomic regions are all of the hypermethylated genomic regions for which the average methylation ratio has been determined, and the second group of genomic regions are all of the hypomethylated genomic regions for which the average methylation ratio has been determined), and
   wherein calculating a reference methylation score using the average methylation ratio for each genomic region comprises:
determining the median (or the mean) of the average methylation ratios for a first group of genomic regions to obtain a first methylation score and/or determining the median (or the mean) of the average methylation ratios for second group of genomic regions to obtain a second methylation score (for example wherein the first group of genomic regions are all of the hypermethylated genomic regions for which the average methylation ratio has been determined, and the second group of genomic regions are all of the hypomethylated genomic regions for which the average methylation ratio has been determined).

§ 8. The method of any one of clauses 1 to 6, wherein calculating a methylation score using the average methylation ratio for each genomic region comprises comparing the average methylation ratio at each genomic region to a reference methylation ratio for each genomic region to determine a methylation ratio score for each genomic region,

and wherein the reference methylation ratio is the average methylation ratio for the same genomic region in or covered by:
a cfDNA sample from a healthy subject, for example a healthy age-matched subject;
a tissue sample from a healthy subject, for example a prostate tissue sample from a healthy subject;
a cancer biopsy sample from a cancer patient, for example a prostate cancer biopsy sample from a prostate cancer patient;
a cancer cell line sample, for example a prostate cancer cell line sample from a prostate cancer cell line;
a sample of white blood cells from a subject, for example the subject or a healthy subject;
a cfDNA sample from a different subject having prostate cancer, wherein preferably the level of prostate cancer fraction in the cfDNA sample from the different subject is known (more preferably multiple cfDNA samples (more preferably multiple cfDNA samples (for example at least 2, 3, 4, 5, 10, 20, 40, 50, 100, 200, 300 or 500 samples) each from a different subject having prostate cancer, wherein preferably the level of prostate cancer fraction in each cfDNA sample from the different subjects is known, and more preferably wherein each cfDNA sample has a different level of prostate cancer fraction);
a characterized methylome sequence of a white blood cell;
a characterized methylome sequence of a prostate cancer cell line;
a characterized methylome sequence of a cancerous prostate cell; and/or
a characterized methylome sequence of a non-cancerous prostate cell.

§ 9. The method of clause 8, wherein analyzing the methylation score to determine the level of prostate cancer DNA comprises determining the number of methylation ratio scores that are indicative of prostate cancer DNA.

§ 10. The method of any one of clauses 1 to 9, wherein the methylome sequence of a cfDNA molecule is determined by using methylation aware sequencing (for example with bisulfite sequencing), methylation-sensitive restriction enzyme digestion, methylation-specific PCR, methylation-dependent DNA precipitation, methylated DNA binding proteins/peptides, or single molecule sequences without sodium bisulfite treatment.

§ 11. The method of any one of clauses 1 to 10, wherein the methylome sequence of a cfDNA molecule is determined by performing methylation aware sequencing, for example wherein the methylation aware sequencing comprises treating the DNA molecule with sodium bisulfite and performing sequencing of the treated DNA molecule.

§ 12. The method of any one of clauses 1 to 11, comprising determining the average methylation ratio at 25 or more, 50 or more, 100 or more, 150 or more, 200 or more, 300 or more, 400 or more, 500 or more, 600 or more, 700 or more, 800 or more, or 900 or more genomic regions (for example comprising determining the average methylation ratio at 25, 50, 100, 150, 200, 300, 400, 500, 600, 700, 800, 900 or 1000 genomic regions).

§ 13. The method of any one of clauses 1 to 12, wherein the genomic regions are selected from:
a 100 to 150 bp region comprising or having a genomic location defined in Tables 1 to 4, and
a 10 to 99 bp region within a genomic location defined in Tables 1 to 4 and comprising at least one CpG locus.

§ 14. The method of any one of clauses 1 to 13, wherein the genomic regions are selected from:
a 100 to 120 bp region comprising or having a genomic location defined in Tables 1 to 4, and
a 50 to 99 bp region within a genomic location defined in Tables 1 to 4 and comprising at least one CpG locus; or
a 100 to 120 bp region comprising or having a genomic location defined in Table 5, and
a 50 to 99 bp region within a genomic location defined in Table 5 and comprising at least one CpG locus; or
a 100 to 120 bp region comprising or having a genomic location defined in Table 6, and
a 50 to 99 bp region within a genomic location defined in Table 6 and comprising at least one CpG locus; or
a 100 to 120 bp region comprising or having a genomic location defined in Table 7, and
a 50 to 99 bp region within a genomic location defined in Table 7 and comprising at least one CpG locus.

§ 15. The method of any one of clauses 1 to 14, wherein the genomic regions have a 100 bp genomic location defined in any one of Tables 1 to 4, Table 5, Table 6 or Table 7.

§ 16. The method of any one of clauses 1 to 15, comprising characterising the average methylation ratio at 50 or more (for example 50), 100 or more (for example 100), 200 or more (for example 200), 500 or more (for example 500), or 800 or more (for example 800 or 1000) genomic regions, wherein the genomic regions each have a genomic location defined in Tables 1 to 4; or

characterising the average methylation ratio at 10 or more (for example 10), 50 or more (for example 50) or 100 or more (for example 100), wherein each of the genomic regions have a genomic location defined in Table 5; or
characterising the average methylation ratio at 10 or more (for example 10), 50 or more (for example 50) or at 100 or more (for example 100), wherein each of the genomic regions have a genomic location defined in Table 6; or
characterising the average methylation ratio at 10 or more (for example 10), 50 or more (for example 50) or at 100 or more (for example 100), wherein each of the genomic regions have a genomic location defined in Table 7.

§ 17. The method of any one of clauses 1 to 16, wherein at least 25% of the genomic regions are prostate cancer specific genomic regions; or wherein at least 25% of the genomic regions are prostate tissue specific genomic regions.

§ 18. The method of any one of clauses 1 to 17, wherein at least 40% of the genomic regions are prostate cancer specific genomic regions, for example at least 50, 60, 70, 80, 90 or 95% (for example 95, 96, 97, 98, 99 and 100%) of the genomic regions are prostate cancer specific genomic regions; or wherein at least 40% of the genomic regions are prostate tissue specific genomic regions, for example at least 50, 60, 70, 80, 90 or 95% (for example 95, 96, 97, 98, 99 and 100%) of the genomic regions are prostate tissue specific genomic regions.

§ 19. The method of any one of clauses 1 to 18, wherein at least 40% of the genomic regions comprise, have or are within genomic locations defined in Tables 1 and/or 2, or Table 5 or Table 6 or Table 7, for example at least 50, 60, 70, 80, 90 or 95% (for example 95, 96, 97, 98, 99 and 100%) of the genomic regions comprise, have or are within a genomic location defined in Tables 1 and/or 2 or Table 5 or Table 6 or Table 7.

§ 20. The method of any one of clauses 1 to 19, wherein a plurality of cfDNA molecules is at least 10,000, at least 50,000, at least 100,000, at least 500,000, at least 1,000,000, at least 5,000,000, at least 10,000,000, or at least 100,000,000 cfDNA molecules.

§ 21. The method of any one of clauses 1 to 20, wherein the prostate cancer is acinar adenocarcinoma prostate cancer, ductal adenocarcinoma prostate cancer, transitional cell cancer of the prostate, squamous cell cancer of the prostate, or small cell prostate cancer (for example wherein the prostate cancer is acinar adenocarcinoma prostate cancer or ductal adenocarcinoma prostate cancer).

§ 22 The method of any one of clauses 1 to 21 wherein the prostate cancer is castration resistant prostate cancer and/or is metastatic prostate cancer.

§ 23. The method of any one of clauses 1 to 22, wherein the sample comprising cfDNA is a blood or plasma sample.

§ 24. The method of any one of clauses 1 to 23, further comprising measuring the level of prostate-specific antigen (PSA) in a sample of blood from the subject, and determining if the subject has an abnormal level of PSA in the blood (for example a level of PSA in the blood of at least 4.0 ng/mL or, if the subject has had a previous PSA test, an increased level of PSA compared to the previous test).

§ 25. The method of clause 24, wherein the subject has an abnormal level of PSA in the blood (for example a level of PSA in the blood of at least 4.0 ng/mL or, if the subject has had a previous PSA test, an increased level of PSA compared to the previous test); or wherein the subject has a normal level of PSA in the blood (for example a level of PSA in the blood of 4.0 ng/mL or less).

§ 26. The method of any one of clauses 1 to 25, further comprising repeating the method on a second sample obtained from the subject after the subject has undergone a treatment for prostate cancer, wherein the second sample comprises cfDNA, and comparing the level of prostate cancer fraction in the two samples.

§ 27. The method of any one of clauses 1 to 26 for screening and/or prognostication of prostate cancer, wherein prostate cancer is predicted when a level of prostate cancer is determined, for example a detectable level of prostate cancer, for example a percentage level of prostate cancer fraction of at least 0.01%.

§ 28. The method of any one of clauses 1 to 27, for detecting, screening and/or prognostication of metastatic prostate cancer, wherein metastatic prostate cancer is predicted when a level of prostate cancer is determined, for example a detectable level of prostate cancer, for example a percentage level of prostate cancer fraction of at least 0.01%.

§ 29. The method of any one of clauses 1 to 28, for detecting, screening and/or prognostication of prostate cancer, wherein metastatic prostate cancer with a poor prognosis is predicted when a level of prostate cancer is determined, for example a detectable level of prostate cancer, for example a percentage level of prostate cancer fraction of at least 0.01%.

§ 30. An in-vitro diagnostic kit for use in the detecting, screening, monitoring, staging, classification, selecting treatment for, ascertaining whether treatment is working in, and/or prognostication of prostate cancer, comprising one or more reagents for detecting the presence or absence of at least 10 DNA molecules having a DNA sequence corresponding to all or part of a genomic location comprising at least one CpG locus defined in Tables 1 to 4, or comprising at least one CpG locus defined in Table 5, or comprising at least one CpG locus defined in Table 6, or comprising at least one CpG locus defined in Table 7.

§ 31. The kit as defined in clause 30, wherein the kit comprises one or more reagents for detecting the presence or absence of at least 15, 20, 30, 40, 50, 75, 100, 150, 200, 250, 300, 400, 500, 600, 700, 800, or 900 DNA molecules (for example 15, 20, 30, 40, 50, 75, 100, 150, 200, 250, 300, 400, 500, 600, 700, 800, 900 or 1000 DNA molecules) having a DNA sequence corresponding to all or part of a genomic location comprising at least one CpG locus defined in Tables 1 to 4.

§ 32. The kit as defined in clause 30 or 31, wherein the kit comprises oligonucleotides for specifically hybridizing to at least a section of the at least 10 DNA molecules (for example, at least 15, 20, 30, 40, 50, 75, 100, 150, 200, 250, 300, 400, 500, 600, 700, 800, or 900 DNA molecules) having a DNA sequence corresponding to all or part of a genomic location defined in Tables 1 to 4.

§ 33. The kit of any one of clauses 30 to 32, wherein at least one of the oligonucleotides for specifically hybridizing to at least a section of a DNA molecule is an amplification primer, for example each of the oligonucleotides for specifically hybridizing to at least a section of a DNA molecule is an amplification primer.

§ 34. A computer product comprising a non-transitory computer readable medium storing a plurality of instructions that when executed control a computer system to perform the method of any one of clauses 1 to 29.

§ 35. A computer-executable software for performing the method of any one of clauses 1 to 29.

§ 36. The kit of any one of clauses 30 to 33, wherein the kit comprises instructions for use which define how to determine the level of prostate cancer fraction in a sample comprising cfDNA from a subject, and/or comprises a computer product as defined in clause 34, and/or a computer-executable software as defined in clause 35.

§ 37. A computer-implemented method for detecting, screening, monitoring, staging, classification, selecting treatment for, ascertaining whether treatment is working in, and/or prognostication of prostate cancer in a sample obtained from a subject, wherein the sample comprises circulating free DNA (cfDNA), the method comprising:
receiving a data set in a computer comprising a processor and a computer readable medium, wherein the data set comprises the methylome sequence of a plurality of cfDNA molecules in the sample;
and wherein the computer readable medium comprises instructions that, when executed by the processor, causes the computer to perform a method of any one of clauses 1 to 29 (for example causes the computer to perform a method comprising the following steps:
characterize the methylome sequence of a plurality of cfDNA molecules in the sample, wherein the methylome sequence of a cfDNA molecule is the DNA sequence and the methylation profile of the molecule;
determine the average methylation ratio at 10 or more genomic regions, each genomic region being selected from the group consisting of:
a 100 to 200 bp region comprising or having a genomic location defined in Tables 1 to 4, and
a 2 to 99 bp region within a genomic location defined in Tables 1 to 4 and comprising at least one CpG locus,
and wherein each of the genomic regions is covered by at least one sequence read of at least one characterized methylome sequence;
calculate a methylation score using the average methylation ratio for each of the genomic regions;
analyze the methylation score to determine the level of prostate cancer fraction in the cfDNA sample).

§ 38. A computer-implemented method for classifying a prostate cancer patient into one or more of a plurality of treatment categories, the method comprising determining the level of prostate cancer DNA in a sample obtained from a subject, wherein the sample comprises circulating free DNA (cfDNA), the method comprising:
receiving a data set in a computer comprising a processor and a computer readable medium, wherein the data set comprises the methylome sequence of a plurality of cfDNA molecules in a sample obtained from a subject, wherein the sample comprises cfDNA;
and wherein the computer readable medium comprises instructions that, when executed by the processors, causes the computer to perform a method of any one of clauses 1 to 29 (for example causes the computer to perform a method comprising the following steps:
characterize the methylome sequence of a plurality of cfDNA molecules in the sample, wherein the methylome sequence of a cfDNA molecule is the DNA sequence and the methylation profile of the molecule;
determine the average methylation ratio at 10 or more genomic regions, each genomic region being selected from the group consisting of:
   a 100 to 200 bp region comprising or having a genomic location defined in Tables 1 to 4, and
   a 2 to 99 bp region within a genomic location defined in Tables 1 to 4 and comprising at least one CpG locus,
and wherein each of the genomic regions is covered by at least one sequence read of at least one characterized methylome sequence;
calculate a methylation score using the average methylation ratio for each genomic region;
analyse the methylation score to determine the level of prostate cancer fraction in the cfDNA sample).

§ 39. The method of any one of clauses 1 to 29, 37 or 38 further comprising treating the subject for prostate cancer using a therapeutic agent for the treatment of prostate cancer;

or ceasing or altering treatment with a therapeutic agent for the treatment of prostate cancer; or initiating a non-therapeutic agent treatment for prostate cancer (for example initiation of treatment by surgery or radiation).

§ 40. A method for treating prostate cancer in a subject comprising the method of one of clauses 1 to 29, 37 or 38 and further comprising treating the subject using a therapeutic agent for the treatment of prostate cancer, surgery, and/or radiotherapy; or a method for treating prostate cancer in a subject, comprising administering to the subject an effective amount of a therapeutic agent for the treatment of prostate cancer after the subject has been determined to have prostate cancer based on a method as defined in one of clauses 1 to 29, 37 or 38.

§ 41. The method of clause 40, wherein the method of clause 1 to 29, 37 or 38 is performed before and/or after treating the subject.

§ 42. A method of any one of clauses 39 to 41, comprising performing the method of clause 1 to 29, 37 or 38 before treating the subject, and subsequently repeating the method of clause 1 to 29, 37 or 38 after the treatment, for example at least 1 week, at least 2 weeks, at least 3 weeks, at least 4 weeks, at least 1 month, at least 2 months, at least 3 months, at least 6 months, at least 9 months, at least 12 months, at least 24 months or at least 36 months after treating the subject.

§ 43. The method of clause 42, wherein the method comprises continuing to treat the subject with the therapeutic agent for the treatment of prostate cancer if the level of prostate cancer fraction is substantially the same in the initial and subsequent method or lower in the subsequent method than in the initial method.

§ 44. The method of clause 42 or 43, wherein the method comprises
ceasing or altering treatment with the therapeutic agent for the treatment of prostate cancer; and/or
initiating treatment with a second therapeutic agent for the treatment of prostate cancer; and/or
initiating a non-therapeutic agent treatment (e.g., surgery or radiation),
   if the level of prostate cancer fraction is substantially the same in the initial and subsequent method or higher in the subsequent method than in the initial method.

§ 45. A method of treating a subject in need of treatment with a therapeutic agent for the treatment of prostate cancer, comprising
i) performing the method of any one of clauses 1 to 29, 37 or 38 to determine the level of prostate cancer fraction in the subject;
ii) administering a therapeutic agent for the treatment of prostate cancer if the subject has a level of prostate cancer fraction (for example 0.01% or more prostate cancer fraction).

§ 46. A therapeutic agent for the treatment of prostate cancer for use in the treatment of prostate cancer, whereby
i) the method of any one of clauses 1 to 29, 37 or 38 is performed to determine the level of prostate cancer prostate cancer DNA in a subject;
ii) the therapeutic agent is administered if the subject has a level of prostate cancer.

§ 47. A method as defined in clause 40 to 45, or a therapeutic agent for the treatment of prostate cancer for use as defined in clause 46, wherein a second therapeutic agent for the treatment of prostate cancer is administered if the subject has a level of prostate cancer DNA (for example a detectable level of prostate cancer DNA, for example 0.01% or more prostate cancer DNA).

§ 48. The method of clause 45, or a therapeutic agent for the treatment of prostate cancer for use as defined in clause 46, wherein
(iii) at least 1 week, at least 2 weeks, at least 3 weeks, at least 4 weeks, at least 1 month, at least 2 months, at least 3 months, at least 6 months, at least 9 months, at least 12 months, at least 24 months, or at least 36 months, after the administration of the therapeutic agent, a further sample comprising cfDNA is obtained from the subject, and the method of any one of clauses 1 to 29, 37 or 38 is performed to determine the level of prostate cancer DNA in the further sample.

§ 49. A method of determining one or more suitable therapeutic agents for the treatment of prostate cancer for a subject having prostate cancer comprising
performing the method of any one of clauses 1 to 29, 37 or 38;
determining the one or more suitable therapeutic agents for the treatment of prostate cancer by reference to the level of prostate cancer, whereby one therapeutic agent is suitable for a subject with no level of prostate cancer fraction (for example an undetectable level of prostate cancer fraction) or a level of prostate cancer fraction of less than 0.01%, and two or more therapeutic agents are suitable for a subject with a level of prostate cancer DNA (for example a percentage level of prostate cancer fraction of at least 0.01%);
or whereby a therapeutic agent selected from a first list of therapeutic agents is suitable for a subject with no level of prostate cancer DNA (for example an undetectable level of prostate cancer DNA) or a level of prostate cancer DNA of less than 0.01%, and a therapeutic agent from a second list of therapeutic agents, or two or more therapeutic agents from the first list, is suitable for a subject with a level of prostate cancer DNA (for example a percentage level of prostate cancer fraction of at least 0.01%).

§ 50. A method of determining a suitable treatment regimen for a subject having prostate cancer comprising
performing the method of any one of clauses 1 to 29, 37 or 38;
determining the treatment regimen by reference to the level of prostate cancer fraction, whereby a standard treatment is suitable for a subject having no level of prostate cancer fraction (for example an undetectable level of prostate cancer fraction) or a percentage level of prostate cancer fraction of less than 0.01%, and a non-standard treatment is suitable for a subject with a level of prostate cancer fraction (for example a detectable level of prostate cancer fraction) or a percentage level of prostate cancer fraction of at least 0.01%.

§ 51. The method as defined in clause 50, wherein the standard treatment is a treatment with a therapeutic agent for the treatment of prostate cancer, and a non-standard treatment is a treatment with two or more therapeutic agents for the treatment of prostate cancer;

or wherein the standard treatment is a treatment with a hormonal agent for the treatment of prostate cancer, and a non-standard treatment is a treatment with a hormonal agent for the treatment of prostate cancer, and a chemotherapeutic agent for the treatment of prostate cancer and/or a immunotherapy treatment of prostate cancer and/or a targeted treatment of prostate cancer and/or a biologic agent treatment of prostate cancer.

§ 52. A computerized method and/or computer-assisted method for determining one or more suitable therapeutic agents for the treatment of prostate cancer in a subject having prostate cancer, the method comprising performing the steps of clause 49; or a computerized method and/or computer-assisted method for determining a suitable treatment regimen for a subject having prostate cancer, the method comprising performing the steps of clause 50 or clause 51.

§ 53. A method or therapeutic agent as defined in any one of clauses 39 to 52, wherein the therapeutic agent for the treatment of prostate cancer is selected from the group consisting of a hormonal agent, a targeted agent, a biologic agent, an immunotherapy agent, a chemotherapy agent;

for example:
a hormonal agent selected from LHRH agonists (for example leuprolide, goserelin, triptorelin, or histrelin), LHRH antagonists (for example degarelix), androgen blockers (for example abiraterone or ketoconazole), anti-androgens (for example flutamide, bicalutamide, nilutamide, enzalutamide, apalutamide or darolutamide), estrogens, and steroids (for example prednisone or dexamethasone);
a targeted agent selected from poly(ADP-ribose) polymerase (PARP) inhibitor (for example olaparib, rucaparib, niraparib or talazoparib), a epidermal growth factor receptor (EGFR) inhibitor (for example gefitinib, erlotinib, afatinib, brigatinib, icotinib, cetuximab, or osimertinib, adavosertib, lapatinib), and a tyrosine kinase inhibitor (for example imatinib, gefitinib, erlotinib, sunitinib);
a biologic agent selected from monoclonal antibodies (for example pertuzumab, trastuzumab and Solitomab), hormones (for example a hormonal agent selected from LHRH agonists (for example leuprolide, goserelin, triptorelin, or histrelin), LHRH antagonists (for example degarelix), androgen blockers (for example abiraterone or ketoconazole), anti-androgens (for example flutamide, bicalutamide, nilutamide, enzalutamide, apalutamide or darolutamide), and estrogens), interferons (for example interferons-α, -β, -γ), and interleukin-based products (for example interleukin-2);
an immunotherapy agent selected from a cancer vaccine (for example sipuleucel-T), T-cell therapy, monoclonal antibody therapy, immune checkpoint therapy (for example a PD-1 inhibitor (e.g pembrolizumab, nivolumab, cemiplimab spartalizumab), a PD-L1 inhibitor (e.g. atezolizumab, avelumab or durvalumab), or a CTLA-4 (e.g. ipilimumab)), and non-specific immunotherapies (for example interferons and inerleukins); or
a chemotherapy agent selected from docetaxel, cabazitaxel, and c-Met inhibitors (for example cabozantinib).

§ 54. A method or therapeutic agent as defined in any one of clauses 39 to 52, wherein the therapeutic agent for the treatment of prostate cancer is a hormonal agent and optionally a chemotherapy agent and/or optionally a further hormonal agent and/or optionally a targeted agent and/or optionally a radionuclide agent and/or an immunotherapy agent (for example a LHRH agonist (for example leuprolide, goserelin, triptorelin, or histrelin) or a LHRH antagonist (for example degarelix), and optionally a chemotherapy agent (for example docetaxel, cabazitaxel, carboplatin) and/or optionally a further hormonal treatment (for example enzalutamide, abiraterone, darolutamide) and/or optionally a radionuclide agent (Radium223, PSMA-labelled radionuclide) and/or optionally a PARP inhibitor (for example olaparib, rucaparib, niraparib or talazoparib) and/or an immunotherapy agent (for example nivolumab, pembroluzimab, ipilumimab, durvalumab)).

§ 55. A method for determining a solid cancer circulating free DNA (cfDNA) methylome signature for use in detecting, screening, monitoring, staging, classification, selecting treatment for, ascertaining whether treatment is working in, prognostication and/or treatment of the solid cancer, the method comprising:
(i) characterizing the methylome sequence of a plurality of cfDNA molecules in a first sample comprising cfDNA from a subject known to have the solid cancer, wherein the methylome sequence of a cfDNA molecule is the DNA sequence and the methylation profile of the molecule;
(ii) determining the respective number of characterised cfDNA molecules corresponding to a CpG locus or a genomic region of 2 to 10,000 bp (preferably 2 to 200 bp) in the first sample by aligning the methylome sequences;
(iii) determining the methylation ratio of each CpG locus and/or average methylation ratio of each genomic region of 2 to 10,000 bp (preferably 2 to 200 bp) in the first sample;
repeating steps (i) to (iii) for one or more further samples comprising cfDNA each from subjects known to have the solid cancer;
performing a variance analysis of all or a selection of the methylation ratios of the CpG loci and/or all or a selection of average methylation ratios of the genomic regions of the samples;
selecting a group of CpG loci and/or genomic regions associated with a feature of the samples; and
selecting CpG loci and/or genomic regions in the group to provide the cfDNA methylome signature.

§ 56. The method of clause 55, wherein the method further comprises aligning the methylome sequences for the first sample with a reference genome for the subject; and aligning the methylome sequences for each of the one or more further samples with the same reference genome.

§ 57. The method of clause 55 or 56, wherein the reference genome is selected from hg38, hg19, hg18, hg17 and hg16.

§ 58. The method of any one of clauses 55 to 57, comprising selecting at least 25 CpG loci (for example at least 50, at least 75, at least 100, at least 200, at least 300, at least 400, at least 500, at least 600, at least 700, at least 800, at least 900, at least 1000 or at least 10,000) and/or at least 25 genomic regions (for example at least 50, at least 75, at least 100, at least 200, at least 300, at least 400, at least 500, at least 600, at least 700, at least 800, at least 900, at least 1000 or at least 10,000) CpG loci and/or genomic regions in the group to provide the cfDNA methylome signature.

§ 59. The method of any one of clauses 55 to 58, wherein the variance analysis performed is a dimensionality reduction.

§ 60. The method as defined in clause 59, wherein the dimensionality reduction is a principal component analysis, a logistic regression analysis, a nearest neighbor analysis, a support vector machine, a neural network model, a NMF (non-negative matrix factorisation), an ICA (independent component analysis) or a FA (factor analysis) is used to determine the level of methylation variance in the samples.

§ 61. The method as defined in clause 60, wherein the variance analysis performed is a principal component analysis.

§ 62. The method as defined in clause 61, wherein selecting a group of CpG loci and/or genomic regions associated with a feature of the samples comprises selecting one of principal component 1, principal component 2, principal component 3, principal component 4, principal component 5, principal component 6, principal component 7, principal component 8 or a higher principal component.

§ 63. The method of any one of clauses 55 to 62, wherein selecting the CpG loci and/or genomic regions in the group to provide the cfDNA methylome signature comprises selecting the CpG loci and/or genomic regions in the group that have strong association with the feature, for example selecting CpG loci and/or genomic regions that are within the top 10,000 CpG loci and/or genomic regions most correlated with the feature in the group (for example selecting CpG loci and/or genomic regions that are within the top 8000, 5000, 3000, 2000, 1000, 800, 500, 400, 300, 250, 200, 150, 100, 50 or 10 CpG loci and/or genomic regions most correlated with the feature in the group).

§ 64. The method of any one of clauses 55 to 63, wherein selecting CpG loci and/or genomic regions in the group to provide the cfDNA methylome signature comprises selecting at least 5 CpG loci (for example at least 8, at least 10, at least 12, at least 15, at least 20, at least 25, at least 30, at least 40, at least 50, at least 75, at least 100, at least 200, at least 300, at least 400, at least 500, at least 600, at least 700, at least 800, at least 900, at least 1000 or at least 10,000) and/or at least 5 genomic regions (for example at least 8, at least 10, at least 12, at least 15, at least 20, at least 25, at least 30, at least 40, at least 50, at least 75, at least 100, at least 200, at least 300, at least 400, at least 500, at least 600, at least 700, at least 800, at least 900, at least 1000 or at least 10,000) in the group to provide a cfDNA methylome signature.

§ 65. The method of clause 61 or 62, or clauses 63 and 64 when dependent on clauses 61 or 62, wherein selecting CpG loci and/or genomic regions in the group to provide the cfDNA methylome signature comprises selecting a plurality of CpG loci and/or genomic regions of principal component 1, 2, 3, 4, 5, 6, 7 or 8, for example selecting CpG loci and/or genomic regions that are within the top 10,000 CpG loci and/or genomic regions of principal component 1, 2, 3, 4, 5, 6, 7 or 8 most correlated with the feature of principal component 1, 2, 3, 4, 5, 6, 7 or 8; or selecting CpG loci and/or genomic regions that are within the top 5000 CpG loci and/or genomic regions of principal component 1, 2, 3, 4, 5, 6, 7 or 8 most correlated with the feature of principal component 1, 2, 3, 4, 5, 6, 7 or 8; selecting CpG loci and/or genomic regions that are within the top 4000 CpG loci and/or genomic regions of principal component 1, 2, 3, 4, 5, 6, 7 or 8 most correlated with the feature of principal component 1, 2, 3, 4, 5, 6, 7 or 8; selecting CpG loci and/or genomic regions that are within the top 3000 CpG loci and/or genomic regions of principal component 1, 2, 3, 4, 5, 6, 7 or 8 most correlated with the feature of principal component 1, 2, 3, 4, 5, 6, 7 or 8; selecting CpG loci and/or genomic regions that are within the top 2000 CpG loci and/or genomic regions of principal component 1, 2, 3, 4, 5, 6, 7 or 8 most correlated with the feature of principal component 1, 2, 3, 4, 5, 6, 7 or 8; selecting CpG loci and/or genomic regions that are within the top 1000 CpG loci and/or genomic regions of principal component 1, 2, 3, 4, 5, 6, 7 or 8 most correlated with the feature of principal component 1, 2, 3, 4, 5, 6, 7 or 8; or selecting CpG loci and/or genomic regions that are within the top 500, 400, 300, 250, 200, 150, 100, 50 or 10 CpG loci and/or genomic regions of principal component 1, 2, 3, 4, 5, 6, 7 or 8 most correlated with the feature of principal component 1, 2, 3, 4, 5, 6, 7 or 8.

§ 66. The method of any one of clauses 55 to 65, wherein the first sample comprising cfDNA and each of the one or more further samples is a blood sample; or wherein the first sample comprising cfDNA and each of the one or more further samples is a plasma sample.

§ 67. The method of any one of clauses 55 to 66, wherein the cancer is prostate cancer.

§ 68. The method of any one of clauses 55 to 67 comprising repeating steps (i) to (iii) for 2 or more further samples, 3 or more further samples, 4 or more further samples, 5 or more further samples, 6 or more further samples, 7 or more further samples, 8 or more further samples, 9 or more further samples, 10 or more further samples, 12 or more further samples, 15 or more further samples, 20 or more further samples, 25 or more further samples, 30 or more further samples, 40 or more further samples, 50 or more further samples, 60 or more further samples, 70 or more further samples, 80 or more further samples, 90 or more further samples, 100 or more further samples, 200 or more further samples, 300 or more further samples, 400 or more further samples, 500 or more further samples or 1000 or more further samples comprising cfDNA each from subjects known to have the solid cancer.

§ 69. The method of any one of clauses 55 to 68, wherein the first sample and one or more of the further samples are from different subjects (for example wherein the first sample and each of the one or more of the further samples are from different subjects) and/or wherein the first sample and one or more of the further samples are from the same subject, for example the same subject but at different time points, for example before treatment, during a treatment, after a treatment, before progression, after progression, and/or after change of the disease to metastatic cancer.

§ 70. The method of any one of clauses 55 to 69, further comprising comparing the methylation state of each of the selected CpG loci and/or genomic regions in the first sample and in the one or more further samples with the methylation state of the same CpG locus and/or genomic region in one or more of the following:
a sample of non-cancerous tissue of origin of the solid cancer;
a sample of the solid cancer;
a cell-line of the solid cancer;
a sample of cfDNA from a subject known to have the solid cancer (for example an age-matched subject known to have the solid cancer, and for example wherein the level of cancer fraction in the cfDNA sample from the different subject is known and/or wherein the sample is known to comprise cfDNA derived from a prostate cancer subtype);
a sample of white blood cells; and/or
a sample of cfDNA from a healthy subject (for example an age-matched healthy subject); and
optionally determining if the selected CpG locus and/or genomic region are associated with methylation patterns in the tissue of origin of the solid cancer and/or the solid cancer.

§ 71. The method of any one of clauses 55 to 70, further comprising determining a reference value (for example one more reference value, e.g. 3 or more, 4 or more, 5 or more, 10 or more, 15 or more, or 20 or more reference values) for each of the selected CpG loci and/or genomic regions, for example wherein a reference value for each of the selected CpG loci and/or genomic regions is the average methylation ratio of the same CpG locus and/or genomic region in or covered by:
a cfDNA sample from a healthy subject, for example a healthy age-matched subject;
a tissue sample from a healthy subject, for example a prostate tissue sample from a healthy subject;
a cancer biopsy sample from a cancer patient, for example a prostate cancer biopsy sample from a prostate cancer patient;
a cancer cell line sample, for example a prostate cancer cell line sample from a prostate cancer cell line;
a sample of white blood cells from a subject, for example the subject or a healthy subject;
a characterized methylome sequence of a white blood cell;
a characterized methylome sequence of a prostate cancer cell line;
a characterized methylome sequence of a cancerous prostate cell;
a characterized methylome sequence of a non-cancerous prostate cell; or
a sample of cfDNA from a subject known to have the solid cancer (for example an age-matched subject known to have the solid cancer, and for example wherein the level of cancer fraction in the cfDNA sample from the different subject is known and/or wherein the sample is known to comprise cfDNA derived from a prostate cancer subtype).

§ 72. The method of any one of clauses 55 to 71, further comprising establishing an algorithm for detecting, screening, monitoring, staging, classification, selecting treatment for, ascertaining whether treatment is working in, prognostication and/or treatment of the solid cancer using the cfDNA methylome signature, for example wherein
the algorithm is for determining the presence of solid cancer in a further sample comprising DNA using the cfDNA methylome signature; and/or
the algorithm is for determining the level of a solid cancer in a further sample comprising DNA using the cfDNA methylome signature, for example the level of solid cancer tumour fraction; and/or
the algorithm is for determining a subtype of solid cancer in a further sample comprising DNA using the cfDNA methylome signature.

§ 73. The method of clause 72, where the algorithm comprises comparing the methylation status, the methylation ratio, or the average methylation ratio, for some or all of the selected CpG loci and/or genomic regions of the cfDNA methylome signature to the methylation status, the methylation ratio, or the average methylation ratio for some or all of the selected CpG loci and/or genomic regions in a further sample comprising DNA; and/or wherein the algorithm comprises comparing the methylation status, the methylation ratio, or the average methylation ratio, for some or all of the selected CpG loci and/or genomic regions of the cfDNA methylome signature to a reference value for each CpG locus and/or genomic region.

§ 74. A computer implemented method for determining a solid cancer cfDNA methylome signature for use in the detecting, screening, monitoring, staging, classification, selecting treatment for, ascertaining whether treatment is working in, and/or prognostication of the solid cancer, the method comprising performing the method of any one of clauses 55 to 73.

§ 75. A computer product comprising a non-transitory computer readable medium storing a plurality of instructions that when executed control a computer system to perform the method of any one of clauses 55 to 73.

§ 76. A computer-executable software for performing the method of any one of clauses 55 to 73.

§ 77. A computer-implemented software for determining a solid cancer cfDNA methylome signature for use in the detecting, screening, monitoring, staging, classification, selecting treatment for, ascertaining whether treatment is working in, and/or prognostication of the solid cancer, the method comprising:
receiving a data set in a computer comprising a processor and a computer readable medium, wherein the data set comprises the methylome sequence of a plurality of cfDNA molecules in a sample from a subject known to have the solid cancer;
and wherein the computer readable medium comprises instructions that, when executed by the processors, causes the computer to perform a method of any one of clauses 55 to 73.

Further aspects of the invention are defined in the following numbered clauses:

§ 1. A method for detecting, screening, monitoring, staging, classification, selecting treatment for, ascertaining whether treatment is working in, and/or prognostication of prostate cancer in a sample obtained from a subject, wherein the sample comprises circulating free DNA (cfDNA), the method comprising:
characterizing the methylome sequence of a plurality of cfDNA molecules in the sample, wherein the methylome sequence of a cfDNA molecule is the DNA sequence and the methylation profile of the molecule;
determining the average methylation ratio at 10 or more genomic regions, each genomic region being selected from the group consisting of:
a 100 to 200 bp region comprising or having a genomic location defined in Table 8, and
a 2 to 99 bp region within a genomic location defined in Table 8 and comprising at least one CpG locus,
and wherein each of the genomic regions is covered by at least one sequence read of at least one characterized methylome sequence;
calculating a methylation score using the average methylation ratio for each of the genomic regions;
analyzing the methylation score to determine whether the sample comprises cfDNA derived from a prostate cancer subtype.

§ 2. The method of § 1, wherein the method comprises determining the level of cfDNA in the sample that is derived from a prostate cancer subtype.

§ 3. The method of § 1 or § 2, wherein each of the genomic regions is covered by at least one sequence read of at least two characterized methylome sequences, for example at least one sequence read of at least 3, 4, 5, 6, 7, 8, 9, 10, 12, 15, 20, 25, 50, 100, 200, 300, 400, 500, or 1000 characterized methylome sequences.

§ 4. The method of any one of § 1 to § 3, wherein each of the genomic regions is covered by at least 10 sequence reads, for example at least 10, 12, 15, 20, 25, 50, 100, 200, 300, 400, 500, or 1000 sequence reads, and preferably wherein each sequence read or the majority of the sequence reads (for example at least 50%, 60%, 70%, 80% or 90% of the sequence reads) are from different characterized methylome sequences.

§ 5. The method of any one of § 1 to § 4, wherein calculating a methylation score using the average methylation ratio for each genomic region comprises:

determining the median (or the mean) of the average methylation ratios for all genomic regions for which the average methylation ratio has been determined; or
determining the median (or the mean) of the average methylation ratios for a first group of genomic regions to obtain a first methylation score and/or determining the median (or the mean) of the average methylation ratios for second group of genomic regions to obtain a second methylation score; or
comparing the average methylation ratio at each genomic region to a reference methylation ratio for each genomic region to determine a methylation ratio score for each genomic region.

§ 6. The method of § 5, wherein the first group of genomic regions are all of the hypermethylated genomic regions for which the average methylation ratio has been determined, and the second group of genomic regions are all of the hypomethylated genomic regions for which the average methylation ratio has been determined.

§ 7. The method of any one of § 1 to § 6, wherein analyzing the methylation score to determine whether the sample comprises cfDNA derived from a prostate cancer subtype comprises comparing the methylation score to one or more reference methylation scores, wherein a reference methylation score is a methylation score calculated for the same genomic regions (for example, calculated using the average methylation ratio for the same genomic regions) in one or more of the following

a cfDNA sample from a healthy subject, for example a healthy age-matched subject;
a tissue sample from a healthy subject, for example a prostate tissue sample from a healthy subject;
a cancer biopsy sample from a cancer patient, for example a prostate cancer biopsy sample from a prostate cancer patient;
a cancer cell line sample, for example a prostate cancer cell line sample from a prostate cancer cell line;
a sample of white blood cells from a subject, for example the subject or a healthy subject;
a cfDNA sample from a different subject having prostate cancer, wherein preferably the sample is known to comprise cfDNA derived from the prostate cancer subtype (more preferably multiple cfDNA samples (for example at least 2, 3, 4, 5, 10, 20, 40, 50, 100, 200, 300 or 500 samples) each from a different subject having prostate cancer, wherein preferably the each sample is known to comprise cfDNA derived from the prostate cancer subtype, and more preferably wherein each cfDNA sample has a different level of cfDNA derived from the prostate cancer subtype);
a characterized methylome sequence of a white blood cell;
a characterized methylome sequence of a prostate cancer cell line;
a characterized methylome sequence of a cancerous prostate cell; and/or
a characterized methylome sequence of a non-cancerous prostate cell.

§ 8. The method of any one of § 1 to § 7, wherein calculating a methylation score using the average methylation ratio for each genomic region comprises:

determining the median (or the mean) of the average methylation ratios for all genomic regions for which the average methylation ratio has been determined, and
wherein calculating a reference methylation score using the average methylation ratio for each genomic region comprises:
determining the median (or the mean) of the average methylation ratios for all genomic regions for which the average methylation ratio has been determined; or
wherein calculating a methylation score using the average methylation ratio for each genomic region comprises
determining the median (or the mean) of the average methylation ratios for a first group of genomic regions to obtain a first methylation score and/or determining the median (or the mean) of the average methylation ratios for second group of genomic regions to obtain a second methylation score (for example wherein the first group of genomic regions are all of the hypermethylated genomic regions, and the second group of genomic regions are all of the hypomethylated genomic regions), and
calculating a reference methylation score using the average methylation ratio for each genomic region comprises:
determining the median (or the mean) of the average methylation ratios for a first group of genomic regions to obtain a first methylation score and/or determining the median (or the mean) of the average methylation ratios for second group of genomic regions to obtain a second methylation score (for example wherein the first group of genomic regions are all of the hypermethylated genomic regions, and the second group of genomic regions are all of the hypomethylated genomic regions).

§ 9. The method of any one of § 1 to § 8, wherein calculating a methylation score using the average methylation ratio for each genomic region comprises comparing the average methylation ratio at each genomic region to a reference methylation ratio for each genomic region to determine a methylation ratio score for each genomic region,

and wherein the reference methylation ratio is the average methylation ratio for the same genomic region in or covered by:
a cfDNA sample from a healthy subject, for example a healthy age-matched subject;
a tissue sample from a healthy subject, for example a prostate tissue sample from a healthy subject;
a cancer biopsy sample from a cancer patient, for example a prostate cancer biopsy sample from a prostate cancer patient;
a cancer cell line sample, for example a prostate cancer cell line sample from a prostate cancer cell line;
a sample of white blood cells from a subject, for example the subject or a healthy subject;
a cfDNA sample from a different subject having prostate cancer, wherein preferably the sample is known to comprise cfDNA derived from the prostate cancer subtype (preferably multiple cfDNA samples (for example at least 2, 3, 4, 5, 10, 20, 40, 50, 100, 200, 300 or 500 samples) each from a different subject having prostate cancer, wherein preferably each sample is known to comprise cfDNA derived from the prostate cancer subtype, and more preferably wherein each cfDNA sample has a different level of cfDNA derived from the prostate cancer subtype);
a characterized methylome sequence of a white blood cell;
a characterized methylome sequence of a prostate cancer cell line;
a characterized methylome sequence of a cancerous prostate cell; and/or
a characterized methylome sequence of a non-cancerous prostate cell.

§ 10. The method of § 9, wherein analyzing the methylation score to determine whether the sample comprises cfDNA derived from a prostate cancer subtype comprises determining the number of methylation ratio scores that are indicative of the prostate cancer subtype.

§ 11. The method of any one of § 1 to § 10, wherein the methylome sequence of a cfDNA molecule is determined by using methylation aware sequencing (for example with bisulfite sequencing), methylation-sensitive restriction enzyme digestion, methylation-specific PCR, methylation-dependent DNA precipitation, methylated DNA binding proteins/peptides, or single molecule sequences without sodium bisulfite treatment.

§ 12. The method of any one of § 1 to § 11, wherein the methylome sequence of a cfDNA molecule is determined by performing methylation aware sequencing, for example wherein the methylation aware sequencing comprises treating the DNA molecule with sodium bisulfite and performing sequencing of the treated DNA molecule.

§ 13. The method of any one of § 1 to § 12, wherein the genomic regions are selected from:

a 100 to 150 bp region comprising or having a genomic location defined in Table 8, and
a 10 to 99 bp region within a genomic location defined in Table 8 and comprising at least one CpG locus; or
a 100 to 150 bp region comprising or having a genomic location defined in Table 9, and
a 10 to 99 bp region within a genomic location defined in Table 9 and comprising at least one CpG locus.

§ 14. The method of any one of § 1 to § 13, wherein the genomic regions are selected from:

a 100 to 120 bp region comprising or having a genomic location defined in Table 8, and
a 50 to 99 bp region within a genomic location defined in Table 8 and comprising at least one CpG locus; or
a 100 to 120 bp region comprising or having a genomic location defined in Table 9, and
a 50 to 99 bp region within a genomic location defined in Table 9 and comprising at least one CpG locus.

§ 15. The method of any one of § 1 to § 14, wherein the genomic regions have a 100 bp genomic location defined in Table 8, or wherein the genomic regions have a 100 bp genomic location defined in Table 9.

§ 16. The method of any one of § 1 to § 15, comprising characterising the average methylation ratio at 25 or more, 50 or more, 100 or more, 150 or more, 200 or more, 300 or more, 400 or more, or 500 or more genomic regions (for example comprising determining the average methylation ratio at 25, 50, 100, 150, 200, 300, 400 or 500 genomic regions), wherein the genomic regions have a genomic location defined in Table 8.

§ 17. The method of any one of § 1 to § 15, comprising characterising the average methylation ratio at 25 or more, 50 or more, 100 or more, 150 or more, 200 or more, 300 or more, 400 or more, or 500 or more genomic regions (for example comprising determining the average methylation ratio at 25, 50, 100, 150, 200, 300, 400 or 500 genomic regions), wherein the genomic regions have a genomic location defined in Table 8.

§ 16. The method of any one of § 1 to § 16, comprising characterising the average methylation ratio at 25 or more, 50 or more, 100 or more, 125 or more, or 150 genomic regions (for example comprising determining the average methylation ratio at 25, 50, 100, 125, or 150 genomic regions), wherein the genomic regions have a genomic location defined in Table 9.

§ 18. The method of any one of § 1 to § 17, wherein at least 25% of the genomic regions are prostate tissue specific genomic regions; or wherein at least 25% of the regions are prostate cancer specific genomic regions.

§ 19. The method of any one of § 1 to § 18, wherein at least 40% of the genomic regions are prostate cancer specific genomic regions, for example at least 50, 60, 70, 80, 90 or 95% (for example 95, 96, 97, 98, 99 and 100%) of the genomic regions are prostate cancer specific genomic regions; or wherein at least 40% of the genomic regions are prostate tissue specific genomic regions, for example at least 50, 60, 70, 80, 90 or 95% (for example 95, 96, 97, 98, 99 and 100%) of the genomic regions are prostate tissue specific genomic regions.

§ 20. The method of any one of § 1 to § 19, wherein a plurality of cfDNA molecules is at least 10,000, at least 50,000, at least 100,000, at least 500,000, at least 1,000,000, at least 5,000,000, at least 10,000,000, or at least 100,000,000 cfDNA molecules.

§ 21. The method of any one of § 1 to § 20, wherein the prostate cancer is acinar adenocarcinoma prostate cancer, ductal adenocarcinoma prostate cancer, transitional cell cancer of the prostate, squamous cell cancer of the prostate, or small cell prostate cancer.

§ 22. The method of any one of § 1 to § 21 wherein the prostate cancer is castration resistant prostate cancer and/or is metastatic prostate cancer.

§ 23. The method of § 1 to § 22, wherein the prostate cancer subtype is one that has an aggressive clinical course and/or androgen receptor (AR) copy number gain, for example an androgen-insensitive prostate cancer subtype.

§ 24. The method of any one of § 1 to § 23, wherein the sample comprising cfDNA is a blood or plasma sample.

§ 25. The method of any one of § 1 to § 24, further comprising measuring the level of prostate-specific antigen (PSA) in a sample of blood from the subject, and determining if the subject has an abnormal level of PSA in the blood (for example a level of PSA in the blood of at least 4.0 ng/mL), or, if the subject has had a previous PSA test, an increased level of PSA compared to the previous test.

§ 26. The method of any one of § 1 to § 25, further comprising repeating the method on a second sample obtained from the subject after the subject has undergone a treatment for prostate cancer, wherein the second sample comprises cfDNA, and comparing the detectable level of cfDNA derived from a prostate cancer subtype in each sample.

§ 27. The method of any one of § 1 to § 26, for screening and/or prognostication of prostate cancer, wherein prostate cancer with a poor prognosis is predicted when cfDNA derived from the prostate cancer subtype is identified in the sample, for example a detectable level of cfDNA derived from the prostate cancer subtype, for example a percentage level of cfDNA derived from the prostate cancer subtype of at least 0.01%.

§ 28. An in-vitro diagnostic kit for use in the detecting, screening, monitoring, staging, classification, selecting treatment for, ascertaining whether treatment is working in, and/or prognostication of prostate cancer, comprising one or more reagents for detecting the presence or absence of at least 10 DNA molecules having a DNA sequence corresponding to all or part of a genomic location comprising at least one CpG locus defined in Table 8 or Table 9.

§ 29. The kit as described in § 28, wherein the kit comprises one or more reagents for detecting the presence or absence of at least 15, 20, 30, 40, 50, 75, 100, 150, 200, 250, 300, 400, 500, 600, 700, 800, or 900 DNA molecules (for example 15, 20, 30, 40, 50, 75, 100, 150, 200, 250, 300, 400, 500, 600, 700, 800, 900 or 1000 DNA molecules) having a DNA sequence corresponding to all or part of a genomic location comprising at least one CpG locus defined in Table 8 or Table 9.

§ 30. The kit as described in § 28 or § 29, wherein the kit comprises oligonucleotides for specifically hybridizing to at least a section of the at least 10 DNA molecules (for example, at least 15, 20, 30, 40, 50, 75, 100, 150, 200, 250, 300, 400, 500, 600, 700, 800, or 900 DNA molecules) having a DNA sequence corresponding to all or part of a genomic location defined in Table 8 or Table 9.

§ 31. The kit of any one of § 28 to § 30, wherein at least one of the oligonucleotides for specifically hybridizing to at least a section of a DNA molecule is an amplification primer, for example each of the oligonucleotides for specifically hybridizing to at least a section of a DNA molecule is an amplification primer.

§ 32. A computer product comprising a non-transitory computer readable medium storing a plurality of instructions that when executed control a computer system to perform the method of any one of § 1 to § 27.

§ 33. A computer-executable software for performing the method of any one of § 1 to § 27.

§ 34. The kit of any one of § 28 to § 31, wherein the kit comprises instructions for use which define how to determine whether a sample comprises cfDNA derived from a prostate cancer subtype and/or the level of cfDNA in the sample that is derived from a prostate cancer subtype, and/or comprises a computer product as defined in § 32, and/or a computer-executable software as defined in § 33.

§ 35. A computer-implemented method for detecting, screening, monitoring, staging, classification, selecting treatment for, ascertaining whether treatment is working in, and/or prognostication of prostate cancer in a sample obtained from a subject, wherein the sample comprises circulating free DNA (cfDNA), the method comprising:

receiving a data set in a computer comprising a processor and a computer readable medium, wherein the data set comprises the methylome sequence of a plurality of cfDNA molecules in the sample;
and wherein the computer readable medium comprises instructions that, when executed by the processors, causes the computer to perform a method of any one of § 1 to § 27 (for example causes the computer to perform a method comprising the following steps:
characterizing the methylome sequence of a plurality of cfDNA molecules in the sample, wherein the methylome sequence of a cfDNA molecule is the DNA sequence and the methylation profile of the molecule;
determining the average methylation ratio at 10 or more genomic regions, each genomic region being selected from the group consisting of:
a 100 to 200 bp region comprising or having a genomic location defined in Table 8, and
a 2 to 99 bp region within a genomic location defined in Table 8 and comprising at least one CpG locus,
and wherein each of the genomic regions is covered by at least one sequence read of at least one characterized methylome sequence;
calculating a methylation score using the average methylation ratio for each of the genomic regions;
analyzing the methylation score to determine whether the sample comprises cfDNA derived from a prostate cancer subtype.

§ 36. A computer-implemented method for classifying a prostate cancer patient into one or more of a plurality of treatment categories, the method comprising determining the level of prostate cancer DNA in a sample obtained from a subject, wherein the sample comprises circulating free DNA (cfDNA), the method comprising:

receiving a data set in a computer comprising a processor and a computer readable medium, wherein the data set comprises the methylome sequence of a plurality of cfDNA molecules in a sample obtained from a subject, wherein the sample comprises cfDNA;
and wherein the computer readable medium comprises instructions that, when executed by the processors, causes the computer to perform a method of any one of § 1 to § 27, for example causes the computer to perform a method comprising the following steps:
characterizing the methylome sequence of a plurality of cfDNA molecules in the sample, wherein the methylome sequence of a cfDNA molecule is the DNA sequence and the methylation profile of the molecule;
determining the average methylation ratio at 10 or more genomic regions, each of the genomic regions being selected from the group consisting of:
a 100 to 200 bp region comprising or having a genomic location defined in Table 8, and
a 2 to 99 bp region within a genomic location defined in Table 8 and comprising at least one CpG locus,
and wherein each of the genomic region is covered by at least one sequence read of at least one characterized methylome sequence;
calculating a methylation score using the average methylation ratio for each of the genomic regions;
analyzing the methylation score to determine whether the sample comprises cfDNA derived from a prostate cancer subtype.

§ 37. The method of any one of § 1 to § 27, § 35 or § 36 further comprising treating the subject for prostate cancer using a therapeutic agent for the treatment of prostate cancer; or ceasing or altering treatment with a therapeutic agent for the treatment of prostate cancer; or initiating a non-therapeutic agent treatment for prostate cancer (for example initiation of treatment by surgery or radiation).

§ 38. A method for treating prostate cancer in a subject comprising the method of § 1 to § 27, § 35 or § 36 and further comprising treating the subject using a therapeutic agent for the treatment of prostate cancer, surgery, and/or radiotherapy; or a method for treating prostate cancer in a subject, comprising administering to the subject an effective amount of a therapeutic agent for the treatment of prostate cancer after the subject has been determined to have prostate cancer subtype based on a method as defined in § 1 to § 27, § 35, or § 36.

§ 39. The method of § 38, wherein the method of § 1 to § 27, § 35, or § 36 is performed before and/or after treating the subject.

§ 40. A method of any one of § 37 to § 39, comprising performing the method of § 1 to § 27, § 35, or § 36 before treating the subject, and subsequently repeating the method of use § 1 to § 27, § 35, or § 36 after the treatment, for example at least 1 week, at least 2 weeks, at least 3 weeks, at least 4 weeks, at least 1 month, at least 2 months, at least 3 months, at least 6 months, at least 9 months, at least 12 months, at least 24 months or at least 36 months after treating the subject.

§ 41. The method of § 40, wherein the method comprises continuing to treat the subject with the therapeutic agent for the treatment of prostate cancer if the cfDNA derived from a prostate cancer subtype is detected in the sample and/or the sample comprises a level of cfDNA derived from the prostate cancer subtype that is substantially the same in the initial and subsequent method or lower in the subsequent method than in the initial method.

§ 42. The method of § 40 or § 41, wherein the method comprises

ceasing or altering treatment with the therapeutic agent for the treatment of prostate cancer; and/or
initiating treatment with a second therapeutic agent for the treatment of prostate cancer; and/or
initiating a non-therapeutic agent treatment (e.g., surgery or radiation),
if the sample comprises cfDNA derived from a prostate cancer subtype and/or the sample comprises a level of cfDNA derived from a prostate cancer subtype that is substantially the same in the initial and subsequent method or higher in the subsequent method than in the initial method.

§ 43. The method of § 42, wherein the second therapeutic agent is a chemotherapeutic agent or a PARP inhibitor.

§ 44. A method of treating a subject in need of treatment with a therapeutic agent for the treatment of prostate cancer, comprising

i) performing the method of any one of § 1 to § 27, § 35, or § 36 to determine if the sample comprises cfDNA derived from a prostate cancer subtype and/or determine the level of cfDNA in the sample derived from a prostate cancer subtype;
ii) administering a therapeutic agent for the treatment of prostate cancer if the sample comprises cfDNA derived from a prostate cancer subtype and/or if the sample comprises a level of cfDNA derived from a prostate cancer subtype (for example 0.01% or more cfDNA derived from a prostate cancer subtype).

§ 45. A therapeutic agent for the treatment of prostate cancer for use in the treatment of prostate cancer, wherein

i) the method of any one of § 1 to § 27, § 35 or § 36 is performed to determine if a sample comprises cfDNA derived from a prostate cancer subtype in a subject and/or determine the level of cfDNA in the sample derived from a prostate cancer subtype in a subject;
ii) the therapeutic agent is administered if the sample comprises cfDNA derived from a prostate cancer subtype in the subject and/or if the sample comprises a level of cfDNA derived from a prostate cancer subtype (for example 0.01% or more cfDNA derived from a prostate cancer subtype).

§ 46. A method as described in § 39 to § 44, or a therapeutic agent for the treatment of prostate cancer for use as described in § 45, wherein a second therapeutic agent for the treatment of prostate cancer is administered if a sample from the subject has cfDNA derived from a prostate cancer subtype and/or has a level of cfDNA derived from a prostate cancer subtype (for example a detectable level of prostate cancer DNA, for example 0.01% or more cfDNA derived from a prostate cancer subtype).

§ 47. The method as described in § 44, or a therapeutic agent for the treatment of prostate cancer for use as described in § 45, wherein

(iii) at least 1 week, at least 2 weeks, at least 3 weeks, at least 4 weeks, at least 1 month, at least 2 months, at least 3 months, at least 6 months, at least 9 months, at least 12 months, at least 24 months, or at least 36 months, after the administration of the therapeutic agent, a further sample comprising cfDNA is obtained from the subject, and the method of any one of § 1 to § 27, § 35, or § 36 is performed to determine if the further sample comprises cfDNA derived from a prostate cancer subtype in a subject and/or determine the level of cfDNA that is derived from a prostate cancer subtype.

§ 48. A method of determining one or more suitable therapeutic agents for the treatment of prostate cancer for a subject having prostate cancer comprising

performing the method of any one of § 1 to § 27, § 35 or § 36;
determining the one or more suitable therapeutic agents for the treatment of prostate cancer by reference to whether the sample comprises cfDNA derived from a prostate cancer subtype and/or the level of cfDNA in the sample that is derived from a prostate cancer subtype, whereby one therapeutic agent is suitable for a subject with a sample having no cfDNA derived from a prostate cancer subtype (for example an undetectable level of cfDNA derived from a prostate cancer subtype) or a level of cfDNA derived from a prostate cancer subtype of less than 0.01%, and two or more therapeutic agents are suitable for a subject with a level of cfDNA derived from a prostate cancer subtype (for example a percentage level of cfDNA derived from a prostate cancer subtype of at least 0.01%);
or whereby a therapeutic agent selected from a first list of therapeutic agents is suitable for a subject with a sample having no cfDNA derived from a prostate cancer subtype (for example an undetectable level of cfDNA derived from a prostate cancer subtype) or a level of cfDNA derived from a prostate cancer subtype of less than 0.01%, and a therapeutic agent from a second list of therapeutic agents, or two or more therapeutic agents from the first list, is suitable for a subject with a level of cfDNA derived from a prostate cancer subtype (for example a percentage level of cfDNA derived from a prostate cancer subtype of at least 0.01%).

§ 49. A method of determining a suitable treatment regimen for a subject having prostate cancer comprising

performing the method of any one of claims § 1 to § 27, § 35 or § 36;
determining the treatment regimen by reference whether the sample comprises cfDNA derived from a prostate cancer subtype and/or the level of cfDNA in the sample that is derived from a prostate cancer subtype, whereby a standard treatment is suitable for a subject with a sample having no cfDNA derived from a prostate cancer subtype (for example an undetectable level of cfDNA derived from a prostate cancer subtype) or a percentage level of cfDNA derived from a prostate cancer subtype in the cfDNA sample of less than 0.01%, and a non-standard treatment is suitable for a subject when a level cfDNA derived from a prostate cancer subtype (for example a detectable level of cfDNA derived from a prostate cancer subtype in the cfDNA sample) or a percentage level of cfDNA derived from a prostate cancer subtype in the cfDNA sample is determined of at least 0.01%.

§ 50. The method as claimed in § 49, wherein the standard treatment is a treatment with a therapeutic agent for the treatment of prostate cancer, and a non-standard treatment is a treatment with two or more therapeutic agents for the treatment of prostate cancer;

or wherein the standard treatment is a treatment with a hormonal agent for the treatment of prostate cancer, and a non-standard treatment is a treatment with a hormonal agent for the treatment of prostate cancer, and a chemotherapeutic agent for the treatment of prostate cancer and/or a immunotherapy treatment of prostate cancer and/or a targeted treatment of prostate cancer and/or a biologic agent treatment of prostate cancer.

§ 51. A computerized method and/or computer-assisted method for determining one or more suitable therapeutic agents for the treatment of prostate cancer for a subject having prostate cancer, the method comprising performing the steps of § 48; or for selecting a treatment regimen for a subject having prostate cancer, the method comprising the steps of § 49 or § 50.

§ 52. A method or therapeutic agent as described in any one of § 37 to § 51, wherein the therapeutic agent for the treatment of prostate cancer is selected from the group consisting of a hormonal agent, a targeted agent, a biologic agent, an immunotherapy agent, a chemotherapy agent;

for example: a hormonal agent selected from LHRH agonists (for example leuprolide, goserelin, triptorelin, or histrelin), LHRH antagonists (for example degarelix), androgen blockers (for example abiraterone or ketoconazole), anti-androgens (for example flutamide, bicalutamide, nilutamide, enzalutamide, apalutamide or darolutamide), estrogens, and steroids (for example prednisone or dexamethasone);
a targeted agent selected from poly(ADP-ribose) polymerase (PARP) inhibitor (for example olaparib, rucaparib, niraparib or talazoparib), a epidermal growth factor receptor (EGFR) inhibitor (for example gefitinib, erlotinib, afatinib, brigatinib, icotinib, cetuximab, or osimertinib, adavosertib, lapatinib), and a tyrosine kinase inhibitor (for example imatinib, gefitinib, erlotinib, sunitinib);
a biologic agent selected from monoclonal antibodies (for example pertuzumab, trastuzumab and Solitomab), hormones (for example a hormonal agent selected from LHRH agonists (for example leuprolide, goserelin, triptorelin, or histrelin), LHRH antagonists (for example degarelix), androgen blockers (for example abiraterone or ketoconazole), anti-androgens (for example flutamide, bicalutamide, nilutamide, enzalutamide, apalutamide or darolutamide), and estrogens), interferons (for example interferons-α, -β, -γ), and interleukin-based products (for example interleukin-2);
an immunotherapy agent selected from a cancer vaccine (for example sipuleucel-T), T-cell therapy, monoclonal antibody therapy, immune checkpoint therapy (for example a PD-1 inhibitor (e.g pembrolizumab, nivolumab, cemiplimab spartalizumab), a PD-L1 inhibitor (e.g. atezolizumab, avelumab or durvalumab), or a CTLA-4 (e.g. ipilimumab)), and non-specific immunotherapies (for example interferons and inerleukins);
a chemotherapy agent selected from docetaxel, cabazitaxel, and c-Met inhibitors (for example cabozantinib).

1. A method for detecting, screening, monitoring, staging, classification, selecting treatment for, ascertaining whether treatment is working in, and/or prognostication of prostate cancer in a sample obtained from a subject, wherein the sample comprises circulating free DNA (cfDNA), the method comprising:
characterizing the methylome sequence of a plurality of cfDNA molecules in the sample, wherein the methylome sequence of a cfDNA molecule is the DNA sequence and the methylation profile of the molecule;
determining the average methylation ratio at 10 or more genomic regions, each genomic region being selected from the group consisting of:
a 100 to 200 bp region comprising or having a genomic location defined in Tables 1 to 4, and
a 2 to 99 bp region within a genomic location defined in Tables 1 to 4 and comprising at least one CpG locus,
and wherein each of the genomic regions is covered by at least one sequence read of at least one characterized methylome sequence;
calculating a methylation score using the average methylation ratio for each of the genomic regions;
analyzing the methylation score to determine the level of prostate cancer fraction in the cfDNA sample.

2. The method of claim 1, wherein each of the genomic regions is covered by at least one sequence read of at least two characterized methylome sequences, for example at least one sequence read of at least 3, 4, 5, 6, 7, 8, 9, 10, 12, 15, 20, 25, 50, 100, 200, 300, 400, 500, or 1000 characterized methylome sequences.

3. The method of claim 1 or 2, wherein each of the genomic regions is covered by at least 10 sequence reads, for example at least 10, 12, 15, 20, 25, 50, 100, 200, 300, 400, 500, or 1000 sequence reads, and preferably wherein each sequence read or the majority of the sequence reads (for example at least 50%, 60%, 70%, 80% or 90% of the sequence reads) are from different characterized methylome sequences.

4. The method of any one of claims 1 to 3, wherein calculating a methylation score using the average methylation ratio for each genomic region comprises:
determining the median (or the mean) of the average methylation ratios for all genomic regions for which the average methylation ratio has been determined; or
determining the median (or the mean) of the average methylation ratios for a first group of genomic regions to obtain a first methylation score and/or determining the median (or the mean) of the average methylation ratios for second group of genomic regions to obtain a second methylation score; or
comparing the average methylation ratio at each genomic region to a reference methylation ratio for each genomic region to determine a methylation ratio score for each genomic region.

5. The method of any one of claims 1 to 4, wherein analyzing the methylation score to determine the level of prostate cancer fraction in the cfDNA sample comprises comparing the methylation score to one or more reference methylation scores, wherein a reference methylation score is a methylation score calculated for the same genomic regions (for example, calculated using the average methylation ratio for the same genomic regions) in one or more of the following
a cfDNA sample from a healthy subject, for example a healthy age-matched subject;
a tissue sample from a healthy subject, for example a prostate tissue sample from a healthy subject;
a cancer biopsy sample from a cancer patient, for example a prostate cancer biopsy sample from a prostate cancer patient;
a cancer cell line sample, for example a prostate cancer cell line sample from a prostate cancer cell line;
a sample of white blood cells from a subject, for example the subject or a healthy subject;
a cfDNA sample from a different subject having prostate cancer, preferably wherein the level of prostate cancer fraction in the cfDNA sample from the different subject is known (more preferably multiple cfDNA samples (for example at least 2, 3, 4, 5, 10, 20, 40, 50, 100, 200, 300 or 500 samples) each from a different subject having prostate cancer, wherein preferably the level of prostate cancer fraction in each cfDNA sample from the different subjects is known, and more preferably wherein each cfDNA sample has a different level of prostate cancer fraction);
a characterized methylome sequence of a white blood cell;
a characterized methylome sequence of a prostate cancer cell line;
a characterized methylome sequence of a cancerous prostate cell; and/or
a characterized methylome sequence of a non-cancerous prostate cell.

6. The method of any one of claims 1 to 5, comprising determining the average methylation ratio at 25 or more, 50 or more, 100 or more, 150 or more, 200 or more, 300 or more, 400 or more, 500 or more, 600 or more, 700 or more, 800 or more, or 900 or more genomic regions (for example comprising determining the average methylation ratio at 25, 50, 100, 150, 200, 300, 400, 500, 600, 700, 800, 900 or 1000 genomic regions).

7. The method of any one of claims 1 to 6, wherein the genomic regions have a 100 bp genomic location defined in any one of Tables 1 to 4, Table 5, Table 6 or Table 7.

8. The method of any one of claims 1 to 7, wherein at least 25% of the genomic regions are prostate tissue specific genomic regions.

9. The method of any one of claims 1 to 8, wherein the prostate cancer is acinar adenocarcinoma prostate cancer, ductal adenocarcinoma prostate cancer, transitional cell cancer of the prostate, squamous cell cancer of the prostate, or small cell prostate cancer (for example wherein the prostate cancer is acinar adenocarcinoma prostate cancer or ductal adenocarcinoma prostate cancer).

10. The method of any one of claims 1 to 9 wherein the prostate cancer is castration resistant prostate cancer and/or is metastatic prostate cancer.

11. The method of any one of claims 1 to 10, wherein the sample comprising cfDNA is a blood or plasma sample.

12. The method of any one of claims 1 to 11, further comprising repeating the method on a second sample obtained from the subject after the subject has undergone a treatment for prostate cancer, wherein the second sample comprises cfDNA, and comparing the level of prostate cancer fraction in the two samples.

13. The method of any one of claims 1 to 12, further comprising treating the subject for prostate cancer using a therapeutic agent for the treatment of prostate cancer;
or ceasing or altering treatment with a therapeutic agent for the treatment of prostate cancer; or
initiating a non-therapeutic agent treatment for prostate cancer (for example initiation of treatment by surgery or radiation).

14. An in-vitro diagnostic kit for use in the detecting, screening, monitoring, staging, classification, selecting treatment for, ascertaining whether treatment is working in, and/or prognostication of prostate cancer, comprising one or more reagents for detecting the presence or absence of at least 10 DNA molecules having a DNA sequence corresponding to all or part of a genomic location comprising at least one CpG locus defined in Tables 1 to 4, or comprising at least one CpG locus defined in Table 5, or comprising at least one CpG locus defined in Table 6, or comprising at least one CpG locus defined in Table 7.

15. A computer product comprising a non-transitory computer readable medium storing a plurality of instructions that when executed control a computer system to perform the method of any one of claims 1 to 12; or a computer-executable software for performing the method of any one of claims 1 to 12 or a computer-implemented method for detecting, screening, monitoring, staging, classification, selecting treatment for, ascertaining whether treatment is working in, and/or prognostication of prostate cancer in a sample obtained from a subject, wherein the sample comprises circulating free DNA (cfDNA), the method comprising:
receiving a data set in a computer comprising a processor and a computer readable medium,
wherein the data set comprises the methylome sequence of a plurality of cfDNA molecules in the sample;
and wherein the computer readable medium comprises instructions that, when executed by the processor, causes the computer to perform a method of any one of claims 1 to 12

16. A therapeutic agent for the treatment of prostate cancer for use in the treatment of prostate cancer, whereby
i) the method of any one of claims 1 to 12 is performed to determine the level of prostate cancer prostate cancer DNA in a subject;
ii) the therapeutic agent is administered if the subject has a level of prostate cancer.

17. A method of determining one or more suitable therapeutic agents for the treatment of prostate cancer for a subject having prostate cancer comprising
performing the method of any one of claims 1 to 12;
determining the one or more suitable therapeutic agents for the treatment of prostate cancer by reference to the level of prostate cancer, whereby one therapeutic agent is suitable for a subject with no level of prostate cancer fraction (for example an undetectable level of prostate cancer fraction) or a level of prostate cancer fraction of less than 0.01%, and two or more therapeutic agents are suitable for a subject with a level of prostate cancer DNA (for example a percentage level of prostate cancer fraction of at least 0.01%);
or whereby a therapeutic agent selected from a first list of therapeutic agents is suitable for a subject with no level of prostate cancer DNA (for example an undetectable level of prostate cancer DNA) or a level of prostate cancer DNA of less than 0.01%, and a therapeutic agent from a second list of therapeutic agents, or two or more therapeutic agents from the first list, is suitable for a subject with a level of prostate cancer DNA (for example a percentage level of prostate cancer fraction of at least 0.01%).

18. A method or therapeutic agent as claimed in any one of claim 16 or 17, wherein the therapeutic agent for the treatment of prostate cancer is selected from the group consisting of a hormonal agent, a targeted agent, a biologic agent, an immunotherapy agent, a chemotherapy agent.

19. A method for determining a solid cancer circulating free DNA (cfDNA) methylome signature for use in detecting, screening, monitoring, staging, classification, selecting treatment for, ascertaining whether treatment is working in, prognostication and/or treatment of the solid cancer, the method comprising:
(i) characterizing the methylome sequence of a plurality of cfDNA molecules in a first sample comprising cfDNA from a subject known to have the solid cancer, wherein the methylome sequence of a cfDNA molecule is the DNA sequence and the methylation profile of the molecule;
(ii) determining the respective number of characterised cfDNA molecules corresponding to a CpG locus or a genomic region of 2 to 10,000 bp (preferably 2 to 200 bp) in the first sample by aligning the methylome sequences;
(iii) determining the methylation ratio of each CpG locus and/or average methylation ratio of each genomic region of 2 to 10,000 bp (preferably 2 to 200 bp) in the first sample;
repeating steps (i) to (iii) for one or more further samples comprising cfDNA each from subjects known to have the solid cancer;
performing a variance analysis of all or a selection of the methylation ratios of the CpG loci and/or all or a selection of average methylation ratios of the genomic regions of the samples;
selecting a group of CpG loci and/or genomic regions associated with a feature of the samples;
selecting CpG loci and/or genomic regions in the group to provide the cfDNA methylome signature.

20. The method of claim 19, wherein the solid cancer is prostate cancer.

21. The method of claim 19 or 20, wherein the variance analysis performed is a dimensionality reduction.

22. The method as claimed in claim 21 wherein the variance analysis performed is a principal component analysis.

23. The method as claimed in claim 22, wherein selecting a group of CpG loci and/or genomic regions associated with a feature of the samples comprises selecting one of principal component 1, principal component 2, principal component 3, principal component 4, principal component 5, principal component 6, principal component 7, principal component 8 or a higher principal component.

24. The method of any one of claims 18 to 23, wherein selecting the CpG loci and/or genomic regions in the group to provide the cfDNA methylome signature comprises selecting the CpG loci and/or genomic regions in the group that have strong association with the feature, for example selecting CpG loci and/or genomic regions that are within the top 10,000 CpG loci and/or genomic regions most correlated with the feature in the group (for example selecting CpG loci and/or genomic regions that are within the top 8000, 5000, 3000, 2000, 1000, 800, 500, 400, 300, 250, 200, 150, 100, 50 or 10 CpG loci and/or genomic regions most correlated with the feature in the group).

25. The method of any one of claims 18 to 24, wherein selecting CpG loci and/or genomic regions in the group to provide the cfDNA methylome signature comprises selecting at least 5 CpG loci (for example at least 8, at least 10, at least 12, at least 15, at least 20, at least 25, at least 30, at least 40, at least 50, at least 75, at least 100, at least 200, at least 300, at least 400, at least 500, at least 600, at least 700, at least 800, at least 900, at least 1000 or at least 10,000) and/or at least 5 genomic regions (for example at least 8, at least 10, at least 12, at least 15, at least 20, at least 25, at least 30, at least 40, at least 50, at least 75, at least 100, at least 200, at least 300, at least 400, at least 500, at least 600, at least 700, at least 800, at least 900, at least 1000 or at least 10,000) in the group to provide a cfDNA methylome signature.

26. The method of claim 22 or 23, or claim 24 or 25 when dependent on claim 22 or 23, wherein selecting CpG loci and/or genomic regions in the group to provide the cfDNA methylome signature comprises selecting a plurality of CpG loci and/or genomic regions of principal component 1, 2, 3, 4, 5, 6, 7 or 8, for example selecting CpG loci and/or genomic regions that are within the top 10,000 CpG loci and/or genomic regions of principal component 1, 2, 3, 4, 5, 6, 7 or 8 most correlated with the feature of principal component 1, 2, 3, 4, 5, 6, 7 or 8; or selecting CpG loci and/or genomic regions that are within the top 5000 CpG loci and/or genomic regions of principal component 1, 2, 3, 4, 5, 6, 7 or 8 most correlated with the feature of principal component 1, 2, 3, 4, 5, 6, 7 or 8;
selecting CpG loci and/or genomic regions that are within the top 4000 CpG loci and/or genomic regions of principal component 1, 2, 3, 4, 5, 6, 7 or 8 most correlated with the feature of principal component 1, 2, 3, 4, 5, 6, 7 or 8; selecting CpG loci and/or genomic regions that are within the top 3000 CpG loci and/or genomic regions of principal component 1, 2, 3, 4, 5, 6, 7 or 8 most correlated with the feature of principal component 1, 2, 3, 4, 5, 6, 7 or 8; selecting CpG loci and/or genomic regions that are within the top 2000 CpG loci and/or genomic regions of principal component 1, 2, 3, 4, 5, 6, 7 or 8 most correlated with the feature of principal component 1, 2, 3, 4, 5, 6, 7 or 8;
selecting CpG loci and/or genomic regions that are within the top 1000 CpG loci and/or genomic regions of principal component 1, 2, 3, 4, 5, 6, 7 or 8 most correlated with the feature of principal component 1, 2, 3, 4, 5, 6, 7 or 8; or selecting CpG loci and/or genomic regions that are within the top 500, 400, 300, 250, 200, 150, 100, 50 or 10 CpG loci and/or genomic regions of principal component 1, 2, 3, 4, 5, 6, 7 or 8 most correlated with the feature of principal component 1, 2, 3, 4, 5, 6, 7 or 8.

27. A method for detecting, screening, monitoring, staging, classification, selecting treatment for, ascertaining whether treatment is working in, and/or prognostication of prostate cancer in a sample obtained from a subject, wherein the sample comprises circulating free DNA (cfDNA), the method comprising:
characterizing the methylome sequence of a plurality of cfDNA molecules in the sample, wherein the methylome sequence of a cfDNA molecule is the DNA sequence and the methylation profile of the molecule;
determining the average methylation ratio at 10 or more genomic regions, each genomic region being selected from the group consisting of:
a 100 to 200 bp region comprising or having a genomic location defined in Table 8, and
a 2 to 99 bp region within a genomic location defined in Table 8 and comprising at least one CpG locus,
and wherein each of the genomic regions is covered by at least one sequence read of at least one characterized methylome sequence;
calculating a methylation score using the average methylation ratio for each of the genomic regions;
analyzing the methylation score to determine whether the sample comprises cfDNA derived from a prostate cancer subtype.

28. The method of claim 27, wherein the prostate cancer is an androgen-insensitive subtype of the prostate cancer.
